(19) United States
(12) Patent Application Publication
(10) Pub. No.: US 2024/0050422 A1
(43) Pub. Date: Feb. 15, 2024

(54) DRUG DELIVERY VIA MONOACYLGLYCEROL AND FREE FATTY ACID-BASED COMPOSITIONS

(71) Applicant: GLYCOSBIO, INC., Houston, TX (US)

(72) Inventors: Daniel J. MONTICELLO, The Woodlands, TX (US); Nathaniel Scott SCHOCKER, Pearland, TX (US); David Michael STEVENS, Houston, TX (US)

(21) Appl. No.: 18/379,449

(22) Filed: Oct. 12, 2023

Related U.S. Application Data
(63) Continuation of application No. PCT/US2022/024709, filed on Apr. 13, 2022.
(60) Provisional application No. 63/174,487, filed on Apr. 13, 2021.
(60) Provisional application No. 63/174,489, filed on Apr. 13, 2021.
(60) Provisional application No. 63/197,896, filed on Jun. 7, 2021.
(60) Provisional application No. 63/197,905, filed on Jun. 7, 2021.
(60) Provisional application No. 63/292,862, filed on Dec. 22, 2021.

(51) Int. Cl. A61K 31/445; A61K 31/167; A61K 31/573; A61K 9/06; A61K 47/44

(52) U.S. Cl. CPC A61K 31/445; A61K 31/167; A61K 31/573; A61K 9/06; A61K 47/44

(57) ABSTRACT

Compositions and methods for the preparation of nanoemulsion from oils are disclosure. These nanoemulsions can be produced without the addition of exogenous surfactants or emulsifiers and can be useful as carriers for active ingredients.

### CROSS-REFERENCE TO RELATED APPLICATIONS

This application is a continuation of International Application No. PCT/US2022/024709, filed Apr. 13, 2022, which claims priority to U.S. Provisional Patent Application No. 63/174,487, filed Apr. 13, 2021, U.S. Provisional Patent Application No. 63/174,489, filed Apr. 13, 2021, U.S. Provisional Patent Application No. 63/197,896, filed Jun. 7, 2021, U.S. Provisional Patent Application No. 63/197,905, filed Jun. 7, 2021, and U.S. Provisional Patent Application No. 63/292,862, filed Dec. 22, 2021, each of which is incorporated herein by reference in its entirety.

### BACKGROUND

There are several methods that can create emulsions outside the body. However, these nanoemulsion formulations generally require addition of other agents (e.g., surfactants such as Tween-80 (polysorbate 80)) to stabilize the nanoemulsions. There is an unmet need for nanoemulsion formulations that do not require additional stabilizers, emulsifiers or surfactants and which are stable.

Emulsions (including nanoemulsions and microemulsions) can be useful as as carriers of active ingredients, such as pharmaceutically active substances.

The enzymatic hydrolysis of commercial source oils, mainly composed of triglycerides, provides access to MAGs and FFAs and can be achieved by selective use of lipases to render the desired overall ratios of materials, while molecular distillation can be used to refine the individual components. Key ingredients present in source oils such as terpenes, terpenoids, sterols, polyphenols, phytosterols, and the like, may contribute properties to the hydrolyzed triglycerides that can enhance the biological activity relative to normally studied individual MAG and FFA components, such as those from distillation.

There is a need for oil formulations, including from specific oil combinations, that can provide a drug delivery system by carrying active ingredients such as pharmaceutically active substances.

### SUMMARY

The present disclosure is directed to oil compositions that can include an enzyme-modified oil (EMO) and/or a free fatty acid (FFA) oil and which can be used in combination with an active ingredient. The oil compositions of the present disclosure can be used in any formulation, but can also be used to formulate nanoemulsions and microemulsions that contain free fatty acids and, optionally, monoacylglycerols (MAGs) and/or diacylglycerols (DAGs), that do not require additional agents to stabilize the nanoemulsions. Methods for making the same are also provided in addition to methods of using, including administering to a subject the formulations containing an active ingredient and/or the oil composition. In certain aspects, the compositions of the present disclosure can provide enhanced permeability for active ingredients.

In some embodiments, a composition is provided that includes an oil dispersed in a liquid, where the oil is in the form of droplets or particles and includes at least 2% free fatty acids (FFAs) by weight of the oil, where the FFAs include two or more different fatty acids, where the oil includes 80% or less triacylglycerols (TAGs) by weight out of the total weight of the oil, and where the droplets or particles have a Z average size of about 300 nm or less as measured by dynamic light scattering.

In some embodiments, a composition is provided that includes an oil dispersed in a liquid, where the oil is in the form of droplets or particles and includes at least 2% FFAs by weight of the oil and 80% or less TAGs by weight of the oil, where the oil includes non-oil ingredients that are naturally present in the oil, and where the droplets or particles have a Z average particle size of about 300 nm or less as measured by dynamic light scattering.

In some embodiments, a composition is provided that includes an oil dispersed in a liquid, where the oil is in the form of droplets or particles and includes at least 2% FFAs by weight of the oil, where the FFAs include two or more different fatty acids, where the oil also includes monoacylglycerols (MAGs) in an amount of at least 5% by weight out of the total weight of the oil, where the MAGs and FFAs are derived from a common, first oil source, and where the droplets or particles have a Z average particle size of about 300 nm or less as measured by dynamic light scattering.

In some embodiments, a composition is provided that includes an oil dispersed in a liquid, where the oil is in the form of droplets or particles and includes at least 2% FFAs by weight of the oil, where the FFAs include two or more different fatty acids, where the oil also includes MAGs in an amount of at least 5% by weight of the total weight of the oil, where the MAGs and FFAs are derived from a first oil source and a second oil source, respectively, and where the droplets or particles have a Z average size of about 300 nm or less as measured by dynamic light scattering.

In some embodiments, a composition is provided that includes an oil dispersed in a liquid, where the oil is in the form of droplets or particles and includes at least 2% FFAs by weight of the oil, where the FFAs include two or more different fatty acids, where the oil also includes monoacylglycerols (MAGs) and diacylglycerols (DAGs) in an amount of at least 5% by weight out of the total weight of the oil, where the MAGs and FFAs are derived from a common, first oil source, and where the droplets or particles have a Z average particle size of about 300 nm or less as measured by dynamic light scattering.

In some embodiments, a composition is provided that includes an oil dispersed in a liquid, where the oil is in the form of droplets or particles and includes at least 2% FFAs by weight of the oil, where the FFAs include two or more different fatty acids, where the oil also includes MAGs and DAGs in an amount of at least 5% by weight of the total weight of the oil, where the MAGs and FFAs are derived from a first oil source and a second oil source, respectively, and where the droplets or particles have a Z average size of about 300 nm or less as measured by dynamic light scattering.

In some embodiments, a method for preparing a nanoemulsion is provided that includes providing an oil of the present disclosure, providing a polar, liquid component, combining the oil and polar, liquid component to form a nanoemulsion pre-mix, and forming a nanoemulsion from the nanoemulsion pre-mix, where the nanoemulsion comprises an oil phase and a polar, liquid phase, where the oil phase is dispersed as droplets or particles within the polar, liquid phase, and where the droplets or particles have a Z average size of about 300 nm or less as measured by dynamic light scattering.

In some embodiments, a composition is provided that includes an oil dispersed in a liquid, where the oil is in the form of droplets or particles and includes at least 2% free fatty acids (FFAs) by weight of the oil, where the FFAs include two or more different fatty acids, where the oil includes 80% or less triacylglycerols (TAGs) by weight out of the total weight of the oil, and where the droplets or particles have a Z average size of greater than about 300 nm as measured by dynamic light scattering.

In some embodiments, a composition is provided that includes an oil dispersed in a liquid, where the oil is in the form of droplets or particles and includes at least 2% FFAs by weight of the oil and 80% or less TAGs by weight of the oil, where the oil includes non-oil ingredients that are naturally present in the oil, and where the droplets or particles have a Z average particle size of greater than about 300 nm as measured by dynamic light scattering.

In some embodiments, a composition is provided that includes an oil dispersed in a liquid, where the oil is in the form of droplets or particles and includes at least 2% FFAs by weight of the oil, where the FFAs include two or more different fatty acids, where the oil also includes monoacylglycerols (MAGs) in an amount of at least 5% by weight out of the total weight of the oil, where the MAGs and FFAs are derived from a common, first oil source, and where the droplets or particles have a Z average particle size of greater than about 300 nm as measured by dynamic light scattering.

In some embodiments, a composition is provided that includes an oil dispersed in a liquid, where the oil is in the form of droplets or particles and includes at least 2% FFAs by weight of the oil, where the FFAs include two or more different fatty acids, where the oil also includes MAGs in an amount of at least 5% by weight of the total weight of the oil, where the MAGs and FFAs are derived from a first oil source and a second oil source, respectively, and where the droplets or particles have a Z average size of greater than about 300 nm as measured by dynamic light scattering.

In some embodiments, a composition is provided that includes an oil dispersed in a liquid, where the oil is in the form of droplets or particles and includes at least 2% FFAs by weight of the oil, where the FFAs include two or more different fatty acids, where the oil also includes monoacylglycerols (MAGs) and diacylglycerols (DAGs) in an amount of at least 5% by weight out of the total weight of the oil, where the MAGs and FFAs are derived from a common, first oil source, and where the droplets or particles have a Z average particle size of greater than about 300 nm as measured by dynamic light scattering.

In some embodiments, a composition is provided that includes an oil dispersed in a liquid, where the oil is in the form of droplets or particles and includes at least 2% FFAs by weight of the oil, where the FFAs include two or more different fatty acids, where the oil also includes MAGs and DAGs in an amount of at least 5% by weight of the total weight of the oil, where the MAGs and FFAs are derived from a first oil source and a second oil source, respectively, and where the droplets or particles have a Z average size of greater than about 300 nm as measured by dynamic light scattering.

In some embodiments, a method for preparing a nanoemulsion or microemulsion is provided that includes providing an oil of the present disclosure, providing a polar, liquid component, combining the oil and polar, liquid component to form a nanoemulsion pre-mix, and forming a nanoemulsion from the nanoemulsion pre-mix, where the nanoemulsion comprises an oil phase and a polar, liquid phase, where the oil phase is dispersed as droplets or particles within the polar, liquid phase, and where the droplets or particles have a Z average size of greater than about 300 nm as measured by dynamic light scattering.

In some embodiments, an oil composition is provided that can include monoacylglycerols (MAGs) in an amount from about 10% to about 80% by weight of the total weight of the oil, free fatty acids (FFAs) in an amount from about 5% to about 75% by weight of the total weight of the oil as applicable based on the percent by weight of MAGs, where the combination of MAGs and FFAs is from about 60% to about 95% by weight of the total weight of the oil, where the FFAs comprise two or more different fatty acids and where the oil is substantially free of triacylglycerols (TAGs).

In some embodiments, a pharmaceutical composition comprising a composition of the present disclosure and an active ingredient is provided.

In some embodiments, a method for delivering an active ingredient to a subject can include administering a pharmaceutical composition of the present disclosure to the subject.

### BRIEF DESCRIPTION OF THE DRAWINGS

FIG. 1 depicts the particle size distribution as measured by dynamic light scattering for Example 2.

FIG. 2A depicts the Z average particle size as measured by dynamic light scattering for flaxseed EMO nanoemulsion samples over time, stored at room temperature (data are represented as average±SD from at least three measurements).

FIG. 2B depicts the Z average particle size as measured by dynamic light scattering for sesame EMO nanoemulsion samples over time, stored at room temperature (data are represented as average±SD from at least three measurements; formulations that precipitated were assigned a Z average size of 1000 nm).

FIG. 2C depicts the Z average particle size as measured by dynamic light scattering for coconut EMO/medium chain triglyceride (MCT) FFA oil nanoemulsion samples over time, stored at room temperature (data are represented as average±SD from at least three measurements; formulations that precipitated were assigned a Z average size of 1000 nm).

FIG. 2D depicts the Z average particle size as measured by dynamic light scattering for MCT EMO/coconut FFA oil nanoemulsion samples over time, stored at room temperature (data are represented as average±SD from at least three measurements; formulations that precipitated were assigned a Z average size of 1000 nm).

FIG. 2E depicts the Z average particle size as measured by dynamic light scattering for fish EMO/flaxseed FFA oil nanoemulsion samples over time, stored at room temperature (data are represented as average±SD from at least three measurements; formulations that precipitated were assigned a Z average size of 1000 nm).

FIG. 2F depicts the Z average particle size as measured by dynamic light scattering for flaxseed EMO/fish FFA oil nanoemulsion samples over time, stored at room temperature (data are represented as average±SD from at least three measurements; formulations that precipitated were assigned a Z average size of 1000 nm).

FIG. 2G depicts the Z average particle size as measured by dynamic light scattering for fish EMO/coconut FFA oil nanoemulsion samples over time, stored at room temperature (data are represented as average±SD from at least three measurements; formulations that precipitated were assigned a Z average size of 1000 nm).

FIG. 2H depicts the Z average particle size as measured by dynamic light scattering for flaxseed EMO/coconut FFA oil nanoemulsion samples over time, stored at room temperature (data are represented as average±SD from at least three measurements; formulations that precipitated were assigned a Z average size of 1000 nm).

FIG. 21 depicts the Z average particle size as measured by dynamic light scattering for coconut EMO/flaxseed FFA oil nanoemulsion samples over time, stored at room temperature (data are represented as average±SD from at least three measurements; formulations that precipitated were assigned a Z average size of 1000 nm).

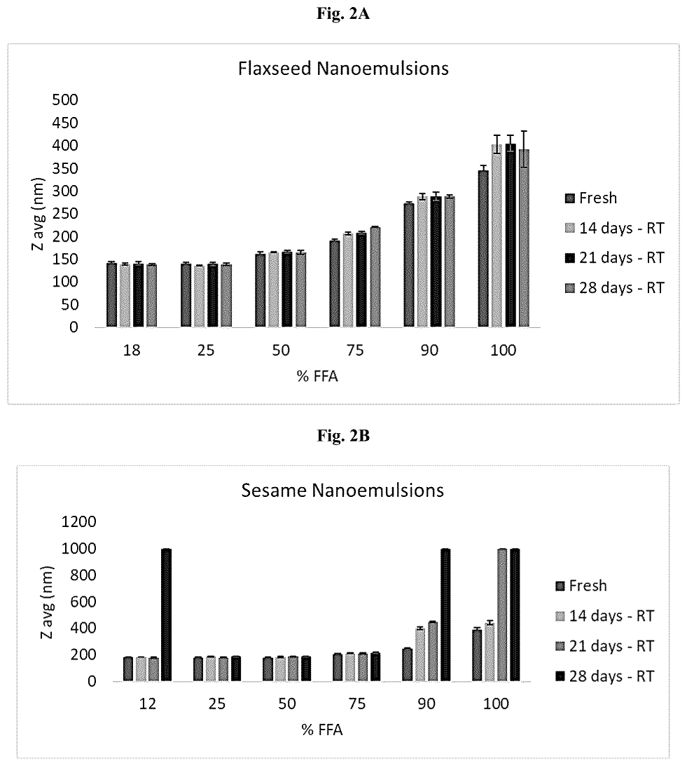

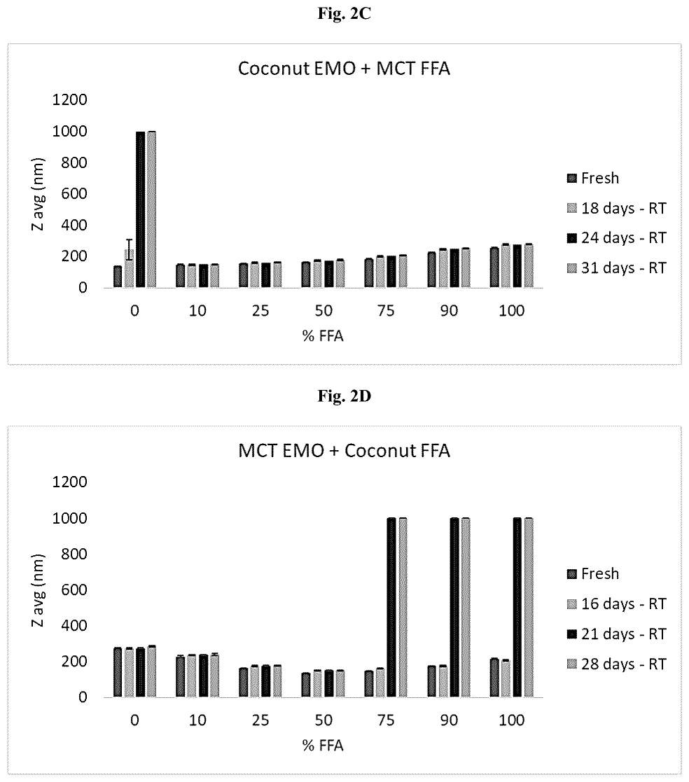

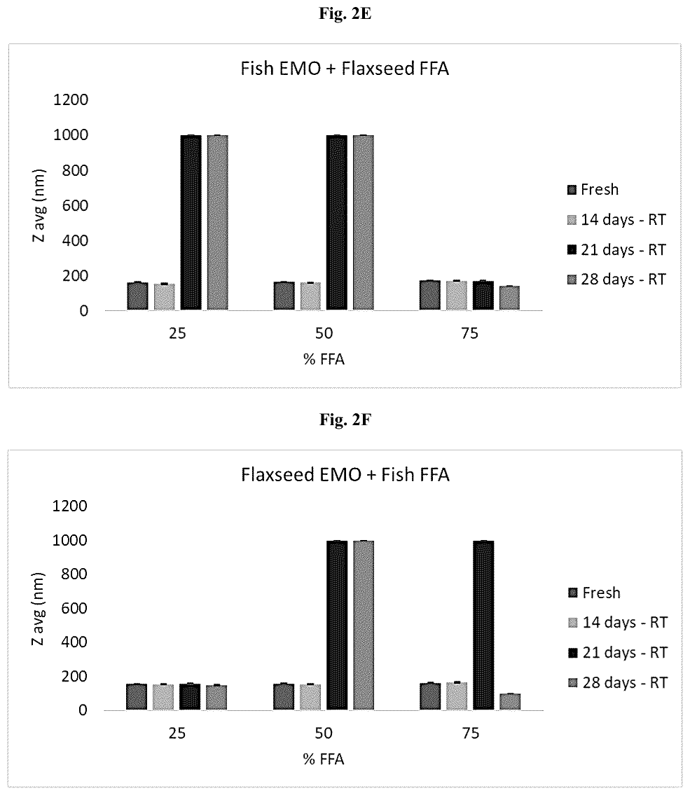

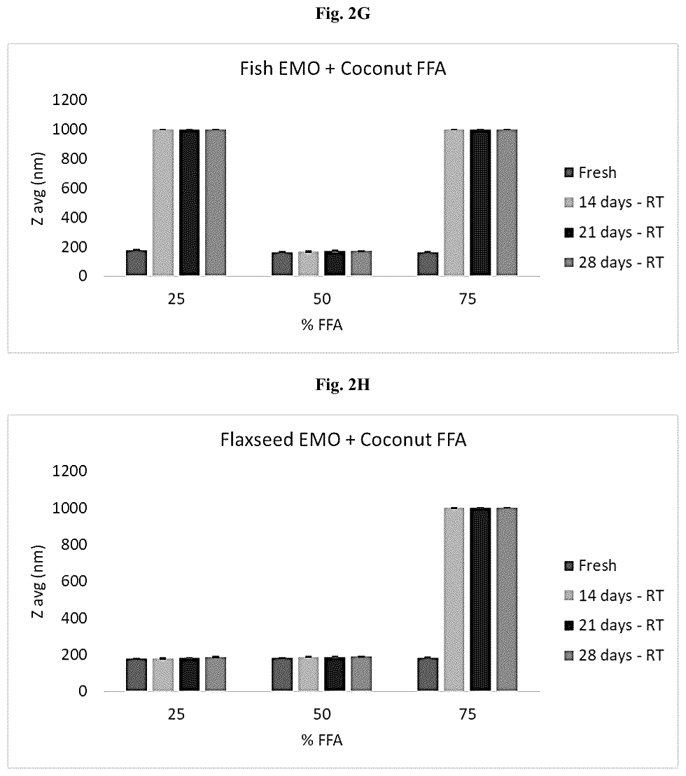

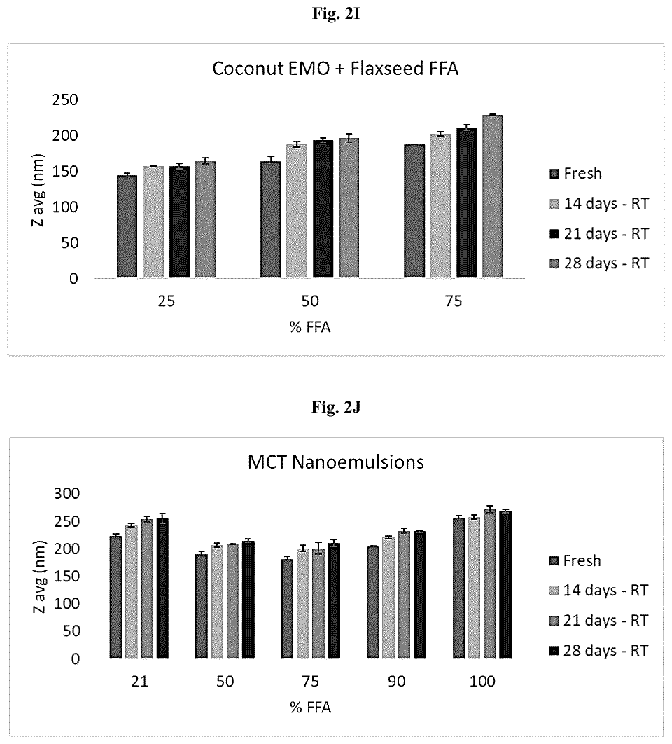

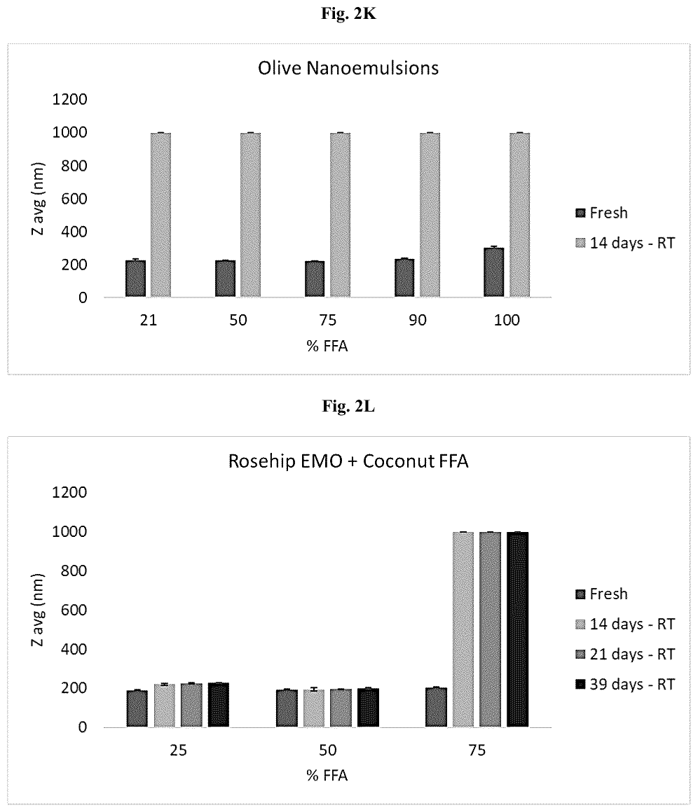

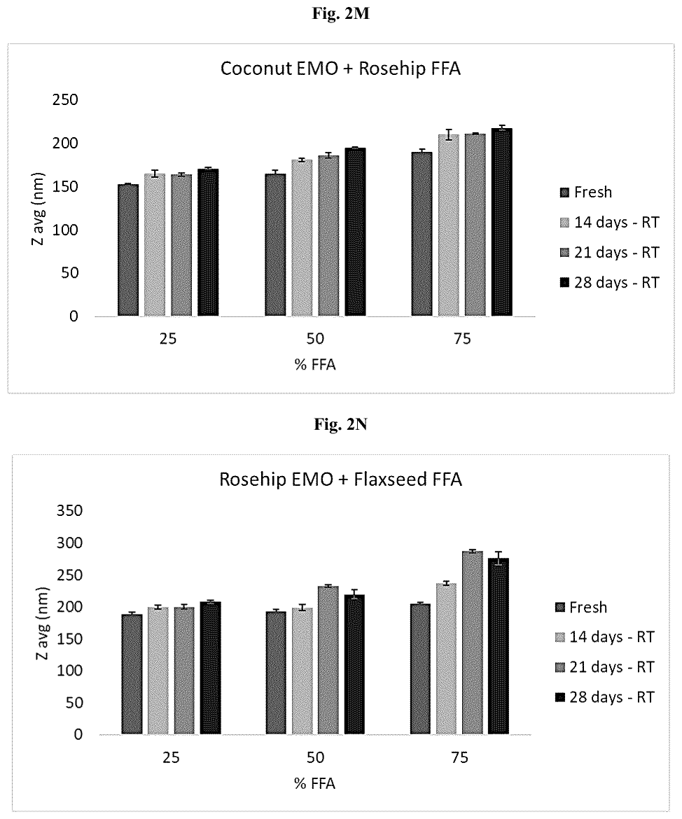

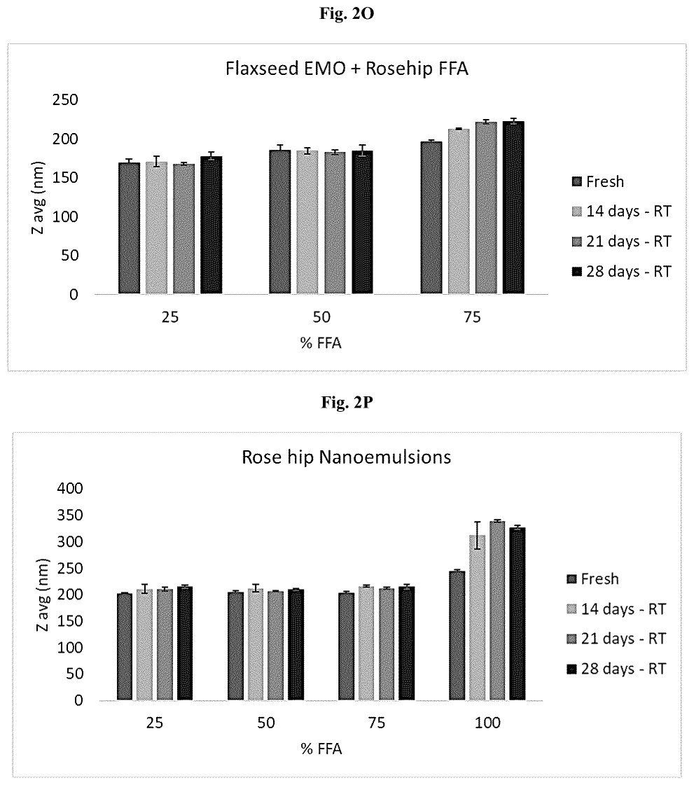

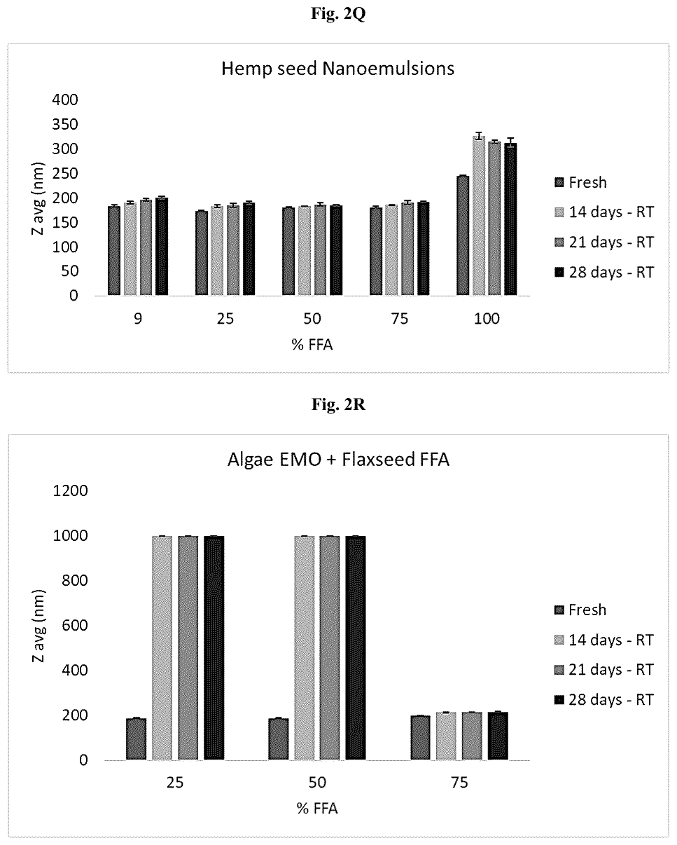

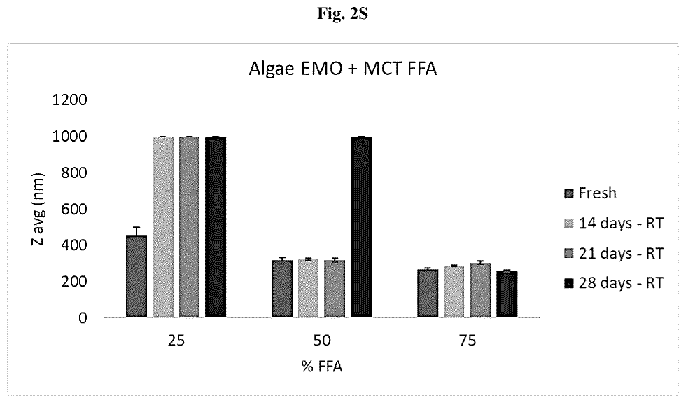

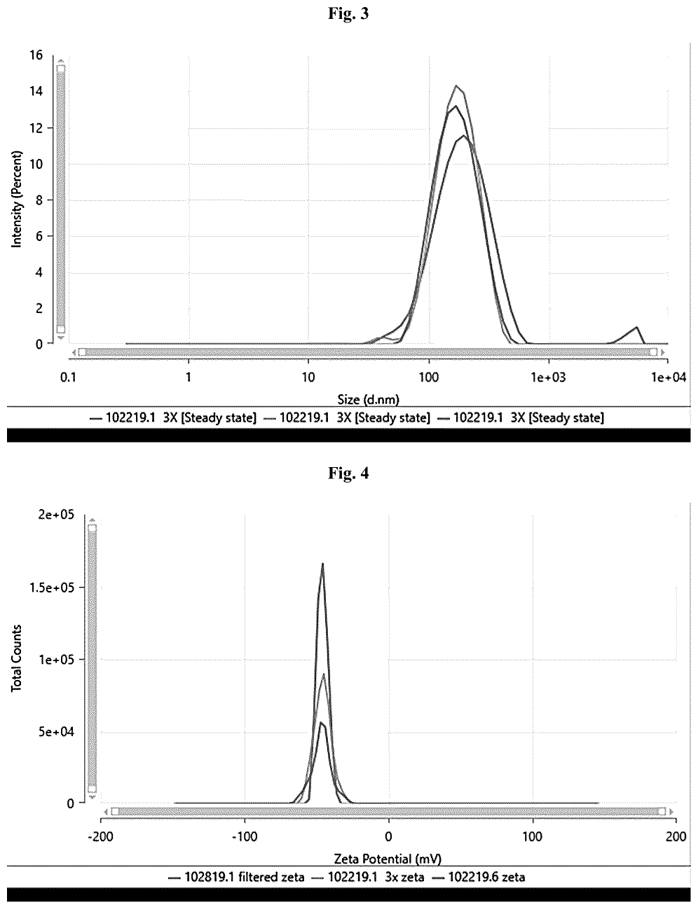

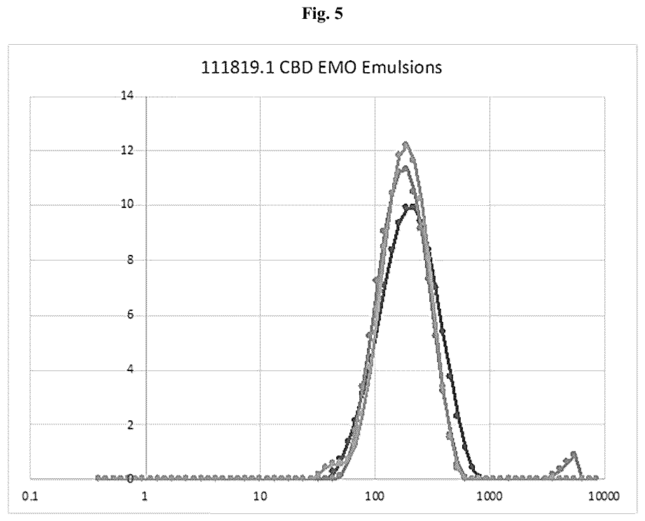

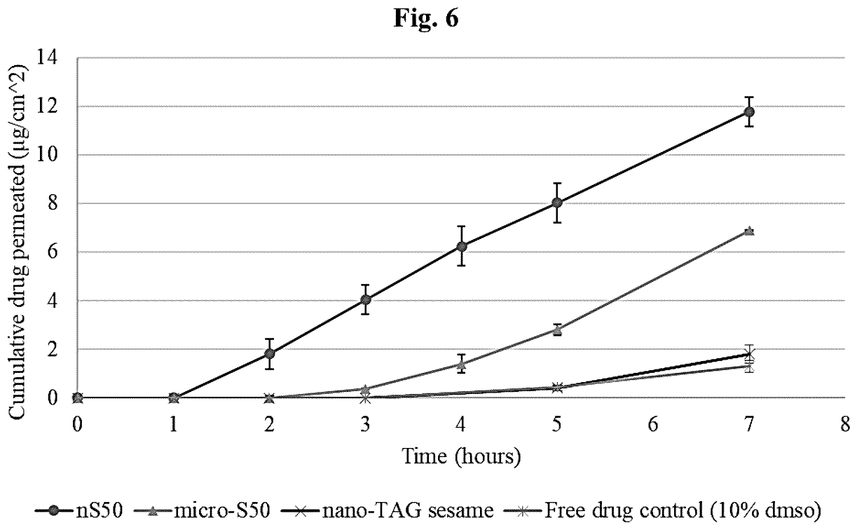

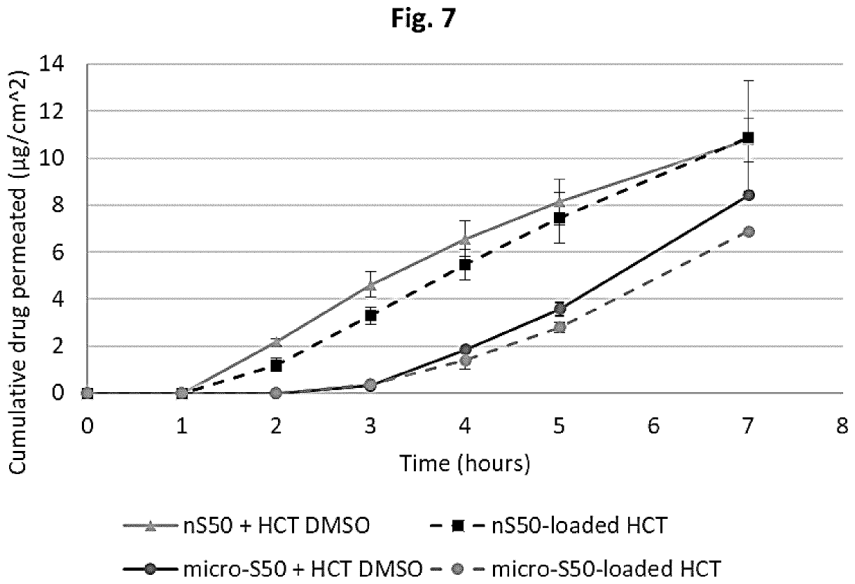

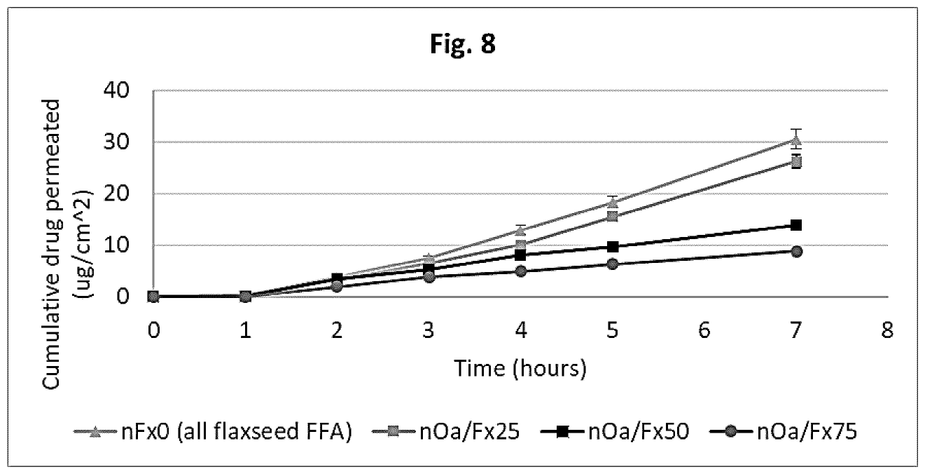

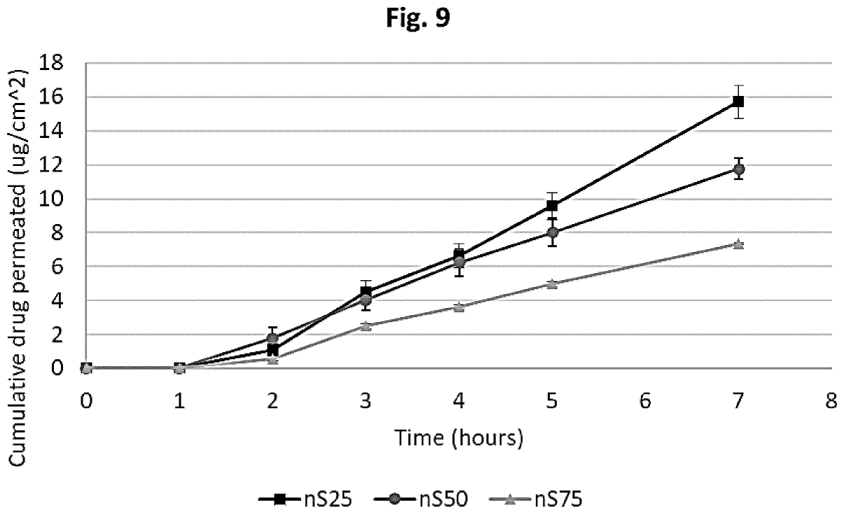

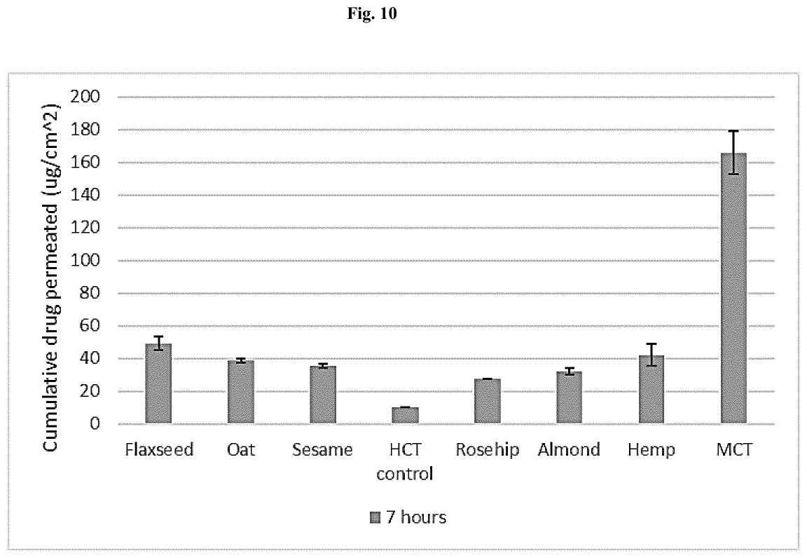

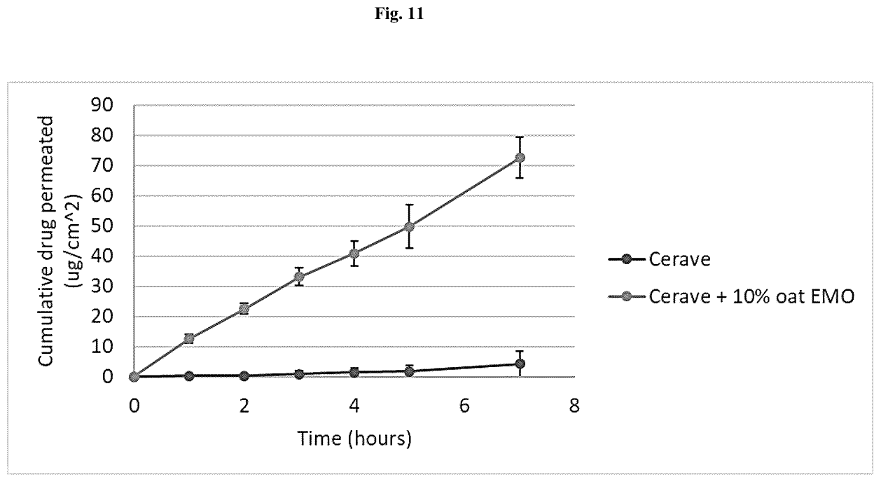

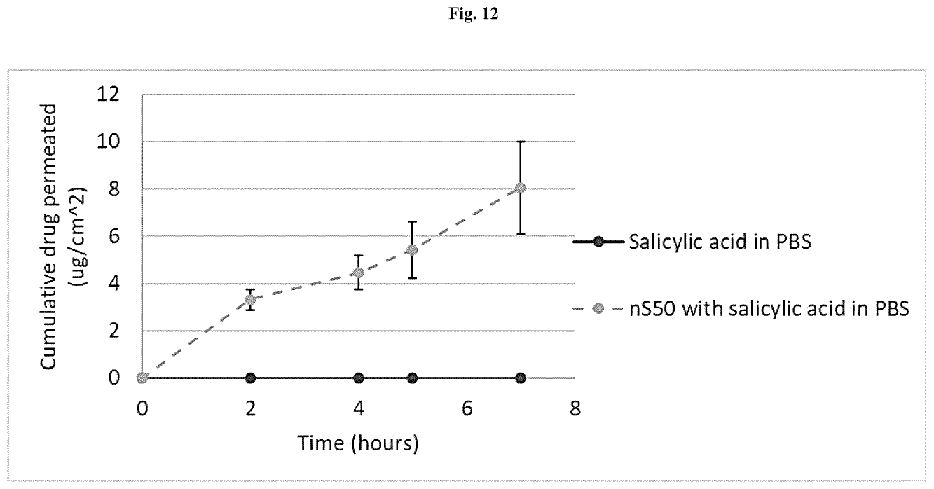

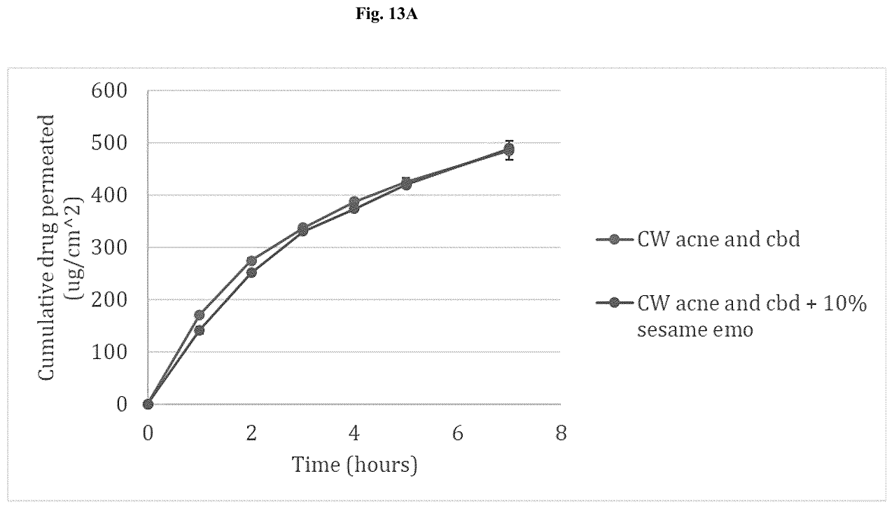

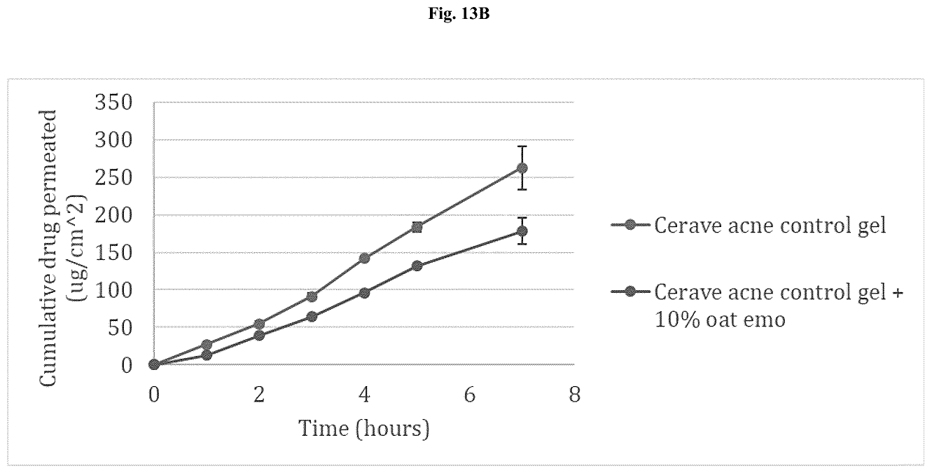

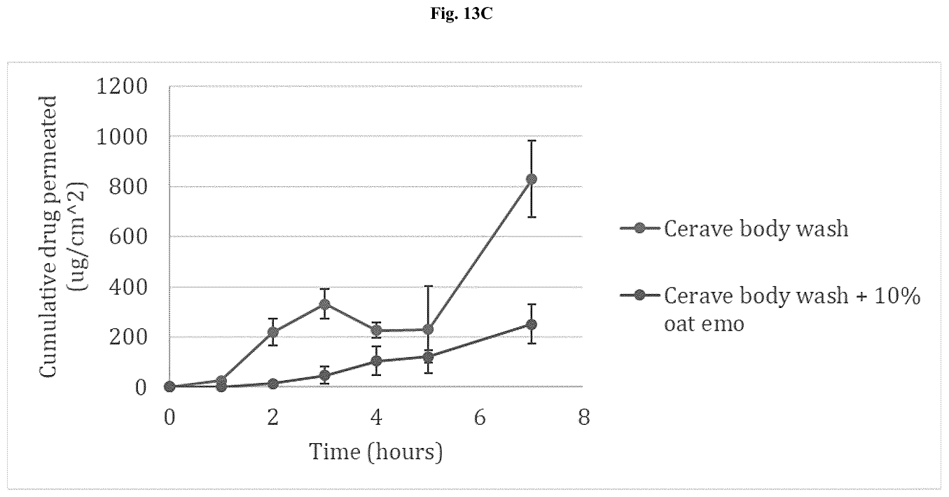

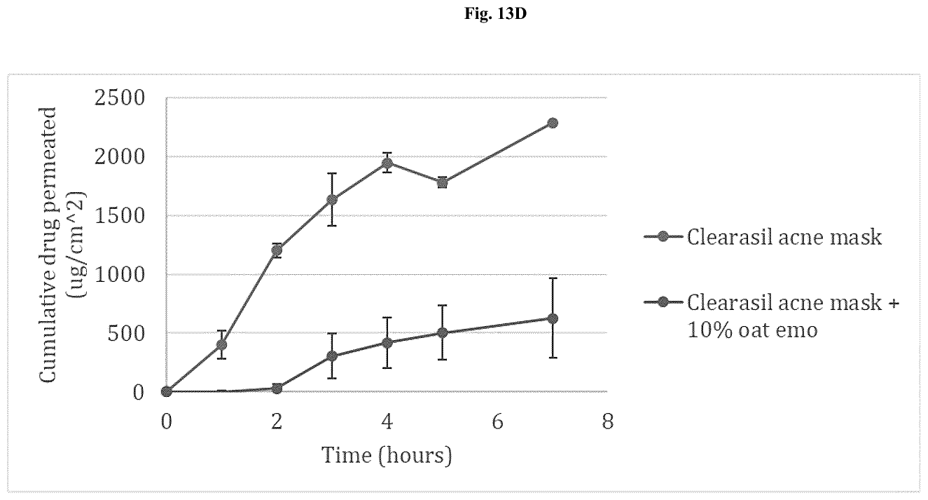

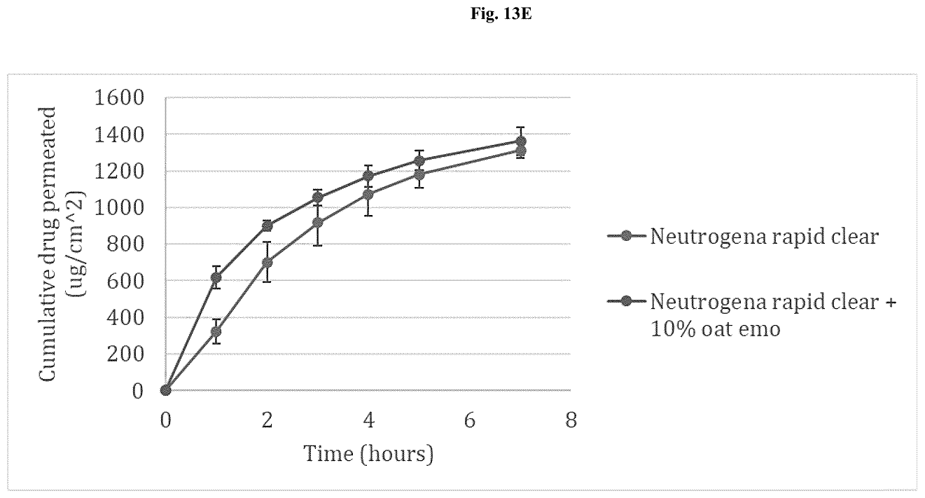

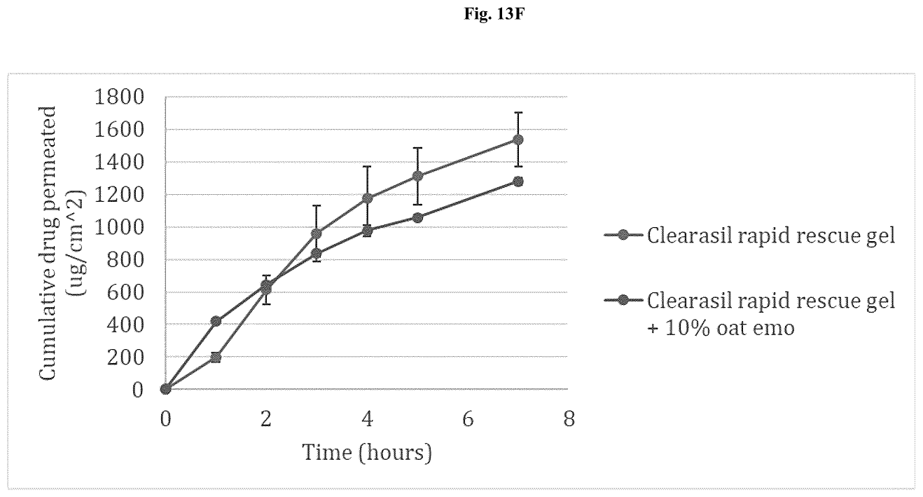

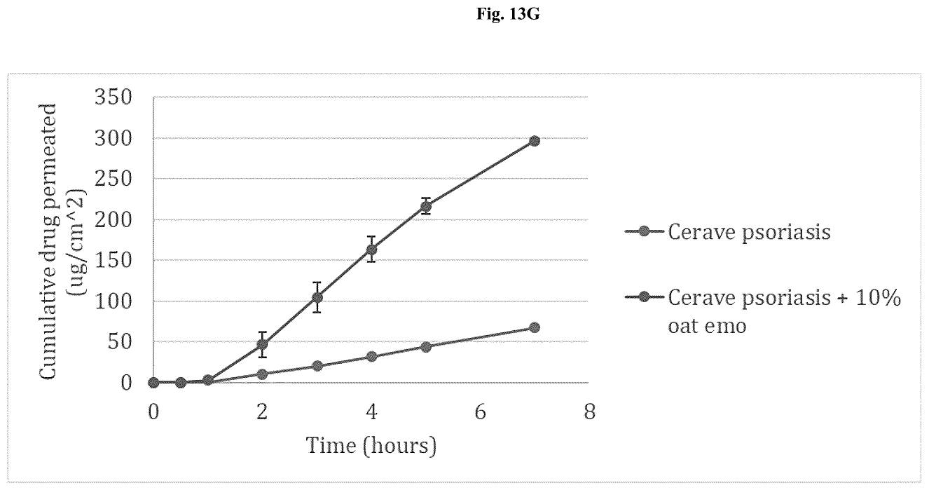

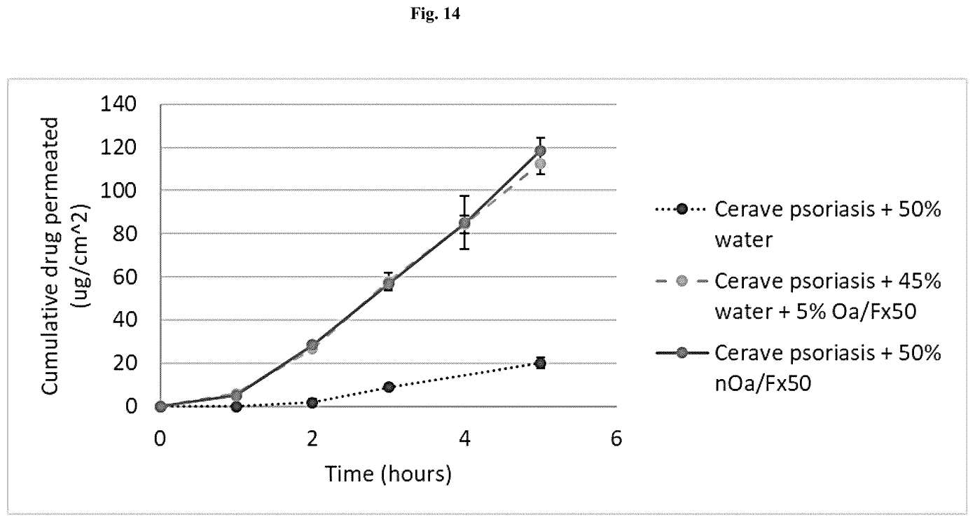

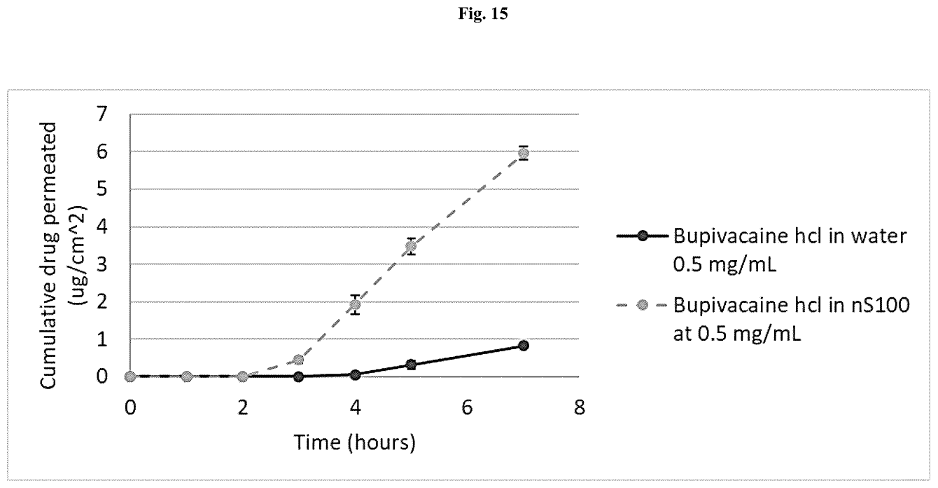

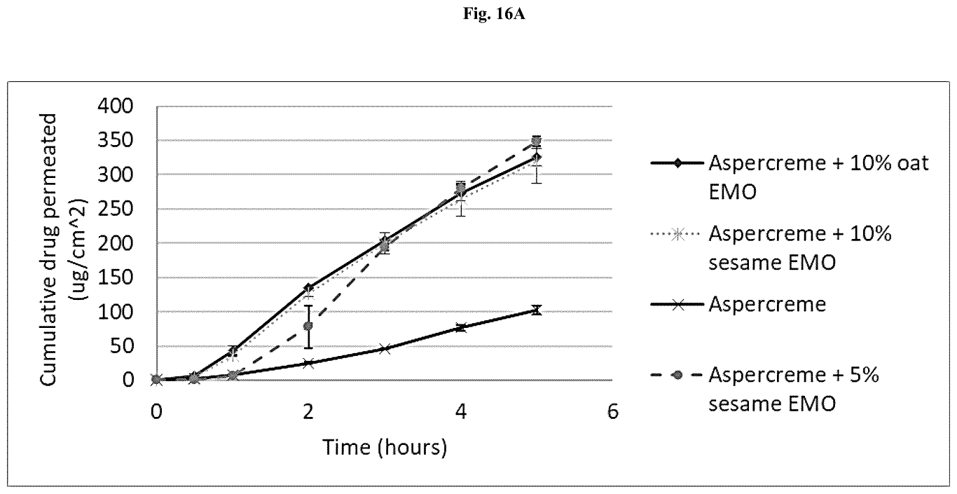

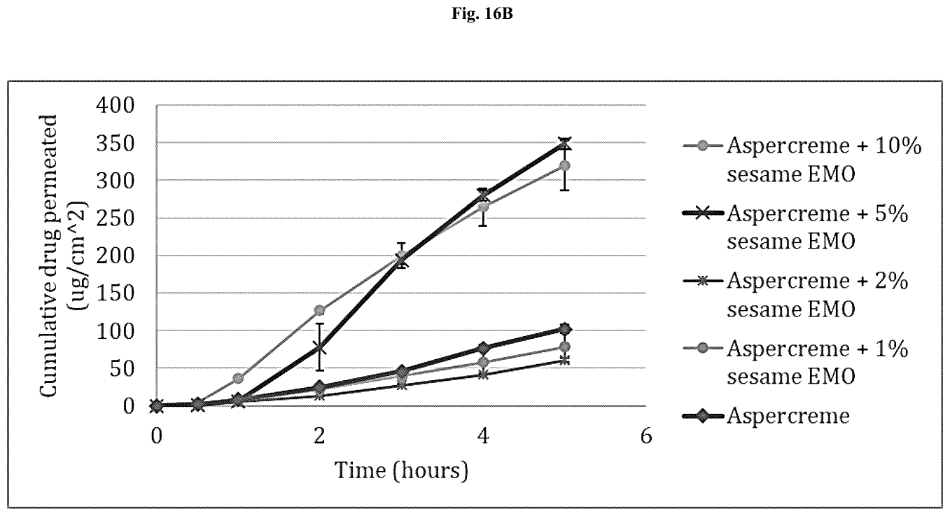

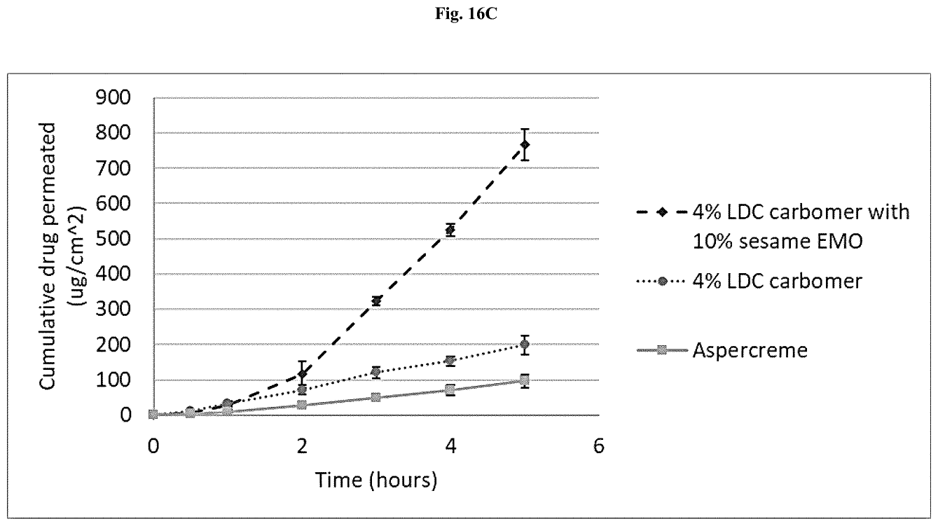

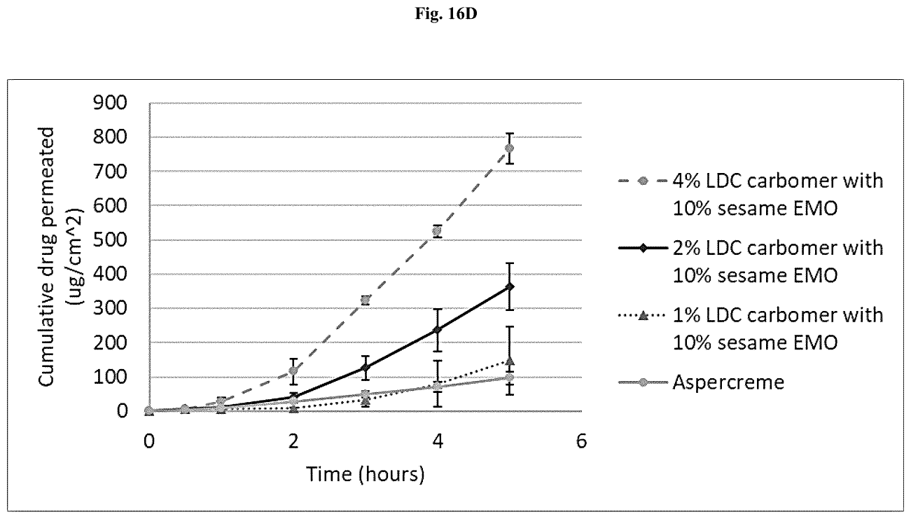

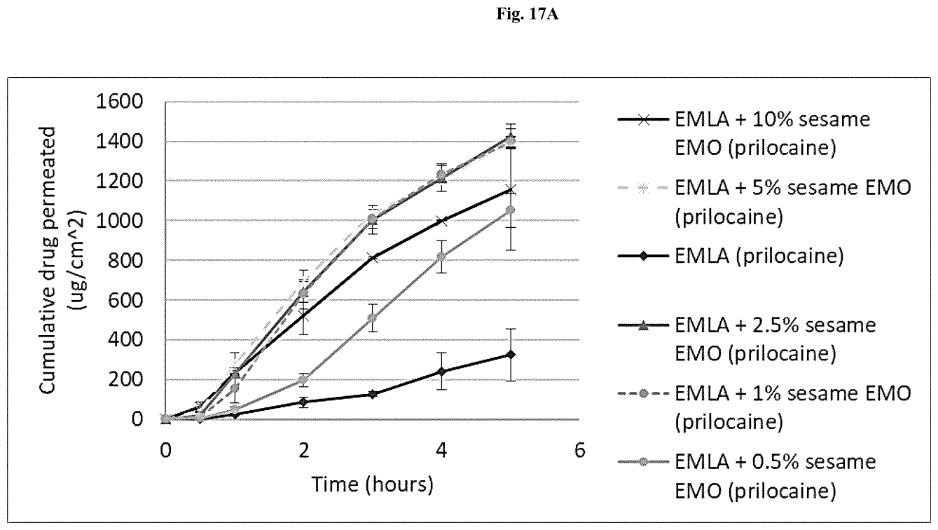

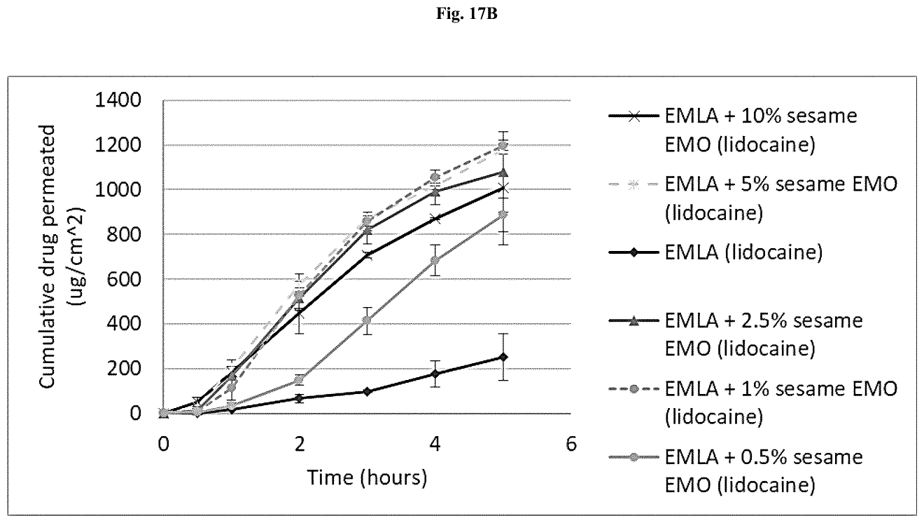

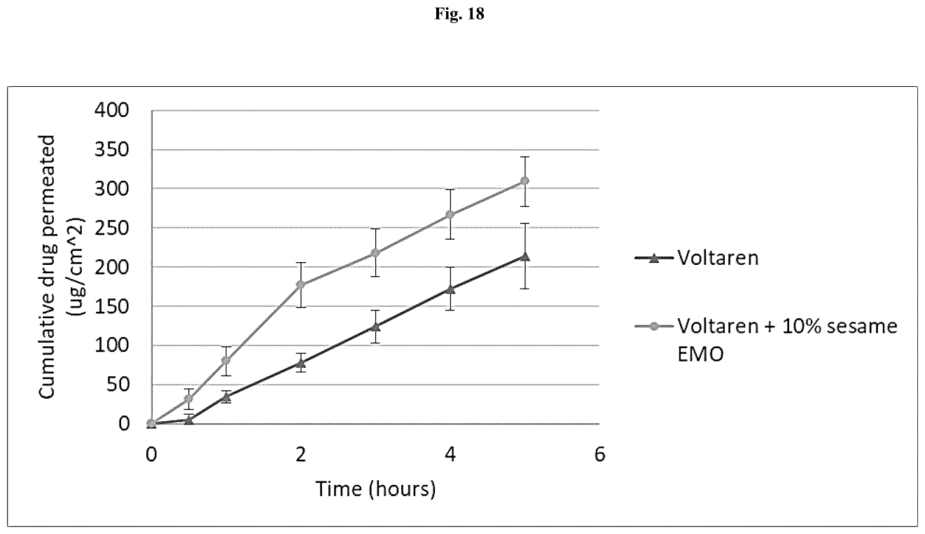

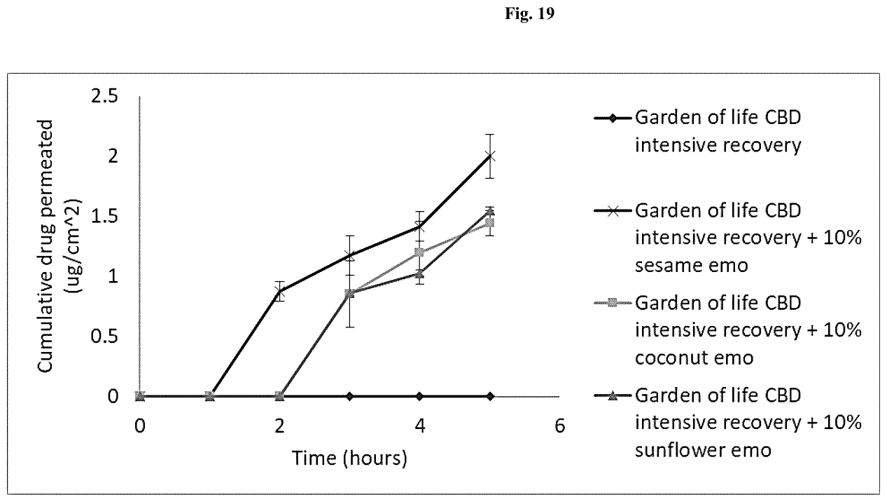

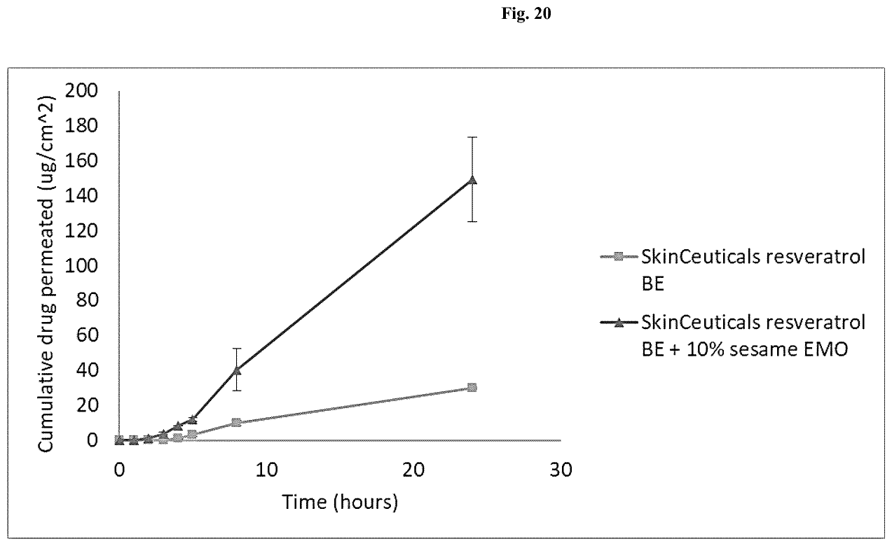

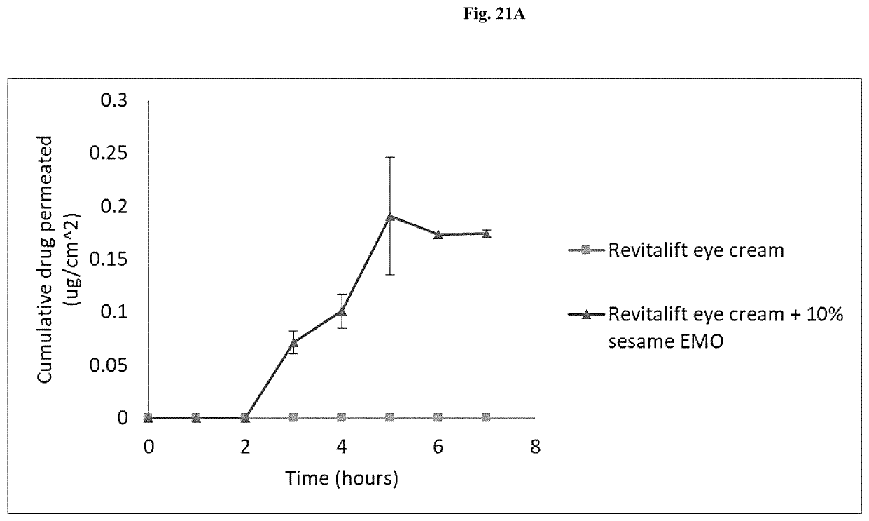

FIG. 2J depicts the Z average particle size as measured by dynamic light scattering for MCT EMO oil nanoemulsion samples over time, stored at room temperature (data are represented as average±SD from at least three measurements; formulations that precipitated were assigned a Z average size of 1000 nm).

FIG. 2K depicts the Z average particle size as measured by dynamic light scattering for olive oil EMO nanoemulsion samples over time, stored at room temperature (data are represented as average±SD from at least three measurements; formulations that precipitated were assigned a Z average size of 1000 nm).

FIG. 2L depicts the Z average particle size as measured by dynamic light scattering for rosehip EMO/coconut FFA oil nanoemulsion samples over time, stored at room temperature (data are represented as average±SD from at least three measurements; formulations that precipitated were assigned a Z average size of 1000 nm).

FIG. 2M depicts the Z average particle size as measured by dynamic light scattering for coconut EMO/rosehip FFA oil nanoemulsion samples over time, stored at room temperature (data are represented as average±SD from at least three measurements; formulations that precipitated were assigned a Z average size of 1000 nm).

FIG. 2N depicts the Z average particle size as measured by dynamic light scattering for rosehip EMO/flaxseed FFA oil nanoemulsion samples over time, stored at room temperature (data are represented as average±SD from at least three measurements; formulations that precipitated were assigned a Z average size of 1000 nm).

FIG. 2O depicts the Z average particle size as measured by dynamic light scattering for flaxseed EMO/rosehip FFA oil nanoemulsion samples over time, stored at room temperature (data are represented as average±SD from at least three measurements; formulations that precipitated were assigned a Z average size of 1000 nm).

FIG. 2P depicts the Z average particle size as measured by dynamic light scattering for rosehip EMO nanoemulsion samples over time, stored at room temperature (data are represented as average±SD from at least three measurements; formulations that precipitated were assigned a Z average size of 1000 nm).

FIG. 2Q depicts the Z average particle size as measured by dynamic light scattering for hemp seed EMO nanoemulsion samples over time, stored at room temperature (data are represented as average±SD from at least three measurements; formulations that precipitated were assigned a Z average size of 1000 nm).

FIG. 2R depicts the Z average particle size as measured by dynamic light scattering for algae EMO/flaxseed FFA oil nanoemulsion samples over time, stored at room temperature (data are represented as average±SD from at least three measurements; formulations that precipitated were assigned a Z average size of 1000 nm).

FIG. 2S depicts the Z average particle size as measured by dynamic light scattering for algae EMO/MCT FFA oil nanoemulsion samples over time, stored at room temperature (data are represented as average±SD from at least three measurements; formulations that precipitated were assigned a Z average size of 1000 nm).

FIG. 3 depicts depicts the size distribution for almond oil mixed micelle nanoemulsion (MMN) 102919.1 3X.

FIG. 4 depicts depicts the zeta potential for almond oil MMN 102919.1 3X.

FIG. 5 depicts depicts the size distribution for almond oil MMN 111819.1.

FIG. 6 depicts the cumulative amount of permeated hydrocortisone over 7 hours through the Strat-M® membrane from nS50, micro-550, nano-TAG, and free drug control (data represented as average±SD; n=2).

FIG. 7 depicts the cumulative amount of permeated hydrocortisone over 7 hours through the Strat-M® membrane from drug loaded (nS50-loaded HCT and micro-S50-loaded HCT) and non-loaded (nS50+HCT DMSO and micro-S50+HCT DMSO) formulations (data represented as average±SD; n=2).

FIG. 8 depicts the cumulative amount of permeated hydrocortisone over 7 hours through the Strat-M® membrane from nFx0, nOa/Fx25, nOA/Fx50 and nOa/Fx75 formulations (data represented as average±SD; n=2).

FIG. 9 depicts the cumulative amount of permeated hydrocortisone over 7 hours through the Strat-M® membrane from nS25, nS50, and nS75 formulations (data represented as average±SD; n=2).

FIG. 10 depicts the cumulative amount of permeated hydrocortisone after 7 hours through the Strat-M® membrane from flaxseed, oat, sesame, rosehip, almond, hemp, and MCT EMO nanoemulsions and HCT control (10% DMSO) (data represented as average±SD; n=2).

FIG. 11 depicts the cumulative amount of permeated hydrocortisone over 7 hours through the Strat-M® membrane from Cerave cream with and without 10% oat EMO (data represented as average±SD; n=2).

FIG. 12 depicts the cumulative salicylic acid permeated over time through a Strat-M® membrane. Data presented as average+/−standard deviation (n=2).

FIG. 13A depicts the cumulative salicylic acid permeated over time for CHARLOTTE'S WEB CBDMEDIC Acne Treatment Medicated Cream with or without 10% sesame EMO. Data presented as average+/−standard deviation (n=2).

FIG. 13B depicts the cumulative salicylic acid permeated over time for CERAVE Acne Control Gel with or without 10% oat EMO. Data presented as average+/−standard deviation (n=2).

FIG. 13C depicts the cumulative salicylic acid permeated over time for CERAVE Body Wash for Rough & Bumpy Skin with or without 10% oat EMO. Data presented as average+/−standard deviation (n=2).

FIG. 13D depicts the cumulative salicylic acid permeated over time for CLEARASIL Stubborn Acne Control+Marks 1 Minute Mask with or without 10% oat EMO. Data presented as average+/−standard deviation (n=2).

FIG. 13E depicts the cumulative salicylic acid permeated over time for NEUTROGENA Rapid Clear Acne Eliminating Spot Gel with or without 10% oat EMO. Data presented as average+/−standard deviation (n=2).

FIG. 13F depicts the cumulative salicylic acid permeated over time for CLEARASIL Rapid Rescue Spot Treatment Gel with or without 10% oat EMO. Data presented as average+/−standard deviation (n=2).

FIG. 13G depicts the cumulative salicylic acid permeated over time for CERAVE Psoriasis Moisturizing Cream with or without 10% oat EMO. Data presented as average+/−standard deviation (n=2).

FIG. 14 depicts the cumulative salicylic acid permeated over time for CERAVE Psoriasis Moisturizing Cream with or without EMO/FFA oil in nanoemulsion or non-nanoemulsion form. Data presented as average+/−standard deviation (n=2).

FIG. 15 depicts the cumulative bupivacaine HCl permeated over time in Example 20 with or without sesame EMO nanoemulsion. Data presented as average+/−standard deviation (n=2).

FIG. 16A depicts the cumulative lidocaine permeated over time in ASPERCREME with or without EMO. Data presented as average+/−standard deviation (n=2).

FIG. 16B depicts the cumulative lidocaine permeated over time in ASPERCREME with or without EMO. Data presented as average+/−standard deviation (n=2).

FIG. 16C depicts the cumulative lidocaine permeated over time from ASPERCREME and lidocaine carbomer formulations with or without sesame EMO. Data presented as average+/−standard deviation (n=2).

FIG. 16D depicts the cumulative lidocaine permeated over time from ASPERCREME and lidocaine carbomer formulations with sesame EMO at varying lidocaine levels. Data presented as average+/−standard deviation (n=2).

FIG. 17A depicts the cumulative prilocaine permeated over time from EMLA with or without sesame EMO. Data presented as average+/−standard deviation (n=2).

FIG. 17B depicts the cumulative lidocaine permeated over time from EMLA with or without sesame EMO. Data presented as average+/−standard deviation (n=2).

FIG. 18 depicts the cumulative diclofenac permeated over time from VOLTAREN cream with and without sesame EMO. Data presented as average+/−standard deviation (n=2).

FIG. 19 depicts cumulative CBD permeated over time from GARDEN OF LIFE Intensive Recovery cream with and without EMO. Data presented as average+/−standard deviation (n=2).

FIG. 20 depicts cumulative resveratrol permeated over time from SkinCeuticals Resveratrol BE cream with and without EMO. Data presented as average+/−standard deviation (n=2).

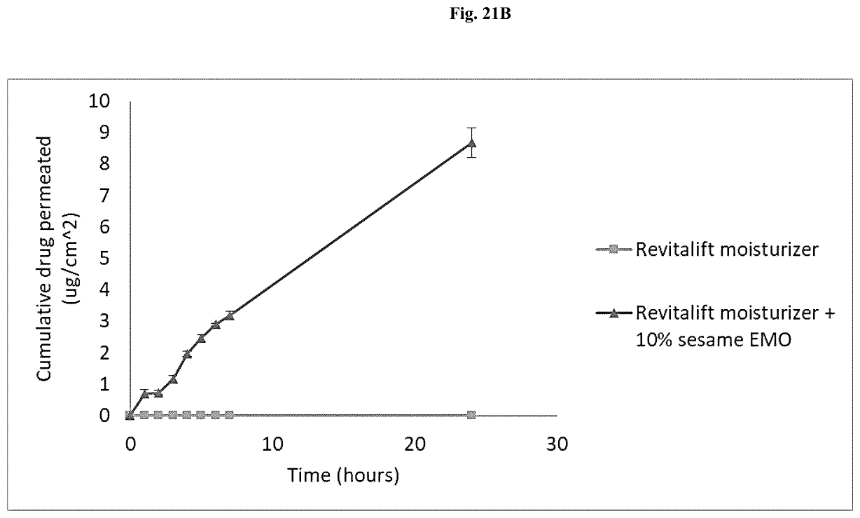

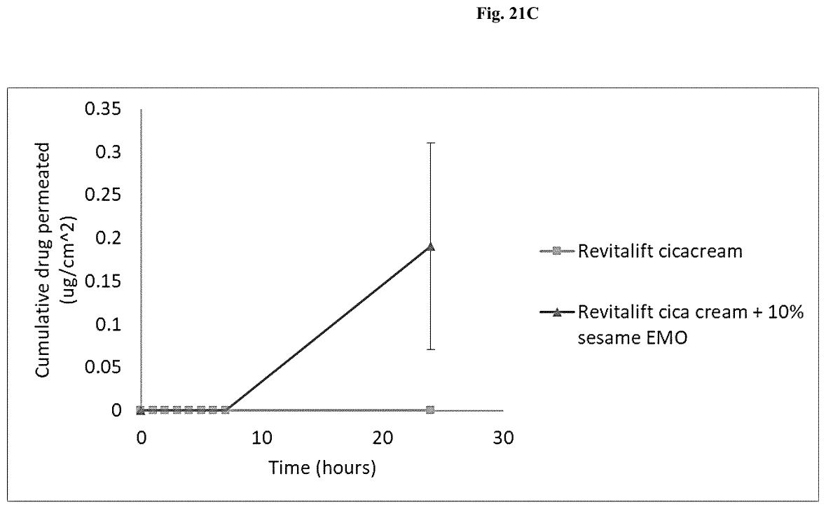

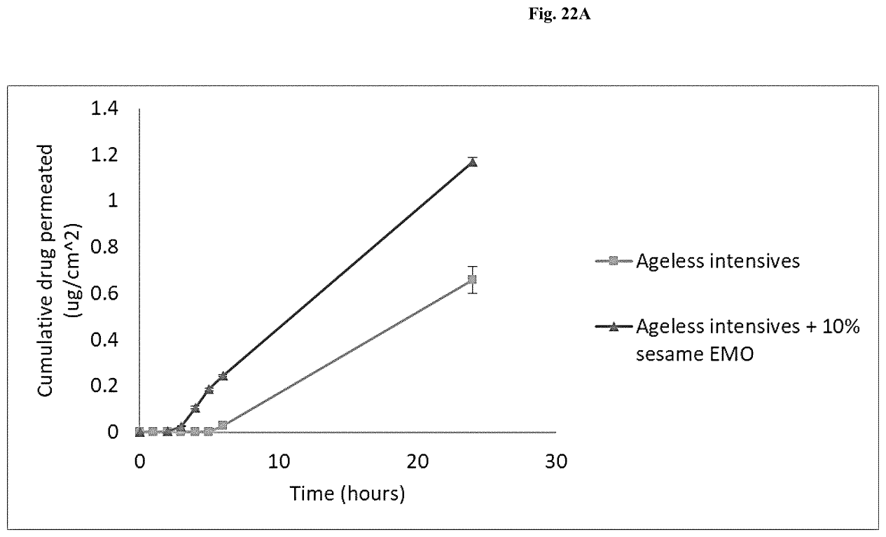

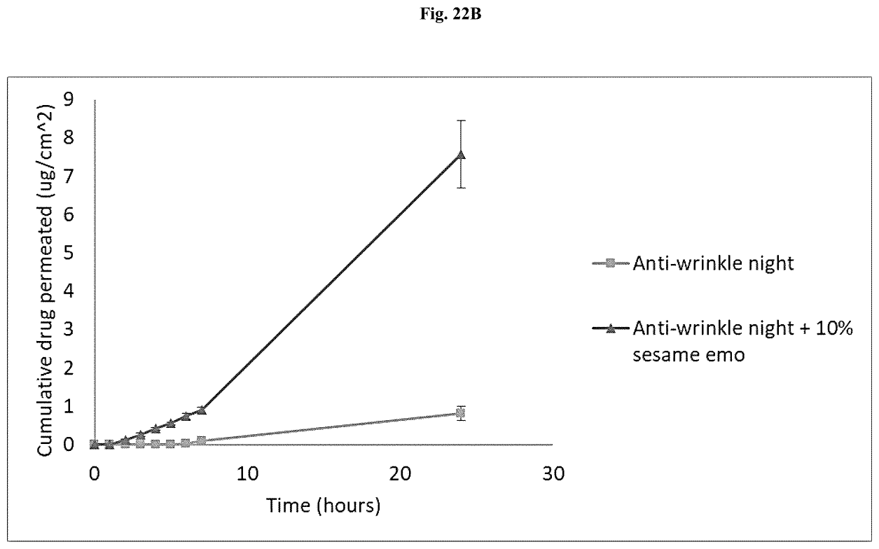

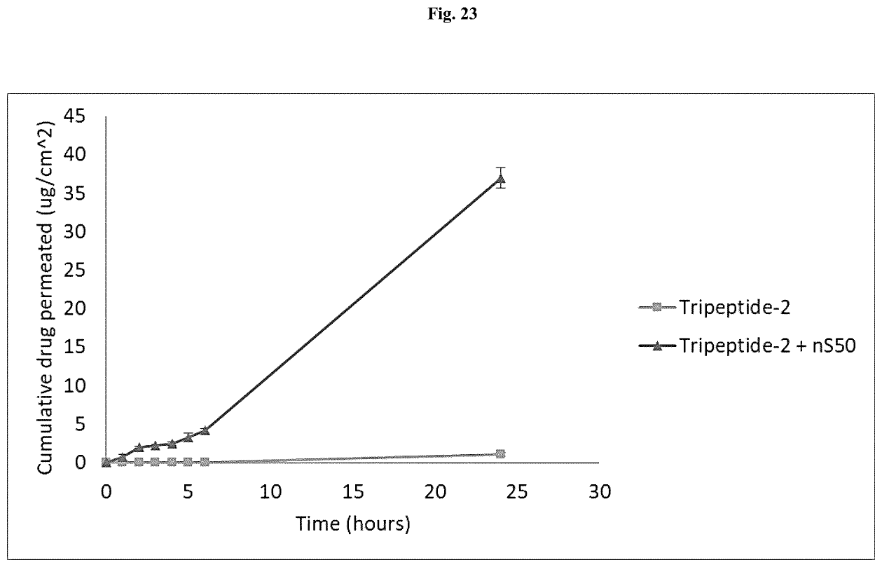

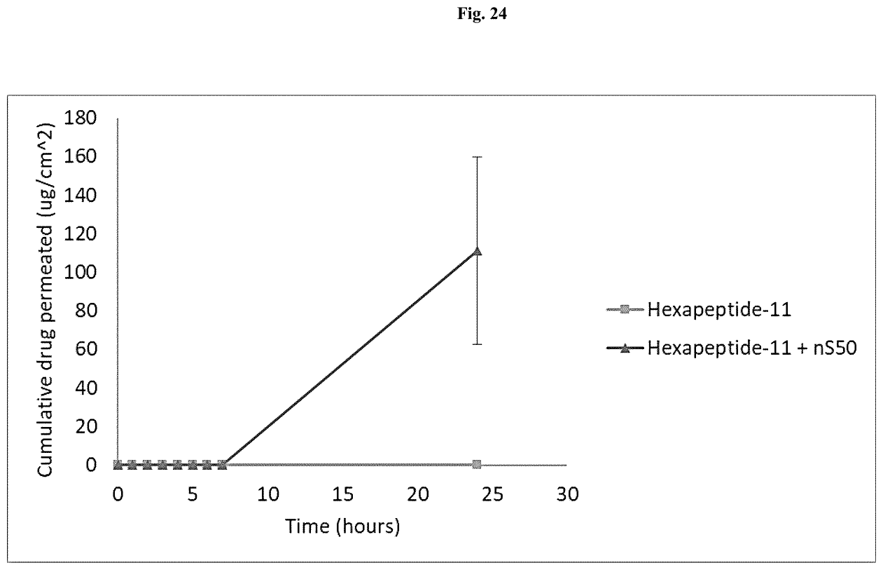

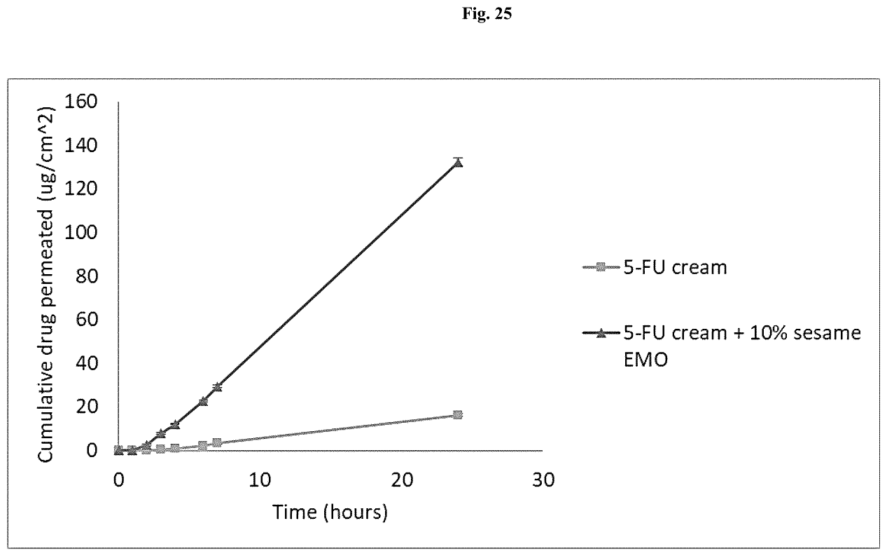

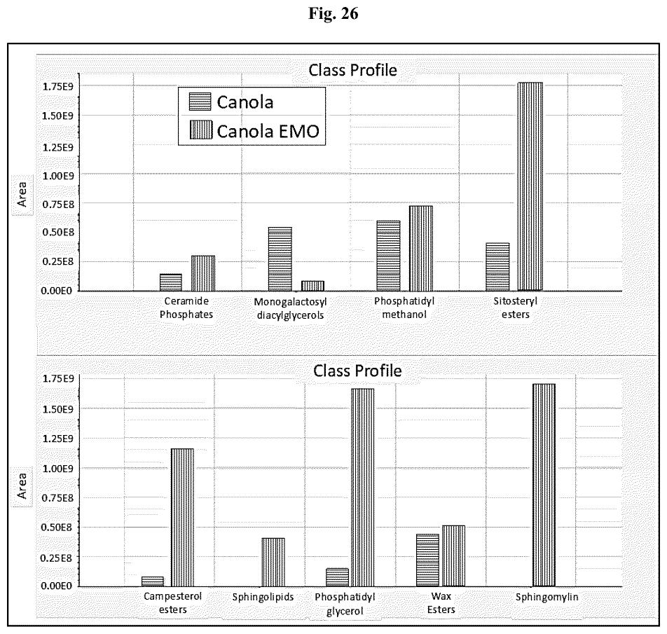

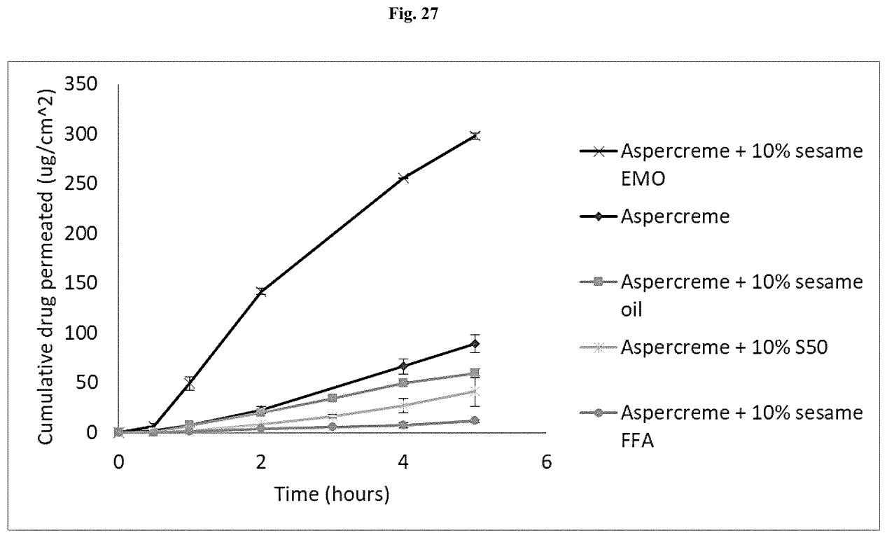

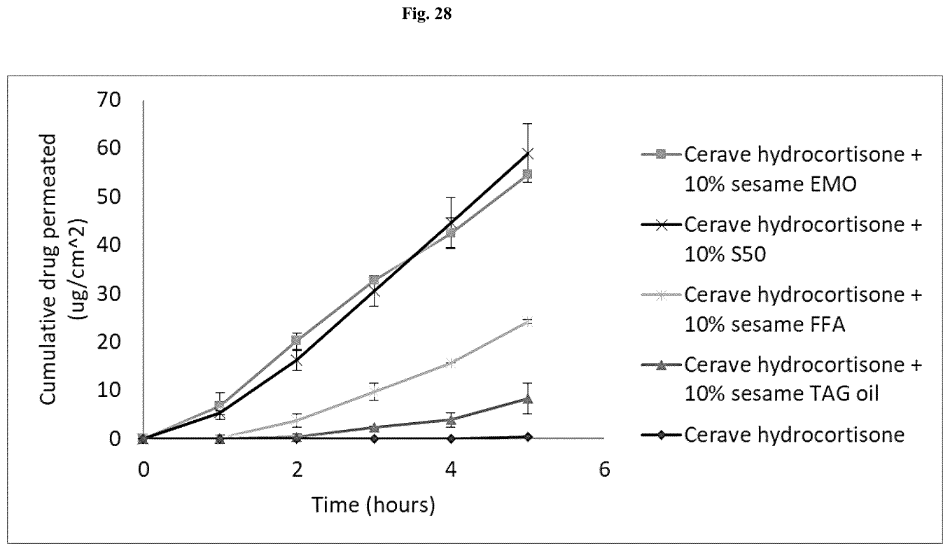

FIG. 21A depicts cumulative retinyl palmitate permeated over time from REVITALIFT anti-wrinkle eye cream with and without oat EMO. Data presented as average+/−standard deviation (n=2).

FIG. 21B depicts cumulative retinyl palmitate permeated over time from REVITALIFT anti-wrinkle moisturizer cream with and without oat EMO. Data presented as average+/−standard deviation (n=2).

FIG. 21C depicts cumulative retinyl palmitate permeated over time from REVITALIFT Cicacream with and without oat EMO. Data presented as average+/−standard deviation (n=2).

FIG. 22A depicts cumulative retinol permeated over time for NEUTROGENA AGELESS INTENSIVES Anti-Wrinkle Cream with and without 10% sesame EMO. Data presented as average+/−standard deviation (n=2).

FIG. 22B depicts cumulative retinol permeated over time for NEUTROGENA Healthy Skin Anti-Wrinkle Night Cream with and without 10% sesame EMO. Data presented as average+/−standard deviation (n=2).

FIG. 23 depicts cumulative trifluoroacetyl tripeptide-2 over time from sesame EMO nanoemulsion and free peptide control. Data presented as average+/−standard deviation (n=2).

FIG. 24 depicts cumulative hexapeptide-11 permeated over time from sesame EMO nanoemulsion and free peptide control. Data presented as average+/−standard deviation (n=2).

FIG. 25 depicts cumulative 5-fluorouracil (5-FU) permeated over time from prescription cream with and without 10% EMO. Data presented as average+/−standard deviation (n=2).

FIG. 26 depicts measurement of non-oil ingredients in unmodified canola oil and canola EMO.

FIG. 27 depicts cumulative lidocaine permeated over time from ASPERCREME with or without sesame EMO, EMO/FFA oil, FFA oil, or TAG oil. Data presented as average+/−standard deviation (n=2).

FIG. 28 depicts cumulative hydrocortisone permeated over time from ASPERCREME with or without sesame EMO, EMO/FFA oil, FFA oil, or TAG oil. Data presented as average+/−standard deviation (n=2).

### DETAILED DESCRIPTION

The present disclosure is directed to oil compositions that can include an enzyme-modified oil (EMO) and/or a free fatty acid (FFA) oil and which can be used in combination with an active ingredient or to make nano- and micro-emulsions. The oil compositions of the present disclosure can be used in any formulation, but can also be used to formulate nanoemulsions and microemulsions that contain free fatty acids and, optionally, monoacylglycerols (MAGs) and/or diacylglycerols (DAGs), that do not require additional agents to stabilize the nanoemulsions. Methods for making the same are also provided in addition to methods of using, including administering to a subject the formulations containing an active ingredient and/or the oil composition. In certain aspects, the compositions of the present disclosure can provide enhanced permeability for active ingredients.

It is to be understood that both the foregoing general description and the following detailed description are exemplary and explanatory only, and are not restrictive of the invention, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

### Definitions

As used herein, “nanoemulsion” refers to a colloidal particulate system consisting of two immiscible liquids, or a liquid and a solid, in which one liquid or the solid (dispersed phase) is dispersed as a droplet or particle, respectively, into the other liquid (continuous phase). A “nanoemulsion” can include droplets or particles having a size from 10 to 1000 nm, but generally nanoemulsions must have small droplet sizes (<300 nm) in order to be kinetically stable. The dispersed phase is also known as the discontinuous phase while the outer phase can be called the continuous phase.

As used herein, “microemulsion” refers to a colloidal particulate system consisting of two immiscible liquids, or a liquid and a solid, in which one liquid or the solid (dispersed phase) is dispersed as a droplet or particle, respective into the other liquid (continuous phase). A “microemulsion” can include droplets or particles have a size greater than 1000 nm to 1000000 nm.

As used herein, “free fatty acid (FFA)” refers to a non-esterified fatty acid in its carboxylic acid form or carboxylate salt form.

As used herein, a “monoacylglycerol (MAG),” also known as a monoglyceride, is a glyceride consisting of one fatty acid chain covalently bonded to a glycerol molecule through an ester linkage.

As used herein, a “diacylglycerol (DAG),” also known as a diglyceride, is a glyceride consisting of two fatty acid chains covalently bonded to a glycerol molecule through ester linkages.

As used herein, a “triacylglycerol (TAG),” also known as a triglyceride, is a glyceride consisting of three fatty acid chains covalently bonded to a glycerol molecule through ester linkages. TAGs may also be classified as having a long or medium chain length. Long chain TAGs contain fatty acids with 14 or more carbons, while medium chain TAGs contain fatty acids with 6 to 12 carbons. Long chain TAGs can include omega-3 and omega-6 fatty acids. Medium chain TAGs have saturated fatty acids and thus do not contain omega-3 or omega-6 fatty acids. Long chain TAGs (LCT) and medium chain triglycerides (MCT) can serve as energy sources.

As used herein, “exogenous additive with surfactant and/or emulsifier properties” refers to a compound acting as a surfactant or emulsifier that is added to an oil but not naturally present in the oil in an amount sufficient to enhance droplet or particle formation and stability in a nanoemulsion. Non-limiting examples of surfactants 4-(5-dodecyl)benzenesulfonate, docusate (dioctyl sodium sulfosuccinate), alkyl ether phosphates, benzalkaonium chloride (BAC), and perfluorooctanesulfonate (PFOS). Non-lirniting examples of emulsifiers include ethoxylated alcohols, carboxylates, sodium isothionate, cetyl alcohol, stearyl alcohol, and silicone emulsifiers such as dimethicones.

As used herein, “exogenous additive with thickening or crystallization inhibiting properties” refers to a thickener or crystallization inhibitor that is added to an oil but not naturally present in the oil in an amount sufficient to thicken a nanoemulsion or inhibit crystallization, respectively. By way of example, but not limitation, such exogenous additives with thickening properties can include methyl cellulose, cornstarch, sodium alginate and gelatin. By way of example, but not limitation, such exogenous additives with crystallization inhibiting properties can include polyglycerol fatty acid esters, sucrose fatty acid esters, and sorbitan fatty acid esters.

As used herein, “surfactant” or “emulsifier” refers to a substance that reduces the surface tension between two liquids or, in the case of a solid lipid nanoemulsion, between the liquid phase and the solid lipid phase. Surfactants, or surface-active agents, are compounds that lower the surface tension between two liquids or between a liquid and a solid. An emulsifier is a surfactant that stabilizes emulsions. Emulsifiers coat droplets within an emulsion and prevent them from coming together, or coalescing.

Surfactants are commonly used as emulsifiers as they are known to stabilize emulsions. By way of example, but not limitation, surfactants (and emulsifiers) can include polysorbates, phospholipids, sterols, cationic lipids, poloxamers, sorbitan esters, sugar esters, polyoxyethylene conjugates, ammonium phosphatidyl lapsidate, polyphenols, polyvinylalcohol, lecithin, ethanol, lysophospholipids, ceramides, Tweens, Spans, Kolliphor, propylene glycol, vitamin D, polyethylene glycol, polyethylene glycol derivatives, pectin, gum acacia, modified gum acacia, agar, ghatti gum, modified ghatti gum, pectin, carrageenan, xanthan gum, modified starches, modified alginate, fatty alcohols, and ethoxylated polyols.

As used herein, “enzyme-modified oil (EMO)” refers to an oil that includes MAGs and, optionally DAGs and/or FFA that results from enzymatic hydrolysis of a starting oil source(s) to yield FFA and glycerol followed by separation of the glycerol, and at least partial enzymatic re-esterification of glycerol with the FFA to yield MAGs and, optionally some DAGs, and, in some instances, some residual FFAs. Unless otherwise stated herein, an EMO should be understood to be substantially free of TAGs (below a detectable level). In some embodiments, an EMO can have less than 5% TAGs.

As used herein, “free fatty acid oil (FFA oil)” refers to an oil that includes free fatty acids that result from enzymatic hydrolysis of TAGs in a starting oil source(s) to FFA and glycerol, with subsequent removal of the glycerol. It should be understood that the FFA oil can include some glycerol which may be dissolved in the FFA oil.

Methods for producing EMO and FFA oil, and properties thereof, are disclosed in International Patent Application Nos. PCT/US2018/055583, filed Oct. 12, 2018, published as WO 2019/075307 and PCT/US2020/014182, filed Jan. 17, 2020, published as WO 2020/150661, and U.S. Patent Application Publication No. 2020/0146307 (U.S. Ser. No. 16/746,700), each of which is incorporated herein by reference in its entirety.

As used herein, “non-oil ingredient” is an ingredient that is naturally present in an oil source that is not a MAG, DAG, TAG, or FFA.

As used herein, a “therapeutically effective amount” refers to an amount of a composition of the present disclosure effective yield a desired therapeutic response. By way of example, but not limitation, an amount effective to delay the growth of or to cause a cancer to shrink or to reduce pain or inflammation can be “therapeutically effective amount.” The specific therapeutically effective amount will vary with such factors as the particular condition being treated, the physical condition of the subject, the type of subject being treated, the duration of the treatment, the nature of concurrent therapy (if any), and the specific formulations employed and the structure of the composition.

As used herein, an “active ingredient” is a substance that is intended for use in the diagnosis, cure, mitigation, treatment or prevention of disease, or to affect the structure or function of the body of a subject, such as a mammal, preferably a human. By way example, but not limitation, an active ingredient can be a pharmaceutically active substance, including, but not limited to, a steroid, peptide, protein, synthetic chemical molecule, a plant extract, vitamin, nucleic acid and antioxidant.

As used herein, a “subject” refers to any living being, such as a human being or animal.

Two critical factors can determine the potential success of emulsion production and stability-interfacial tension and repulsion by electric charge.

The surface tension of the fluids determines their propensity to form emulsions. The surface tension can be lowered by adding surfactants. Most surfactants are amphiphilic compounds. The emulsifying agents can concentrate at the oil-water interface, producing a significant reduction of the interfacial tension and consequently significantly reduce the energy to form emulsions. Despite a lowering of interfacial tension when surface-active agents are added, the free energy of the interface remains positive, leaving a persisting state of thermodynamic instability.

The stability of an emulsion can be improved by adding emulsifiers. Emulsifiers frequently have a hydrophobic end (e.g. a long chain hydrocarbon) and a charged end (for example a carboxylic acid). Emulsifiers coat the surface of the droplets or particles. Emulsion stability is often explained by the presence of repulsive electrical charges on the surfaces of emulsion droplets. If the magnitude of this energy barrier exceeds the kinetic energy of the particles, the suspension is stable. ζ-potential (“zeta potential”) is a typical measure of repulsion. Generally, a zeta potential of +/−30 mV or more ensures good stability.

The size of the nanoemulsion discontinuous phase particles/droplets can typically be measured using Dynamic Light Scattering (DLS). DLS is a non-invasive, well-established technique for measuring the size and size distribution of molecules and particles typically in the submicron region. Brownian motion of particles or molecules in suspension causes laser light to be scattered at different intensities, varying over time because of constructive and destructive interference. Analysis of these intensity fluctuations yields the underlying diffusion coefficient and, hence, the particle size using the Stokes-Einstein relationship. A typical results can include: (1) a mean or “Z average size”, i.e. an overall average size defined via a simple cumulant fit; (2) a polydispersity index (PDI), i.e. an indication of how broad a (forced) single Gaussian peak would have to be to fit the result (0.0 is perfectly monodisperse, 1.0 is extremely polydisperse); or (3) a size distribution obtained from a multipeak analysis with the mean peak size and standard deviation for each peak indicated, as well as the relative signal intensity (% intensity) originating from the species. Sizes can generally be expressed as “diameter in nanometers” (d, nm).

Zeta potential can be defined as the potential difference between the dispersion medium and the stationary layer of fluid attached to the particle and can be measured by Electrophoretic Light Scattering (ELS). ELS is a technique that can be used to measure the electrophoretic mobility of particles in dispersion, or molecules in solution. This mobility is often converted to zeta potential to enable comparison of materials under different experimental condition. The concept is similar to DLS, yet the movement and intensity fluctuations are studied under the influence of an applied electric field. The fundamental physical principle is that of electrophoresis. A dispersion is introduced into a cell containing two electrodes. An electrical field is then applied to the electrodes, and particles or molecules that have a net charge will migrate towards the oppositely charged electrode with a velocity, known as the mobility, that is related to their zeta potential. A typical result can include: (1) zeta potential, i.e. the overall average zeta potential of the dispersed, e.g. oil, particles in the sample; or (2) conductivity, the electrophoretic mobility is often also reported in mS/cm.

Nanoemulsion Compositions

In some embodiments, a composition is provided that includes an oil dispersed in a liquid, where the oil is in the form of droplets or particles and includes at least 2% free fatty acids (FFAs) by weight of the oil, where the FFAs include two or more different fatty acids, where the oil includes 80% or less triacylglycerols (TAGs) by weight out of the total weight of the oil, and where the droplets or particles have a Z average size of about 300 nm or less as measured by dynamic light scattering.

In some embodiments, a composition is provided that includes an oil dispersed in a liquid, where the oil is in the form of droplets or particles and includes at least 2% FFAs by weight of the oil and 80% or less TAGs by weight of the oil, where the oil includes non-oil ingredients that are naturally present in the oil, and where the droplets or particles have a Z average particle size of about 300 nm or less as measured by dynamic light scattering.

In some embodiments, a composition is provided that includes an oil dispersed in a liquid, where the oil is in the form of droplets or particles and includes at least 2% FFAs by weight of the oil, where the FFAs include two or more different fatty acids, where the oil also includes monoacylglycerols (MAGs) in an amount of at least 5% by weight out of the total weight of the oil, where the MAGs and FFAs are derived from a common, first oil source, and where the droplets or particles have a Z average particle size of about 300 nm or less as measured by dynamic light scattering.

In some embodiments, a composition is provided that includes an oil dispersed in a liquid, where the oil is in the form of droplets or particles and includes at least 2% FFAs by weight of the oil, where the FFAs include two or more different fatty acids, where the oil also includes MAGs in an amount of at least 5% by weight of the total weight of the oil, where the MAGs and FFAs are derived from a first oil source and a second oil source, respectively, and where the droplets or particles have a Z average size of about 300 nm or less as measured by dynamic light scattering.

In some embodiments, a composition is provided that includes an oil dispersed in a liquid, where the oil is in the form of droplets or particles and includes at least 2% FFAs by weight of the oil, where the FFAs include two or more different fatty acids, where the oil also includes monoacylglycerols (MAGs) and diacylglycerols (DAGs) in an amount of at least 5% by weight out of the total weight of the oil, where the MAGs and FFAs are derived from a common, first oil source, and where the droplets or particles have a Z average particle size of about 300 nm or less as measured by dynamic light scattering.

In some embodiments, a composition is provided that includes an oil dispersed in a liquid, where the oil is in the form of droplets or particles and includes at least 2% FFAs by weight of the oil, where the FFAs include two or more different fatty acids, where the oil also includes MAGs and DAGs in an amount of at least 5% by weight of the total weight of the oil, where the MAGs and FFAs are derived from a first oil source and a second oil source, respectively, and where the droplets or particles have a Z average size of about 300 nm or less as measured by dynamic light scattering.

In any of the foregoing embodiments, the oil can include at least 2% FFAs by weight of the oil. By way of example, but not limitation, the oil can include at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% FFAs by weight of the oil. By way of further example, but not limitation, the oil can include about 2% to about 99%, about 5% to about 99%, about 10% to about 99%, about 15% to about 99%, about 20% to about 99%, about 25% to about 99%, about 30% to about 99%, about 35% to about 99%, about 40% to about 99%, about 50% to about 99%, about 60% to about 99%, about 70% to about 99%, about 80% to about 99%, about 90% to about 99%, about 5% to about 90%, about 5% to about 80%, about 5% to about 75%, about 5% to about 70%, about 5% to about 60%, about 5% to about 50%, about 5% to about 40%, about 5% to about 30%, about 5% to about 25%, about 5% to about 20%, about 5% to about 10%, about 10% to about 90%, about 10% to about 80%, about 10% to about 75%, about 10% to about 70%, about 10% to about 60%, about 10% to about 50%, about 10% to about 40%, about 10% to about 30%, about 10% to about 25%, about 10% to about 20%, about 20% to about 90%, about 20% to about 80%, about 20% to about 75%, about 20% to about 70%, about 20% to about 60%, about 20% to about 50%, about 20% to about 40%, about 20% to about 30%, about 20% to about 25%, about 25% to about 90%, about 25% to about 80%, about 25% to about 75%, about 25% to about 70%, about 25% to about 60%, about 25% to about 50%, about 25% to about 40%, about 25% to about 30%, about 30% to about 90%, about 30% to about 80%, about 30% to about 75%, about 30% to about 70%, about 30% to about 60%, about 30% to about 50%, about 30% to about 40%, about 40% to about 90%, about 40% to about 80%, about 40% to about 75%, about 40% to about 70%, about 40% to about 60%, about 40% to about 50%, about 50% to about 90%, about 50% to about 80%, about 50% to about 75%, about 50% to about 70%, about 50% to about 60%, about 60% to about 90%, about 60% to about 80%, about 60% to about 75%, about 60% to about 70%, about 70% to about 90%, about 70% to about 80%, about 80% to about 90%, about 9% to about 37%, about 2%, 3%, 4%, 5%, 6%, 7%, 8%, 9%, 10%, 15%, 20%, 25%, 30%, 40%, 50%, 60%, 70%, 75%, 80%, 90%, 95%, or 99% FFAs by weight of the oil. By way of still further example, but not limitation, in any of the foregoing embodiments where a minimum amount of MAGs and/or DAGs is not recited, the FFA can be present at up to about 100% by weight of the oil.

In any of the foregoing embodiments, the oil can include 80% or less, 70% or less, 60% or less, 50% or less, 40% or less, 30% or less, 25% or less, 20% or less, 15% or less, 10% or less, 5% or less, 4% or less, 3% or less, 2% or less, 1% or less, or is substantially free of triacylglycerols (TAGs). By way of further example, the oil can have no measurable TAGs as assessed qualitatively by Thin Layer Chromatography (TLC). In this method, components of the oil are separated using TLC plates (Analtech Uniplate Silica Gel GIL with inorganic binder, 20×20 cm, 250 μm), using a solvent (Hexane: Diethyl Ether:Acetic Acid (70:30:1) solution. Typical sample sizes are 0.2 μL. After the solvent front runs to near the top of the plate (˜1 cm), plates are removed from the TLC tank and the solvent evaporated in a fume hood. The components are visualized with iodine vapors (at room temperature) in a TLC tank and relative intensities estimated by colorimetric imaging. Components can also be measure quantitatively by standard HPLC and GC/MS methods. By way of still further example, the oil can have no measurable TAGs as assessed by gas chromatography (GC) or high performance liquid chromatography (HPLC). By way of example, but not limitation, HPLC can be performed under the following HPLC conditions: C18 column, 4.6 mm I.D.×250 mm, 5 um; column temp at 30° C.; charged aerosol detector, mobile phase A consisting of 75% acetonitrile, 12% methanol, 8% water, 4% tetrahydrofuran, and 0.3% acetic acid; mobile phase B consisting of 90% acetone and 10% acetonitrile; a gradient profile of: 100% A at 1.5 mL/min from 0-5 min, 100% A to 96% A at 1.5 mL/min from 5 to 11 min, 96% A to 37% A at 1.5 mL/min from 11 to 24 min, 37% A to 32% A at 1.5 mL/min from 24 to 35 min, 32% A to 18% A at 1.5 mL/min from 35 to 45 min, 18% A to 15% A at 1.9 mL/min from 45 to 58 min, 15% A to 5% A at 1.9 mL/min from 58 to 60 min, 5% A to 100% A at 1.5 mL/min from 60 to 63 min, 100% A at 1.5 mL/min from 63 to 65 min, using MAG, DAG, TAG and FFA calibration standards. Methods of quantitative measurements of glycerides and free fatty acids are described, by way of example but not limitation, Simultaneous Analysis of Glycerides (Mono-, Di-, and Triglycerides) and Free Fatty Acids in Palm Oil, Application Note 1039, Thermo Fisher Scientific, Inc., 2012, Annex 5.

In any of the foregoing embodiments, the oil can include two or more different fatty acids. By way of example, but not limitation, the oil can include two, three, four, five or more different fatty acids.

In any of the foregoing embodiments, the free fatty acids, including the two or more different fatty acids can be, by way of example but not limitation, selected from the group consisting of caproic acid, enanthic acid, caprylic acid, pelargonic acid, capric acid, undecylic acid, lauric acid, tridecylic acid, myristic acid, pentadecylic acid, palmitic acid, palmitoleic acid, margaric acid, heptadecenoic acid, stearic acid, nonadecylic acid, vaccenic acid, oleic acid, linoleic acid, linolenic acid, stearidonic acid, octadecatetraenoic acid, arachidic acid, heneicosylic acid, gondoic acid, eicosadienoic acid, paullinic acid, behenic acid, tricosylic acid, erucic acid, lignoceric acid, docosahexaenoic acid, eicosapentaenoic acid, nervonic acid, and isomers thereof. Alternatively, the fatty acids, including the two or more different fatty acids can each independently be selected from the group consisting of a C6 fatty acid such as, by way of example but not limitation, C6:0, a C7 fatty acid, a C8 fatty acid such as, by way of example but not limitation, C8:0 fatty acid, a C9 fatty acid, a C10 fatty acid, a C11 fatty acid, a C12 fatty acid, a C13 fatty acid, a C14 fatty acid, a C15 fatty acid, a C16 fatty acid, a C17 fatty acid, a C18 fatty acid such as, by way of example, but not limitation, a C18:0 fatty acid, a C18:0 fatty acid, a C18:1 fatty acid, a C18:2 fatty acid, or a C18:3 fatty acid, a C19 fatty acid, a C20 fatty acid such as, by way of example, but not limitation, C20:5 fatty acid, a C21 fatty acid, a C22 fatty acid such as, by way of example, but not limitation, C22:6 fatty acid, a C23 fatty acid, and a C24 fatty acid. As used herein, C18 should be understood to refer to stearic acid (C18:0).

In any of the foregoing embodiments, the oil can include non-oil ingredients that are naturally present in the oil. By way of example, but not limitation, the non-oil ingredients that are naturally present in the oil can include one or more of glycosphingolipids such as ceramide phosphates, glycoglycerolipids such as monogalactosyl diacylglycerols, phophatidyl alcohols such as phosphatidyl methanol, steroids such as sitosteryl esters, natural lipids such as campesterol esters, sphingolipids, phosphatidyl glycerol, wax esters, sphingomyelin, phosphatides, phytosterols (campesterol, stigmasterol, sitosterol), cholesterol, tocopherols, tocotrienols, carotenes, xanthophylls, betaxanthins, chlorophyll, long chain alcohols, polyphenols, terpenes (cycloartanol, cycloarterenol, 24-methylene cycloarterenol), terpenoids (squalene), tocopherol, vitamins, paraffins, isothiocyanates, oxazolidinthiones, glucosinolates, sanguinarine, gossypols, glycosylated alkaloids, phospholipids, sphingosine, diacylglyceride ethers, glucolipids, phytic acid, quinic acid, oxalic acid, tartaric acid, anacardic acid, malic acid, caftaric acid, coutaric acid, fertaric acid, phenylethanoids, hydroxycinnamic acids, phenolic acids, phytoestrogens, lignans, isoflavonoids, flavonoids, monoterpenes, diterpenes, triterpenoids, coenzyme, or combinations thereof. In any of the foregoing embodiments, the oil can include non-oil ingredients derived from and naturally present in the oil or oil source(s) and selected from the group consisting of α-tocopherol, β-tocopherol, δ-tocopherol, γ-tocopherol, α-tocotrienol, β-tocotrienol, δ-tocotrienol, and γ-tocotrienol. In any of the foregoing embodiments, the oil can include non-oil ingredients derived from and naturally present in the oil source(s) and selected from the group consisting of ceramide phosphate, monoglactodiacylglycerol, phosphatidylmethanol, sitosteryl ester, campesterol ester, sphingolipids, phosphatidyl glycerol, wax esters, sphingomyelin, and combinations thereof. It should be understood that the level of the non-oil ingredients can be the same as or greater than that in the oil source. By way of example, but not limitation, the non-oil components can be determined by LC/MS/MS analysis.

In any of the foregoing embodiments, the oil can further include monoacylglycerols (MAGs). By way of example, but not limitation, the MAGs can be present in the oil in an amount of at least 5%, at least 10%, at least 15%, at least 20%, at least 25%, at least 30%, at least 40%, at least 50%, at least 60%, at least 70%, at least 80%, at least 90%, about 5%, about 10%, about 15%, about 20%, about 25%, about 30%, about 40%, about 50%, about 60%, about 70%, about 75%, about 80%, about 90%, about 95%, about 98%, about 5% to about 98%, about 5% to about 95%, about 5% to about 90%, about 5% to about 80%, about 5% to about 75%, about 5% to about 70%, about 5% to about 60%, about 5% to about 50%, about 5% to about 40%, about 5% to about 30%, about 5% to about 25%, about 5% to about 20%, about 5% to about 10%, about 10% to about 98%, about 10% to about 95%, about 10% to about 90%, about 10% to about 80%, about 10% to about 75%, about 10% to about 70%, about 10% to about 60%, about 10% to about 50%, about 10% to about 40%, about 10% to about 30%, about 10% to about 25%, about 10% to about 20%, about 20% to about 98%, about 20% to about 95%, about 20% to about 90%, about 20% to about 80%, about 20% to about 75%, about 20% to about 70%, about 20% to about 60%, about 20% to about 50%, about 20% to about 40%, about 20% to about 30%, about 20% to about 25%, about 25% to about 98%, about 25% to about 95%, about 25% to about 90%, about 25% to about 80%, about 25% to about 75%, about 25% to about 70%, about 25% to about 60%, about 25% to about 50%, about 25% to about 40%, about 25% to about 30%, about 30% to about 98%, about 30% to about 95%, about 30% to about 90%, about 30% to about 80%, about 30% to about 75%, about 30% to about 70%, about 30% to about 60%, about 30% to about 50%, about 30% to about 40%, about 40% to about 98%, about 40% to about 95%, about 40% to about 90%, about 40% to about 80%, about 40% to about 75%, about 40% to about 70%, about 40% to about 60%, about 40% to about 50%, about 50% to about 98%, about 50% to about 95%, about 50% to about 90%, about 50% to about 80%, about 50% to about 75%, about 50% to about 70%, about 50% to about 60%, about 60% to about 98%, about 60% to about 95%, about 60% to about 90%, about 60% to about 80%, about 60% to about 75%, about 60% to about 70%, about 70% to about 98%, about 70% to about 95%, about 70% to about 90%, about 70% to about 80%, about 70% to about 75%, about 75% to about 98%, about 75% to about 95%, about 75% to about 90%, about 75% to about 80%, about 80% to about 98%, about 80% to about 95%, about 80% to about 90%, about 90% to about 98%, about 90% to about 95%, or about 95% to about 98% by weight out of the total weight of the oil. In any of the foregoing embodiments, the oil can further include diacylglycerols (DAGs). By way of example, but not limitation, the DAGs can be present in the oil in an amount of greater than about 5%, from about 5% to about 66%, about 10% to about 66%, about 20% to about 66%, about 30% to about 66%, about 30% to about 66%, about 40% to about 66%, about 50% to about 66%, about 5% to about 50%, about 10% to about 50%, about 20% to about 50%, about 30% to about 50%, about 40% to about 50%, about 5% to about 40%, about 10% to about 40%, about 20% to about 40%, about 30% to about 40%, about 5% to about 30%, about 10% to about 30%, about 20% to about 30%, about 5% to about 20%, about 10% to about 20%, about 5% to about 10%, or about 5%, 10%, 15%, 20%, 25%, 30%, 35%, 40%, 45%, 50%, 55%, 60%, or 66% by weight out of the total weight of the oil. Alternatively, in any of the foregoing embodiments, the oil can further include MAGs and DAGs. By way of example, but not limitation, the MAGs and DAGs can be present in the oil in an amount of at least 5%, at least 10%, at least 15%, at least 20%, at least 25%, at least 30%, at least 40%, at least 50%, at least 60%, at least 70%, at least 80%, at least 90%, about 5%, about 10%, about 15%, about 20%, about 25%, about 30%, about 40%, about 50%, about 60%, about 70%, about 75%, about 80%, about 90%, about 95%, about 98%, about 5% to about 98%, about 5% to about 95%, about 5% to about 90%, about 5% to about 80%, about 5% to about 75%, about 5% to about 70%, about 5% to about 60%, about 5% to about 50%, about 5% to about 40%, about 5% to about 30%, about 5% to about 25%, about 5% to about 20%, about 5% to about 10%, about 10% to about 98%, about 10% to about 95%, about 10% to about 90%, about 10% to about 80%, about 10% to about 75%, about 10% to about 70%, about 10% to about 60%, about 10% to about 50%, about 10% to about 40%, about 10% to about 30%, about 10% to about 25%, about 10% to about 20%, about 20% to about 98%, about 20% to about 95%, about 20% to about 90%, about 20% to about 80%, about 20% to about 75%, about 20% to about 70%, about 20% to about 60%, about 20% to about 50%, about 20% to about 40%, about 20% to about 30%, about 20% to about 25%, about 25% to about 98%, about 25% to about 95%, about 25% to about 90%, about 25% to about 80%, about 25% to about 75%, about 25% to about 70%, about 25% to about 60%, about 25% to about 50%, about 25% to about 40%, about 25% to about 30%, about 30% to about 98%, about 30% to about 95%, about 30% to about 90%, about 30% to about 80%, about 30% to about 75%, about 30% to about 70%, about 30% to about 60%, about 30% to about 50%, about 30% to about 40%, about 40% to about 98%, about 40% to about 95%, about 40% to about 90%, about 40% to about 80%, about 40% to about 75%, about 40% to about 70%, about 40% to about 60%, about 40% to about 50%, about 50% to about 98%, about 50% to about 95%, about 50% to about 90%, about 50% to about 80%, about 50% to about 75%, about 50% to about 70%, about 50% to about 60%, about 60% to about 98%, about 60% to about 95%, about 60% to about 90%, about 60% to about 80%, about 60% to about 75%, about 60% to about 70%, about 70% to about 98%, about 70% to about 95%, about 70% to about 90%, about 70% to about 80%, about 70% to about 75%, about 75% to about 98%, about 75% to about 95%, about 75% to about 90%, about 75% to about 80%, about 80% to about 98%, about 80% to about 95%, about 80% to about 90%, about 90% to about 98%, about 90% to about 95%, or about 95% to about 98% by weight out of the total weight of the oil.

In any of the foregoing embodiments, the FFA can be present as applicable based on the percent by weight of the MAGs and vice versa. In any of the foregoing embodiments, the combination of MAGs and FFAs can be from about 60% to about 95% by weight of the total weight of the oil. By way of example, but not limitation, the total combination of MAGs and FFAs can be from about 60% to about 95%, about 70% to about 95%, about 75% to about 95%, about 80% to about 95%, about 90% to about 95%, about 60% to about 90%, about 70% to about 90%, about 75% to about 90%, about 80% to about 90%, about 60% to about 80%, about 70% to about 80%, about 75% to about 80%, about 60% to about 75%, about 70% to about 75%, about 60% to about 70%, or about 60%, 65%, 70%, 75%, 80%, 85%, 90%, or 95% by weight out of the total weight of the oil.

In any of the foregoing embodiments, the MAGs in the oil can have a sn-1 substitution to sn-2 substitution ratio of greater than 1. By way of example but not limitation, the MAGs in the oil can have a sn-1 substitution to sn-2 substitution ratio of greater than 1, 1.1, 1.2, 1.3, 1.4, 1.5, 1.6, 1.7, 1.8, 1.9, 2, 2.25, 2.5, 2.75, 3, 3.25, 3.5, 3.75, 4, 4.25, 4.5, 4.75, 5, 6, 7, 8, 9, 10, 15, 20, 25, 30, 40, 50 or 100.

In any of the foregoing embodiments, the oil can have a fatty acid profile comprising two, three, four, five, six, seven, eight, nine, ten, eleven, or twelve of C6:0, C8:0, C10, C12, C14, C16, C18, C18:1, C18:2, C18:3, C20 and C22, wherein a range of each fatty is acid is between about 0.1% and 85% out of the total fatty acid content. By way of example, but not limitation, for each fatty acid, the amount can be from about 0.1% to about 85%, about 0.5% to about 85%, about 1% to about 85%, about 2% to about 85%, about 3% to about 85%, about 4% to about 85%, about 5% to about 85%, about 10% to about 85%, about 15% to about 85%, about 20% to about 85%, about 25% to about 85%, about 30% to about 85%, about 35% to about 85%, about 40% to about 85%, about 45% to about 85%, about 50% to about 85%, about 55% to about 85%, about 60% to about 85%, about 65% to about 85%, about 70% to about 85%, about 75% to about 85%, about 80% to about 85%, about 0.1% to about 80%, about 0.5% to about 80%, about 1% to about 80%, about 2% to about 80%, about 3% to about 80%, about 4% to about 80%, about 5% to about 80%, about 10% to about 80%, about 15% to about 80%, about 20% to about 80%, about 25% to about 80%, about 30% to about 80%, about 35% to about 80%, about 40% to about 80%, about 45% to about 80%, about 50% to about 80%, about 55% to about 80%, about 60% to about 80%, about 65% to about 80%, about 70% to about 80%, about 75% to about 80%, about 0.1% to about 75%, about 0.5% to about 75%, about 1% to about 75%, about 2% to about 75%, about 3% to about 75%, about 4% to about 75%, about 5% to about 75%, about 10% to about 75%, about 15% to about 75%, about 20% to about 75%, about 25% to about 75%, about 30% to about 75%, about 35% to about 75%, about 40% to about 75%, about 45% to about 75%, about 50% to about 75%, about 55% to about 75%, about 60% to about 75%, about 65% to about 75%, about 70% to about 75%, about 0.1% to about 70%, about 0.5% to about 70%, about 1% to about 70%, about 2% to about 70%, about 3% to about 70%, about 4% to about 70%, about 5% to about 70%, about 10% to about 70%, about 15% to about 70%, about 20% to about 70%, about 25% to about 70%, about 30% to about 70%, about 35% to about 70%, about 40% to about 70%, about 45% to about 70%, about 50% to about 70%, about 55% to about 70%, about 60% to about 70%, about 65% to about 70%, about 0.1% to about 65%, about 0.5% to about 65%, about 1% to about 65%, about 2% to about 65%, about 3% to about 65%, about 4% to about 65%, about 5% to about 65%, about 10% to about 65%, about 15% to about 65%, about 20% to about 65%, about 25% to about 65%, about 30% to about 65%, about 35% to about 65%, about 40% to about 65%, about 45% to about 65%, about 50% to about 65%, about 55% to about 65%, about 60% to about 65%, about 0.1% to about 60%, about 0.5% to about 60%, about 1% to about 60%, about 2% to about 60%, about 3% to about 60%, about 4% to about 60%, about 5% to about 60%, about 10% to about 60%, about 15% to about 60%, about 20% to about 60%, about 25% to about 60%, about 30% to about 60%, about 35% to about 60%, about 40% to about 60%, about 45% to about 60%, about 50% to about 60%, about 55% to about 60%, about 0.1% to about 55%, about 0.5% to about 55%, about 1% to about 55%, about 2% to about 55%, about 3% to about 55%, about 4% to about 55%, about 5% to about 55%, about 10% to about 55%, about 15% to about 55%, about 20% to about 55%, about 25% to about 55%, about 30% to about 55%, about 35% to about 55%, about 40% to about 55%, about 45% to about 55%, about 50% to about 55%, about 0.1% to about 50%, about 0.5% to about 50%, about 1% to about 50%, about 2% to about 50%, about 3% to about 50%, about 4% to about 50%, about 5% to about 50%, about 10% to about 50%, about 15% to about 50%, about 20% to about 50%, about 25% to about 50%, about 30% to about 50%, about 35% to about 50%, about 40% to about 50%, about 45% to about 50%, about 0.1% to about 45%, about 0.5% to about 45%, about 1% to about 45%, about 2% to about 45%, about 3% to about 45%, about 4% to about 45%, about 5% to about 45%, about 10% to about 45%, about 15% to about 45%, about 20% to about 45%, about 25% to about 45%, about 30% to about 45%, about 35% to about 45%, about 40% to about 45%, about 0.1% to about 40%, about 0.5% to about 40%, about 1% to about 40%, about 2% to about 40%, about 3% to about 40%, about 4% to about 40%, about 5% to about 40%, about 10% to about 40%, about 15% to about 40%, about 20% to about 40%, about 25% to about 40%, about 30% to about 40%, about 35% to about 40%, about 0.1% to about 35%, about 0.5% to about 35%, about 1% to about 35%, about 2% to about 35%, about 3% to about 35%, about 4% to about 35%, about 5% to about 35%, about 10% to about 35%, about 15% to about 35%, about 20% to about 35%, about 25% to about 35%, about 30% to about 35%, about 0.1% to about 30%, about 0.5% to about 30%, about 1% to about 30%, about 2% to about 30%, about 3% to about 30%, about 4% to about 30%, about 5% to about 30%, about 10% to about 30%, about 15% to about 30%, about 20% to about 30%, about 25% to about 30%, about 0.1% to about 25%, about 0.5% to about 25%, about 1% to about 25%, about 2% to about 25%, about 3% to about 25%, about 4% to about 25%, about 5% to about 25%, about 10% to about 25%, about 15% to about 25%, about 20% to about 25%, about 0.1% to about 20%, about 0.5% to about 20%, about 1% to about 20%, about 2% to about 20%, about 3% to about 20%, about 4% to about 20%, about 5% to about 20%, about 10% to about 20%, about 15% to about 20%, about 0.1% to about 15%, about 0.5% to about 15%, about 1% to about 15%, about 2% to about 15%, about 3% to about 15%, about 4% to about 15%, about 5% to about 15%, about 10% to about 15%, about 0.1% to about 10%, about 0.5% to about 10%, about 10% to about 10%, about 2% to about 10%, about 3% to about 10%, about 4% to about 10%, about 5% to about 10%, about 0.1% to about 5%, about 0.5% to about 5%, about 1% to about 5%, about 2% to about 5%, about 3% to about 5%, about 4% to about 5%, about 0.1% to about 4%, about 0.5% to about 4%, about 10% to about 4%, about 2% to about 4%, about 30% to about 4%, about 0.10% to about 3%, about 0.5% to about 3%, about 1% to about 3%, about 2% to about 3%, about 0.1% to about 2%, about 0.5% to about 2%, about 10% to about 2%, about 0.10% to about 10%, about 0.5% to about 10%, or about 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80% or 85% out of the total fatty acid content.

By way of example, but not limitation, where the oil is derived from flaxseed oil, the two or more different fatty acids can comprise two or more fatty acids from C18:1 in an amount from about 10% to about 20%, C18:2 in an amount from about 10% to about 20%, and C18:3 in an amount from about 50% to about 65%.

By way of example, but not limitation, where the oil is derived from MCT oil, the two or more different fatty acids can comprise two or more fatty acids from C8:0 in an amount from about 50% to about 65% out of the total fatty acid content of the oil and C10 in an amount from about 35% to about 50% out of the total fatty acid content of the oil.

By way of example, but not limitation, where the oil is derived from rosehip oil, the two or more different fatty acids can comprise two or more fatty acids from C18:1 in an amount from about 15% to about 25% out of the total fatty acid content of the oil, C18:2 in an amount from about 45% to about 55% out of the total fatty acid content of the oil, and C18:3 in an amount from about 15% to about 25% out of the total fatty acid content of the oil.

By way of example, but not limitation, where the oil is derived from sesame oil, the two or more different fatty acids can comprise two or more fatty acids from C18 in an amount from about 10% to about 20% out of the total fatty acid content, C18:1 in an amount from about 35% to about 45% out of the total fatty acid content, C18:2 in an amount from about 30% to about 45% out of the total fatty acid content of the oil.

By way of example, but not limitation, where the oil is derived from hemp seed oil, the two or more different fatty acids can comprise two or more fatty acids from C18:1 in an amount from about 5% to about 20% out of the total fatty acid content, C18:2 in an amount from about 50% to about 65% out of the total fatty acid content, C18:3 in an amount from about 15% to about 25% out of the total fatty acid content of the oil.

By way of example, but not limitation, where the oil is derived from sunflower oil, the two or more different fatty acids can comprise two or more fatty acids from C18:1 in an amount from about 15% to about 25% out of the total fatty acid content and C18:2 in an amount from about 60% to about 80% out of the total fatty acid content of the oil.

By way of example, but not limitation, where the oil is derived from canola oil, the two or more different fatty acids can comprise two or more fatty acids from C18:1 in an amount from about 55% to about 70% out of the total fatty acid content and C18:2 in an amount from about 15% to about 30% out of the total fatty acid content of the oil.

By way of example, but not limitation, where the oil is derived from almond oil, the two or more different fatty acids can comprise two or more fatty acids C18:1 in an amount from about 60% to about 80% out of the total fatty acid content and C18:2 in an amount from about 10% to about 25% out of the total fatty acid content of the oil.

By way of example, but not limitation, where the oil is derived from olive oil, the two or more different fatty acids can comprise two or more fatty acids from C16 in an amount from about 5% to about 20% out of the total fatty acid content, C18:1 in an amount from about 65% to about 80% out of the total fatty acid content, and C18:2 in an amount from about 5% to about 15% out of the total fatty acid content of the oil.

By way of example, but not limitation, where the oil is derived from oat oil, the two or more different fatty acids can comprise two or more fatty acids from C16 in an amount from about 5% to about 20% out of the total fatty acid content, C18:1 in an amount from about 15% to about 30% out of the total fatty acid content, and C18:2 in an amount from about 45% to about 60% out of the total fatty acid content of the oil.

The oils described herein, including those used to generate the EMOs, FFAs and nanoemulsions of the present disclosure, can be a MCT oil comprising from about 55-65% C8, 35-45% C10, and from about 0% to about 10% of a different chain length, but preferably 2% or less of other chain lengths.

In any of the foregoing embodiments, the droplets or particles can have an average zeta potential of less than about −30 mV. By way of example, but not limitation, the droplets or particles can have an average zeta potential of less than about −35 mV, less than about −40 mV, less than about −45 mV, less than about −50 mV, less than about −60 mV, less than about −70 mV, about −30 mV to about −80 mV, about −30 mV to about −75 mV, about −30 mV to about −70 mv, about −30 mV to about −60 mV, about −30 mV to about −50 mV, about −30 mV to about −40 mV, about −40 mV to about −80 mV, about −40 mV to about −75 mV, about −40 mV to about −70 mV, about −40 mV to about −60 mV, about −40 mV to about −50 mV, about −50 mV to about −80 mV, about −50 mV to about −75 mV, about −50 mV to about −70 mV, about −50 mV to about −60 mV, about −60 mV to about −80 mV, about −60 mV to about −75 mV, about −60 mV to about −70 mV, about −70 mV to about −80 mV, about −70 mV to about −75 mV, about −30 mV, −35 mV, −40 mV, −45 mV, −50 mV, −60 mV, −70 mV, −75 mV, or −90 mV. In certain aspects, the zeta potential can be as measured by electrophoretic light scattering.

In any of the foregoing embodiments, the droplets or particles can have a polydispersity index (PDI) of less than about 0.3. By way of example, but not limitation, the droplets or particles can have a PDI of less than about 0.3, less than about 0.25, less than about 0.2, less than about 0.175, about 0.15 to about 0.3, about 0.2 to about 0.3, about 0.25 to about 0.3, about 0.15, 0.16, 0.17, 0.18, 0.19, 0.2, 0.21, 0.22, 0.23, 0.24, 0.25, 0.26, 0.27, 0.28, 0.29 or 0.3. In certain aspects, the PDI can be as measured by dynamic light scattering.

In any of the foregoing embodiments, the droplets or particles can have a Z average size of about 300 nm or less. By way of example, but not limitation, the droplets or particles can have a Z average size of about 300 nm or less, about 275 nm or less, about 250 nm or less, about 225 nm or less, about 200 nm or less, about 175 nm or less, about 150 nm or less, about 100 nm to about 300 nm, about 100 nm to about 250 nm, about 100 nm to about 200 nm, about 150 nm to about 300 nm, about 150 nm to about 250 nm, about 150 nm to about 200 nm, about 200 nm to about 300 nm, about 200 nm to about 250 nm, about 250 nm to about 300 nm, about 150 nm, about 160 nm, about 170 nm, about 175 nm, about 180 nm, about 190 nm, about 200 nm, about 210 nm, about 220 nm, about 225 nm, about 230 nm, about 240 nm, about 250 nm, about 260 nm, about 270 nm, about 275 nm, about 280 nm, about 290 nm, or about 300 nm. In certain aspects, the Z average size can be as measured by dynamic light scattering.

In any of the foregoing embodiments, the oil can include a processed oil from an oil source that has been subjected to an enzymatic treatment selected from the group consisting of enzymatic hydrolysis to yield free fatty acids and glycerol with removal of glycerol to yield FFA oil, enzymatic re-esterification of FFA to yield monoacylglycerols (MAGs) and free fatty acids and, optionally, diacylglycerols (DAGs), and both enzymatic hydrolysis to yield free fatty acids and glycerol with removal of glycerol to yield FFA oil and enzymatic re-esterification of FFA to yield monoacylglycerols (MAGs) and free fatty acids and, optionally, diacylglycerols (DAGs). By way of example, but not limitation, the oil or a portion thereof can be an enzyme-modified oil (EMO) that has been subjected to enzymatic hydrolysis to yield free fatty acids and glycerol, separation of the glycerol from the free fatty acid oil, and subsequent enzymatic re-esterification to yield monoacylglycerols, free fatty acids and, optionally, diacylglcyerols. It should be understood that the oil so processed can be from a single oil source or a combination of oil sources which are subjected to the enzymatic treatment(s). It should be further understood that the EMO and/or FFA oil from a single or multiple oil sources can be blended after the enzymatic treatment(s) to formulate the oil in the compositions of the present disclosure. Thus, in some embodiments, the oil can include a blend of EMO and FFA oil from one, two, three, four, five or more oil sources. It should likewise be understood that EMOs and/or FFA oils can also be blended with unmodified, TAG oils to yield the oil in the compositions of the present disclosure. Thus, in some embodiments, the oil can include a blend of two or more processed oils, from each of a first oil source and a second oil source. By way of example, but not limitation, the oil can include a blend of two, three, four or more processed oils.

Thus, in some embodiments, the MAGs and, optionally, DAGs, or a portion thereof, in the composition can be derived from a first oil source. In further embodiments, the FFA, or a portion thereof, can be derived from a second oil source. In other embodiments, the FFA, or portion thereof, is derived from the same source as the first oil source. It should be understood that various combinations of MAGs, DAGs, TAGs and FFA from different oils can be used. In any of the foregoing embodiments, the oil can be derived from a single oil source, two oil sources, three oil sources, four oil sources, five oil sources or more.

In any of the foregoing embodiments, the MAGs can include two or more different MAGs. By way of example, but not limitation, the MAGs can include two, three, four, five or more different MAGs. By way of further example, but not limitation, the MAGs can be selected from the group consisting of monocaproin, monocaprylin, monocaprin, monolaurin, monomyristin, monopalmitin, monostearin, monoolein, monolinolein, monolinolenin, monoeicosapentaenoin, monodocosahexaenoin, and combinations thereof.

While the oil can be from a single oil source and type, such as, by way of example, but not limitation, EMO from a single oil source, as noted, the oil can include components from multiple oil sources. By way of example, but not limitation, the oil can include an EMO from a first oil source and a FFA oil from a second oil source. By way of further example, but not limitation, the oil can include a first EMO from a first oil source and a second EMO from a second oil source. By way of still further example, the oil can include a first EMO from a first oil source, a second EMO from a second oil source, and a FFA oil from a third oil source. Thus, it should be understood that any combination of oil components from oil sources, even unmodified TAG oils, that is used to form a nanoemulsion of the present disclosure is within the scope of the present disclosure. By way of even further example, where an EMO is used, the EMO can be derived from two or more oil sources. It should be understood that where the first oil source is EMO, at least a portion of the FFA in the composition can be derived from the EMO and the first oil source.

In any of the foregoing embodiments, the oil, or a portion thereof such as, by way of example, but not limitation, an EMO and/or a FFA oil, can be derived, from an oil source selected from a plant, an animal, a fish, an algal oil, and combinations thereof. In any of the foregoing embodiments, by way of example but not limitation, the oil can be derived from olive oil, almond oil, canola oil, coconut oil, cottonseed oil, palm kernel oil, palm olein oil, palm stearin oil, peanut oil, flaxseed oil, sunflower seed oil, corn oil, grapeseed oil, pomegranate oil, rose hip oil, hemp seed oil, prickly pear oil, medium chain triglyceride oil, safflower oil, sesame oil, walnut oil, palm oil, soybean oil, fish oil, sardine oil, anchovy oil, algal oil, chicken fat, lard, krill oil, avocado oil, mustard oil, rice bran oil, oat oil, nutmeg butter, macadamia nut oil, cacao butter, rapeseed oil, poppy seed oil, castor oil, edible oils, medicinal oils, and combinations thereof. By way of example, but not limitation, where a first and second oil source are utilized, these can be derived from olive oil, almond oil, canola oil, coconut oil, cottonseed oil, palm kernel oil, palm olein oil, palm stearin oil, peanut oil, flaxseed oil, sunflower seed oil, corn oil, grapeseed oil, pomegranate oil, rose hip oil, hemp seed oil, prickly pear oil, medium chain triglyceride oil, safflower oil, sesame oil, walnut oil, palm oil, soybean oil, fish oil, sardine oil, anchovy oil, algal oil, chicken fat, lard, krill oil, avocado oil, mustard oil, rice bran oil, oat oil, nutmeg butter, macadamia nut oil, cacao butter, rapeseed oil, poppy seed oil, castor oil, edible oils, medicinal oils, and combinations thereof. It should be further understood that the oil of the compositions of the present disclosure can include two, three, four or more oils from various source oils.

In some embodiments, the oil can include an unmodified, TAG oil component. The TAG oil component can be an oil selected from a plant, an animal, a fish, an algal oil, and combinations thereof. By way of example, but not limitation, the unmodified, TAG oil component can be olive oil, almond oil, canola oil, coconut oil, cottonseed oil, palm kernel oil, palm olein oil, palm stearin oil, peanut oil, flaxseed oil, sunflower seed oil, corn oil, grapeseed oil, pomegranate oil, rose hip oil, hemp seed oil, prickly pear oil, medium chain triglyceride oil, safflower oil, sesame oil, walnut oil, palm oil, soybean oil, fish oil, sardine oil, anchovy oil, algal oil, chicken fat, lard, krill oil, avocado oil, mustard oil, rice bran oil, oat oil, nutmeg butter, macadamia nut oil, cacao butter, rapeseed oil, poppy seed oil, castor oil, edible oils, medicinal oils, and combinations thereof. By way of example, but not limitation, where a first and second oil source are utilized, these can be derived from olive oil, almond oil, canola oil, coconut oil, cottonseed oil, palm kernel oil, palm olein oil, palm stearin oil, peanut oil, flaxseed oil, sunflower seed oil, corn oil, grapeseed oil, pomegranate oil, rose hip oil, hemp seed oil, prickly pear oil, medium chain triglyceride oil, safflower oil, sesame oil, walnut oil, palm oil, soybean oil, fish oil, sardine oil, anchovy oil, algal oil, chicken fat, lard, krill oil, avocado oil, mustard oil, rice bran oil, oat oil, nutmeg butter, macadamia nut oil, cacao butter, rapeseed oil, poppy seed oil, castor oil, edible oils, medicinal oils, and combinations thereof. It should be further understood that the oil of the compositions of the present disclosure can include two, three, four or more oils from various oil sources. In any of the foregoing embodiments, the TAG oil component can be present at up to 80% by weight of the oil. By way of example, but not limitation, the TAG oil component can be present at about 0.1% to about 80%, about 1% to about 80%, about 5% to about 80%, about 10% to about 80%, about 20% to about 80%, about 30% to about 80%, about 40% to about 80%, about 50% to about 80%, about 60% to about 80%, about 70% to about 80%, about 0.1% to about 70%, about 1% to about 70%, about 5% to about 70%, about 10% to about 70%, about 20% to about 70%, about 30% to about 70%, about 40% to about 70%, about 50% to about 70%, about 60% to about 70%, about 0.1% to about 60%, about 1% to about 60%, about 5% to about 60%, about 10% to about 60%, about 20% to about 60%, about 30% to about 60%, about 40% to about 60%, about 50% to about 60%, about 0.1% to about 50%, about 1% to about 50%, about 5% to about 50%, about 10% to about 50%, about 20% to about 50%, about 30% to about 50%, about 40% to about 50%, about 0.1% to about 40%, about 1% to about 40%, about 5% to about 40%, about 10% to about 40%, about 20% to about 40%, about 30% to about 40%, about 0.1% to about 30%, about 1% to about 30%, about 5% to about 30%, about 10% to about 30%, about 20% to about 30%, about 0.1% to about 20%, about 1% to about 20%, about 5% to about 20%, about 10% to about 20%, about 0.1% to about 10%, about 10% to about 10%, about 5% to about 10%, about 0.1% to about 5%, about 1% to about 5%, about 0.1% to about 10%, or about 0.10%, 10%, 2%, 3%, 4%, 5%, 10%, 15%, 20%, 25%, 30%, 40%, 50%, 60%, 70%, or 80% by weight of the oil.

In any of the foregoing embodiments, the oil can include a fatty acid profile that is within about 10% of the fatty acid profile from the oil source used to yield the oil for at least one, two or more fatty acids. In aspects where more than one oil source is used to form the oil, the fatty acid profile can be within about 10% of the fatty acid profile from the oil sources according to the respective amounts of each oil source used to form the oil for at least one, two or more fatty acids. By way of example, but not limitation, the fatty acid profile of the oil for one, two, three, four, five, six, seven, eight, nine, ten or more fatty acids can be within 10% of the fatty acid profile for the same fatty acids in the oil source(s), according to the respective amounts of each oil source used to form the oil if two or more oil sources are used, or for the oil source where only one oil source is used to form the oil. By way of further example, but not limitation, the fatty acid profile can be within about 10%, 9%, 8%, 7%, 6%, 5%, 4%, 3%, 2%, or 1% for each fatty acid in the oil as compared to the oil source(s). For example, if the first oil source has 15% C18 fatty acid, the EMO can have between about 5-25%, 6-24%, 7-23%, 8-22%, 9-21%, 10-20%, 11-19%, 12-18%, 13-17%, 1⁴-16% or about 15% C18 fatty acid. It should be understood that where the oil is a blend of EMO and FFA oil from different sources, the amounts of fatty acid will vary according to the ratio of the EMO and FFA oil and the starting fatty acid profile. Exemplary fatty acid profiles are provided in the Tables below which also include exemplary EMO compositions based on experimental data. It should be understood that for fatty acid profiles, amounts of fatty acid can naturally vary by about 3-5% depending on species, growing conditions and other variables It should be understood that the fatty acid profile that is expected can be estimated based on known fatty acid profiles and the amount of EMO and/or FFA oil in the oil.

| Fatty | Flaxseed | MCT | Rosehip | Sesame | Hempseed | Coconut |
|---|---|---|---|---|---|---|
| Acid | Oil | Oil | Oil | Oil | Oil | Oil* |
| C6:0 | 0.0 | 0.06 | 0.0 | 0.0 | 0.0 | 0.0 |
| C8:0 | 0.0 | 57.42 | 0.0 | 0.0 | 0.0 | 0.0 |
| C10 | 0.0 | 41.96 | 0.0 | 0.0 | 0.0 | 6.0 |
| C12 | 0.0 | 0.32 | 0.0 | 0.0 | 0.0 | 47.0 |
| C14 | 0.0 | 0.0 | 0.0 | 0.0 | 0.0 | 18.0 |
| C16 | 5.2 | 0.0 | 4.5 | 7.7 | 6.6 | 9.0 |
| C18 | 3.3 | 0.0 | 2.2 | 14.3 | 2.6 | 3.0 |
| C18:1 | 15.4 | 0.0 | 19.9 | 40.1 | 11.5 | 2.0 |
| C18:2 | 16.0 | 0.0 | 50.8 | 37.7 | 58.6 | 2.0 |
| C18:3 | 59.7 | 0.0 | 20.7 | 0.2 | 19.2 | 0.0 |
| C20 | 0.4 | 0.0 | 1.9 | 0.0 | 1.5 | 0.0 |
| C22 | 0.0 | 0.0 | 0.0 | 0.0 | 0.0 | 0.0 |
| | EMO | | | | | |
| MAG | 57.9 | est. 64 | 54.6 | 62.5 | 69.9 | est. 72 |
| DAG | 16.5 | est. 15 | 16.5 | 17.9 | 11 | est. 16 |
| TAG | BLD | BLD | BLD | BLD | BLD | BLD |
| FFA | 18 | 21 | 16 | 12 | 9 | 12 |

| Fatty | Fish | Algae | Oat | Canola | Olive | Almond |
|---|---|---|---|---|---|---|
| Acid | Oil* | Oil* | Oil | Oil | Oil* | Oil |
| C6:0 | 0.0 | 0.0 | 0.0 | 0.0 | 0.0 | 0.0 |
| C8:0 | 0.0 | 0.0 | 0.0 | 0.0 | 0.0 | 0.0 |
| C10 | 0.0 | 0.0 | 0.0 | 0.0 | 0.0 | 0.0 |
| C12 | 0.0 | 0.0 | 0.0 | 0.0 | 0.0 | 0.0 |
| C14 | 9.4 | 1.0 | 0.0 | 0.0 | 0.0 | 0.0 |
| C16 | 26.9 | 18.5 | 11.4 | 4.6 | 13.0 | 7.0 |
| C18 | 0.0 | 1.9 | 4.3 | 1.8 | 3.0 | 2.0 |
| C18:1 | 21.8 | 30.6 | 22.5 | 62.8 | 71.0 | 69.0 |
| C18:2 | 2.5 | 22.8 | 53.8 | 21.8 | 10.0 | 17.0 |
| C18:3 | 1.7 | 16.5 | 7.2 | 6.5 | 1.0 | 0.0 |
| C20 | 14.5 | 1.5 | 0.8 | 2.6 | 0.0 | 0.0 |
| C22 | 23.2 | 0.5 | 0.0 | 0.0 | 0.0 | 0.0 |
| | EMO | | | | | |
| MAG | est. 56 | | 65.9 | 60.9 | 55.9 | 74.9 |
| DAG | est. 14 | | 9.4 | 16.3 | 14.4 | 12.5 |
| TAG | 16 | | BLD | BLD | BLD | BLD |
| FFA | 12 | | 13 | 13.9 | 20 | 10 |

| Fatty | Cotton- | Peanut | Sunflower | Corn | Safflower | Soybean |
|---|---|---|---|---|---|---|
| Acid | seed Oil* | Oil* | Oil* | Oil* | Oil* | Oil* |
| C6:0 | 0.0 | 0.0 | 0.0 | 0.0 | 0.0 | 0.0 |
| C8:0 | 0.0 | 0.0 | 0.0 | 0.0 | 0.0 | 0.0 |
| C10 | 0.0 | 0.0 | 0.0 | 0.0 | 0.0 | 0.0 |
| C12 | 0.0 | 0.0 | 0.0 | 0.0 | 0.0 | 0.0 |
| C14 | 1.0 | 0.0 | 0.0 | 0.0 | 0.0 | 0.0 |
| C16 | 22.0 | 11.0 | 7.0 | 11.0 | 7.0 | 11.0 |
| C18 | 3.0 | 2.0 | 5.0 | 2.0 | 2.0 | 4.0 |
| C18:1 | 19.0 | 48.0 | 19.0 | 28.0 | 13.0 | 24.0 |
| C18:2 | 54.0 | 32.0 | 68.0 | 58.0 | 78.0 | 54.0 |
| C18:3 | 1.0 | 0.0 | 1.0 | 1.0 | 0.0 | 7.0 |
| C20 | 0.0 | 0.0 | 0.0 | 0.0 | 0.0 | 0.0 |
| C22 | 0.0 | 0.0 | 0.0 | 0.0 | 0.0 | 0.0 |
| | EMO | | | | | |
| MAG | | | 58.3 | | | |
| DAG | | | 25.5 | | | |
| TAG | BLD | | | | | |
| FFA | | | 9 | | | |

| Fatty | Cacao | Grapeseed | Palm | Palm | Palm Kernel | Shea | Walnut | Macadamia |
|---|---|---|---|---|---|---|---|---|
| Acid | Oil* | Oil* | Oil* | Olein* | Oil* | Oil* | Oil* | Nut Oil |
| C6:0 | 0.0 | 0.0 | 0.0 | 0.0 | 0.0 | 0.0 | 0.0 | 0.0 |
| C8:0 | 0.0 | 0.0 | 0.0 | 0.0 | 0.0 | 0.0 | 0.0 | 0.0 |
| C10 | 0.0 | 0.0 | 0.0 | 0.0 | 4.0 | 0.0 | 0.0 | 0.0 |
| C12 | 0.0 | 0.0 | 0.0 | 0.0 | 48.0 | 1.0 | 0.0 | 0.0 |
| C14 | 0.0 | 0.0 | 1.0 | 1.0 | 16.0 | 0.0 | 0.0 | 1.2 |
| C16 | 25.0 | 8.0 | 45.0 | 37.0 | 8.0 | 4.0 | 11.0 | 41 |
| C18 | 38.0 | 4.0 | 4.0 | 4.0 | 3.0 | 39.0 | 5.0 | 5.4 |
| C18:1 | 32.0 | 15.0 | 40.0 | 46.0 | 15.0 | 44.0 | 28.0 | 41 |
| C18:2 | 3.0 | 73.0 | 10.0 | 11.0 | 2.0 | 5.0 | 51.0 | 2.7 |
| C18:3 | 0.0 | 0.0 | 0.0 | 0.0 | 0.0 | 0.0 | 5.0 | 1 |
| C20 | 0.0 | 0.0 | 0.0 | 0.0 | 0.0 | 0.0 | 0.0 | 6.5 |
| C22 | 0.0 | 0.0 | 0.0 | 0.0 | 0.0 | 0.0 | 0.0 | 1.1 |
| | EMO | | | | | | | |
| MAG | | | | | | | | |
| DAG | | | | | | | | |
| TAG | | | | | | | | |
| FFA | | | | | | | | |

*Fatty acid profile values derived from literature; BLD=below limit of detection

In any of the foregoing embodiments, the oil can be about 0.00100 to about 900% by weight out of the total weight of the composition. By way of example, but not limitation, the oil can be about 0.0010% to about 90%, about 0.010% to about 90%, about 0.10% to about 900%, about 100 to about 900%, about 20% to about 900%, about 300 to about 900%, about 4% to about 90%, about 500 to about 90%, about 1000 to about 90%, about 20% to about 9000, about 3000 to about 9000, about 4000 to about 9000, about 5000 to about 9000, about 60% to about 90%, about 70% to about 90%, about 80% to about 90%, about 0.0010% to about 80%, about 0.0100 to about 80%, about 0.100 to about 80%, about 100 to about 80%, about 20% to about 800%, about 300 to about 800%, about 400 to about 800%, about 500 to about 80%, about 10% to about 80%, about 20% to about 80%, about 30% to about 80%, about 400% to about 800%, about 500% to about 800%, about 600% to about 800%, about 700% to about 80%, about 0.00100 to about 70%, about 0.010% to about 70%, about 10% to about 70%, about 20% to about 700%, about 300 to about 700%, about 400 to about 700%, about 500 to about 700%, about 10% to about 70%, about 20% to about 70%, about 30% to about 70%, about 40% to about 70%, about 50% to about 70%, about 60% to about 70%, about 0.001% to about 60%, about 0.01% to about 60%, about 0.1% to about 60%, about 1% to about 60%, about 2% to about 60%, about 3% to about 60%, about 4% to about 60%, about 5% to about 60%, about 10% to about 60%, about 20% to about 60%, about 30% to about 60%, about 40% to about 60%, about 50% to about 60%, about 0.001% to about 50%, about 0.01% to about 50%, about 0.1% to about 50%, about 1% to about 50%, about 2% to about 50%, about 3% to about 50$, about 4% to about 50%, about 5% to about 50%, about 10% to about 50%, about 20% to about 50%, about 30% to about 50%, about 30% to about 50%, about 0.001% to about 40%, about 0.01% to about 40%, about 0.1% to about 40%, about 1% to about 40%, about 2% to about 40%, about 3% to about 40%, about 4% to about 40%, about 5% to about 40%, about 10% to about 40%, about 20% to about 40%, about 30% to about 40%, about 0.001% to about 30%, about 0.01% to about 30%, about 0.1% to about 30%, about 1% to about 30%, about 2% to about 30%, about 3% to about 30%, about 4% to about 30%, about 5% to about 30%, about 10% to about 30%, about 20% to about 30%, about 0.001% to about 20%, about 0.01% to about 20%, about 0.1% to about 20%, about 1% to about 20%, about 2% to about 20%, about 3% to about 20%, about 4% to about 20%, about 5% to about 20%, about 10% to about 20%, about 0.001% to about 15%, about 0.01% to about 15%, about 0.1% to about 15%, about 1% to about 15%, about 2% to about 15%, about 3% to about 15%, about 4% to about 15%, about 5% to about 15%, about 6% to about 15%, about 7% to about 15%, about 8% to about 15%, about 9% to about 5%, about 10% to about 15%, about 11% to about 15%, about 12% to about 15%, about 13% to about 15%, about 14% to about 15%, about 0.001% to about 10%, about 0.01% to about 10%, about 0.1% to about 10%, about 1% to about 10%, about 2% to about 10%, about 3% to about 10%, about 4% to about 10%, about 5% to about 10%, about 0.001% to about 5%, about 0.01% to about 5%, about 0.1% to about 5%, about 1% to about 5%, about 2% to about 10%, about 2% to about 5%, about 3% to about 10%, about 3% to about 5%, about 4% to about 10%, about 4% to about 5%, about 1%, 2%, 3%, 4%, 5%, 6%, 7%, 8%, 9%, 10, 1%, 20%, 25%, 30%, 40%, 50%, 60%, 70%, 75%, 80%, or 90% by weight out of the total weight of the composition.

In any of the foregoing embodiments, the liquid can be water or any suitable polar medium. By way of example, but not limitation, the polar medium can be water, deionized water, saline, buffered water (e.g., phosphate buffered saline (PBS), citric acid buffer, sodium acetate buffer), salt solutions (e.g., sodium chloride or potassium chloride) and other non-aqueous solutions.

In any of the foregoing embodiments, the Z average size of the droplets or particles does not change by more than 15% over at least 14 days when stored at room temperature (22° C.). By way of example, but not limitation, the Z average size of the droplets or particles does not change by more than 15% over at least 14 days, 21 days, or 28 days when stored at room temperature (22° C.). By way of further example, but not limitation, the Z average size of the droplets or particles does not change by more than 15%, by more than 10%, or by more than 5% over 14 days, 21 days or 28 days when stored at room temperature (22° C.).

In any of the foregoing embodiments, the Z average size of the droplets or particles does not change by more than 15% over at least 14 days when stored at 4° C. By way of example, but not limitation, the Z average size of the droplets or particles does not change by more than 15% over at least 14 days, 21 days, or 28 days when stored at 4° C. By way of further example, but not limitation, the Z average size of the droplets or particles does not change by more than 15%, by more than 10%, or by more than 5% over 14 days, 21 days or 28 days when stored at 4° C.

In any of the foregoing embodiments, the oil can be in the form of droplets. Alternatively, in any of the foregoing embodiments, the oil can be in the form of particles. For example, where the nanoemulsion is a solid lipid nanoemulsion (SLN), the oil would be in the form of particles. It should be understood that droplets refers to liquid droplets, while particles refers to solid particles.

In any of the foregoing embodiments, the composition can not include any exogenous additive with surfactant and/or emulsifier properties that is not naturally present in the oil in an amount sufficient to enhance droplet or particle stability. By way of example, but not limitation, the composition can not include polysorbates, phospholipids, sterols, cationic lipids, poloxamers, sorbitan esters, sugar esters, polyoxyethylene conjugates, ammonium phosphatidyl lapsidate, polyphenols, polyvinylalcohol, lecithin, ethanol, lysophospholipids, ceramides, Tweens, Spans, Kolliphor, propylene glycol, vitamin D, polyethylene glycol, polyethylene glycol derivatives, gum acacia, modified gum acacia, agar, ghatti gum, modified ghatti gum, pectin, carrageenan, xanthan gum, modified starches, modified alginate, fatty alcohols, ethoxylated polyols, and combinations thereof.

In any of the foregoing embodiments, the composition can not include any exogenous additive with thickening or crystallization inhibiting properties that is not naturally present in the oil in an amount sufficient to thicken the composition or inhibit crystal formation, respectively. By way of example, but not limitation, the composition can not include methyl cellulose, cornstarch, sodium alginate, or gelatin. By way of further example, but not limitation, the composition can not include polyglycerol fatty acid esters, sucrose fatty acid esters, sorbitan fatty acid esters, and combinations thereof. In certain aspects, the composition can not include any exogenous additive with thickening properties that is not naturally present in the oil in an amount sufficient to thicken the composition. In certain aspects, the composition can not include any exogenous additive with crystallization inhibiting properties that is not naturally present in the oil in an amount sufficient to inhibit crystal formation. In other aspects, the composition can not include any exogenous additive with thickening or crystallization inhibiting properties that is not naturally present in the oil in an amount sufficient to thicken the composition or inhibit crystal formation, respectively.

In any of the foregoing embodiments, the EMO, FFA oil and/or oil can be substantially free of 3-monochloropanediol and glycidol equivalents.

In any of the foregoing embodiments, the composition can not include a quaternary amine.

In any of the foregoing embodiments, the composition can not include a polyhydric alcohol.

In any of the foregoing embodiments, the composition can not include bile salts.

In any of the foregoing embodiments, the droplets can be mixed micelles. Alternatively, in any of the foregoing embodiments, the particles can be solid lipid particles.

In some embodiments, a method for preparing a nanoemulsion is provided that includes providing an oil of the present disclosure, providing a polar liquid component, combining the oil and polar liquid component to form a nanoemulsion pre-mix, and forming a nanoemulsion from the nanoemulsion pre-mix, where the nanoemulsion comprises an oil phase and an polar, liquid phase, where the oil phase is dispersed as droplets or particles within the polar, liquid phase, and where the droplets or particles have a Z average size of about 300 nm or less as measured by dynamic light scattering. In certain aspects, the oil can be heated, for example to 60° C., prior to combining with the polar, liquid component which itself can be pre-heated, for example to 60° C. By way of further example, but not limitation, the oil can be heated to about 40° C. to about 80° C., to about 40° C. to about 70° C., to about about 50° C. to about 80° C., to about 50° C. to about 70° C., to about 55° C. to about 65° C., to about 40° C., 45° C., 50° C., 55° C., 60° C., 65° C., 70° C., 75° C. or 80° C. By way of still further example, but not limitation, the polar liquid can be pre-heated to about 40° C. to about 80° C., to about 40° C. to about 70° C., to about 50° C. to about 80° C., to about 50° C. to about 70° C., to about 55° C. to about 65° C., to about 40° C., 45° C., 50° C., 55° C., 60° C., 65° C., 70° C., 75° C. or 80° C.

In any of the foregoing method embodiments, the nanoemulsion can be formed under conditions sufficient to have the properties of any of the compositions of the foregoing embodiments.

In any of the foregoing method embodiments, the method can further include a step of forming the oil by blending one or more oil components. In some embodiments, the one or more oil components are derived from different oil sources or combinations thereof as described in the present disclosure. In some embodiments, the one or more oil components are derived from the same oil source. By way of example, but not limitation, the one or more oil components can be selected from an EMO, a FFA oil, an unmodified, TAG oil, and combinations thereof.

It should be understood that the polar, liquid medium can be any suitable polar, liquid medium, including those disclosed in the present disclosure with respect to compositions of the present disclosure.

By way of example, but not limitation, the nanoemulsion can be formed by adding the polar, liquid phase rapidly to the oil phase or by homogenization after combining to form a coarse emulsion and sonicating the coarse emulsion to form the nanoemulsion. By way of further example, but not limitation, the nanoemulsion can be formed by adding the oil dropwise to the polar, liquid phase to form a coarse emulsion and sonicating the coarse emulsion to form the nanoemulsion.

It should be understood that other methods for manufacturing nanoemulsions are well-known and can be used by one of skill in the art to prepare nanoemulsions of the present disclosure. Such methods include, but are not limited to, high shear homogenization, microfluidization, bath sonication, solvent evaporation, supercritical fluid methods, spray drying methods and double emulsion methods.

Microemulsions and Larger Nanoemulsions

It should also be understood that the compositions of the present disclosure, although disclosed as being nanoemulsions, can also be formulated as larger nanoemulsions or microemulsions depending on the application. In any of the foregoing embodiments, rather than the Z average size of the droplets or particles being about 300 nm or less, the droplets or particles can have a Z average size of greater than about 300 nm or greater than about 1000 nm. By way of example, but not limitation, in addition to the size of the nanoemulsion compositions already disclosed, the droplets or particles can have a Z average size of greater than about 300 nm, greater than about 400 nm, greater than about 500 nm, greater than about 600 nm, greater than about 700 nm, greater than about 800 nm, greater than about 900 nm, greater than about 1000 nm, greater than about 10000 nm, greater than about 100000 nm, from about 300 nm to about 1000 nm, from about 500 nm to about 1000 nm, from about 750 to about 1000 nm, from about 1000 nm to about 999999 nm, from about 1000 nm to about 100000 nm, from about 1000 nm to about 10000 nm, from about 10000 to about 5000 nm, from about 1000 to about 3000 nm, about 300 nm, about 400 nm, about 500 nm, about 600 nm, about 700 nm, about 800 nm, about 900 nm, about 1000 nm, about 2000 nm, about 3000 nm, about 4000 nm, about 5000 nm, about 10000 nm, about 20000 nm, about 30000 nm, about 40000 nm, about 50000 nm, about 100000 nm, or about 1000000 nm. By way of further example, but not limitation, for certain active ingredient delivery applications, a microemulsion may be used instead of a nanoemulsion. By way of example, but not limitation, the size of microemulsions and larger nanoemulsions can be measured by microscopy or dynamic light scattering.

Methods of making microemulsions and larger nanoemulsions are well-known in the art and include, by way of example, but not limitation, homogenization, sonication, solvent evaporation, supercritical fluid methods, spray drying methods and double emulsion methods. By way of further example, but not limitation, the step of forming coarse nanoemulsions by homogenization disclosed in the present application can be used to form microemulsions and larger nanoemulsions. In methods of the present disclosure for forming nanoemulsions, the methods can be modified to form microemulsions by using conventional methods for forming microemulsions instead of forming a nanoemulsion. It should be understood that similar methods can be used to obtain larger nanoemulsions if desired. It should also be understood that methods for preparing nanoemulsions of the present disclosure can be used to the extent that they produce larger nanoemulsions or microemulsions. By way of example, but not limitation, to form a larger nanoemulsion or microemulsion, the oil can optionally be heated first and then combined with a polar liquid and subjected to homogenization. By way of further example, but not limitation, the polar liquid can be pre-heated prior to combining with the oil.

Non-Nanoemulsion Compositions

It should be understood that in compositions, including the nanoemulsions and microemulsions disclosed, that the oil can be either an EMO, a FFA oil or a combination of both. It should be further understood that compositions of the present disclosure encompass any of the foregoing embodiments where the composition has any of the properties recited but where it does not have the droplet or particles and is an oil composition. Thus, the only properties which would differ in such embodiments are that the composition is not a nanoemulsion or microemulsion and does not have the size, zeta potential, PDI or stability characteristics recited. It should likewise be understood that, in such embodiments, the composition may include the oil but does not necessarily require a liquid as disclosed in certain nanoemulsion embodiments.

In some embodiments, an oil composition is provided which includes from about 10% to about 80% by weight monoacylglycerols (MAGs) by weight of the oil and free fatty acids in an amount of 5% to about 75% by weight of the oil, where the combination of the MAGs and free fatty acids is from about 60% to about 95% by weight out of the total weight of the oil, where the free fatty acids comprise two or more different fatty acids and where the oil is substantially free of triacylglycerols (TAGs). By way of example, but not limitation, the oil composition can include from about 10% to about 80%, about 15% to about 80%, about 20% to about 80%, about 25% to about 80%, about 30% to about 80%, about 40% to about 80%, about 50% to about 80%, about 60% to about 80%, about 70% to about 80%, about 75% to about 80%, about 10% to about 75%, about 15% to about 75%, about 20% to about 75%, about 25% to about 75%, about 30% to about 75%, about 40% to about 75%, about 50% to about 75%, about 60% to about 75%, about 70% to about 75%, about 10% to about 70%, about 15% to about 70%, about 20% to about 70% about 25% to about 70%, about 30% to about 70%, about 40% to about 70%, about 50% to about 70%, about 60% to about 70%, about 10% to about 60%, about 15% to about 60%, about 20% to about 60%, about 25% to about 60%, about 30% to about 60%, about 40% to about 60%, about 50% to about 60%, about 10% to about 50%, about 15% to about 50%, about 20% to about 50%, about 25% to about 50%, about 30% to about 50%, about 40% to about 50%, about 10% to about 40%, about 15% to about 40%, about 20% to about 40%, about 25% to about 40%, about 30% to about 40%, about 10% to about 30%, about 15%, to about 30%, about 20% to about 30%, about 25% to about 30%, about 10% to about 25%, about 15% to about 25%, about 20% to about 25%, about 10% to about 20%, about 15% to about 20%, about 10% to about 15%, or about 5%, 10%, 15%, 20%, 25%, 30%, 40%, 50%, 60%, 70%, 75%, or 80% MAGs by weight out of the total weight of the oil. By way of example, but not limitation, the free fatty acids can be present in an amount of from about 5% to about 75%, about 10% to about 75%, about 15% to about 75%, about 20% to about 75%, about 25% to about 75%, about 30% to about 75%, about 40% to about 75%, about 50% to about 75%, about 60% to about 75%, about 70% to about 75%, about 10% to about 70%, about 15% to about 75%, about 20% to about 75%, about 30% to about 75%, about 40% to about 75%, about 50% to about 75%, about 60% to about 75%, about 70% to about 75%, about 10% to about 70%, about 15% to about 70%, about 20% to about 70% about 25% to about 70%, about 30% to about 70%, about 40% to about 70%, about 50% to about 70%, about 60% to about 70%, about 10% to about 60%, about 15% to about 60%, about 20% to about 60%, about 25% to about 60%, about 30% to about 60%, about 40% to about 60%, about 50% to about 60%, about 10% to about 50%, about 15% to about 50%, about 20% to about 50%, about 25% to about 50%, about 30% to about 50%, about 40% to about 50%, about 10% to about 40%, about 15% to about 40%, about 20% to about 40%, about 25% to about 40%, about 30% to about 40%, about 10% to about 30%, about 15%, to about 30%, about 20% to about 30%, about 25% to about 30%, about 10% to about 25%, about 15% to about 25%, about 20% to about 25%, about 10% to about 20%, about 15% to about 20%, about 10% to about 15%, or about 5%10%, 15%, 20%, 25%, 30%, 40%, 50%, 60%, 70%, or 75% FFA by weight out of the total weight of the oil. By way of example, but not limitation, the combined amount of the MAGs and FFA can be from about 60% to about 95%, about 70% to about 95%, about 75% to about 95%, about 80% to about 95%, about 90% to about 95%, about 60% to about 90%, about 70% to about 90%, about 75% to about 90%, about 80% to about 90%, about 60% to about 80%, about 70% to about 80%, about 75% to about 80%, about 60% to about 75%, about 70% to about 75%, about 60% to about 70%, or 60%, 65%, 70%, 75%, 80%, 85%, 90%, or 95% by weight out of the total weight of the oil.

In any of the foregoing embodiments, the oil composition can be a mixture of EMO and FFA oil. In other embodiments, the oil composition is an EMO. In still other embodiments, the oil composition is a FFA oil.

In any of the foregoing embodiments, the oil can have a fatty acid profile comprising two, three, four, five, six, seven, eight, nine, ten, eleven, or twelve of C6:0, C8:0, C10, C12, C14, C16, C18, C18:1, C18:2, C18:3, C20 and C22, wherein a range of each fatty is acid is between about 0.1% and 85% out of the total fatty acid content. By way of example, but not limitation, for each fatty acid, the amount can be from about 0.1% to about 85%, about 0.5% to about 85%, about 1% to about 85%, about 2% to about 85%, about 3% to about 85%, about 4% to about 85%, about 5% to about 85%, about 10% to about 85%, about 15% to about 85%, about 20% to about 85%, about 25% to about 85%, about 30% to about 85%, about 35% to about 85%, about 40% to about 85%, about 45% to about 85%, about 50% to about 85%, about 55% to about 85%, about 60% to about 85%, about 65% to about 85%, about 70% to about 85%, about 75% to about 85%, about 80% to about 85%, about 0.1% to about 80%, about 0.5% to about 80%, about 1% to about 80%, about 2% to about 80%, about 3% to about 80%, about 4% to about 80%, about 5% to about 80%, about 10% to about 80%, about 15% to about 80%, about 20% to about 80%, about 25% to about 80%, about 30% to about 80%, about 35% to about 80%, about 40% to about 80%, about 45% to about 80%, about 50% to about 80%, about 55% to about 80%, about 60% to about 80%, about 65% to about 80%, about 70% to about 80%, about 75% to about 80%, about 0.1% to about 75%, about 0.5% to about 75%, about 1% to about 75%, about 2% to about 75%, about 3% to about 75%, about 4% to about 75%, about 5% to about 75%, about 10% to about 75%, about 15% to about 75%, about 20% to about 75%, about 25% to about 75%, about 30% to about 75%, about 35% to about 75%, about 40% to about 75%, about 45% to about 75%, about 50% to about 75%, about 55% to about 75%, about 60% to about 75%, about 65% to about 75%, about 70% to about 75%, about 0.1% to about 70%, about 0.5% to about 70%, about 1% to about 70%, about 2% to about 70%, about 3% to about 70%, about 4% to about 70%, about 5% to about 70%, about 10% to about 70%, about 15% to about 70%, about 20% to about 70%, about 25% to about 70%, about 30% to about 70%, about 35% to about 70%, about 40% to about 70%, about 45% to about 70%, about 50% to about 70%, about 55% to about 70%, about 60% to about 70%, about 65% to about 70%, about 0.1% to about 65%, about 0.5% to about 65%, about 1% to about 65%, about 2% to about 65%, about 3% to about 65%, about 4% to about 65%, about 5% to about 65%, about 10% to about 65%, about 15% to about 65%, about 20% to about 65%, about 25% to about 65%, about 30% to about 65%, about 35% to about 65%, about 40% to about 65%, about 45% to about 65%, about 50% to about 65%, about 55% to about 65%, about 60% to about 65%, about 0.1% to about 60%, about 0.5% to about 60%, about 1% to about 60%, about 2% to about 60%, about 3% to about 60%, about 4% to about 60%, about 5% to about 60%, about 10% to about 60%, about 15% to about 60%, about 20% to about 60%, about 25% to about 60%, about 30% to about 60%, about 35% to about 60%, about 40% to about 60%, about 45% to about 60%, about 50% to about 60%, about 55% to about 60%, about 0.1% to about 55%, about 0.5% to about 55%, about 1% to about 55%, about 2% to about 55%, about 3% to about 55%, about 4% to about 55%, about 5% to about 55%, about 10% to about 55%, about 15% to about 55%, about 20% to about 55%, about 25% to about 55%, about 30% to about 55%, about 35% to about 55%, about 40% to about 55%, about 45% to about 55%, about 50% to about 55%, about 0.1% to about 50%, about 0.5% to about 50%, about 1% to about 50%, about 2% to about 50%, about 3% to about 50%, about 4% to about 50%, about 5% to about 50%, about 10% to about 50%, about 15% to about 50%, about 20% to about 50%, about 25% to about 50%, about 30% to about 50%, about 35% to about 50%, about 40% to about 50%, about 45% to about 50%, about 0.1% to about 45%, about 0.5% to about 45%, about 1% to about 45%, about 2% to about 45%, about 3% to about 45%, about 4% to about 45%, about 5% to about 45%, about 10% to about 45%, about 15% to about 45%, about 20% to about 45%, about 25% to about 45%, about 30% to about 45%, about 35% to about 45%, about 40% to about 45%, about 0.1% to about 40%, about 0.5% to about 40%, about 1% to about 40%, about 2% to about 40%, about 3% to about 40%, about 4% to about 40%, about 5% to about 40%, about 10% to about 40%, about 15% to about 40%, about 20% to about 40%, about 25% to about 40%, about 30% to about 40%, about 35% to about 40%, about 0.1% to about 35%, about 0.5% to about 35%, about 1% to about 35%, about 2% to about 35%, about 3% to about 35%, about 4% to about 35%, about 5% to about 35%, about 10% to about 35%, about 15% to about 35%, about 20% to about 35%, about 25% to about 35%, about 30% to about 35%, about 0.1% to about 30%, about 0.5% to about 30%, about 1% to about 30%, about 2% to about 30%, about 3% to about 30%, about 4% to about 30%, about 5% to about 30%, about 10% to about 30%, about 15% to about 30%, about 20% to about 30%, about 25% to about 30%, about 0.1% to about 25%, about 0.5% to about 25%, about 1% to about 25%, about 2% to about 25%, about 3% to about 25%, about 4% to about 25%, about 5% to about 25%, about 10% to about 25%, about 15% to about 25%, about 20% to about 25%, about 0.1% to about 20%, about 0.5% to about 20%, about 1% to about 20%, about 2% to about 20%, about 3% to about 20%, about 4% to about 20%, about 5% to about 20%, about 10% to about 20%, about 15% to about 20%, about 0.1% to about 15%, about 0.5% to about 15%, about 1% to about 15%, about 2% to about 15%, about 3% to about 15%, about 4% to about 15%, about 5% to about 15%, about 10% to about 15%, about 0.1% to about 10%, about 0.5% to about 10%, about 10% to about 10%, about 2% to about 10%, about 3% to about 10%, about 4% to about 10%, about 5% to about 10%, about 0.1% to about 5%, about 0.5% to about 5%, about 1% to about 5%, about 2% to about 5%, about 3% to about 5%, about 4% to about 5%, about 0.1% to about 4%, about 0.5% to about 4%, about 1% to about 4%, about 2% to about 4%, about 3% to about 4%, about 0.10% to about 3%, about 0.5% to about 3%, about 1% to about 3%, about 2% to about 3%, about 0.1% to about 2%, about 0.5% to about 2%, about 1% to about 2%, about 0.10% to about 1%, about 0.5% to about 1%, or about 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80% or 85% out of the total fatty acid content.

By way of example, but not limitation, where the oil is derived from flaxseed oil, the two or more different fatty acids can comprise two or more fatty acids from C18:1 in an amount from about 10% to about 20%, C18:2 in an amount from about 10% to about 20%, and C18:3 in an amount from about 50% to about 65%.

By way of example, but not limitation, where the oil is derived from MCT oil, the two or more different fatty acids can comprise two or more fatty acids from C8:0 in an amount from about 50% to about 65% out of the total fatty acid content of the oil and C10 in an amount from about 35% to about 50% out of the total fatty acid content of the oil.

By way of example, but not limitation, where the oil is derived from rosehip oil, the two or more different fatty acids can comprise two or more fatty acids from C18:1 in an amount from about 15% to about 25% out of the total fatty acid content of the oil, C18:2 in an amount from about 45% to about 55% out of the total fatty acid content of the oil, and C18:3 in an amount from about 15% to about 25% out of the total fatty acid content of the oil.

By way of example, but not limitation, where the oil is derived from sesame oil, the two or more different fatty acids can comprise two or more fatty acids from C18 in an amount from about 10% to about 20% out of the total fatty acid content, C18:1 in an amount from about 35% to about 45% out of the total fatty acid content, C18:2 in an amount from about 30% to about 45% out of the total fatty acid content of the oil.

By way of example, but not limitation, where the oil is derived from hemp seed oil, the two or more different fatty acids can comprise two or more fatty acids from C18:1 in an amount from about 5% to about 20% out of the total fatty acid content, C18:2 in an amount from about 50% to about 65% out of the total fatty acid content, C18:3 in an amount from about 15% to about 25% out of the total fatty acid content of the oil.

By way of example, but not limitation, where the oil is derived from sunflower oil, the two or more different fatty acids can comprise two or more fatty acids from C18:1 in an amount from about 15% to about 25% out of the total fatty acid content and C18:2 in an amount from about 60% to about 80% out of the total fatty acid content of the oil.

By way of example, but not limitation, where the oil is derived from canola oil, the two or more different fatty acids can comprise two or more fatty acids from C18:1 in an amount from about 55% to about 70% out of the total fatty acid content and C18:2 in an amount from about 15% to about 30% out of the total fatty acid content of the oil.

By way of example, but not limitation, where the oil is derived from almond oil, the two or more different fatty acids can comprise two or more fatty acids C18:1 in an amount from about 60% to about 80% out of the total fatty acid content and C18:2 in an amount from about 10% to about 25% out of the total fatty acid content of the oil.

By way of example, but not limitation, where the oil is derived from olive oil, the two or more different fatty acids can comprise two or more fatty acids from C16 in an amount from about 5% to about 20% out of the total fatty acid content, C18:1 in an amount from about 65% to about 80% out of the total fatty acid content, and C18:2 in an amount from about 5% to about 15% out of the total fatty acid content of the oil.

By way of example, but not limitation, where the oil is derived from oat oil, the two or more different fatty acids can comprise two or more fatty acids from C16 in an amount from about 5% to about 20% out of the total fatty acid content, C18:1 in an amount from about 15% to about 30% out of the total fatty acid content, and C18:2 in an amount from about 45% to about 60% out of the total fatty acid content of the oil.

In any of the foregoing embodiments, the EMO and/or FFA oil can have a triacylglycerol (TAG) content of 5% or less TAGs by weight based on the total weight of the EMO and/or FFA oil, respectively. By way of example, but not limitation, the TAG content can be equal to or less than 4%, equal to or less than 3%, equal to or less than 2%, equal to or less than 1% by weight based on the total weight of the EMO, FFA oil, or the oil composition, respectively, or the EMO and/or the FFA oil can be substantially free of TAGs.

It should be understood that the EMO and/or FFA oil can be derived from the oil sources described herein, including the first oil source and the second oil source, respectively, as provided in the foregoing embodiments.

In any of the foregoing embodiments, the EMO and/or FFA oil can further include non-oil ingredients derived from and naturally present in the first oil source or the second oil source, respectively. By way of example, but not limitation, the non-oil ingredients derived from and naturally present in the oil source, the first oil source or the second oil source can include glycosphingolipids such as ceramide phosphates, glycoglycerolipids such as monoglactosyl diacylglycerols, phosphatidyl alcohols such as phosphatidyl methanol, steroids such as sitosteryl esters, natural lipids such as campesterol esters, sphingolipids, phosphatidyl glycerol, wax esters, sphingomyelin, phosphatides, phytosterols (campesterol, stigmasterol, sitosterol), cholesterol, tocopherols, tocotrienols, carotenes, xanthophylls, betaxanthins, chlorophyll, long chain alcohols, polyphenols, terpenes (cycloartanol, cycloarterenol, 24-methylene cycloarterenol), terpenoids (squalene), tocopherol, vitamins, paraffins, isothiocyanates, oxazolidinthiones, glucosinolates, sanguinarine, gossypols, glycosylated alkaloids, phospholipids, sphingosine, diacylglyceride ethers, glucolipids, phytic acid, quinic acid, oxalic acid, tartaric acid, anacardic acid, malic acid, caftaric acid, coutaric acid, fertaric acid, phenylethanoids, hydroxycinnamic acids, phenolic acids, phytoestrogens, lignans, isoflavonoids, flavonoids, monoterpenes, diterpenes, triterpenoids, coenzyme, or combinations thereof. By way of further example, but not limitation, the non-oil ingredients derived from and naturally present in the first oil source or the second oil source can include ceramide phosphate, monoglactodiacylglycerol, phosphatidylmethanol, sitosteryl ester, campesterol ester, sphingolipids, phosphatidyl glycerol, wax esters, sphingomyelin, or combinations thereof. By way of still further example, but not limitation, the non-oil ingredients derived from and naturally present in the first oil source or the second oil source can include α-tocopherol, β-tocopherol, δ-tocopherol, γ-tocopherol, α-tocotrienol, β-tocotrienol, δ-tocotrienol, γ-tocotrienol, or combinations thereof. In certain aspects, the non-oil ingredients derived from and naturally present in the first oil source or the second oil source are present in the EMO or FFA oil in an amount or relative amount characteristic of or increased relative to the amounts or relative amounts, respectively, in the first oil source or the second oil source, respectively. It should be understood that the level of the non-oil ingredients can generally be the same as or greater than that in the oil source. By way of example, but not limitation, the non-oil components can be determined by LC/MS/MS analysis.

In any of the foregoing embodiments, the MAGs in the EMO (or oil composition) can have a sn-1 substitution to sn-2 substitution ratio of of greater than 1. By way of example but not limitation, the MAGs in the EMO (or oil composition) can have a sn-1 substitution to sn-2 substitution ratio of greater than 1, 1.1, 1.2, 1.3, 1.4, 1.5, 1.6, 1.7, 1.8, 1.9, 2, 2.25, 2.5, 2.75, 3, 3.25, 3.5, 3.75, 4, 4.25, 4.5, 4.75, 5, 6, 7, 8, 9, 10, 15, 20, 25, 30, 40, 50 or 100. By way of example, but not limitation, sn-1/sn-2 ratio can be determined by TLC or HPLC.

In any of the foregoing embodiments, the EMO can include free fatty acids (FFA). In any of the foregoing embodiments, the EMO can include a FFA content equal to or greater than 5% by weight of the total weight of the EMO. By way of example, but not limitation, the EMO can include a FFA content of about 5% to about 66%, about 10% to about 66%, about 20% to about 66%, about 30% to about 66%, about 40% to about 66%, about 50% to about 66%, about 5% to about 60%, about 10% to about 60%, about 20% to about 60%, about 30% to about 60%, about 40% to about 60%, about 50% to about 60%, about 5% to about 50%, about 10% to about 50%, about 20% to about 50%, about 30% to about 50%, about 40% to about 50%, about 5% to about 40%, about 10% to about 40%, about 20% to about 40%, about 30% to about 40%, 5% to about 30%, about 10% to about 30%, about 20% to about 30%, about 5% to about 20%, about 10% to about 20%, about 5% to about 10%, or about 5%, 10%, 15%, 20%, 25%, 30%, 35%, 40%, 45%, 50%, 55%, 60%, or 66% by weight of the total weight of the EMO.

In any of the foregoing embodiments, the FFA oil can include free fatty acids (FFA). In any of the foregoing embodiments, the FFA oil can include a FFA content equal to or greater than 1% by weight of the total weight of the FFA oil. By way of example, but not limitation, the FFA oil can include a FFA content equal of about 1% to about 95%, about 5% to about 95%, about 10% to about 95%, about 15% to about 95%, about 20% to about 95%, about 25% to about 95%, about 30% to about 95%, about 40% to about 95%, about 50% to about 95%, about 60% to about 95%, about 70% to about 95%, about 75% to about 95%, about 80% to about 95%, about 90% to about 95%, about 1% to about 90%, about 5% to about 90%, about 10% to about 90%, about 15% to about 90%, about 20% to about 90%, about 25% to about 90%, about 30% to about 90%, about 40% to about 90%, about 50% to about 90%, about 60% to about 90%, about 70% to about 90%, about 75% to about 90%, about 80% to about 90%, about 1% to about 80%, about 5% to about 80%, about 10% to about 80%, about 15% to about 80%, about 20% to about 80%, about 25% to about 80%, about 30% to about 80%, about 40% to about 80%, about 50% to about 80%, about 60% to about 80%, about 70% to about 80%, about 75% to about 80%, about 1% to about 75%, about 5% to about 75%, about 10% to about 75%, about 15% to about 75%, about 20% to about 75%, about 25% to about 75%, about 30% to about 75%, about 40% to about 75%, about 50% to about 75%, about 60% to about 75%, about 70% to about 75%, about 1% to about 70%, about 5% to about 70%, about 10% to about 70%, about 15% to about 70%, about 20% to about 70%, about 25% to about 70%, about 30% to about 70%, about 40% to about 70%, about 50% to about 70%, about 60% to about 70%, about 1% to about 60%, about 5% to about 60%, about 10% to about 60%, about 15% to about 60%, about 20% to about 60%, about 25% to about 60%, about 30% to about 60%, about 40% to about 60%, about 50% to about 60%, about 1% to about 50%, about 5% to about 50%, about 10% to about 50%, about 15% to about 50%, about 20% to about 50%, about 25% to about 50%, about 30% to about 50%, about 40% to about 50%, about 1% to about 40%, about 5% to about 40%, about 10% to about 40%, about 15% to about 40%, about 20% to about 40%, about 25% to about 40%, about 30% to about 40%, about 1% to about 30%, about 5% to about 30%, about 10% to about 30%, about 15% to about 30%, about 20% to about 30%, about 25% to about 30%, about 1% to about 25%, about 5% to about 25%, about 10% to about 25%, about 15% to about 25%, about 20% to about 25%, about 1% to about 20%, about 5% to about 20%, about 10% to about 20%, about 15% to about 20%, about 1% to about 15%, about 5% to about 15%, about 10% to about 15%, about 1% to about 10%, about 5% to about 10%, about 1% to about 5%, about 1%, 5%, 10%, 15%, 20%, 25%, 30%, 40%, 50%, 60%, 70%, 80%, 90%, 91%, 92%, 93%, 94%, or 95%, or greater than 1%, 5%, 10%, 15%, 20%, 25%, 30%, 40%, 50%, 60%, 70%, 80%, 90%, 91%, 92%, 93%, 94%, or 95%.

In any of the foregoing embodiments, the EMO (and thus the oil composition, if EMO is present) can include monoacylglycerols (MAGs). In any of the foregoing embodiments, the EMO can include a MAG content equal to or greater than 30% by weight of the total weight of the EMO. By way of example, but not limitation, the EMO can include a MAG content of about 30% to about 95%, about 40% to about 95%, about 50% to about 95%, about 60% to about 95%, about 70% to about 95%, about 80% to about 95%, about 90% to about 95%, about 30% to about 90%, about 40% to about 90%, about 50% to about 90%, about 60% to about 90%, about 80% to about 90%, about 30% to about 80%, about 40% to about 80%, about 50% to about 80%, about 60% to about 80%, about 70% to about 80%, about 30% to about 70%, about 40% to about 70%, about 50% to about 70%, about 50% to about 60%, about 30% to about 50%, about 40% to about 50%, or about 30%, 35%, 40%, 45%, 50%, 55%, 60%, 65%, 70%, 75%, 80%, 85%, 90%, or 95% by weight of the EMO.

In any of the foregoing embodiments, the EMO (and thus the oil composition, if EMO is present) can include diacylglycerols (DAGs). In any of the foregoing embodiments, the EMO can include a DAG content equal to or greater than 5% by weight of the total weight of the EMO. By way of example, but not limitation, the EMO can include a DAG content of about 5% to about 66%, about 10% to about 66%, about 20% to about 66%, about 30% to about 66%, about 40% to about 66%, about 50% to about 66%, about 5% to about 60%, about 10% to about 60%, about 20% to about 60%, about 30% to about 60%, about 40% to about 60%, about 50% to about 60%, about 5% to about 50%, about 10% to about 50%, about 20% to about 50%, about 30% to about 50%, about 40% to about 50%, about 5% to about 40%, about 10% to about 40%, about 20% to about 40%, about 30% to about 40%, 5% to about 30%, about 10% to about 30%, about 20% to about 30%, about 5% to about 20%, about 10% to about 20%, about 5% to about 10%, or about 5%, 10%, 15%, 20%, 25%, 30%, 35%, 40%, 45%, 50%, 55%, 60%, or 66% by weight of the total weight of the EMO.

In any of the foregoing embodiments, the EMO can have fatty acid profile comprising an amount of two or more fatty acids, wherein the amount of each of the two or more fatty acids within 10% of the amount of each of the two or more fatty acids in the first oil source. By way of example, but not limitation, the EMO can have a fatty acid profile comprising an amount of three or more, four or more, five or more, six or more, seven or more, eight or more, nine or more, ten, eleven, or twelve of C6:0, C8:0, C10, C12, C14, C16, C18, C18:1, C18:2, C18:3, C20 and C22, wherein the amount of each of the fatty acids is within 10% of the amount of each of the fatty acids, 9%, 8%, 7%, 6%, 5%, 4%, 3%, 2%, or 1% or be about the same amount. For example, if the first oil source has 15% C18 fatty acid, the EMO can have between about 5-25%, 6-24%, 7-23%, 8-22%, 9-21%, 10-20%, 11-19%, 12-18%, 13-17%, 14-16% or about 15% C18 fatty acid.

It should be understood, in any of the foregoing embodiments, that the first oil source and the second oil source can be the same type of oil or different types of oil and that each of the first oil source and the second oil source can be a single type of oil (e.g. oat oil or flaxseed oil) or a combination of oils as described further herein. It should likewise be further understood that the EMO and/or FFA oil can be the result of a blend of EMO and/or FFA oils, respectively, which have been processed independently and later combined such that the first oil source or the second oil source are each the combination of oil sources used to produce the different EMO and/or FFA oils that have been combined.

In any of the foregoing embodiments, the EMO and the FFA oil can each comprise two or more different fatty acids. In any of the foregoing embodiments, the two or more different fatty acids can be, by way of example but not limitation, selected from the group consisting of caproic acid, enanthic acid, caprylic acid, pelargonic acid, capric acid, undecylic acid, lauric acid, tridecylic acid, myristic acid, pentadecylic acid, palmitic acid, palmitoleic acid, margaric acid, heptadecenoic acid, stearic acid, nonadecylic acid, vaccenic acid, oleic acid, linoleic acid, linolenic acid, stearidonic acid, octadecatetraenoic acid, arachidic acid, heneicosylic acid, gondoic acid, eicosadienoic acid, paullinic acid, behenic acid, tricosylic acid, erucic acid, lignoceric acid, docosahexaenoic acid, eicosapentaenoic acid, nervonic acid, and isomers thereof. Alternatively, the fatty acids, including the two or more different fatty acids can each independently be selected from the group consisting of a C6 fatty acid such as, by way of example but not limitation, C6:0, a C7 fatty acid, a C8 fatty acid such as, by way of example but not limitation, C8:0 fatty acid, a C9 fatty acid, a C10 fatty acid, a C11 fatty acid, a C12 fatty acid, a C13 fatty acid, a C14 fatty acid, a C15 fatty acid, a C16 fatty acid, a C17 fatty acid, a C18 fatty acid such as, by way of example, but not limitation, a C18:0 fatty acid, a C18:0 fatty acid, a C18:1 fatty acid, a C18:2 fatty acid, or a C18:3 fatty acid, a C19 fatty acid, a C20 fatty acid such as, by way of example, but not limitation, C20:5 fatty acid, a C21 fatty acid, a C22 fatty acid such as, by way of example, but not limitation, C22:6 fatty acid, a C23 fatty acid, and a C24 fatty acid. It should be understood that the two or more different fatty acids can be the same between the EMO and FFA oil or can be different.

In any of the foregoing embodiments, the two or more different fatty acids can be present in the EMO or FFA oil in an amount that is within 10%, 9%, 8%, 7%, 6%, 5%, 4%, 3%, 2% or 1% of the amount of that fatty acid in the a fatty acid profile of the first oil source or the second oil source, respectively. In any of the foregoing embodiments, the two or more different fatty acids, can be two fatty acids, three fatty acids, four fatty acids, five fatty acids, six fatty acids, seven fatty acids, eight fatty acids, nine fatty acides, ten fatty acids or more. For example, if a first oil source has 20% C18:1 fatty acid and 20% C12 fatty acid, then the EMO would have 10-30% C18:1 and 10-30% C12 if it is within 10% of the amount of the fatty acids in the fatty acid profile of the first source oil. Thus, where the oil composition is a blend of EMO and FFA oil from different oil sources, the fatty acid profile of the oil composition will vary according to the ratio of EMO:FFA oil and the expected ranges can be calculated by one of skill in the art. However, where the EMO and FFA are derived from the same source oil, the fatty acid profile of the oil composition would be expected to be similar to that of the source oil.

In any of the foregoing embodiments, the EMO and/or the FFA oil (and thus the oil composition) can be substantially free of 3-monochloropropanediol and glycidol equivalents.

Where a blend of EMO (first amount) and FFA oil (second amount) is used in any of the foregoing embodiments, the first amount as a percentage of the total of the first amount and the second amount (i.e. the amount of EMO relative to the amount of EMO and FFA oil) can be from about 0.1% to about 99.9%, about 0.1% to about 99.8%, about 0.1% to about 99.5%, about 0.1% to about 99%, about 0.1% to about 98%, about 0.1% to about 97%, about 0.1% to about 96%, about 0.1% to about 95%, about 0.1% to about 94%, about 0.1% to about 93%, about 0.1% to about 92%, about 0.1% to about 91%, about 0.1% to about 90%, about 0.1% to about 80%, about 0.1% to about 75%, about 0.1% to about 70%, about 0.1% to about 60%, about 0.1% to about 50%, about 0.1% to about 40%, about 0.1% to about 30%, about 0.1% to about 25%, about 0.1% to about 20%, about 0.1% to about 15%, about 0.1% to about 10%, about 0.1% to about 5%, about 0.1% to about 4%, about 0.1% to about 3%, about 0.1% to about 2%, about 0.1% to about 1%, about 0.1% to about 0.5%, about 0.1% to about 0.2%, 0.2% to about 99.9%, about 0.2% to about 99.8%, about 0.2% to about 99.5%, about 0.2% to about 99%, about 0.2% to about 98%, about 0.2% to about 97%, about 0.2% to about 96%, about 0.2% to about 95%, about 0.2% to about 94%, about 0.2% to about 93%, about 0.2% to about 92%, about 0.2% to about 91%, about 0.2% to about 90%, about 0.2% to about 80%, about 0.2% to about 75%, about 0.2% to about 70%, about 0.2% to about 60%, about 0.2% to about 50%, about 0.2% to about 40%, about 0.2% to about 30%, about 0.2% to about 25%, about 0.2% to about 20%, about 0.2% to about 15%, about 0.2% to about 10%, about 0.2% to about 5%, about 0.2% to about 4%, about 0.2% to about 3%, about 0.2% to about 2%, about 0.2% to about 1%, about 0.2% to about 0.5%, 0.5% to about 99.9%, about 0.5% to about 99.8%, about 0.5% to about 99.5%, about 0.5% to about 99%, about 0.5% to about 98%, about 0.5% to about 97%, about 0.5% to about 96%, about 0.5% to about 95%, about 0.5% to about 94%, about 0.5% to about 93%, about 0.5% to about 92%, about 0.5% to about 91%, about 0.5% to about 90%, about 0.5% to about 80%, about 0.5% to about 75%, about 0.5% to about 70%, about 0.5% to about 60%, about 0.5% to about 50%, about 0.5% to about 40%, about 0.5% to about 30%, about 0.5% to about 25%, about 0.5% to about 20%, about 0.5% to about 15%, about 0.5% to about 10%, about 0.5% to about 5%, about 0.5% to about 4%, about 0.5% to about 3%, about 0.5% to about 2%, about 0.5% to about 1%, about 1% to about 99.9%, about 1% to about 99.8%, about 1% to about 99.5%, about 1% to about 99%, about 1% to about 98%, about 1% to about 97%, about 1% to about 96%, about 1% to about 95%, about 1% to about 94%, about 1% to about 93%, about 1% to about 92%, about 1% to about 91%, about 1% to about 90%, about 1% to about 80%, about 1% to about 75%, about 1% to about 70%, about 1% to about 60%, about 1% to about 50%, about 1% to about 40%, about 1% to about 30%, about 1% to about 25%, about 1% to about 20%, about 1% to about 15%, about 1% to about 10%, about 1% to about 5%, about 1% to about 4%, about 1% to about 3%, about 1% to about 2%, about 2% to about 99.9%, about 2% to about 99.8%, about 2% to about 99.5%, about 2% to about 99%, about 2% to about 98%, about 2% to about 97%, about 2% to about 96%, about 2% to about 95%, about 2% to about 94%, about 2% to about 93%, about 2% to about 92%, about 2% to about 91%, about 2% to about 90%, about 2% to about 80%, about 2% to about 75%, about 2% to about 70%, about 2% to about 60%, about 2% to about 50%, about 2% to about 40%, about 2% to about 30%, about 2% to about 25%, about 2% to about 20%, about 2% to about 10%, about 2% to about 5%, about 5% to about 99.9%, about 5% to about 99.8%, about 5% to about 99.5%, about 5% to about 99%, about 5% to about 98%, about 5% to about 97%, about 5% to about 96%, about 5% to about 95%, about 5% to about 94%, about 5% to about 93%, about 5% to about 92%, about 5% to about 91%, about 5% to about 90%, about 5% to about 80%, about 5% to about 75%, about 5% to about 70%, about 5% to about 60%, about 5% to about 50%, about 5% to about 40%, about 5% to about 30%, about 5% to about 25%, about 5% to about 20%, about 5% to about 10%, about 10% to about 99.9%, about 10% to about 99.8%, about 10% to about 99.5%, about 10% to about 99%, about 10% to about 98%, about 10% to about 97%, about 10% to about 96%, about 10% to about 95%, about 10% to about 94%, about 10% to about 93%, about 10% to about 92%, about 10% to about 91%, about 10% to about 90%, about 10% to about 80%, about 10% to about 75%, about 10% to about 70%, about 10% to about 60%, about 10% to about 50%, about 10% to about 40%, about 10% to about 30%, about 10% to about 25%, about 10% to about 20%, about 20% to about 99.9%, about 20% to about 99.8%, about 20% to about 99.5%, about 20% to about 99%, about 20% to about 98%, about 20% to about 97%, about 20% to about 96%, about 20% to about 95%, about 20% to about 94%, about 20% to about 93%, about 20% to about 92%, about 20% to about 91%, about 20% to about 90%, about 20% to about 80%, about 20% to about 75%, about 20% to about 70%, about 20% to about 60%, about 20% to about 50%, about 20% to about 40%, about 20% to about 30%, about 20% to about 25%, about 25% to about 99.9%, about 25% to about 99.8%, about 25% to about 99.5%, about 25% to about 99%, about 25% to about 98%, about 25% to about 97%, about 25% to about 96%, about 25% to about 95%, about 25% to about 94%, about 25% to about 93%, about 25% to about 92%, about 25% to about 91%, about 25% to about 90%, about 25% to about 80%, about 25% to about 75%, about 25% to about 70%, about 25% to about 60%, about 25% to about 50%, about 25% to about 40%, about 25% to about 30%, about 30% to about 99.9%, about 30% to about 99.8%, about 30% to about 99.5%, about 30% to about 99%, about 30% to about 98%, about 30% to about 97%, about 30% to about 96%, about 30% to about 95%, about 30% to about 94%, about 30% to about 93%, about 30% to about 92%, about 30% to about 91%, about 30% to about 90%, about 30% to about 80%, about 30% to about 75%, about 30% to about 70%, about 30% to about 60%, about 30% to about 50%, about 30% to about 40%, about 40% to about 99.9%, about 40% to about 99.8%, about 40% to about 99.5%, about 40% to about 99%, about 40% to about 98%, about 40% to about 97%, about 40% to about 96%, about 40% to about 95%, about 40% to about 94%, about 40% to about 93%, about 40% to about 92%, about 40% to about 91%, about 40% to about 90%, about 40% to about 80%, about 40% to about 75%, about 40% to about 70%, about 40% to about 60%, about 40% to about 50%, about 50% to about 99.9%, about 50% to about 99.8%, about 50% to about 99.5%, about 50% to about 99%, about 50% to about 98%, about 50% to about 97%, about 50% to about 96%, about 50% to about 95%, about 50% to about 94%, about 50% to about 93%, about 50% to about 92%, about 50% to about 91%, about 50% to about 90%, about 50% to about 80%, about 50% to about 75%, about 50% to about 70%, about 50% to about 60%, about 60% to about 99.9%, about 60% to about 99.8%, about 60% to about 99.5%, about 60% to about 99%, about 60% to about 98%, about 60% to about 97%, about 60% to about 96%, about 60% to about 95%, about 60% to about 94%, about 60% to about 93%, about 60% to about 92%, about 60% to about 91%, about 60% to about 90%, about 60% to about 80%, about 60% to about 75%, about 60% to about 70%, about 70% to about 99.9%, about 70% to about 99.8%, about 70% to about 99.5%, about 70% to about 99%, about 70% to about 98%, about 70% to about 97%, about 70% to about 96%, about 70% to about 95%, about 70% to about 94%, about 70% to about 93%, about 70% to about 92%, about 70% to about 91%, about 70% to about 90%, about 70% to about 80%, about 80% to about 99.9%, about 80% to about 99.8%, about 80% to about 99.5%, about 80% to about 99%, about 80% to about 98%, about 80% to about 97%, about 80% to about 96%, about 80% to about 95%, about 80% to about 94%, about 80% to about 93%, about 80% to about 92%, about 80% to about 91%, about 80% to about 90%, about 90% to about 99.9%, about 90% to about 99.8%, about 90% to about 99.5%, about 90% to about 99%, about 90% to about 98%, about 90% to about 97%, about 90% to about 96%, about 90% to about 95%, about 90% to about 94%, about 90% to about 93%, about 90% to about 92%, about 90% to about 91%, about 91% to about 99.9%, about 91% to about 99.8%, about 91% to about 99.5%, about 91% to about 99%, about 91% to about 98%, about 91% to about 97%, about 91% to about 96%, about 91% to about 95%, about 91% to about 94%, about 91% to about 93%, about 91% to about 92%, about 92% to about 99.9%, about 92% to about 99.8%, about 92% to about 99.5%, about 92% to about 99%, about 92% to about 98%, about 92% to about 97%, about 92% to about 96%, about 92% to about 95%, about 92% to about 94%, about 92% to about 93%, about 93% to about 99.9%, about 93% to about 99.8%, about 93% to about 99.5%, about 93% to about 99%, about 93% to about 98%, about 93% to about 97%, about 93% to about 96%, about 93% to about 95%, about 93% to about 94%, about 94% to about 99.9%, about 94% to about 99.8%, about 94% to about 99.5%, about 94% to about 99%, about 94% to about 98%, about 94% to about 97%, about 94% to about 96%, about 94% to about 95%, about 95% to about 99.9%, about 95% to about 99.8%, about 95% to about 99.5%, about 95% to about 99%, about 95% to about 98%, about 95% to about 97%, about 95% to about 96%, about 96% to about 99.9%, about 96% to about 99.8%, about 96% to about 99.5%, about 96% to about 99%, about 96% to about 98%, about 96% to about 97%, about 97% to about 99.9%, about 97% to about 99.8%, about 97% to about 99.5%, about 97% to about 99%, about 97% to about 98%, about 98% to about 99.9%, about 98% to about 99.8%, about 98% to about 99.5%, about 98% to about 99%, about 99% to about 99.9%, about 99% to about 99.8%, about 99% to about 99.5%, about 99.5% to about 99.9%, about 99.5% to about 99.8%, about 99.8% to about 99.9%, or about 0.1%, 0.2%, 0.5%, 1%, 2%, 3%, 4%, 5%, 6%, 7%, 8%, 9%, 10%, 15%, 20%, 25%, 30%, 40%, 50%, 60%, 70%, 75%, 80%, 90%, 91%, 92%, 93%, 94%, 95%, 96%, 97%, 98%, 99%, 99.5%, 99.8%, or 99.9%. By way of still further example, the first amount as a percentage of the total of the first amount and the second amount can be from about 25% to about 75%. Alternatively, the first amount as a percentage of the total of the first amount the second amount can be 0% or 100%, i.e. either pure EMO or pure FFA oil.

It should be understood throughout the disclosure that the EMO, FFA oil or oil compositions, whether nanoemulsion, microemulsion or otherwise, can include a fatty acid profile that is within 10%, 9%, 8%, 7%, 6%, 5%, 4%, 3%, 2%, or 1% for two or more different fatty acids and that such fatty acid profiles for single oil sources are provided herein and known to those of skill in the art. For blends of EMO and FFA oil, the fatty acid profile can be calculated based on the ratio of the EMO to FFA oil.

In any of the foregoing embodiments, including those with nanoemulsions or microemulsions, the following tables provide preferred oil sources and ranges.

| Oil Source | EMO (%) (balance FFA oil) | Total FFA (%) |
|---|---|---|
| Flaxseed | 100-0 | 18-100 |
| MCT | 100-0 | 21-100 |
| Rosehip | 75-0 | 25-100 |
| Sesame | 75-25 | 25-75 |
| Hemp | 100-0 | 8-100 |

*By example, flaxseed EMO can contain 18% FFA, thus a 100% EMO compositon would still include 18% FFA but could include more if spiked with FFA oil.

| EMO (%) | FFA oil (%) |
|---|---|
| Flaxseed 75-50 | Coconut 25-50 |
| Flaxseed 75 | Fish 25 |
| Flaxseed 75-25 | Rosehip 25-75 |
| MCT 75-50 | Coconut 25-50 |
| Coconut 75-25 | Flaxseed 25-75 |
| Coconut 75-25 | MCT 25-75 |
| Coconut 75-25 | Rosehip 25-75 |
| Coconut 75 | Oat 25 |
| Fish 25 | Flaxseed 75 |
| Fish 50 | Coconut 50 |
| Rosehip 75-25 | Flaxseed 25-50 |
| Rosehip 75-50 | Coconut 25-50 |
| Algae 25 | Flaxseed 75 |
| Algae 25 | MCT 75 |
| Oat 75-25 | Flaxseed 25-75 |

Drug-Delivery Methods and Compositions

In some embodiments, an active composition can include any composition, including a nanoemulsion (or microemulsion or larger nanoemulsion) or non-nanoemulsion composition of the present disclosure and an active ingredient, of any of the foregoing embodiments. In any of the foregoing embodiments, the active ingredient can be present in the oil of the composition, including the droplets or particles nanoemulsion or microemulsion. Alternatively, the active ingredient can be present in an aqueous phase of the composition, including of the nanoemulsion or microemulsion.

In some embodiments, an active composition can include a first component, a second component and a third component, where the first component is selected from the group consisting of a cream, a lotion, an eye drop, an ear drop, a sinus rinse, a spray such as for nasal, oral mucosal, skin or foot treatment, an ointment, a deodorant, a body wash, a shampoo, a scalp treatment, a mouthwash, a toothpaste, a lozenge, a capsule, a lubricant, a beverage or a powder; wherein the second component is an additive comprising the composition of any of the foregoing embodiments, including the nanoemulsions, microemulsions and non-nanoemulsion compositions; wherein the third component is an active ingredient selected from the group consisting of an antibiotic, an anti-viral drug, an anti-parasitic drug, an anti-fungal drug, an anti-cancer drug, a steroid drug, a nonsteroidal drug, a narcotic analgesic drug, an immunosuppressant drug, a central nervous system drug, a cardiovascular drug, a diabetes drug, a nucleic acid, a peptide, a protein, a synthetic chemical molecule, a plant extract, an antioxidant, and a vitamin or nutritional supplement.

In any of the foregoing embodiments, the active ingredient can be any active ingredient that can be formulated with the nanoemulsions (and microemulsions) and non-nanoemulsion compositions of the present disclosure. By way of example, but not limitation, the active ingredient can be a small molecule drug, a peptide, a protein, including an antibody, a nucleic acid or a vaccine. By way of example, but not limitation, the active ingredient can be an antibiotic, an anti-viral drug, an anti-parasitic drug, an anti-fungal drug, an anti-cancer drug, a steroid drug, a nonsteroidal drug, a narcotic analgesic drug, an immunosuppressant drug, a central nervous system drug, a cardiovascular drug, a diabetes drug, a nucleic acid, a peptide, a protein, a synthetic chemical molecule, a plant extract, an antioxidant, and a vitamin or nutritional supplement. By way of further example, but not limitation, the active ingredient can be an antibiotic, anti-viral drug, anti-parasitic drug, anti-fungal drug, anti-cancer drug, steroid drug, nonsteroidal drug, narcotic analgesic drug, immunosuppressant drug, central nervous system (CNS) drug, cardiovascular drugs, diabetes drugs, or a vitamin or nutritional supplement. Non-limiting examples of antibiotics include: clarithromycin A, bacitracin, neomycin, polymyxin B, mupirocin, or cyclosporine. Non-limiting examples of anti-viral drugs include: acyclovir, famciclovir, valacyclovir, famciclovir, penciclovir, cidofovir, or foscarnet. Non-limiting examples of anti-parasitic drugs include: levamisole, niclosamide, praziquantel, albendazole, diethylcarbamazine, ivermectin, tiabendazole, chloroquine, or hydroxychloroquine. Non-limiting examples of anti-fungal drugs include: amphotericin B, ketoconazole, or ciclopirox. Non-limiting examples of anti-cancer drugs include: 5-fluorouracil, paclitaxel, gemcitabine, 5-aminolevulinic acid, tamoxifen, rapamycin, chloroquine, or hydroxychloroquine. Non-limiting examples of steroid drugs include: hydrocortisone, prednisone, prednisolone, clobetasol propionate, triamcinolone, triamcinolone acetonide, testosterone, prasterone, or estradiol. Non-limiting examples of nonsteroidal drugs include: ibuprofen, crisaborole, diclofenac, aceclofenac, celecoxib, ketoprofen, meloxicam, piroxicam, benvitimod, aspirin, salicylic acid, or naproxen. Non-limiting examples of narcotic analgesic drugs include: fentanyl, sufentanil, morphine, codeine, hydromorphone, hydrocodone, oxycodone, oxymorphone, or tramadol. Non-limiting examples of immunosuppressant drugs include: tacrolimus, pimecrolimus, ciclosporin, azathioprine, rapamycin, everolimus, or methotrexate. Non-limiting examples of central nervous system drugs include: riluzole, ropinirole, caffeine, thiocolchicoside, clozapine, imipramine, doxepin, or nicotine. Non-limiting examples of cardiovascular drugs include: olmesartan or varvedilol. A non-limiting example of a diabetes drug is glibenclamide. Non-limiting examples of vitamins or nutritional supplements include: vitamin A, B vitamins, vitamin C, vitamin D, vitamin E, vitamin K, curcumin, cannabidiol (CBD), cannabinoids, tetrahydrocannabinol (THC), avenanthramides, epigallocatechin gallate (EGCG), berberine, inulin, glycyrrhizin, caffeine, capsaicin, or resveratrol. Non-limiting examples of proteins and peptides include: thyrotropin releasing hormone analogs, antigenic peptides, human growth hormone, human immunoglobin G (IgG), insulin, parathyroid hormone, interferon-α, luteinizing hormone releasing hormone, or duplimumab. By way of further example, but not limitation, the active ingredient can be a drug suitable to treat or prevent acne, pain, inflammation, cancer or a vitamin or anti-aging compound. By way of still further example, the active ingredient can be lidocaine, bupivacaine, prilocaine, or a combination thereof, aspartyl-alanyl-diketopiperazine (DA-DKP), retinol, retinyl palmitate, trilfuoroacetyl tripeptide-2, or hexapeptide-11. In certain aspects, the active ingredient is fat-soluble. In certain aspects, the active ingredient is hydrophobic. By way of example, but not limitation, the active ingredient can have a log P (octanol-water partition coefficient) value of about 1 to about 6. By way of further example, but not limitation, the active ingredient can have a log P value of about 1 to about 6, about 1 to about 5, about 1 to about 4, about 1 to about 3, about 1 to about 2, about 2 to about 6, about 2 to about 5, about 2 to about 4, about 2 to about 3, about 3 to about 6, about 3 to about 5, about 3 to about 4, about 4 to about 6, about 4 to about 5, about 1, 1.5, 2, 2.5, 3, 3.5, 4, 4.5, 5, 5.5 or 6. By way of still further example, but not limitation, the active ingredient can be cannabidiol (CBD), vitamin E or carotene. It should be understood, however, that the active ingredient, in certain aspects, can be hydrophilic. By way of example, but limitation, for transdermal applications, the active ingredient can be hydrophilic. By way of still further example, but not limitation, the hydrophilic active ingredient can have a log P of less than 1, such as from about −3 to about 1, about −2 to about 1, about −1 to about 1, about 0 to about 1, about −3, −2.5, −2, −1.5, −1, −0.5, 0, 0.5 or 1. It should be understood that the log P can be as measured or predicted. Where the active ingredient is hydrophilic, it should be understood that it can be present in an aqueous phase of the nanoemulsion or microemulsion of the present disclosure. By way of example, but not limitation, such a composition can be used for transdermal delivery of the active ingredient.

In any of the foregoing embodiments, the active ingredient can be present in the active composition at a concentration of about 0.001 mg/mL to about 10 mg/mL or more. By way of example, but not limitation, the active ingredient can be present in the active composition at a concentration of about 0.001 to about 10 mg/mL, about 0.01 to about 10 mg/mL, about 0.1 to about 10 mg/mL, about 0.5 to about 10 mg/mL, about 1 to about 10 mg/mL, about 2.5 to about 10 mg/mL, about 5 to about 10 mg/mL, about 7.5 to about 10 mg/mL, about 0.001 to about 5 mg/mL, about 0.01 to about 5 mg/mL, about 0.1 to about 5 mg/mL, about 0.5 to about 5 mg/mL, about 1 to about 5 mg/mL, about 2 to about 5 mg/mL, about 3 to about 5 mg/mL, about 4 to about 5 mg/mL, about 1, 1.5, 2, 2.5, 3, 3.5, 4, 4.5, 5, 5.5, 6, 6.5, 7, 7.5, 8, 8.5, 9, 9.5, 10 mg/mL or more. By way of further example, but not limitation, the active ingredient can be present in the active composition at higher than 10 mg/mL, such as about 10, 12.5, 15, 17.5, 20, 25 mg/mL or more. In any of the foregoing embodiments, the active ingredient can be present in the active composition in a therapeutically relevant concentration. A therapeutically relevant concentration can be understood as a concentration sufficient to have a desired therapeutic effect. For example, the desired therapeutic effect can be to diagnose, cure, mitigate, treat or prevent disease or to affect the structure or function of the body of a subject a change in the structure or function of the body of a subject.

In any of the foregoing embodiments, for compositions and active compositions of the present disclosure, the active ingredient can have a skin permeability of at least 0.1 μg/cm² for the active ingredient over a period of 4, 5, 6, 7, 8 or 24 hours. By way of example, but not limitation, the skin permeability can be as measured by HPLC across artificial skin, such as a Strat-M membrane. By way of further example, but not limitation, the active composition can have a skin permeability of at least 0.1 μg/cm², at least 0.2 μg/cm², at least 0.3 μg/cm², at least 0.4 μg/cm², at least 0.5 μg/cm², at least 0.75 μg/cm², at least 1.0 μg/cm², at least 1.5 μg/cm², at least 2.0 μg/cm², at least 2.5 μg/cm², at least 3.0 μg/cm², at least 3.5 μg/cm², at least 4.0 μg/cm², at least 4.5 μg/cm², at least 5.0 μg/cm², at least 6 μg/cm², at least 7 μg/cm², at least 8 μg/cm², at least 9 μg/cm², at least 10 μg/cm², at least 20 μg/cm², at least 30 μg/cm², at least 40 μg/cm², at least 50 μg/cm², at least 60 μg/cm², at least 70 μg/cm², at least 80 μg/cm², at least 90 μg/cm², at least 100 μg/cm², at least 110 μg/cm², at least 120 μg/cm², at least 130 μg/cm², at least 140 μg/cm², at least 150 μg/cm², at least 160 μg/cm², at least 170 μg/cm², at least 180 μg/cm², at least 190 μg/cm², at least 200 μg/cm², at least 250 μg/cm², at least 300 μg/cm², at least 350 μg/cm², at least 400 μg/cm², at least 450 μg/cm², at least 500 μg/cm², at least 1000 μg/cm², from about 0.1 to about 2000 μg/cm², from about 0.1 to about 1500 μg/cm², from about 0.1 to about 1000 μg/cm², from about 0.1 to about 500 μg/cm², from about 0.1 to about 450 μg/cm², from about 0.1 to about 400 μg/cm², from about 0.1 to about 350 μg/cm², from about 0.1 to about 300 μg/cm², from about 0.1 to about 250 μg/cm², from about 0.1 to about 200 μg/cm², from about 0.1 to about 190 μg/cm², from about 0.1 to about 180 μg/cm², from about 0.1 to about 170 μg/cm², from about 0.1 to about 160 μg/cm², from about 0.1 to about 150 μg/cm², from about 0.1 to about 140 μg/cm², from about 0.1 to about 130 μg/cm², from about 0.1 to about 120 μg/cm², from about 0.1 to about 110 μg/cm², from about 0.1 to about 100 μg/cm², from about 0.1 to about 90 μg/cm², from about 0.1 to about 80 μg/cm², from about 0.1 to about 70 μg/cm², from about 0.1 to about 60 μg/cm², from about 0.1 to about 50 μg/cm², from about 0.1 to about 40 μg/cm², from about 0.1 to about 30 μg/cm², from about 0.1 to about 20 μg/cm², from about 0.1 to about 10 μg/cm², from about 0.1 to about 9 μg/cm², from about 0.1 to about 8 μg/cm², from about 0.1 to about 7 μg/cm², from about 0.1 to about 6 μg/cm², from about 0.1 to about 5 μg/cm², from about 0.1 to about 4.5 μg/cm², from about 0.1 to about 4 μg/cm², from about 0.1 to about 3.5 μg/cm², from about 0.1 to about 3 μg/cm², from about 0.1 to about 2.5 μg/cm², from about 0.1 to about 2 μg/cm², from about 0.1 to about 1.5 μg/cm², from about 0.1 to about 1 μg/cm², from about 0.1 to about 0.75 μg/cm², from about 0.1 to about 0.5 μg/cm², from about 0.1 to about 0.4 μg/cm², from about 0.1 to about 0.3 μg/cm², from about 0.1 to about 0.2 μg/cm², from about 0.2 to about 2000 μg/cm², from about 0.2 to about 1500 μg/cm², from about 0.2 to about 1000 μg/cm², from about 0.2 to about 500 μg/cm², from about 0.2 to about 450 μg/cm², from about 0.2 to about 400 μg/cm², from about 0.2 to about 350 μg/cm², from about 0.2 to about 300 μg/cm², from about 0.2 to about 250 μg/cm², from about 0.2 to about 200 μg/cm², from about 0.2 to about 190 μg/cm², from about 0.2 to about 180 μg/cm², from about 0.2 to about 170 μg/cm², from about 0.2 to about 160 μg/cm², from about 0.2 to about 150 μg/cm², from about 0.2 to about 140 μg/cm², from about 0.2 to about 130 μg/cm², from about 0.2 to about 120 μg/cm², from about 0.2 to about 110 μg/cm², from about 0.2 to about 100 μg/cm², from about 0.2 to about 90 μg/cm², from about 0.2 to about 80 μg/cm², from about 0.2 to about 70 μg/cm², from about 0.2 to about 60 μg/cm², from about 0.2 to about 50 μg/cm², from about 0.2 to about 40 μg/cm², from about 0.2 to about 30 μg/cm², from about 0.2 to about 20 μg/cm², from about 0.2 to about 10 μg/cm², from about 0.2 to about 9 μg/cm², from about 0.2 to about 8 μg/cm², from about 0.2 to about 7 μg/cm², from about 0.2 to about 6 μg/cm², from about 0.2 to about 5 μg/cm², from about 0.2 to about 4.5 μg/cm², from about 0.2 to about 4 μg/cm², from about 0.2 to about 3.5 μg/cm², from about 0.2 to about 3 μg/cm², from about 0.2 to about 2.5 μg/cm², from about 0.2 to about 2 μg/cm², from about 0.2 to about 1.5 μg/cm², from about 0.2 to about 1 μg/cm², from about 0.2 to about 0.75 μg/cm², from about 0.2 to about 0.5 μg/cm², from about 0.2 to about 0.4 μg/cm², from about 0.2 to about 0.3 μg/cm², from about 0.3 to about 2000 μg/cm², from about 0.3 to about 1500 μg/cm², from about 0.3 to about 1000 μg/cm², from about 0.3 to about 500 μg/cm², from about 0.3 to about 450 μg/cm², from about 0.3 to about 400 μg/cm², from about 0.3 to about 350 μg/cm², from about 0.3 to about 300 μg/cm², from about 0.3 to about 250 μg/cm², from about 0.3 to about 200 μg/cm², from about 0.3 to about 190 μg/cm², from about 0.3 to about 180 μg/cm², from about 0.3 to about 170 μg/cm², from about 0.3 to about 160 μg/cm², from about 0.3 to about 150 μg/cm², from about 0.3 to about 140 μg/cm², from about 0.3 to about 130 μg/cm², from about 0.3 to about 120 μg/cm², from about 0.3 to about 110 μg/cm², from about 0.3 to about 100 μg/cm², from about 0.3 to about 90 μg/cm², from about 0.3 to about 80 μg/cm², from about 0.3 to about 70 μg/cm², from about 0.3 to about 60 μg/cm², from about 0.3 to about 50 μg/cm², from about 0.3 to about 40 μg/cm², from about 0.3 to about 30 μg/cm², from about 0.3 to about 20 μg/cm², from about 0.3 to about 10 μg/cm², from about 0.3 to about 9 μg/cm², from about 0.3 to about 8 μg/cm², from about 0.3 to about 7 μg/cm², from about 0.3 to about 6 μg/cm², from about 0.3 to about 5 μg/cm², from about 0.3 to about 4.5 μg/cm², from about 0.3 to about 4 μg/cm², from about 0.3 to about 3.5 μg/cm², from about 0.3 to about 3 μg/cm², from about 0.3 to about 2.5 μg/cm², from about 0.3 to about 2 μg/cm², from about 0.3 to about 1.5 μg/cm², from about 0.3 to about 1 μg/cm², from about 0.3 to about 0.75 μg/cm², from about 0.3 to about 0.5 μg/cm², from about 0.3 to about 0.4 μg/cm², from about 0.4 to about 2000 μg/cm², from about 0.4 to about 1500 μg/cm², from about 0.4 to about 1000 μg/cm², from about 0.4 to about 500 μg/cm², from about 0.4 to about 450 μg/cm², from about 0.4 to about 400 μg/cm², from about 0.4 to about 350 μg/cm², from about 0.4 to about 300 μg/cm², from about 0.4 to about 250 μg/cm², from about 0.4 to about 200 μg/cm², from about 0.4 to about 190 μg/cm², from about 0.4 to about 180 μg/cm², from about 0.4 to about 170 μg/cm², from about 0.4 to about 160 μg/cm², from about 0.4 to about 150 μg/cm², from about 0.4 to about 140 μg/cm², from about 0.4 to about 130 μg/cm², from about 0.4 to about 120 μg/cm², from about 0.4 to about 110 μg/cm², from about 0.4 to about 100 μg/cm², from about 0.4 to about 90 μg/cm², from about 0.4 to about 80 μg/cm², from about 0.4 to about 70 μg/cm², from about 0.4 to about 60 μg/cm², from about 0.4 to about 50 μg/cm², from about 0.4 to about 40 μg/cm², from about 0.4 to about 30 μg/cm², from about 0.4 to about 20 μg/cm², from about 0.4 to about 10 μg/cm², from about 0.4 to about 9 μg/cm², from about 0.4 to about 8 μg/cm², from about 0.4 to about 7 μg/cm², from about 0.4 to about 6 μg/cm², from about 0.4 to about 5 μg/cm², from about 0.4 to about 4.5 μg/cm², from about 0.4 to about 4 μg/cm², from about 0.4 to about 3.5 μg/cm², from about 0.4 to about 3 μg/cm², from about 0.4 to about 2.5 μg/cm², from about 0.4 to about 2 μg/cm², from about 0.4 to about 1.5 μg/cm², from about 0.4 to about 1 μg/cm², from about 0.4 to about 0.75 μg/cm², from about 0.4 to about 0.5 μg/cm², from about 0.5 to about 2000 μg/cm², from about 0.5 to about 1500 μg/cm², from about 0.5 to about 1000 μg/cm², from about 0.5 to about 500 μg/cm², from about 0.5 to about 450 μg/cm², from about 0.5 to about 400 μg/cm², from about 0.5 to about 350 μg/cm², from about 0.5 to about 300 μg/cm², from about 0.5 to about 250 μg/cm², from about 0.5 to about 200 μg/cm², from about 0.5 to about 190 μg/cm², from about 0.5 to about 180 μg/cm², from about 0.5 to about 170 μg/cm², from about 0.5 to about 160 μg/cm², from about 0.5 to about 150 μg/cm², from about 0.5 to about 140 μg/cm², from about 0.5 to about 130 μg/cm², from about 0.5 to about 120 μg/cm², from about 0.5 to about 110 μg/cm², from about 0.5 to about 100 μg/cm², from about 0.5 to about 90 μg/cm², from about 0.5 to about 80 μg/cm², from about 0.5 to about 70 μg/cm², from about 0.5 to about 60 μg/cm², from about 0.5 to about 50 μg/cm², from about 0.5 to about 40 μg/cm², from about 0.5 to about 30 μg/cm², from about 0.5 to about 20 μg/cm², from about 0.5 to about 10 μg/cm², from about 0.5 to about 9 μg/cm², from about 0.5 to about 8 μg/cm², from about 0.5 to about 7 μg/cm², from about 0.5 to about 6 μg/cm², from about 0.5 to about 5 μg/cm², from about 0.5 to about 4.5 μg/cm², from about 0.5 to about 4 μg/cm², from about 0.5 to about 3.5 μg/cm², from about 0.5 to about 3 μg/cm², from about 0.5 to about 2.5 μg/cm², from about 0.5 to about 2 μg/cm², from about 0.5 to about 1.5 μg/cm², from about 0.5 to about 1 μg/cm², from about 0.5 to about 0.75 μg/cm², from about 1 to about 2000 μg/cm², from about 1 to about 1500 μg/cm², from about 1 to about 1000 μg/cm², from about 1 to about 500 μg/cm², from about 1 to about 450 μg/cm², from about 1 to about 400 μg/cm², from about 1 to about 350 μg/cm², from about 1 to about 300 μg/cm², from about 1 to about 250 μg/cm², from about 1 to about 200 μg/cm², from about 1 to about 190 μg/cm², from about 1 to about 180 μg/cm², from about 1 to about 170 μg/cm², from about 1 to about 160 μg/cm², from about 1 to about 150 μg/cm², from about 1 to about 140 μg/cm², from about 1 to about 130 μg/cm², from about 1 to about 120 μg/cm², from about 1 to about 110 μg/cm², from about 1 to about 100 μg/cm², from about 1 to about 90 μg/cm², from about 1 to about 80 μg/cm², from about 1 to about 70 μg/cm², from about 1 to about 60 μg/cm², from about 1 to about 50 μg/cm², from about 1 to about 40 μg/cm², from about 1 to about 30 μg/cm², from about 1 to about 20 μg/cm², from about 1 to about 10 μg/cm², from about 1 to about 9 μg/cm², from about 1 to about 8 μg/cm², from about 1 to about 7 μg/cm², from about 1 to about 6 μg/cm², from about 1 to about 5 μg/cm², from about 1 to about 4.5 μg/cm², from about 1 to about 4 μg/cm², from about 1 to about 3.5 μg/cm², from about 1 to about 3 μg/cm², from about 1 to about 2.5 μg/cm², from about 1 to about 2 μg/cm², from about 1 to about 1.5 μg/cm², from about 2 to about 2000 μg/cm², from about 2 to about 1500 μg/cm², from about 2 to about 1000 μg/cm², from about 2 to about 500 μg/cm², from about 2 to about 450 μg/cm², from about 2 to about 400 μg/cm², from about 2 to about 350 μg/cm², from about 2 to about 300 μg/cm², from about 2 to about 250 μg/cm², from about 2 to about 200 μg/cm², from about 2 to about 190 μg/cm², from about 2 to about 180 μg/cm², from about 2 to about 170 μg/cm², from about 2 to about 160 μg/cm², from about 2 to about 150 μg/cm², from about 2 to about 140 μg/cm², from about 2 to about 130 μg/cm², from about 2 to about 120 μg/cm², from about 2 to about 110 μg/cm², from about 2 to about 100 μg/cm², from about 2 to about 90 μg/cm², from about 2 to about 80 μg/cm², from about 2 to about 70 μg/cm², from about 2 to about 60 μg/cm², from about 2 to about 50 μg/cm², from about 2 to about 40 μg/cm², from about 2 to about 30 μg/cm², from about 2 to about 20 μg/cm², from about 2 to about 10 μg/cm², from about 2 to about 9 μg/cm², from about 2 to about 8 μg/cm², from about 2 to about 7 μg/cm², from about 2 to about 6 μg/cm², from about 2 to about 5 μg/cm², from about 2 to about 4.5 μg/cm², from about 2 to about 4 μg/cm², from about 2 to about 3.5 μg/cm², from about 2 to about 3 μg/cm², from about 2 to about 2.5 μg/cm², from about 3 to about 2000 μg/cm², from about 3 to about 1500 μg/cm², from about 3 to about 1000 μg/cm², from about 3 to about 500 μg/cm², from about 3 to about 450 μg/cm², from about 3 to about 400 μg/cm², from about 3 to about 350 μg/cm², from about 3 to about 300 μg/cm², from about 3 to about 250 μg/cm², from about 3 to about 200 μg/cm², from about 3 to about 190 μg/cm², from about 3 to about 180 μg/cm², from about 3 to about 170 μg/cm², from about 3 to about 160 μg/cm², from about 3 to about 150 μg/cm², from about 3 to about 140 μg/cm², from about 3 to about 130 μg/cm², from about 3 to about 120 μg/cm², from about 3 to about 110 μg/cm², from about 3 to about 100 μg/cm², from about 3 to about 90 μg/cm², from about 3 to about 80 μg/cm², from about 3 to about 70 μg/cm², from about 3 to about 60 μg/cm², from about 3 to about 50 μg/cm², from about 3 to about 40 μg/cm², from about 3 to about 30 μg/cm², from about 3 to about 20 μg/cm², from about 3 to about 10 μg/cm², from about 3 to about 9 μg/cm², from about 3 to about 8 μg/cm², from about 3 to about 7 μg/cm², from about 3 to about 6 μg/cm², from about 3 to about 5 μg/cm², from about 3 to about 4.5 μg/cm², from about 3 to about 4 μg/cm², from about 3 to about 3.5 μg/cm², from about 4 to about 2000 μg/cm², from about 4 to about 1500 μg/cm², from about 4 to about 1000 μg/cm², from about 4 to about 500 μg/cm², from about 4 to about 450 μg/cm², from about 4 to about 400 μg/cm², from about 4 to about 350 μg/cm², from about 4 to about 300 μg/cm², from about 4 to about 250 μg/cm², from about 4 to about 200 μg/cm², from about 4 to about 190 μg/cm², from about 4 to about 180 μg/cm², from about 4 to about 170 μg/cm², from about 4 to about 160 μg/cm², from about 4 to about 150 μg/cm², from about 4 to about 140 μg/cm², from about 4 to about 130 μg/cm², from about 4 to about 120 μg/cm², from about 4 to about 110 μg/cm², from about 4 to about 100 μg/cm², from about 4 to about 90 μg/cm², from about 4 to about 80 μg/cm², from about 4 to about 70 μg/cm², from about 4 to about 60 μg/cm², from about 4 to about 50 μg/cm², from about 4 to about 40 μg/cm², from about 4 to about 30 μg/cm², from about 4 to about 20 μg/cm², from about 4 to about 10 μg/cm², from about 4 to about 9 μg/cm², from about 4 to about 8 μg/cm², from about 4 to about 7 μg/cm², from about 4 to about 6 μg/cm², from about 4 to about 5 μg/cm², from about 4 to about 4.5 μg/cm², from about 5 to about 2000 μg/cm², from about 5 to about 1500 μg/cm², from about 5 to about 1000 μg/cm², from about 5 to about 500 μg/cm², from about 5 to about 450 μg/cm², from about 5 to about 400 μg/cm², from about 5 to about 350 μg/cm², from about 5 to about 300 μg/cm², from about 5 to about 250 μg/cm², from about 5 to about 200 μg/cm², from about 5 to about 190 μg/cm², from about 5 to about 180 μg/cm², from about 5 to about 170 μg/cm², from about 5 to about 160 μg/cm², from about 5 to about 150 μg/cm², from about 5 to about 140 μg/cm², from about 5 to about 130 μg/cm², from about 5 to about 120 μg/cm², from about 5 to about 110 μg/cm², from about 5 to about 100 μg/cm², from about 5 to about 90 μg/cm², from about 5 to about 80 μg/cm², from about 5 to about 70 μg/cm², from about 5 to about 60 μg/cm², from about 5 to about 50 μg/cm², from about 5 to about 40 μg/cm², from about 5 to about 30 μg/cm², from about 5 to about 20 μg/cm², from about 5 to about 10 μg/cm², from about 5 to about 9 μg/cm², from about 5 to about 8 μg/cm², from about 5 to about 7 μg/cm², from about 5 to about 6 μg/cm², from about 6 to about 2000 μg/cm², from about 6 to about 1500 μg/cm², from about 6 to about 1000 μg/cm², from about 6 to about 500 μg/cm², from about 6 to about 450 μg/cm², from about 6 to about 400 μg/cm², from about 6 to about 350 μg/cm², from about 6 to about 300 μg/cm², from about 6 to about 250 μg/cm², from about 6 to about 200 μg/cm², from about 6 to about 190 μg/cm², from about 6 to about 180 μg/cm², from about 6 to about 170 μg/cm², from about 6 to about 160 μg/cm², from about 6 to about 150 μg/cm², from about 6 to about 140 μg/cm², from about 6 to about 130 μg/cm², from about 6 to about 120 μg/cm², from about 6 to about 110 μg/cm², from about 6 to about 100 μg/cm², from about 6 to about 90 μg/cm², from about 6 to about 80 μg/cm², from about 6 to about 70 μg/cm², from about 6 to about 60 μg/cm², from about 6 to about 50 μg/cm², from about 6 to about 40 μg/cm², from about 6 to about 30 μg/cm², from about 6 to about 20 μg/cm², from about 6 to about 10 μg/cm², from about 6 to about 9 μg/cm², from about 6 to about 8 μg/cm², from about 6 to about 7 μg/cm², from about 7 to about 2000 μg/cm², from about 7 to about 1500 μg/cm², from about 7 to about 1000 μg/cm², from about 7 to about 500 μg/cm², from about 7 to about 450 μg/cm², from about 7 to about 400 μg/cm², from about 7 to about 350 μg/cm², from about 7 to about 300 μg/cm², from about 7 to about 250 μg/cm², from about 7 to about 200 μg/cm², from about 7 to about 190 μg/cm², from about 7 to about 180 μg/cm², from about 7 to about 170 μg/cm², from about 7 to about 160 μg/cm², from about 7 to about 150 μg/cm², from about 7 to about 140 μg/cm², from about 7 to about 130 μg/cm², from about 7 to about 120 μg/cm², from about 7 to about 110 μg/cm², from about 7 to about 100 μg/cm², from about 7 to about 90 μg/cm², from about 7 to about 80 μg/cm², from about 7 to about 70 μg/cm², from about 7 to about 60 μg/cm², from about 7 to about 50 μg/cm², from about 7 to about 40 μg/cm², from about 7 to about 30 μg/cm², from about 7 to about 20 μg/cm², from about 7 to about 10 μg/cm², from about 7 to about 9 μg/cm², from about 7 to about 8 μg/cm², from about 8 to about 2000 μg/cm², from about 8 to about 1500 μg/cm², from about 8 to about 1000 μg/cm², from about 8 to about 500 μg/cm², from about 8 to about 450 μg/cm², from about 8 to about 400 μg/cm², from about 8 to about 350 μg/cm², from about 8 to about 300 μg/cm², from about 8 to about 250 μg/cm², from about 8 to about 200 μg/cm², from about 8 to about 190 μg/cm², from about 8 to about 180 μg/cm², from about 8 to about 170 μg/cm², from about 8 to about 160 μg/cm², from about 8 to about 150 μg/cm², from about 8 to about 140 μg/cm², from about 8 to about 130 μg/cm², from about 8 to about 120 μg/cm², from about 8 to about 110 μg/cm², from about 8 to about 100 μg/cm², from about 8 to about 90 μg/cm², from about 8 to about 80 μg/cm², from about 8 to about 70 μg/cm², from about 8 to about 60 μg/cm², from about 8 to about 50 μg/cm², from about 8 to about 40 μg/cm², from about 8 to about 30 μg/cm², from about 8 to about 20 μg/cm², from about 8 to about 10 μg/cm², from about 8 to about 9 μg/cm², from about 9 to about 2000 μg/cm², from about 9 to about 1500 μg/cm², from about 9 to about 1000 μg/cm², from about 9 to about 500 μg/cm², from about 9 to about 450 μg/cm², from about 9 to about 400 μg/cm², from about 9 to about 350 μg/cm², from about 9 to about 300 μg/cm², from about 9 to about 250 μg/cm², from about 9 to about 200 μg/cm², from about 9 to about 190 μg/cm², from about 9 to about 180 μg/cm², from about 9 to about 170 μg/cm², from about 9 to about 160 μg/cm², from about 9 to about 150 μg/cm², from about 9 to about 140 μg/cm², from about 9 to about 130 μg/cm², from about 9 to about 120 μg/cm², from about 9 to about 110 μg/cm², from about 9 to about 100 μg/cm², from about 9 to about 90 μg/cm², from about 9 to about 80 μg/cm², from about 9 to about 70 μg/cm², from about 9 to about 60 μg/cm², from about 9 to about 50 μg/cm², from about 9 to about 40 μg/cm², from about 9 to about 30 μg/cm², from about 9 to about 20 μg/cm², from about 9 to about 10 μg/cm², from about 10 to about 2000 μg/cm², from about 10 to about 1500 μg/cm², from about 10 to about 1000 μg/cm², from about 10 to about 500 μg/cm², from about 10 to about 450 μg/cm², from about 10 to about 400 μg/cm², from about 10 to about 350 μg/cm², from about 10 to about 300 μg/cm², from about 10 to about 250 μg/cm², from about 10 to about 200 μg/cm², from about 10 to about 190 μg/cm², from about 10 to about 180 μg/cm², from about 10 to about 170 μg/cm², from about 10 to about 160 μg/cm², from about 10 to about 150 μg/cm², from about 10 to about 140 μg/cm², from about 10 to about 130 μg/cm², from about 10 to about 120 μg/cm², from about 10 to about 110 μg/cm², from about 10 to about 100 μg/cm², from about 10 to about 90 μg/cm², from about 10 to about 80 μg/cm², from about 10 to about 70 μg/cm², from about 10 to about 60 μg/cm², from about 10 to about 50 μg/cm², from about 10 to about 40 μg/cm², from about 10 to about 30 μg/cm², from about 10 to about 20 μg/cm², from about 20 to about 2000 μg/cm², from about 20 to about 1500 μg/cm², from about 20 to about 1000 μg/cm², from about 20 to about 500 μg/cm², from about 20 to about 450 μg/cm², from about 20 to about 400 μg/cm², from about 20 to about 350 μg/cm², from about 20 to about 300 μg/cm², from about 20 to about 250 μg/cm², from about 20 to about 200 μg/cm², from about 20 to about 190 μg/cm², from about 20 to about 180 μg/cm², from about 20 to about 170 μg/cm², from about 20 to about 160 μg/cm², from about 20 to about 150 μg/cm², from about 20 to about 140 μg/cm², from about 20 to about 130 μg/cm², from about 20 to about 120 μg/cm², from about 20 to about 110 μg/cm², from about 20 to about 100 μg/cm², from about 20 to about 90 μg/cm², from about 20 to about 80 μg/cm², from about 20 to about 70 μg/cm², from about 20 to about 60 μg/cm², from about 20 to about 50 μg/cm², from about 20 to about 40 μg/cm², from about 20 to about 30 μg/cm², from about 30 to about 2000 μg/cm², from about 30 to about 1500 μg/cm², from about 30 to about 1000 μg/cm², from about 30 to about 500 μg/cm², from about 30 to about 450 μg/cm², from about 30 to about 400 μg/cm², from about 30 to about 350 μg/cm², from about 30 to about 300 μg/cm², from about 30 to about 250 μg/cm², from about 30 to about 200 μg/cm², from about 30 to about 190 μg/cm², from about 30 to about 180 μg/cm², from about 30 to about 170 μg/cm², from about 30 to about 160 μg/cm², from about 30 to about 150 μg/cm², from about 30 to about 140 μg/cm², from about 30 to about 130 μg/cm², from about 30 to about 120 μg/cm², from about 30 to about 110 μg/cm², from about 30 to about 100 μg/cm², from about 30 to about 90 μg/cm², from about 30 to about 80 μg/cm², from about 30 to about 70 μg/cm², from about 30 to about 60 μg/cm², from about 30 to about 50 μg/cm², from about 30 to about 40 μg/cm², from about 40 to about 2000 μg/cm², from about 40 to about 1500 μg/cm², from about 40 to about 1000 μg/cm², from about 40 to about 500 μg/cm², from about 40 to about 450 μg/cm², from about 40 to about 400 μg/cm², from about 40 to about 350 μg/cm², from about 40 to about 300 μg/cm², from about 40 to about 250 μg/cm², from about 40 to about 200 μg/cm², from about 40 to about 190 μg/cm², from about 40 to about 180 μg/cm², from about 40 to about 170 μg/cm², from about 40 to about 160 μg/cm², from about 40 to about 150 μg/cm², from about 40 to about 140 μg/cm², from about 40 to about 130 μg/cm², from about 40 to about 120 μg/cm², from about 40 to about 110 μg/cm², from about 40 to about 100 μg/cm², from about 40 to about 90 μg/cm², from about 40 to about 80 μg/cm², from about 40 to about 70 μg/cm², from about 40 to about 60 μg/cm², from about 40 to about 50 μg/cm², from about 50 to about 2000 μg/cm², from about 50 to about 1500 μg/cm², from about 50 to about 1000 μg/cm², from about 50 to about 500 μg/cm², from about 50 to about 450 μg/cm², from about 50 to about 400 μg/cm², from about 50 to about 350 μg/cm², from about 50 to about 300 μg/cm², from about 50 to about 250 μg/cm², from about 50 to about 200 μg/cm², from about 50 to about 190 μg/cm², from about 50 to about 180 μg/cm², from about 50 to about 170 μg/cm², from about 50 to about 160 μg/cm², from about 50 to about 150 μg/cm², from about 50 to about 140 μg/cm², from about 50 to about 130 μg/cm², from about 50 to about 120 μg/cm², from about 50 to about 110 μg/cm², from about 50 to about 100 μg/cm², from about 50 to about 90 μg/cm², from about 50 to about 80 μg/cm², from about 50 to about 70 μg/cm², from about 50 to about 60 μg/cm², from about 60 to about 2000 μg/cm², from about 60 to about 1500 μg/cm², from about 60 to about 1000 μg/cm², from about 60 to about 500 μg/cm², from about 60 to about 450 μg/cm², from about 60 to about 400 μg/cm², from about 60 to about 350 μg/cm², from about 60 to about 300 μg/cm², from about 60 to about 250 μg/cm², from about 60 to about 200 μg/cm², from about 60 to about 190 μg/cm², from about 60 to about 180 μg/cm², from about 60 to about 170 μg/cm², from about 60 to about 160 μg/cm², from about 60 to about 150 μg/cm², from about 60 to about 140 μg/cm², from about 60 to about 130 μg/cm², from about 60 to about 120 μg/cm², from about 60 to about 110 μg/cm², from about 60 to about 100 μg/cm², from about 60 to about 90 μg/cm², from about 60 to about 80 μg/cm², from about 60 to about 70 μg/cm², from about 70 to about 2000 μg/cm², from about 70 to about 1500 μg/cm², from about 70 to about 1000 μg/cm², from about 70 to about 500 μg/cm², from about 70 to about 450 μg/cm², from about 70 to about 400 μg/cm², from about 70 to about 350 μg/cm², from about 70 to about 300 μg/cm², from about 70 to about 250 μg/cm², from about 70 to about 200 μg/cm², from about 70 to about 190 μg/cm², from about 70 to about 180 μg/cm², from about 70 to about 170 μg/cm², from about 70 to about 160 μg/cm², from about 70 to about 150 μg/cm², from about 70 to about 140 μg/cm², from about 70 to about 130 μg/cm², from about 70 to about 120 μg/cm², from about 70 to about 110 μg/cm², from about 70 to about 100 μg/cm², from about 70 to about 90 μg/cm², from about 70 to about 80 μg/cm², from about 80 to about 2000 μg/cm², from about 80 to about 1500 μg/cm², from about 80 to about 1000 μg/cm², from about 80 to about 500 μg/cm², from about 80 to about 450 μg/cm², from about 80 to about 400 μg/cm², from about 80 to about 350 μg/cm², from about 80 to about 300 μg/cm², from about 80 to about 250 μg/cm², from about 80 to about 200 μg/cm², from about 80 to about 190 μg/cm², from about 80 to about 180 μg/cm², from about 80 to about 170 μg/cm², from about 80 to about 160 μg/cm², from about 80 to about 150 μg/cm², from about 80 to about 140 μg/cm², from about 80 to about 130 μg/cm², from about 80 to about 120 μg/cm², from about 80 to about 110 μg/cm², from about 80 to about 100 μg/cm², from about 80 to about 90 μg/cm², from about 90 to about 2000 μg/cm², from about 90 to about 1500 μg/cm², from about 90 to about 1000 μg/cm², from about 90 to about 500 μg/cm², from about 90 to about 450 μg/cm², from about 90 to about 400 μg/cm², from about 90 to about 350 μg/cm², from about 90 to about 300 μg/cm², from about 90 to about 250 μg/cm², from about 90 to about 200 μg/cm², from about 90 to about 190 μg/cm², from about 90 to about 180 μg/cm², from about 90 to about 170 μg/cm², from about 90 to about 160 μg/cm², from about 90 to about 150 μg/cm², from about 90 to about 140 μg/cm², from about 90 to about 130 μg/cm², from about 90 to about 120 μg/cm², from about 90 to about 110 μg/cm², from about 90 to about 100 μg/cm², from about 100 to about 2000 μg/cm², from about 100 to about 1500 μg/cm², from about 100 to about 1000 μg/cm², from about 100 to about 500 μg/cm², from about 100 to about 450 μg/cm², from about 100 to about 400 μg/cm², from about 100 to about 350 μg/cm², from about 100 to about 300 μg/cm², from about 100 to about 250 μg/cm², from about 100 to about 200 μg/cm², from about 100 to about 190 μg/cm², from about 100 to about 180 μg/cm², from about 100 to about 170 μg/cm², from about 100 to about 160 μg/cm², from about 100 to about 150 μg/cm², from about 100 to about 140 μg/cm², from about 100 to about 130 μg/cm², from about 100 to about 120 μg/cm², from about 100 to about 110 μg/cm², from about 110 to about 2000 μg/cm², from about 110 to about 1500 μg/cm², from about 110 to about 1000 μg/cm², from about 110 to about 500 μg/cm², from about 110 to about 450 μg/cm², from about 110 to about 400 μg/cm², from about 110 to about 350 μg/cm², from about 110 to about 300 μg/cm², from about 110 to about 250 μg/cm², from about 110 to about 200 μg/cm², from about 110 to about 190 μg/cm², from about 110 to about 180 μg/cm², from about 110 to about 170 μg/cm², from about 110 to about 160 μg/cm², from about 110 to about 150 μg/cm², from about 110 to about 140 μg/cm², from about 110 to about 130 μg/cm², from about 110 to about 120 μg/cm², from about 120 to about 2000 μg/cm², from about 120 to about 1500 μg/cm², from about 120 to about 1000 μg/cm², from about 120 to about 500 μg/cm², from about 120 to about 450 μg/cm², from about 120 to about 400 μg/cm², from about 120 to about 350 μg/cm², from about 120 to about 300 μg/cm², from about 120 to about 250 μg/cm², from about 120 to about 200 μg/cm², from about 120 to about 190 μg/cm², from about 120 to about 180 μg/cm², from about 120 to about 170 μg/cm², from about 120 to about 160 μg/cm², from about 120 to about 150 μg/cm², from about 120 to about 140 μg/cm², from about 120 to about 130 μg/cm², from about 130 to about 2000 μg/cm², from about 130 to about 1500 μg/cm², from about 130 to about 1000 μg/cm², from about 130 to about 500 μg/cm², from about 130 to about 450 μg/cm², from about 130 to about 400 μg/cm², from about 130 to about 350 μg/cm², from about 130 to about 300 μg/cm², from about 130 to about 250 μg/cm², from about 130 to about 200 μg/cm², from about 130 to about 190 μg/cm², from about 130 to about 180 μg/cm², from about 130 to about 170 μg/cm², from about 130 to about 160 μg/cm², from about 130 to about 150 μg/cm², from about 130 to about 140 μg/cm², from about 140 to about 2000 μg/cm², from about 140 to about 1500 μg/cm², from about 140 to about 1000 μg/cm², from about 140 to about 500 μg/cm², from about 140 to about 450 μg/cm², from about 140 to about 400 μg/cm², from about 140 to about 350 μg/cm², from about 140 to about 300 μg/cm², from about 140 to about 250 μg/cm², from about 140 to about 200 μg/cm², from about 140 to about 190 μg/cm², from about 140 to about 180 μg/cm², from about 140 to about 170 μg/cm², from about 140 to about 160 μg/cm², from about 140 to about 150 μg/cm², from about 150 to about 2000 μg/cm², from about 150 to about 1500 μg/cm², from about 150 to about 1000 μg/cm², from about 150 to about 500 μg/cm², from about 150 to about 450 μg/cm², from about 150 to about 400 μg/cm², from about 150 to about 350 μg/cm², from about 150 to about 300 μg/cm², from about 150 to about 250 μg/cm², from about 150 to about 200 μg/cm², from about 150 to about 190 μg/cm², from about 150 to about 180 μg/cm², from about 150 to about 170 μg/cm², from about 150 to about 160 μg/cm², from about 160 to about 2000 μg/cm², from about 160 to about 1500 μg/cm², from about 160 to about 1000 μg/cm², from about 160 to about 500 μg/cm², from about 160 to about 450 μg/cm², from about 160 to about 400 μg/cm², from about 160 to about 350 μg/cm², from about 160 to about 300 μg/cm², from about 160 to about 250 μg/cm², from about 160 to about 200 μg/cm², from about 160 to about 190 μg/cm², from about 160 to about 180 μg/cm², from about 160 to about 170 μg/cm², from about 170 to about 2000 μg/cm², from about 170 to about 1500 μg/cm², from about 170 to about 1000 μg/cm², from about 170 to about 500 μg/cm², from about 170 to about 450 μg/cm², from about 170 to about 400 μg/cm², from about 170 to about 350 μg/cm², from about 170 to about 300 μg/cm², from about 170 to about 250 μg/cm², from about 170 to about 200 μg/cm², from about 170 to about 190 μg/cm², from about 170 to about 180 μg/cm², from about 180 to about 2000 μg/cm², from about 180 to about 1500 μg/cm², from about 180 to about 1000 μg/cm², from about 180 to about 500 μg/cm², from about 180 to about 450 μg/cm², from about 180 to about 400 μg/cm², from about 180 to about 350 μg/cm², from about 180 to about 300 μg/cm², from about 180 to about 250 μg/cm², from about 180 to about 200 μg/cm², from about 180 to about 190 μg/cm², from about 190 to about 2000 μg/cm², from about 190 to about 1500 μg/cm², from about 190 to about 1000 μg/cm², from about 190 to about 500 μg/cm², from about 190 to about 450 μg/cm², from about 190 to about 400 μg/cm², from about 190 to about 350 μg/cm², from about 190 to about 300 μg/cm², from about 190 to about 250 μg/cm², from about 190 to about 200 μg/cm², from about 200 to about 2000 μg/cm², from about 200 to about 1500 μg/cm², from about 200 to about 1000 μg/cm², from about 200 to about 500 μg/cm², from about 200 to about 450 μg/cm², from about 200 to about 400 μg/cm², from about 200 to about 350 μg/cm², from about 200 to about 300 μg/cm², from about 200 to about 250 μg/cm², from about 250 to about 2000 μg/cm², from about 250 to about 1500 μg/cm², from about 250 to about 1000 μg/cm², from about 250 to about 500 μg/cm², from about 250 to about 450 μg/cm², from about 250 to about 400 μg/cm², from about 250 to about 350 μg/cm², from about 250 to about 300 μg/cm², from about 300 to about 2000 μg/cm², from about 300 to about 1500 μg/cm², from 300 to about 1300 μg/cm², from about 300 to about 1000 μg/cm², from about 300 to about 500 μg/cm², from about 300 to about 450 μg/cm², from about 300 to about 400 μg/cm², from about 300 to about 350 μg/cm², from about 350 to about 2000 μg/cm², from about 350 to about 1500 μg/cm², from about 350 to about 1000 μg/cm², from about 350 to about 500 μg/cm², from about 350 to about 450 μg/cm², from about 350 to about 400 μg/cm², from about 400 to about 2000 μg/cm², from about 400 to about 1500 μg/cm², from about 400 to about 1000 μg/cm², from about 400 to about 500 μg/cm², from about 400 to about 450 μg/cm², from about 450 to about 2000 μg/cm², from about 450 to about 1500 μg/cm², from about 450 to about 1000 μg/cm², from about 450 to about 500 μg/cm², from about 500 to about 2000 μg/cm², from about 500 to about 1500 μg/cm², from about 500 to about 1000 μg/cm², from about 1000 to about 2000 μg/cm², from about 1000 to about 1500 μg/cm², from about 1500 to about 2000 μg/cm², or about 0.1, 02., 0.3, 0.4, 0.5, 0.75, 1, 1.5, 2, 2.5, 3, 3.5, 4, 4.5, 5, 6, 7, 8, 9, 10, 20, 30, 40, 50, 60 70, 80, 90, 100, 110, 120, 130, 140, 150, 160, 170, 180, 190, 200, 250, 500, 1000, 1100, 1200, 1300, 1400, 1500 or 200 μg/cm² or more over a period of 4, 5, 6, 7, 8, or 24 hours.

In any of the foregoing embodiments, the active composition can be in a liquid form or a solid form. By way of example, but not limitation, the active composition can be in the form of a cream, a lotion, an eye drop, an ear drop, a sinus rinse, a spray such as for nasal, oral mucosal, skin or foot treatment, an ointment, a deodorant, a body wash, a shampoo, a scalp treatment, a mouthwash, a toothpaste, a lozenge, a beverage, a capsule, a lubricant, or a powder, such as produced by spray drying a nanoemulsion (or microemulsion) of the present disclosure. In some embodiments, the active composition further includes a carrier formulation. Thus, while in some embodiments, the nanoemulsion (or microemulsion) of the present disclosure can not include any exogenous additive with surfactant and/or emulsifier properties that is not naturally present in the oil in an amount sufficient to enhance droplet or particle stability, any exogenous additive with thickening or crystallization inhibiting properties that is not naturally present in the oil in an amount sufficient to thicken the composition or inhibit crystal formation, respectively, a polyhydric alcohol, or bile salts, the carrier formulation can include these agents. By way of example, but not limitation, the active composition can be formulated as a cream which may require a thickener or other agent, however, the nanoemulsion (or microemulsion) component can, in some embodiments, not include such an agent. In some embodiments, the active composition does not include any exogenous additive with surfactant and/or emulsifier properties that is not naturally present in the oil in an amount sufficient to enhance droplet or particle stability, any exogenous additive with thickening or crystallization inhibiting properties that is not naturally present in the oil in an amount sufficient to thicken the composition or inhibit crystal formation, respectively, a polyhydric alcohol, or bile salts.

In any of the foregoing embodiments, the active composition can contain the compositions of the present disclosure including the oil, at about 1% to about about 15% by weight out of the total weight of the active composition. By way of example, but not limitation, the compositions of the present disclosure can be present at between about 1% and about 15%, about 2% and about 15%, about 3% and about 15%, about 4% and about 15%, about 5% and about 15%, about 10% and about 15%, about 1% and about 10%, about 2% and about 10%, about 3% and about 10%, about 4% and about 10%, about 5% and about 10%, about 1% and about 5%, about 2% and about 5%, about 3% and about 5%, about 4% and about 5%, about 1% and about 4%, about 2% and about 4%, about 3% and about 4%, about 1% and about 3%, about 2% and about 3%, about 1% and anout 2%, or about 1%, 2%, 3%, 4%, 5%, 6%, 7%, 8%, 9%, 10%, or 15% by weight out of the total weight of the active composition.

In some embodiments, a method is provided for administering an active ingredient to a subject which includes a step of administering an active composition of any of the foregoing embodiments to the subject. By way of example, but not limitation, the step of administering can be performed orally, by injection, such as intravenously, intramuscularly, intrathecally or subcutaneously, sublingually, buccally, rectally, vaginally, by the ocular route, by the otic route, nasally, such as by spray, inhalation, such as by nebulization, cutaneously, such as by topical application, via implantation, transdermally, or systemically.

In some embodiments, a method is provided for preparing an active composition of the present disclosure which includes combining the active ingredient with the oil and forming an emulsion, thereby yielding the active ingredient in the oil of an active composition of the present disclosure. It should be understood that the active ingredient can be added to the oil before forming the emulsion or to the composition after forming the emulsion and that the emulsion can have the properties of the the nanoemulsions and microemulsions of the present disclosure. By way of example, but not limitation, the method of preparing an active composition of the present disclosure can include providing an oil of the present disclosure, combining the oil and the active ingredient, providing a polar liquid, combining the oil and the polar liquid to form an emulsion pre-mix, and forming an emulsion from the nanoemulsion pre-mix. Alternatively, the active ingredient can be added after formation of the nanoemulsion or microemulsion. By way of further example, but not limitation, the emulsion can be nanoemulsion or microemulsion as described in the present disclosure. The method for forming the emulsion can be any method known in the art or as described in the present disclosure. It should be understood that the method for preparing an active composition of the present disclosure can, in certain aspects, further include adding a carrier formulation to the active composition. While in some embodiments, the nanoemulsion (or microemulsion) can not include any exogenous additive with surfactant and/or emulsifier properties that is not naturally present in the oil in an amount sufficient to enhance droplet or particle stability, any exogenous additive with thickening or crystallization inhibiting properties that is not naturally present in the oil in an amount sufficient to thicken the composition or inhibit crystal formation, respectively, a polyhydric alcohol, or bil salts, the carrier formulation can include these agents. By way of example, but not limitation, the active composition can be further formulated into cream which may require a thickener or other agent, however, the nanoemulsion (or microemulsion) component can, in some embodiments, not include such an agent. In some embodiments, the active composition does not include any exogenous additive with surfactant and/or emulsifier properties that is not naturally present in the oil in an amount sufficient to enhance droplet or particle stability, any exogenous additive with thickening or crystallization inhibiting properties that is not naturally present in the oil in an amount sufficient to thicken the composition or inhibit crystal formation, respectively, a polyhydric alcohol, or bile salts.

In some embodiments, an active composition can include a first component which includes the nanoemulsion (or microemulsion) of the present disclosure, and a second component which includes a carrier formulation. In such embodiments, the first component can further include an active ingredient and the active ingredient can be present in the oil of the nanoemulsion (or microemulsion) of the present disclosure. Alternatively, the active ingredient can be present in an aqueous phase of the nanoemulsion or microemulsion. While in some embodiments, the nanoemulsion (or microemulsion) can not include any exogenous additive with surfactant and/or emulsifier properties that is not naturally present in the oil in an amount sufficient to enhance droplet or particle stability, any exogenous additive with thickening or crystallization inhibiting properties that is not naturally present in the oil in an amount sufficient to thicken the composition or inhibit crystal formation, respectively, a polyhydric alcohol, or bil salts, the carrier formulation can include these agents. By way of example, but not limitation, the active composition can be further formulated into cream which may require a thickener or other agent, however, the nanoemulsion (or microemulsion) component can, in some embodiments, not include such an agent. In some embodiments, the active composition does not include any exogenous additive with surfactant and/or emulsifier properties that is not naturally present in the oil in an amount sufficient to enhance droplet or particle stability, any exogenous additive with thickening or crystallization inhibiting properties that is not naturally present in the oil in an amount sufficient to thicken the composition or inhibit crystal formation, respectively, a polyhydric alcohol, or bile salts.

Methods of Treatment

In some embodiments, a method of treating or preventing acne in a subject in need thereof can include administering an active composition of the present disclosure to the subject where the active ingredient is administered in a therapeutically effective amount and is suitable to treat or prevent acne. In some embodiments, the active ingredient is salicylic acid.

In some embodiments, a method of treating or preventing pain in a subject in need thereof can include administering an active composition of the present disclosure to the subject where the active ingredient is administered in a therapeutically effective amount and is suitable to treat or prevent pain. In some embodiments, the active ingredient is lidocaine, prilocaine, bupivacaine, or a combination thereof.

In some embodiments, a method of treating or preventing inflammation in a subject in need thereof can include administering an active composition of the present disclosure to the subject where the active ingredient is administered in a therapeutically effective amount and is suitable to treat or prevent inflammation. In some embodiments, the active ingredient is diclofenac, cannabidiol, aspartyl-alanyl-diketopiperazine, hydrocortisone, or a combination thereof.

In some embodiments, a method of treating or preventing cancer in a subject in need thereof can include administering an active composition of the present disclosure to the subject where the active ingredient is administered in a therapeutically effective amount and is suitable to treat or prevent cancer. In some embodiments, the active ingredient is 5-fluorouracil.

In some embodiments a method of administering an anti-aging compound or vitamin to a subject in need thereof can include administering an active composition of the present disclosure to the subject where the active ingredient is in a therapeutically effective amount. In some embodiments, the active ingredient is trifluoroacetyl tripeptide-2, hexapeptide-11, retinol or retinyl palmitate.

It should be understood that the disclosure herein is not limited to the use of EMO and FFA oils and that any oil may be used that meets the requirements of the present disclosure.

### EXAMPLES

In the following examples, unless otherwise noted, the MAG, DAG, TAG and FFA content and fatty acid profiles can be as described in Table 1 below or in the foregoing tables of fatty acid content and exemplary EMO in the description. Generally, FFA oils can be assumed to be >90% FFA, usually about 93%+/−2%, unless otherwise noted.

Fatty Acid Oil Preparation

Fatty acid oils in the following examples (except Example 1) were prepared by the following procedure unless otherwise specified. For all examples, FFA oils were assumed to contain 93%+/−2% FFA with the exception of fish FFA oil which contains 77%+/−2% FFA and algal FFA oil which contains 61%+/−2% FFA. However, longer reaction times and different conditions can yield higher FFA if desired. It should be understood that as used herein, for each composition the nomenclature is n(Oil)-non-FFA % if from a single oil source and n(EMO)/(FFA)−EMO %. For example, an oil made with sesame EMO and FFA oil to yield a total of 50% FFA would be nS50 while an oil made with sesame EMO and FFA oil to yield a total of 75% FFA would be nS25. For further example, an oil made with 25% oat EMO and 75% flaxseed FFA oil would be nOa/Fx-25 and the combination would be on a weight basis.

Fatty acid oils were prepared by the complete hydrolysis of the triacylglcyerides (TAGs) in the source oil to free fatty acids (FFA) and glycerol, followed by subsequent separation of the FFA and glycerol.

Free Fatty Acid (FFA) Oil Preparation

Free fatty acid oil was prepared by the complete hydrolysis of TAGs to FFA and glycerol followed by subsequent separation of the glycerol from the FFA oil. FFA oils were prepared in a 5 L stirred, jacketed glass reactor. Citric acid buffer (100 mM, pH 5.8) was prepared by combining 18.5 grams of food grade citric acid with 1.50 g of food grade sodium hydroxide in 963 mL of distilled de-ionized water. Amano Lipase AY (0.78 g) was dissolved in the solution which was then heated to 33° C. and agitated at 300 rpm. After a brief (10 min.) vacuum degassing, 780 g of starting oil was added to the reactor. The reaction volume was overlaid with nitrogen and the reaction proceeded for 24 hours until all of the TAGs had been converted to fatty acids and glycerol. Once the reaction was complete, the mixture was heated to 70° C. for 1 hour to inactivate the lipase. Agitation was stopped. After the two phases had separated, the upper phase (the free fatty acid oil) was removed and stored at 4° C. in opaque plastic bottles under nitrogen. Components of the oil samples were separated using TLC plates (Analtech Uniplate Silica Gel GHL with inorganic binder, 20×20 cm, 250 μm). The solvent was hexane:diethyl ether:acetic acid (70:30:1) solution. Typical sample sizes were 0.2 μL. After the solvent front ran to near the top of the plate (˜1 cm), plates were removed from the TLC tank and the solvent evaporated in a fume hood. The components were visualized with iodine vapors (at room temperature) in a TLC tank and relative intensities estimated by colorimetric imaging (Amersham 600 Imager). After 15 minutes in the tank, plates were removed and photographed. The intensity of the spots diminished after 30-60 minutes.

Enzyme-Modified Oil (EMO) Preparation

Enzyme-modified oil was produced from the FFA oil through the selective re-esterification of the FFA to glycerol to form MAG and (to a much lesser extent) DAG. FFA oil as described above was combined with 1,560 g of food grade glycerol and heated to 30° C. with agitation (300 rpm). Vacuum was applied (˜720 mmHg) to degas the material and remove residual water. Amano Lipase G (1.56 g in 50 mL DI water) was then added to the reactor and the vacuum was reapplied (˜740 mmHg). The reaction was allowed to proceed for 72 hours with progress monitored by TLC. Once the level of FFA was reduced to below 20%, the reaction was stopped by removing the vacuum, overlaying the reactor with nitrogen and heating the system to 70° C. for an hour to inactivate the enzyme. Fatty acid concentrations were measured by titrimetry. Samples were dissolved in a solvent (95% ethanol/diethyl ether, 1/1, v/v) and titrated to neutrality (indicated with phenolphthalein) with 0.1M KOH in ethanol. Table salt (sodium chloride) was then added to the reactor. Once the salt was suspended, the agitation was stopped. After the two phases had separated (˜3 hours), the upper phase (the EMO) was removed. Antioxidant was added (tocopherol, 200 ppm) and the material was stored at 4° C. in opaque plastic bottles under nitrogen.

Analysis of EMOs was performed using an HPLC method described previously (Bruno Lima dos Santos, Kissya Kropf da Silva, Amanda Pereira Franco dos Santos, Debora Franga de Andrade & Luiz Antonio d'Avila (2016) Simultaneous analysis of esters and acylglycerols in biodiesel by high-performance liquid chromatography with refractive index detection, Journal of Liquid Chromatography & Related Technologies, 39:13, 620-626, DOI: 10.1080/10826076.2016.1225221).

The HPLC system consisted of an LC-20AD pump, SIL-20AC HT autosampler, CTO-20A column oven, and RID-10A refractive index detector (Shimadzu Scientific Instruments, Inc.). HPLC conditions were: 20 uL injection volume, isocratic elution of mobile phase consisting of 85% methanol, 10% isopropanol, and 5% hexanes, flow rate of 0.800 mL/min, and column temperature of 40° C. Column used was a SUPELCOSIL LC-18 column (250 mm×4.6 mm; 5 um particle size). Samples were prepared in mobile phase. A mobile phase blank chromatogram was used to correct for background.

In general, the retention times were 3.4-4.0 min for glycerol, 4.0-6.2 min for MAGs and FFAs, 6.3-10.8 min for DAGs, and 11.0-50.0 min for TAGs.

Integration of these peaks provided relative ratios, or area-%, of glycerol, MAG+FFA, DAG, and TAG.

Since the MAG and FFA peaks overlapped, FFA content was determined by titrimetry. Samples were dissolved in a solvent (95% ethanol/diethyl ether, 1/1, v/v) and titrated to neutrality (indicated with phenolphthalein) with 0.1M KOH in ethanol. This value is reported as a weight percent.

MAG content was calculated by subtracting the FFA content from the MAG+FFA determined by HPLC.

Table 1 below provides the relative glycerol, MAG, DAG, TAG, and FFA content of the resulting EMOs which are used in the following Examples unless otherwise noted

TABLE 1
Glycerol, MAG, DAG, TAG and FFA Content of EMOs

| | MAG | DAG | TAG | FFA |
|---|---|---|---|---|
| EMO | (area-%) | (area-%) | (area-%) | (wt-%) |
| Sunflower | 58.3 | 25.5 | BLD | 9 |
| Canola | 60.9 | 16.3 | BLD | 13.9 |
| Almond | 74.9 | 12.5 | BLD | 10 |
| Flaxseed | 57.9 | 16.5 | BLD | 18 |
| Sesame | 62.5 | 17.9 | BLD | 12 |
| Olive | 55.9 | 14.4 | BLD | 20 |
| Rosehip | 54.6 | 16.5 | BLD | 16 |
| Hemp | 69 | 11 | 2.1 | 9 |
| Oat | 65.9 | 9.4 | BLD | 13 |
| Algae | 3.2 | 28 | 32.1 | 36.6 |
| Fish | Est. 56 | Est. 14 | 15.7 | 12 |
| MCT | Est. 64 | Est. 15 | BLD | 21 |
| Coconut | Est. 72 | Est. 16 | BLD | 12 |

BLD = below limit of detection
*MAG and DAG peaks overlapped

Nanoemulsion Preparation

Nanoemulsions were prepared from either EMOs or a combination of EMO and FFA oil(s). 1.5 g of total oils was weighed into a 50 mL metal beaker and heated to 60° C. A coarse emulsion was prepared by adding 13.5 mL water (pre-heated to 60° C.) to the oil. The mixture was subjected to high-sheer homogenizer for 30 seconds, resulting in a milky coarse emulsion. The coarse emulsion was then transferred to QSonica Q700CA sonicator with 1%2 inch probe. The emulsion was processed for 2 cycles (5 minutes sonication/2 minutes cooling) at 90% power.

### Example 1: Production of Almond Fatty Acid Oil and Enzyme-Modified Oil (EMO)

Fatty acid oil was prepared by complete hydrolysis of the TAGs to FFA and glycerol and their subsequent separation. Almond fatty acid oil was prepared in a 20 L stirred, jacketed glass reactor. Citric acid buffer (100 mM, pH 5.8) was prepared by combining 133 grams of food grade citric acid with 75 g of food grade sodium hydroxide in 7,000 mL of distilled de-ionized water. Amano Lipase AY (5.8 g) was dissolved in the solution which was then heated to 33° C. and agitated at 300 rpm. After a brief (10 min.) vacuum degassing, 5,600 g of almond oil was added to the reactor. The reaction volume was overlaid with nitrogen and the reaction proceeded for 12 hours until all of the TAGs had been converted to fatty acids and glycerol.

Once the reaction was complete, the mixture was heated to 70° C. for 1 hour to inactivate the lipase. Agitation was stopped. After the two phases had separated, the upper phase (the fatty acid oil) was removed and stored at room temperature in opaque plastic bottles under nitrogen.

Components of the oil samples were separated using TLC plates (Analtech Uniplate Silica Gel GHL with inorganic binder, 20×20 cm, 250 μm). The solvent was hexane:diethyl ether:acetic acid (70:30:1) solution. Typical sample sizes were 0.2 μL. After the solvent front ran to near the top of the plate (˜1 cm), plates were removed from the TLC tank and the solvent evaporated in a fume hood. The components were visualized with iodine vapors (at room temperature) in a TLC tank and relative intensities estimated by colorimetric imaging (Amersham 600 Imager). After 15 minutes in the tank, plates were removed and photographed. The intensity of the spots diminished after 30-60 minutes.

EMO was produced from the fatty acid oil through the selective re-esterification of the FFA to glycerol to form MAG and (to a much lesser extent) DAG. Fatty acid oil as described above was combined with 10,260 g of food grade glycerol and heated to 30° C. with agitation (300 rpm). Vacuum was applied (˜720 mmHg) to degas the material and remove residual water. Amano Lipase G (5 g in 50 mL DI water) was then added to the reactor and the vacuum was reapplied (˜740 mmHg). The reaction was allowed to proceed for 72 hours with progress monitored by TLC.

Once the level of FFA was reduced to below 15%, the reaction was stopped by removing the vacuum, overlaying the reactor with nitrogen and heating the system to 70° C. for an hour to inactivate the enzyme.

Fatty acid concentrations were measured by titrimetry. Samples were dissolved in a solvent (95% ethanol/diethyl ether, 1/1, v/v) and titrated to neutrality (indicated with phenolphthalein) with 0.1M KOH in ethanol.

Table salt (sodium chloride) was then added to the reactor. Once the salt was suspended, the agitation was stopped. After the two phases had separated (3 hours), the upper phase (the acylglycerol oil) was removed. Antioxidant was added (tocopherol, 200 ppm) and the material was stored at room temperature in opaque plastic bottles under nitrogen.

Lipid components including C10:0 Capric Acid, C12:0 Lauric Acid, C14:0 Myristic Acid, C16:0 Palmitic Acid, C18:0 Stearic Acid, C18:1 Oleic Acid, C18:2 Linoleic Acid, and C18:3 Alpha Linolenic Acid were analyzed after derivatization as the fatty acid methyl esters and compared to standards. For the derivatization, a sample (500 μl) was added to a 5-ml reaction tube containing 2 ml boron trifluoride solution (12% in methanol), 20 μl dimethoxypropane and 100 μl of a tridecanoic acid internal standard solution (10 mg/ml). The reaction tube was vortexed and incubated in a heating block at 60° C. for 30 minutes.

The reaction tube was removed from the heating block and allowed to cool for 15 minutes. Then, 1 ml of distilled water was added to quench the reaction, followed by 1 ml of hexane. The reaction tube was vortexed for 60 seconds and the phases were allowed to separate for 3 minutes. The top (hydrophobic) phase was removed to a 1.5-ml tube containing about 50 mg sodium sulfate (anhydrous). After vortexing for 60 seconds, the 1.5-ml tube was centrifuged and ˜500 μl of the clarified, dried hydrophobic phase was transferred to a gas chromatography sample vial.

Samples were analyzed using an Agilent 7890A gas chromatograph with Flame ionization detector and Agilent Openlab CDS Chemstation software. GC Column: Omegawax 100 (15 m×0.1 mm×0.1 um) column. Results were converted to weight % by internal standard reference.

MCT, canola and almond oils were processed according to the manufacturing method of the present disclosure. Fatty acid profiles for each were measured in both the starting oil and the MAG oil as shown in Table 2 below.

As shown, both the starting oil and EMO contained substantially the same fatty acid profiles.

A separate experiment was run using almond and canola oil to obtain the data in Table 3 which show the same result for oleic, linoleic and linolenic acid.

TABLE 2
Conservation of Fatty acid profile

| | | MCT | | Canola | | Almond |
|---|---|---|---|---|---|---|
| Compound | MCT | MAG | Canola | MAG | Almond | MAG |
| C6:0 (Caproic acid) | 5.43% | 0.06% | | | | |
| C8:0 (Caprylic acid) | 58.41% | 57.42% | | | | |
| C10:0 (Capric acid) | 40.96% | 41.96% | | | | |
| C11:0 (Undecanoic acid) | 0.07% | 0.07% | | | | |
| C12:0 (Lauric Acid) | 0.32% | 0.32% | | | | |
| C14:0 (Myristic acid) | 0.03% | 0.03% | 0.05% | 0.06% | 0.06% | 0.06% |
| C16:0 (Palmitic Acid) | | | 4.06% | 4.06% | 6.61% | 6.65% |
| C16:1 Total (Palmitoleic Acid + isomers) | | | 0.30% | 0.30% | 0.57% | 0.57% |
| C17:0 (Margaric Acid) | | | 0.04% | 0.04% | 0.06% | 0.06% |
| C17:1 (Heptadecenoic Acid) | | | 0.05% | 0.06% | 0.11% | 0.11% |
| C18:0 (Stearic Acid) | | | 1.78% | 1.78% | 1.56% | 1.57% |
| C18:1 (Vaccenic acid) | | | 3.28% | 3.27% | 1.38% | 1.39% |
| C18:1, Total (Oleic Acid + isomers) | 0.04% | 0.06% | 62.78% | 62.66% | 64.79% | 64.94% |
| C18:2, Total (Linoleic Acid + isomers) | 0.04% | 0.04% | 19.07% | 19.07% | 25.28% | 25.14% |
| C18:3, Total (Linolenic Acid + isomers) | | | 8.26% | 8.22% | 0.29% | 0.28% |
| C18:4 Total (Octadecatetraenoic Acid) | | | 0.04% | 0.04% | 0.09% | 0.08% |
| C20:0 (Arachidic Acid) | | | 0.57% | 0.58% | 0.08% | 0.07% |
| C20:1 Total (Gondoic Acid + isomers) | | | 2.10% | 2.08% | 0.14% | 0.14% |
| C20:2 Total (Eicosadienoic Acid) | | | 0.11% | 0.18% | 0.12% | 0.14% |
| C22:0 (Behenic Acid) | | | 0.31% | 0.31% | 0.03% | 0.04% |
| C22:1 Total (Erucic Acid + isomers) | | | 0.02% | | | |
| C24:0 (Lignoceric Acid) | | | 0.15% | 0.16% | | |
| C24:1 Total (Nervonic Acid + isomers) | | | 0.16% | 0.16% | | |

TABLE 3
Conservation of poly unsaturated Fatty acids

| | | | Almond | Canola |
|---|---|---|---|---|
| FATTY ACID | Almond | Canola | MAG | MAG |
| C18:1cis (n-9) Oleic | 63.2 | 59.33 | 63.31 | 59.33 |
| C18:2cis (n-6) Linoleic | 25.28 | 19.07 | 25.14 | 19.07 |
| C18:3 (n-3) Linolenic | 0.29 | 8.26 | 0.2 | 8.22 |
| Oleic/Linoleic ratio | 2.5 | 3.1 | 2.5 | 3.1 |
| Oleic/Linolenic ratio | 218 | 7 | 317 | 7 |

These results demonstrate that the manufacturing process of the present disclosure is able to preserve the fatty acid profile of the starting oil in the EMO. This result is different than for conventional “distilled” oils whose fatty acid profile differs significantly from the starting oil based on the functional use of the distilled oil.

### Example 2: Preparation of Nanoemulsions from a Single Oil Source

Nanoemulsions were prepared from Medium Chain Triglyceride (MCT) enzyme-modified oil as described above. Briefly, MCT oil was subjected to complete enzymatic hydrolysis to yield a FFA oil containing free fatty acids and subsequently was further enzymatically processed in the presence of glycerol to re-esterify the FFA to MAGs and DAGs to yield a processed oil containing 79% MAGs and DAGs and 21% FFA.

To prepare the first nanoemulsion, 1.5 g of the processed MCT oil was weighed into a 50 mL beaker and heated to 60° C. A coarse emulsion was prepared by adding 13.5 mL of water (pre-heated to 60° C.) to the oil. The mixture as subjected to high sheer using a high-sheer homogenizer for 30 seconds, resulting in a milky coarse emulsion. The coarse emulsion was then transferred to a QSonica Q700CA sonicator with a ½ inch probe. The emulsion was processed for 2 cycles (5 minutes sonication/2 minutes cooling) at 90% power.

Additional nanoemulsions were prepared by combining the processed MCT oil (including MAGs and DAGs) and the MCT FFA oil yielded after enzymatic hydrolysis to achieve various ratios of MAG and DAG to FFA as shown in Table 4 below. These nanoemulsions were assessed for Z average size, polydispersity index and average zeta potential of the droplets using a Zetasizer Ultra (Malvern Panalytical) according to the manufacturer's instructions. Measurements were made in triplicate.

TABLE 4
Medium Chain Triglyceride (MCT) EMO Nanoemulsions (data
represented as average ± SD from at least three measurements);
MCT is representative of oils with medium chain
fats (C6, C8, C10, C12) and also for saturated fats

| | EMO (non- | | | | |
|---|---|---|---|---|---|
| | FFA | | | | Zeta |
| | component) | | | | potential |
| Oil | % | Total FFA % | Z avg (nm) | PDI | (mV) |
| MCT | 79 | 21 | 233 ± 5 | 0.252 ± | −31.9 ± 12.5 |
| | | | | 0.006 | |
| MCT | 50 | 50 | 191 ± 4 | 0.217 ± | −35.5 ± 9.4 |
| | | | | 0.007 | |
| MCT | 25 | 75 | 182 ± 5 | 0.213 ± | −32.0 ± 0.5 |
| | | | | 0.008 | |
| MCT | 10 | 90 | 205 ± 1 | 0.238 ± | −34.9 ± 0.3 |
| | | | | 0.019 | |
| MCT | 0 | 100 | 257 ± 4 | 0.290 ± | −40.7 ± 4.2 |
| | | | | 0.022 | |

A corresponding process was performed using olive oil with the resulting data shown in Table 5 below.

TABLE 5
Olive EMO Nanoemulsions (data represented as average ± SD from
at least three measurements); Olive oil is representative for
mono-unsaturated fats (high in oleic acid C18:1)

| | EMO (non- | | | | |
|---|---|---|---|---|---|
| | FFA | | | | Zeta |
| | component) | Total FFA | | | potential |
| Oil | % | % | Z avg (nm) | PDI | (mV) |
| Olive | 80 | 20 | 229 ± 5 | 0.187 ± 0.035 | −52.8 ± 0.8 |
| Olive | 50 | 50 | 225 ± 4 | 0.224 ± 0.018 | −63.3 ± 1.1 |
| Olive | 25 | 75 | 221 ± 2 | 0.220 ± 0.063 | −60.7 ± 1.1 |
| Olive | 10 | 90 | 237 ± 2 | 0.198 ± 0.019 | −57.7 ± 1.8 |
| Olive | 0 | 100 | 305 ± 6 | 0.169 ± 0.022 | −65.2 ± 1.1 |

A corresponding process was performed using flaxseed oil with the resulting data shown in Table 6 below.

TABLE 6
Flaxseed EMO Nanoemulsions (data represented as average ± SD from
at least three measurements); Flaxseed oil is representative for poly-
unsaturated fats (high in alpha-linoleic, C18:3)

| | EMO (non- | | | | |
|---|---|---|---|---|---|
| | FFA | | | | Zeta |
| | component) | Total FFA | | | potential |
| Oil | % | % | Z avg (nm) | PDI | (mV) |
| Flaxseed | 82 | 18 | 142 ± 3 | 0.205 ± 0.013 | −49.5 ± 1.0 |
| Flaxseed | 75 | 25 | 141 ± 3 | 0.208 ± 0.012 | −55.8 ± 0.8 |
| Flaxseed | 50 | 50 | 162 ± 5 | 0.179 ± 0.027 | −55.1 ± 3.8 |
| Flaxseed | 25 | 75 | 191 ± 3 | 0.192 ± 0.012 | −75.5 ± 0.9 |
| Flaxseed | 10 | 90 | 273 ± 3 | 0.28 ± 0.043 | −74.7 ± 1.5 |
| Flaxseed | 0 | 100 | 345 ± 12 | 0.232 ± 0.066 | −74.1 ± 1.8 |

A corresponding process was performed using sesame oil with the resulting data shown in Table 7 below.

TABLE 7
Sesame EMO Nanoemulsions (data represented as average ± SD from
at least three measurements); Sesame oil is representative for
unsaturated fats (high in linoleic acid (C18:2) and oleic acid (C18:1))

| | EMO (non- | Total | | | Zeta |
|---|---|---|---|---|---|
| | FFA | FFA | Z | | potential |
| Oil | component) % | % | avg (nm) | PDI | (mV) |
| Sesame | 88 | 12 | 181 ± 4 | 0.186 ± 0.015 | −61.4 ± 2.5 |
| Sesame | 75 | 25 | 180 ± 3 | 0.182 ± 0.023 | −64.0 ± 1.4 |
| Sesame | 50 | 50 | 180 ± 3 | 0.184 ± 0.025 | −72.4 ± 0.6 |
| Sesame | 25 | 75 | 203 ± 4 | 0.174 ± 0.022 | −66.1 ± 1.4 |
| Sesame | 10 | 90 | 250 ± 3 | 0.200 ± 0.027 | −66.2 ± 1.5 |
| Sesame | 0 | 100 | 392 ± 15 | 0.236 ± 0.088 | −70.1 ± 0.2 |

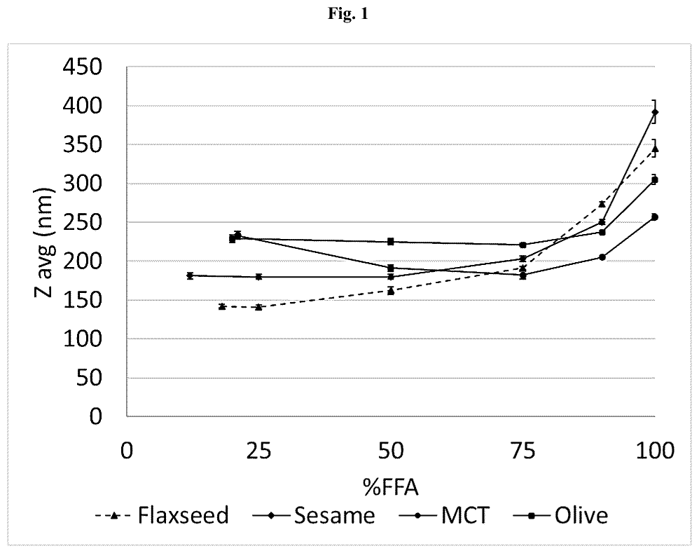

FIG. 1 depicts the Z average size of the droplets in the nanoemulsions produced from the four different starting oils as a function of % FFA.

A corresponding process was performed using rosehip oil with the resulting data shown in Table 8 below.

TABLE 8
Rosehip EMO Nanoemulsions (data represented as average ±
SD from at least three measurements); Rosehip oil is
representative for polyunsaturated fats
(high in linoleic acid (C18:2) and linolenic acid (C18:3))

| | EMO (non- | | | | |
|---|---|---|---|---|---|
| | FFA | | | | Zeta |
| | component) | Total FFA | | | potential |
| Oil | % | % | Z avg (nm) | PDI | (mV) |
| Rosehip | 75 | 25 | 203 ± 1 | 0.187 ± 0.019 | −53.3 ± 0.4 |
| Rosehip | 50 | 50 | 205 ± 3 | 0.174 ± 0.012 | −57.3 ± 1.7 |
| Rosehip | 25 | 75 | 204 ± 3 | 0.231 ± 0.026 | −54.5 ± 1.1 |
| Rosehip | 0 | 100 | 245 ± 3 | 0.200 ± 0.010 | −59.0 ± 0.3 |

A corresponding process was performed using hemp seed oil with the resulting data shown in Table 9 below.

TABLE 9
Hemp seed EMO Nanoemulsions (data represented as average ± SD from
at least three measurements); Hemp seed oil is representative for
unsaturated fats (high in linoleic acid (C18:2) and linolenic acid (C18:3))

| | EMO (non- | | | | |
|---|---|---|---|---|---|
| | FFA | | | | Zeta |
| | component) | Total FFA | | | potential |
| Oil | % | % | Z avg (nm) | PDI | (mV) |
| Hemp | 91 | 9 | 183 ± 4 | 0.215 ± 0.026 | −52.7 ± 0.6 |
| Hemp | 75 | 25 | 174 ± 1 | 0.200 ± 0.019 | −57.3 ± 1.1 |
| Hemp | 50 | 50 | 180 ± 2 | 0.199 ± 0.023 | −58.8 ± 1.9 |
| Hemp | 25 | 75 | 181 ± 2 | 0.180 ± 0.041 | −63.0 ± 1.4 |
| Hemp | 0 | 100 | 245 ± 1 | 0.197 ± 0.001 | −61.6 ± 0.4 |

A corresponding process was performed using sunflower oil with the resulting data shown in Table 10 below.

TABLE 10
Sunflower EMO Nanoemulsions (data represented as average ±
SD from at least three measurements); Sunflower oil is
representative for unsaturated fats (high in linoleic acid (C18:2))

| | EMO (non- | | | | |
|---|---|---|---|---|---|
| | FFA | Total | | | Zeta |
| | component) | FFA | | | potential |
| Oil | % | % | Z avg (nm) | PDI | (mV) |
| Sunflower | 75 | 25 | 194 ± 4 | 0.140 ± 0.016 | −59.4 ± 0.5 |
| Sunflower | 50 | 50 | 211 ± 2 | 0.158 ± 0.017 | −63.0 ± 0.2 |
| Sunflower | 25 | 75 | 228 ± 6 | 0.156 ± 0.023 | −64.4 ± 0.2 |
| Sunflower | 0 | 100 | 320 ± 9 | 0.181 ± 0.032 | −64.8 ± 1.3 |

A corresponding process was performed using oat oil with the resulting data shown in Table 11 below.

TABLE 11
Oat EMO Nanoemulsions (data represented as average ± SD from at
least three measurements); Oat oil is representative for
unsaturated fats (high in linoleic acid (C18:2) and oleic acid (C18:1))

| | EMO (non- | Total | | | Zeta |
|---|---|---|---|---|---|
| | FFA | FFA | | | potential |
| Oil | component) % | % | Z avg (nm) | PDI | (mV) |
| Oat | 87 | 13 | 166 ± 3 | 0.193 ± 0.021 | −61.6 ± 0.4 |
| Oat | 75 | 25 | 177 ± 5 | 0.184 ± 0.010 | −62.8 ± 0.2 |
| Oat | 50 | 50 | 197 ± 6 | 0.144 ± 0.041 | −62.9 ± 1.0 |
| Oat | 25 | 75 | 221 ± 5 | 0.187 ± 0.014 | −63.8 ± 1.3 |
| Oat | 0 | 100 | 361 ± 10 | 0.072 ± 0.051 | −63.7 ± 0.9 |

The foregoing results demonstrate that different ratios of MAGs and DAGs and FFAs can be utilized to prepare nanoemulsions from different starting oils. In some instances, the FFA oil alone can form a nanoemulsion without the addition of the enzyme-modified oil that includes MAGs, and to some extent, DAGs. In all cases the TAG content would be low or non-existent since the TAGs from the starting oil have been substantially or completely hydrolyzed in the first processing step. It should be understood that, where the oil in the nanoemulsion includes EMO, the EMO can be derived from a single starting oil source or from two, three or more starting oil sources. For example, the starting oil sources can be blended prior to enzymatic treatment to obtain the EMO.

### Example 3: Preparation of Nanoemulsions from Different Oil Sources

Nanoemulsions from combinations of EMOs and FFA oils from different oils at different ratios were prepared and analyzed as described above.

For the combination of coconut EMO and MCT FFA oil, the resulting data are shown in Table 12 below.

TABLE 12
Coconut EMO/MCT FFA Oil Nanoemulsions (data represented
as average ± SD from at least three measurements); This is a
representative blend for medium chain fatty acids and lauric acid

| | | | | | Zeta |
|---|---|---|---|---|---|
| | EMO | FFA | Z | | potential |
| EMO/FFA | % | oil % | avg (nm) | PDI | (mV) |
| Coconut | 100 | 0 | 138 ± 2 | 0.193 ± | −38.6 ± 0.5 |
| | | | | 0.020 | |
| Coconut/MCT | 90 | 10 | 147 ± 2 | 0.19 ± 0.012 | −30.4 ± 0.3 |
| Coconut/MCT | 75 | 25 | 154 ± 2 | 0.186 ± | −30.8 ± 0.8 |
| | | | | 0.019 | |
| Coconut/MCT | 50 | 50 | 163 ± 2 | 0.196 ± | −31.1 ± 0.5 |
| | | | | 0.015 | |
| Coconut/MCT | 25 | 75 | 184 ± 2 | 0.23 ± 0.018 | −32.6 ± 1.3 |
| Coconut/MCT | 10 | 90 | 224 ± 3 | 0.268 ± | −37.9 ± 5.6 |
| | | | | 0.012 | |
| MCT | 0 | 100 | 255 ± 4 | 0.296 ± | −35.3 ± 0.4 |
| | | | | 0.053 | |

For the combination of MCT EMO and Coconut FFA oil, the resulting data are shown in Table 13 below.

TABLE 13
MCT EMO/Coconut FFA Oil Nanoemulsions (data represented as
average ± SD from at least three measurements); This is a
representative blend for medium chain fatty acids and lauric acid (C12)

| | | | | | Zeta |
|---|---|---|---|---|---|
| | EMO | FFA | Z avg | | potential |
| EMO/FFA | % | oil % | (nm) | PDI | (mV) |
| MCT | 100 | 0 | 272 ± 6 | 0.276 ± | −30.2 ± 8.0 |
| | | | | 0.028 | |
| MCT/Coconut | 90 | 10 | 226 ± 6 | 0.248 ± | −37.6 ± 11.6 |
| | | | | 0.014 | |
| MCT/Coconut | 75 | 25 | 162 ± 3 | 0.200 ± | −32.8 ± 0.4 |
| | | | | 0.020 | |
| MCT/Coconut | 50 | 50 | 135 ± 2 | 0.193 ± | −37.3 ± 0.9 |
| | | | | 0.017 | |
| MCT/Coconut | 25 | 75 | 147 ± 2 | 0.219 ± | −41.6 ± 1.4 |
| | | | | 0.013 | |
| MCT/Coconut | 10 | 90 | 173 ± 3 | 0.220 ± | −46.3 ± 1.0 |
| | | | | 0.017 | |
| Coconut | 0 | 100 | 215 ± 5 | 0.233 ± | −51.3 ± 0.8 |
| | | | | 0.019 | |

For the combination offish EMO and flaxseed FFA oil, the resulting data are shown in Table 14 below.

TABLE 14
Fish EMO/Flaxseed FFA Oil Nanoemulsions (data represented as
average ± SD from at least three measurements); This blend is
representative for polyunsaturated fatty acids, specifically alpha-
linolenic acid (C18:3), EPA (C20:5) and DHA (C22:6)

| | EMO | FFA | Z avg | | Zeta potential |
|---|---|---|---|---|---|
| EMO/FFA | % | oil % | (nm) | PDI | (mV) |
| Fish/Flaxseed | 75 | 25 | 161 ± 4 | 0.233 ± 0.010 | −54.6 ± 0.3 |
| Fish/Flaxseed | 50 | 50 | 164 ± 3 | 0.199 ± 0.013 | −56.4 ± 1.6 |
| Fish/Flaxseed | 25 | 75 | 172 ± 3 | 0.197 ± 0.021 | −72.0 ± 4.0 |

For the combination of flaxseed EMO and fish FFA oil, the resulting data are shown in Table 15 below.

TABLE 15
Flaxseed EMO/Fish FFA Oil Nanoemulsions (data represented as
average ± SD from at least three measurements); This blend is
representative for polyunsaturated fatty acids, specifically alpha-
linolenic acid (C18:3), EPA (C20:5) and DHA (C22:6)

| | EMO | FFA | Z avg | | Zeta potential |
|---|---|---|---|---|---|
| EMO/FFA | % | oil % | (nm) | PDI | (mV) |
| Flaxseed/Fish | 75 | 25 | 155 ± 2 | 0.215 ± 0.024 | −56.3 ± 1.2 |
| Flaxseed/Fish | 50 | 50 | 157 ± 2 | 0.213 ± 0.011 | −56.7 ± 1.7 |
| Flaxseed/Fish | 25 | 75 | 162 ± 3 | 0.229 ± 0.013 | −55.1 ± 0.7 |

For the combination offish EMO and coconut FFA oil, the resulting data are shown in Table 16 below.

TABLE 16
Fish EMO/Coconut FFA Oil Nanoemulsions (data
represented as average ± SD from at least three measurements);
This blend is representative for polyunsaturated
fatty acids (specifically EPA (C20:5) and DHA (C22:6))
with medium chain fatty acids (specifically lauric acid (C12))

| | EMO | FFA | Z avg | | Zeta potential |
|---|---|---|---|---|---|
| EMO/FFA | % | oil % | (nm) | PDI | (mV) |
| Fish/Coconut | 75 | 25 | 177 ± 4 | 0.237 ± 0.017 | −40.7 ± 0.6 |
| Fish/Coconut | 50 | 50 | 165 ± 4 | 0.230 ± 0.018 | −43.4 ± 0.7 |
| Fish/Coconut | 25 | 75 | 164 ± 3 | 0.232 ± 0.013 | −46.2 ± 1.0 |

For the combination of flaxseed EMO and coconut FFA oil, the resulting data are shown in Table 17 below.

TABLE 17
Flaxseed EMO/Coconut FFA Oil Nanoemulsions (data
represented as average ± SD from at least three
measurements); This blend is representative for polyunsaturated
fatty acids (specifically alpha linolenic acid (C18:3))
with medium chain fatty acids (specifically lauric acid (C12))

| | | | | | Zeta |
|---|---|---|---|---|---|
| | EMO | FFA | Z avg | | potential |
| EMO/FFA | % | oil % | (nm) | PDI | (mV) |
| Flaxseed/coconut | 75 | 25 | 179 ± 2 | 0.227 ± 0.010 | −45.7 ± 1.4 |
| Flaxseed/coconut | 50 | 50 | 182 ± 1 | 0.232 ± 0.017 | −50.1 ± 1.6 |
| Flaxseed/coconut | 25 | 75 | 184 ± 2 | 0.242 ± 0.009 | −48.0 ± 0.2 |

For the combination of coconut EMO and flaxseed FFA oil, the resulting data are shown in Table 18 below.

TABLE 18
Coconut EMO/Flaxseed FFA Oil Nanoemulsions (data represented
as average ± SD from at least three measurements);
The blend is representative for polyunsaturated fatty acids
(specifically alpha linolenic acid (C18:3)) with medium
chain fatty acids (specifically lauric acid (C12))

| | | FFA | Z | | Zeta |
|---|---|---|---|---|---|
| | EMO | oil | avg | | potential |
| EMO/FFA | % | % | (nm) | PDI | (mV) |
| Coconut/flaxseed | 75 | 25 | 145 ± 3 | 0.228 ± 0.022 | −45.6 ± 1.9 |
| Coconut/flaxseed | 50 | 50 | 165 ± 2 | 0.167 ± 0.039 | −47.4 ± 1.9 |
| Coconut/flaxseed | 25 | 75 | 188 ± 2 | 0.163 ± 0.006 | −50.7 ± 1.1 |

For the combination of coconut EMO and rosehip FFA oil, the resulting data are shown in Table 19 below.

TABLE 19
Coconut EMO/Rosehip FFA Oil Nanoemulsions (data represented
as average ± SD from at least three measurements);
The blend is representative for polyunsaturated fats (high
in linoleic acid (C18:2) and linolenic acid (C18:3)) with
medium chain fatty acids (specifically lauric acid (C12))

| | | FFA | Z | | Zeta |
|---|---|---|---|---|---|
| | EMO | oil | avg | | potential |
| EMO/FFA | % | % | (nm) | PDI | (mV) |
| Coconut/rosehip | 75 | 25 | 153 ± 1 | 0.228 ± 0.024 | −44.3 ± 1.3 |
| Coconut/rosehip | 50 | 50 | 165 ± 4 | 0.208 ± 0.031 | −46.7 ± 0.7 |
| Coconut/rosehip | 25 | 75 | 190 ± 3 | 0.227 ± 0.024 | −52.2 ± 0.9 |

For the combination of rosehip EMO and coconut FFA oil, the resulting data are shown in Table 20 below.

TABLE 20
Rosehip EMO/coconut FFA Oil Nanoemulsions (data represented
as average ± SD from at least three measurements);
The blend is representative for polyunsaturated fats (high
in linoleic acid (C18:2) and linolenic acid (C18:3)) with
medium chain fatty acids (specifically lauric acid (C12))

| | | FFA | Z | | Zeta |
|---|---|---|---|---|---|
| | EMO | oil | avg | | potential |
| EMO/FFA | % | % | (nm) | PDI | (mV) |
| Rosehip/coconut | 75 | 25 | 210 ± 3 | 0.260 ± 0.007 | −46.9 ± 0.9 |
| Rosehip/coconut | 50 | 50 | 195 ± 2 | 0.244 ± 0.014 | −46.7 ± 0.8 |
| Rosehip/coconut | 25 | 75 | 191 ± 2 | 0.224 ± 0.011 | −48.0 ± 0.5 |

For the combination of rosehip EMO and flaxseed FFA oil, the resulting data are shown in Table 21 below.

TABLE 21
Rosehip EMO/flaxseed FFA Oil Nanoemulsions (data represented
as average ± SD from at least three measurements);
The blend is representative for unsaturated fats

| | | FFA | Z | | Zeta |
|---|---|---|---|---|---|
| | EMO | oil | avg | | potential |
| EMO/FFA | % | % | (nm) | PDI | (mV) |
| Rosehip/flaxseed | 75 | 25 | 189 ± 3 | 0.184 ± 0.032 | −52.2 ± 0.6 |
| Rosehip/flaxseed | 50 | 50 | 193 ± 3 | 0.177 ± 0.007 | −58.4 ± 0.6 |
| Rosehip/flaxseed | 25 | 75 | 205 ± 2 | 0.203 ± 0.025 | −55.6 ± 2.6 |

For the combination of flaxseed EMO and rosehip FFA oil, the resulting data are shown in Table 22 below.

TABLE 22
Flaxseed EMO/rosehip FFA Oil Nanoemulsions (data represented
as average ± SD from at least three measurements);
The blend is representative for unsaturated fats

| | | FFA | Z | | Zeta |
|---|---|---|---|---|---|
| | EMO | oil | avg | | potential |
| EMO/FFA | % | % | (nm) | PDI | (mV) |
| Flaxseed/rosehip | 75 | 25 | 170 ± 4 | 0.179 ± 0.040 | −60.4 ± 2.2 |
| Flaxseed/rosehip | 50 | 50 | 186 ± 6 | 0.187 ± 0.010 | −55.0 ± 0.7 |
| Flaxseed/rosehip | 25 | 75 | 197 ± 2 | 0.208 ± 0.017 | −61.7 ± 0.4 |

For the combination of algae EMO and flaxseed FFA oil, the resulting data are shown in Table 23 below.

TABLE 23
Algae EMO/flaxseed FFA Oil Nanoemulsions (data represented as
average ± SD from at least three measurements); The blend
is representative for polyunsaturated fatty acids, specifically
alpha-linolenic acid (C18:3), EPA (C20:5) and DHA (C22:6)

| | | FFA | Z | | Zeta |
|---|---|---|---|---|---|
| | EMO | oil | avg | | potential |
| EMO/FFA | % | % | (nm) | PDI | (mV) |
| Algae/flaxseed | 75 | 25 | 186 ± 4 | 0.197 ± 0.016 | Ppt before |
| | | | | | measuring |
| Algae/flaxseed | 50 | 50 | 187 ± 4 | 0.180 ± 0.029 | −47.0 ± 1.7 |
| Algae/flaxseed | 25 | 75 | 198 ± 1 | 0.187 ± 0.034 | −42.9 ± 1.0 |

For the combination of algae EMO and MCT FFA oil, the resulting data are shown in Table 24 below.

TABLE 24
Algae EMO/MCT FFA Oil Nanoemulsions (data represented
as average ± SD from at least three measurements);
The blend is representative for polyunsaturated fatty
acids (specifically EPA (C20:5) and DHA (C22:6)) in
combination with medium chain fatty acids.

| | | FFA | Z | | Zeta |
|---|---|---|---|---|---|
| | EMO | oil | avg | | potential |
| EMO/FFA | % | % | (nm) | PDI | (mV) |
| Algae/MCT | 75 | 25 | 452 ± 49 | 0.504 ± 0.445 | −45.8 ± 1.1 |
| Algae/MCT | 50 | 50 | 317 ± 17 | 0.077 ± 0.097 | −40.2 ± 0.6 |
| Algae/MCT | 25 | 75 | 270 ± 5 | 0.244 ± 0.044 | −39.5 ± 1.0 |

For the combination of Oat EMO and flaxseed FFA oil, the resulting data are shown in Table 25 below.

TABLE 25
Oat EMO/Flaxseed FFA Oil Nanoemulsions (data represented
as average ± SD from at least three measurements).

| | | FFA | Z | | Zeta |
|---|---|---|---|---|---|
| | EMO | oil | avg | | potential |
| EMO/FFA | % | % | (nm) | PDI | (mV) |
| Oat/Flaxseed | 75 | 25 | 182 ± 2 | 0.152 ± 0.021 | −62.4 ± 1.3 |
| Oat/Flaxseed | 50 | 50 | 186 ± 5 | 0.179 ± 0.008 | −62.3 ± 1.2 |
| Oat/Flaxseed | 25 | 75 | 195 ± 2 | 0.172 ± 0.035 | −62.6 ± 0.4 |

For the combination of Flaxseed EMO and Oat FFA oil, the resulting data are shown in Table 26 below.

TABLE 26
Flaxseed EMO/Oat FFA Oil Nanoemulsions (data represented
as average ± SD from at least three measurements).

| | | FFA | Z | | Zeta |
|---|---|---|---|---|---|
| | EMO | oil | avg | | potential |
| EMO/FFA | % | % | (nm) | PDI | (mV) |
| Flaxseed/Oat | 75 | 25 | 172 ± 2 | 0.191 ± 0.022 | −62.1 ± 1.3 |
| Flaxseed/Oat | 50 | 50 | 189 ± 1 | 0.181 ± 0.005 | −63.6 ± 0.9 |
| Flaxseed/Oat | 25 | 75 | 215 ± 7 | 0.183 ± 0.018 | −64.1 ± 1.8 |

### Example 4: Stability of Nanoemulsions

Nanoemulsions prepared in Examples 2 and 3 were stored at room temperature or refrigerated (4° C.). At various timepoints, as indicated in the tables below, the samples were re-tested for Z average particle size, polydispersity index and zeta potential (“Fresh” indicates the starting nanoemulsion).

TABLE 27
Stability of Coconut EMO/MCT FFA Oil Nanoemulsions stored at
room temperature (22° C.) (data represented as
average ± SD from at least three measurements;
Ppt denotes visible precipitation of the formulation)

| | | | Fresh | | 18 days | | 24 days | | 31 days | |
|---|---|---|---|---|---|---|---|---|---|---|
| | | FFA | Z | | Z | | Z | | Z | |
| | EMO | oil | avg | | avg | | avg | | avg | |
| EMO/FFA | % | % | (nm) | PDI | (nm) | PDI | (nm) | PDI | (nm) | PDI |
| Coconut | 100 | 0 | 137 ± | 0.193 ± | 243 ± | 0.549 ± | Ppt | Ppt | Ppt | Ppt |
| | | | 2 | 0.020 | 66 | 0.096 | | | | |
| Coconut/MCT | 90 | 10 | 147 ± | 0.190 ± | 147 ± | 0.193 ± | 149 ± | 0.208 ± | 148 ± | 0.185 ± |
| | | | 2 | 0.012 | 4 | 0.018 | 2 | 0.012 | 2 | 0.004 |
| Coconut/MCT | 75 | 25 | 154 ± | 0.186 ± | 159 ± | 0.183 ± | 160 ± | 0.199 ± | 163 ± | 0.178 ± |
| | | | 2 | 0.019 | 5 | 0.013 | 1 | 0.016 | 2 | 0.023 |
| Coconut/MCT | 50 | 50 | 163 ± | 0.196 ± | 173 ± | 0.191 ± | 175 ± | 0.202 ± | 177 ± | 0.189 ± |
| | | | 2 | 0.015 | 4 | 0.018 | 3 | 0.012 | 3 | 0.013 |
| Coconut/MCT | 25 | 75 | 184 ± | 0.230 ± | 202 ± | 0.182 ± | 204 ± | 0.191 ± | 208 ± | 0.214 ± |
| | | | 2 | 0.018 | 4 | 0.027 | 4 | 0.009 | 3 | 0.018 |
| Coconut/MCT | 10 | 90 | 224 ± | 0.268 ± | 245 ± | 0.239 ± | 249 ± | 0.216 ± | 252 ± | 0.200 ± |
| | | | 3 | 0.012 | 5 | 0.023 | 5 | 0.001 | 3 | 0.023 |
| MCT | 0 | 100 | 255 ± | 0.296 ± | 276 ± | 0.235 ± | 278 ± | 0.186 ± | 277 ± | 0.205 ± |
| | | | 4 | 0.053 | 3 | 0.064 | 3 | 0.063 | 2 | 0.013 |

TABLE 28
Stability of Coconut EMO/MCT FFA Oil Nanoemulsions stored at 4° C.
(data represented as average ± SD from at least three measurements;
Ppt denotes visible precipitation of the formulation)

| | | | Fresh | | 18 days | | 24 days | | 31 days | |
|---|---|---|---|---|---|---|---|---|---|---|
| | | FFA | Z | | Z | | Z | | Z | |
| | EMO | oil | avg | | avg | | avg | | avg | |
| EMO/FFA | % | % | (nm) | PDI | (nm) | PDI | (nm) | PDI | (nm) | PDI |
| Coconut | 100 | 0 | 137 ± | 0.193 ± | 947 ± | 0.964 ± | Ppt | Ppt | Ppt | Ppt |
| | | | 2 | 0.020 | 526 | 0.062 | | | | |
| Coconut/MCT | 90 | 10 | 147 ± | 0.190 ± | 620 ± | 0.932 ± | 149 ± | 0.185 ± | Ppt | Ppt |
| | | | 2 | 0.012 | 132 | 0.118 | 3 | 0.043 | | |
| Coconut/MCT | 75 | 25 | 154 ± | 0.186 ± | 278 ± | 0.560 ± | 182 ± | 0.410 ± | 157 ± | 0.305 ± |
| | | | 2 | 0.019 | 118 | 0.134 | 15 | 0.032 | 6 | 0.074 |
| Coconut/MCT | 50 | 50 | 163 ± | 0.196 ± | 172 ± | 0.197 ± | 173 ± | 0.231 ± | 175 ± | 0.204 ± |
| | | | 2 | 0.015 | 7 | 0.022 | 5 | 0.009 | 2 | 0.009 |
| Coconut/MCT | 25 | 75 | 184 ± | 0.230 ± | 198 ± | 0.192 ± | 197 ± | 0.231 ± | 202 ± | 0.203 ± |
| | | | 2 | 0.018 | 5 | 0.017 | 5 | 0.015 | 2 | 0.032 |
| Coconut/MCT | 10 | 90 | 224 ± | 0.268 ± | 236 ± | 0.209 ± | 242 ± | 0.216 ± | 246 ± | 0.213 ± |
| | | | 3 | 0.012 | 3 | 0.045 | 4 | 0.049 | 7 | 0.031 |
| MCT | 0 | 100 | 255 ± | 0.296 ± | 274 ± | 0.241 ± | 265 ± | 0.166 ± | 267 ± | 0.264 ± |
| | | | 4 | 0.053 | 3 | 0.049 | 4 | 0.033 | 5 | 0.027 |

TABLE 29
Stability of MCT EMO/Coconut FFA Oil Nanoemulsions stored at room
temperature (data represented as average ± SD from at least three
measurements; Ppt denotes visible precipitation of the formulation)

| | | | Fresh | | 16 days | | 21 days | | 28 days | |
|---|---|---|---|---|---|---|---|---|---|---|
| | | FFA | Z | | Z | | Z | | Z | |
| | EMO | oil | avg | | avg | | avg | | avg | |
| EMO/FFA | % | % | (nm) | PDI | (nm) | PDI | (nm) | PDI | (nm) | PDI |
| MCT | 100 | 0 | 272 ± | 0.276 ± | 271 ± | 0.256 ± | 274 ± | 0.220 ± | 284 ± | 0.233 ± |
| | | | 6 | 0.028 | 4 | 0.040 | 3 | 0.011 | 3 | 0.014 |
| MCT/Coconut | 90 | 10 | 226 ± | 0.248 ± | 237 ± | 0.228 ± | 236 ± | 0.195 ± | 238 ± | 0.195 ± |
| | | | 6 | 0.014 | 2 | 0.014 | 3 | 0.019 | 6 | 0.007 |
| MCT/Coconut | 75 | 25 | 162 ± | 0.200 ± | 175 ± | 0.168 ± | 177 ± | 0.168 ± | 177 ± | 0.200 ± |
| | | | 3 | 0.020 | 3 | 0.027 | 2 | 0.013 | 3 | 0.026 |
| MCT/Coconut | 50 | 50 | 135 ± | 0.193 ± | 150 ± | 0.201 ± | 151 ± | 0.180 ± | 151 ± | 0.190 ± |
| | | | 2 | 0.017 | 2 | 0.004 | 2 | 0.014 | 2 | 0.027 |
| MCT/Coconut | 25 | 75 | 147 ± | 0.219 ± | 162 ± | 0.212 ± | Ppt | Ppt | Ppt | Ppt |
| | | | 2 | 0.013 | 3 | 0.011 | | | | |
| MCT/Coconut | 10 | 90 | 173 ± | 0.220 ± | 174 ± | 0.238 ± | Ppt | Ppt | Ppt | Ppt |
| | | | 3 | 0.017 | 4 | 0.020 | | | | |
| Coconut | 0 | 100 | 214 ± | 0.233 ± | 205 ± | 0.217 ± | Ppt | Ppt | Ppt | Ppt |
| | | | 5 | 0.019 | 4 | 0.008 | | | | |

TABLE 30
Stability of MCT EMO/Coconut FFA Oil Nanoemulsions stored at 4° C.
(data represented as average ± SD from at least three measurements;
Ppt denotes visible precipitation of the formulation)

| | | | Fresh | | 16 days | | 21 days | | 28 days | |
|---|---|---|---|---|---|---|---|---|---|---|
| | | FFA | Z | | Z | | Z | | Z | |
| | EMO | oil | avg | | avg | | avg | | avg | |
| EMO/FFA | % | % | (nm) | PDI | (nm) | PDI | (nm) | PDI | (nm) | PDI |
| MCT | 100 | 0 | 272 ± | 0.276 ± | 251 ± | 0.230 ± | 250 ± | 0.210 ± | 248 ± | 0.196 ± |
| | | | 6 | 0.028 | 7 | 0.009 | 3 | 0.018 | 2 | 0.011 |
| MCT/Coconut | 90 | 10 | 226 ± | 0.248 ± | 229 ± | 0.229 ± | 232 ± | 0.232 ± | 227 ± | 0.222 ± |
| | | | 6 | 0.014 | 7 | 0.033 | 3 | 0.016 | 5 | 0.013 |
| MCT/Coconut | 75 | 25 | 162 ± | 0.200 ± | 170 ± | 0.189 ± | 169 ± | 0.189 ± | 172 ± | 0.204 ± |
| | | | 3 | 0.020 | 2 | 0.025 | 4 | 0.018 | 1 | 0.026 |
| MCT/Coconut | 50 | 50 | 135 ± | 0.193 ± | 131 ± | 0.267 ± | Ppt | Ppt | Ppt | Ppt |
| | | | 2 | 0.017 | 1 | 0.025 | | | | |
| MCT/Coconut | 25 | 75 | 147 ± | 0.219 ± | 139 ± | 0.329 ± | Ppt | Ppt | Ppt | Ppt |
| | | | 2 | 0.013 | 16 | 0.083 | | | | |
| MCT/Coconut | 10 | 90 | 173 ± | 0.220 ± | 141 ± | 0.245 ± | Ppt | Ppt | Ppt | Ppt |
| | | | 3 | 0.017 | 1 | 0.010 | | | | |
| Coconut | 0 | 100 | 214 ± | 0.233 ± | 177 ± | 0.225 ± | Ppt | Ppt | Ppt | Ppt |
| | | | 5 | 0.019 | 3 | 0.026 | | | | |

TABLE 31
Stability of Flaxseed and Sesame EMO Nanoemulsions stored at room temperature (data represented as
average ± SD from at least three measurements; Ppt denotes visible precipitation of the formulation)

| | EMO | | Fresh | | 14 days | | 21 days | | 28 days | |
|---|---|---|---|---|---|---|---|---|---|---|
| | (non-FFA | | Z | | Z | | Z | | Z | |
| | compo- | Total | avg | | avg | | avg | | avg | |
| Oil | nent) % | FFA % | (nm) | PDI | (nm) | PDI | (nm) | PDI | (nm) | PDI |
| Flaxseed | 82 | 18 | 142 ± 3 | 0.205 ± 0.013 | 139 ± 2 | 0.208 ± 0.013 | 141 ± 4 | 0.212 ± 0.012 | 138 ± 3 | 0.207 ± 0.007 |
| Flaxseed | 75 | 25 | 141 ± 3 | 0.208 ± 0.012 | 136 ± 1 | 0.192 ± 0.009 | 141 ± 4 | 0.206 ± 0.028 | 139 ± 3 | 0.190 ± 0.021 |
| Flaxseed | 50 | 50 | 162 ± 5 | 0.179 ± 0.027 | 166 ± 1 | 0.196 ± 0.014 | 166 ± 3 | 0.210 ± 0.036 | 165 ± 4 | 0.180 ± 0.018 |
| Flaxseed | 25 | 75 | 191 ± 3 | 0.192 ± 0.012 | 206 ± 3 | 0.222 ± 0.014 | 208 ± 3 | 0.209 ± 0.032 | 221 ± 1 | 0.220 ± 0.037 |
| Flaxseed | 10 | 90 | 273 ± 2 | 0.281 ± 0.043 | 288 ± 7 | 0.211 ± 0.064 | 289 ± 9 | 0.160 ± 0.025 | 287 ± 4 | 0.167 ± 0.051 |
| Flaxseed | 0 | 100 | 345 ± 12 | 0.232 ± 0.066 | 403 ± 20 | 0.227 ± 0.066 | 405 ± 18 | 0.142 ± 0.030 | 392 ± 40 | 0.488 ± 0.290 |
| Sesame | 88 | 12 | 181 ± 4 | 0.186 ± 0.015 | 183 ± 2 | 0.179 ± 0.012 | 179 ± 4 | 0.195 ± 0.015 | Ppt | Ppt |
| Sesame | 75 | 25 | 180 ± 3 | 0.182 ± 0.023 | 186 ± 4 | 0.193 ± 0.017 | 180 ± 3 | 0.167 ± 0.012 | 187 ± 4 | 0.178 ± 0.004 |
| Sesame | 50 | 50 | 180 ± 3 | 0.184 ± 0.025 | 184 ± 5 | 0.187 ± 0.007 | 188 ± 3 | 0.211 ± 0.018 | 188 ± 3 | 0.195 ± 0.018 |
| Sesame | 25 | 75 | 203 ± 4 | 0.174 ± 0.022 | 211 ± 2 | 0.177 ± 0.010 | 210 ± 6 | 0.169 ± 0.023 | 216 ± 4 | 0.209 ± 0.031 |
| Sesame | 10 | 90 | 248 ± 3 | 0.200 ± 0.027 | 400 ± 9 | 0.111 ± 0.130 | 447 ± 5 | 0.092 ± 0.092 | Ppt | Ppt |
| Sesame | 0 | 100 | 392 ± 15 | 0.236 ± 0.088 | 444 ± 16 | 0.595 ± 0.315 | Ppt | Ppt | Ppt | Ppt |

TABLE 32
Stability of Flaxseed and Sesame EMO Nanoemulsions stored at 4° C. (data represented as average ±
SD from at least three measurements; Ppt denotes visible precipitation of the formulation)

| | | | Fresh | | 14 days | | 21 days | | 28 days | |
|---|---|---|---|---|---|---|---|---|---|---|
| | EMO | | Z | | Z | | Z | | Z | |
| | (non-FFA | Total | avg | | avg | | avg | | avg | |
| Oil | component) % | FFA % | (nm) | PDI | (nm) | PDI | (nm) | PDI | (nm) | PDI |
| Flaxseed | 82 | 18 | 142 ± 3 | 0.205 ± 0.013 | 141 ± 3 | 0.234 ± 0.009 | 170 ± 1 | 0.204 ± 0.013 | Ppt | Ppt |
| Flaxseed | 75 | 25 | 141 ± 3 | 0.208 ± 0.012 | 142 ± 4 | 0.205 ± 0.010 | 144 ± 1 | 0.210 ± 0.006 | 141 ± 1 | 0.206 ± 0.022 |
| Flaxseed | 50 | 50 | 162 ± 5 | 0.179 ± 0.027 | 165 ± 1 | 0.208 ± 0.017 | 171 ± 3 | 0.223 ± 0.019 | Ppt | Ppt |
| Flaxseed | 25 | 75 | 191 ± 3 | 0.192 ± 0.012 | 200 ± 1 | 0.216 ± 0.032 | Ppt | Ppt | Ppt | Ppt |
| Flaxseed | 10 | 90 | 273 ± 2 | 0.281 ± 0.043 | 258 ± 1 | 0.246 ± 0.020 | Ppt | Ppt | Ppt | Ppt |
| Flaxseed | 0 | 100 | 345 ± 12 | 0.232 ± 0.066 | 284 ± 11 | 0.185 ± 0.075 | Ppt | Ppt | Ppt | Ppt |
| Sesame | 88 | 12 | 181 ± 4 | 0.186 ± 0.015 | Ppt | Ppt | Ppt | Ppt | Ppt | Ppt |
| Sesame | 75 | 25 | 180 ± 3 | 0.182 ± 0.023 | Ppt | Ppt | Ppt | Ppt | Ppt | Ppt |
| Sesame | 50 | 50 | 180 ± 3 | 0.184 ± 0.025 | Ppt | Ppt | Ppt | Ppt | Ppt | Ppt |
| Sesame | 25 | 75 | 203 ± 4 | 0.174 ± 0.022 | Ppt | Ppt | Ppt | Ppt | Ppt | Ppt |
| Sesame | 10 | 90 | 248 ± 3 | 0.200 ± 0.027 | Ppt | Ppt | Ppt | Ppt | Ppt | Ppt |
| Sesame | 0 | 100 | 392 ± 15 | 0.236 ± 0.088 | Ppt | Ppt | Ppt | Ppt | Ppt | Ppt |

TABLE 33
Stability of Rosehip and Hemp seed EMO Nanoemulsions stored at room temperature (data represented as
average ± SD from at least three measurements; Ppt denotes visible precipitation of the formulation)

| | | | Fresh | | 14 days | | 21 days | | 28 days | |
|---|---|---|---|---|---|---|---|---|---|---|
| | EMO | | Z | | Z | | Z | | Z | |
| | (non-FFA | Total | avg | | avg | | avg | | avg | |
| Oil | component) % | FFA % | (nm) | PDI | (nm) | PDI | (nm) | PDI | (nm) | PDI |
| Rosehip | 75 | 25 | 203 ± 1 | 0.187 ± 0.019 | 211 ± 8 | 0.163 ± 0.021 | 210 ± 4 | 0.209 ± 0.015 | 215 ± 3 | 0.205 ± 0.016 |
| Rosehip | 50 | 50 | 205 ± 3 | 0.174 ± 0.012 | 212 ± 7 | 0.186 ± 0.021 | 207 ± 1 | 0.191 ± 0.038 | 210 ± 2 | 0.203 ± 0.032 |
| Rosehip | 25 | 75 | 204 ± 3 | 0.231 ± 0.026 | 216 ± 2 | 0.217 ± 0.023 | 212 ± 2 | 0.216 ± 0.032 | 215 ± 5 | 0.218 ± 0.013 |
| Rosehip | 0 | 100 | 245 ± 3 | 0.200 ± 0.010 | 312 ± 26 | 0.142 ± 0.024 | 339 ± 3 | 0.234 ± 0.051 | 327 ± 4 | 0.162 ± 0.082 |
| Hemp | 91 | 9 | 183 ± 4 | 0.215 ± 0.026 | 191 ± 3 | 0.242 ± 0.011 | 197 ± 3 | 0.249 ± 0.016 | 201 ± 3 | 0.256 ± 0.009 |
| seed | | | | | | | | | | |
| Hemp | 75 | 25 | 174 ± 1 | 0.200 ± 0.019 | 183 ± 3 | 0.201 ± 0.022 | 185 ± 4 | 0.226 ± 0.005 | 190 ± 3 | 0.233 ± 0.023 |
| seed | | | | | | | | | | |
| Hemp | 50 | 50 | 180 ± 2 | 0.199 ± 0.023 | 183 ± 1 | 0.205 ± 0.024 | 187 ± 3 | 0.192 ± 0.025 | 185 ± 2 | 0.182 ± 0.016 |
| seed | | | | | | | | | | |
| Hemp | 25 | 75 | 181 ± 2 | 0.180 ± 0.041 | 186 ± 1 | 0.207 ± 0.018 | 191 ± 4 | 0.181 ± 0.036 | 192 ± 1 | 0.194 ± 0.016 |
| seed | | | | | | | | | | |
| Hemp | 0 | 100 | 245 ± 1 | 0.197 ± 0.001 | 327 ± 7 | 0.110 ± 0.086 | 315 ± 4 | 0.143 ± 0.093 | 313 ± 9 | 0.100 ± 0.089 |
| seed | | | | | | | | | | |

TABLE 34
Stability of MCT EMO Nanoemulsions stored at room temperature (data represented as average ±
SD from at least three measurements; Ppt denotes visible precipitation of the formulation)

| | | | Fresh | | 14 days | | 21 days | | 28 days | |
|---|---|---|---|---|---|---|---|---|---|---|
| | EMO | | Z | | Z | | Z | | Z | |
| | (non-FFA | Total | avg | | avg | | avg | | avg | |
| Oil | component) % | FFA % | (nm) | PDI | (nm) | PDI | (nm) | PDI | (nm) | PDI |
| MCT | 79 | 21 | 233 ± 5 | 0.252 ± 0.006 | 243 ± 3 | 0.231 ± 0.005 | 254 ± 5 | 0.232 ± 0.026 | 255 ± 9 | 0.208 ± 0.010 |
| MCT | 50 | 50 | 191 ± 4 | 0.217 ± 0.007 | 207 ± 4 | 0.176 ± 0.051 | 209 ± 1 | 0.165 ± 0.049 | 215 ± 3 | 0.156 ± 0.052 |
| MCT | 25 | 75 | 182 ± 5 | 0.213 ± 0.008 | 201 ± 6 | 0.182 ± 0.028 | 201 ± 11 | 0.288 ± 0.081 | 211 ± 6 | 0.143 ± 0.010 |
| MCT | 10 | 90 | 205 ± 1 | 0.238 ± 0.019 | 221 ± 2 | 0.219 ± 0.027 | 233 ± 4 | 0.186 ± 0.025 | 232 ± 2 | 0.201 ± 0.023 |
| MCT | 0 | 100 | 257 ± 4 | 0.290 ± 0.022 | 258 ± 4 | 0.224 ± 0.043 | 272 ± 7 | 0.198 ± 0.042 | 269 ± 3 | 0.181 ± 0.029 |

TABLE 35
Stability of MCT EMO Nanoemulsions stored at 4° C. (data represented as average ±
SD from at least three measurements; Ppt denotes visible precipitation of the formulation)

| | | | Fresh | | 14 days | | 21 days | | 28 days | |
|---|---|---|---|---|---|---|---|---|---|---|
| | EMO | | Z | | Z | | Z | | Z | |
| | (non-FFA | Total | avg | | avg | | avg | | avg | |
| Oil | component) % | FFA % | (nm) | PDI | (nm) | PDI | (nm) | PDI | (nm) | PDI |
| MCT | 79 | 21 | 233 ± 5 | 0.252 ± 0.006 | 228 ± 2 | 0.240 ± 0.017 | 224 ± 2 | 0.241 ± 0.006 | 236 ± 4 | 0.327 ± 0.038 |
| MCT | 50 | 50 | 191 ± 4 | 0.217 ± 0.007 | 199 ± 2 | 0.174 ± 0.045 | 200 ± 1 | 0.187 ± 0.021 | 204 ± 1 | 0.199 ± 0.033 |
| MCT | 25 | 75 | 182 ± 5 | 0.213 ± 0.008 | 194 ± 2 | 0.188 ± 0.009 | 196 ± 3 | 0.174 ± 0.017 | 200 ± 1 | 0.149 ± 0.049 |
| MCT | 10 | 90 | 205 ± 1 | 0.238 ± 0.019 | 214 ± 7 | 0.239 ± 0.014 | 197 ± 2 | 0.215 ± 0.024 | 226 ± 3 | 0.189 ± 0.010 |
| MCT | 0 | 100 | 257 ± 4 | 0.290 ± 0.022 | 260 ± 3 | 0.242 ± 0.037 | 257 ± 4 | 0.220 ± 0.038 | 276 ± 1 | 0.219 ± 0.005 |

TABLE 36
Stability of Nanoemulsions from Flaxseed EMO and FFAs of fish, coconut, and rosehip stored at room temperature (data
represented as average ± SD from at least three measurements; Ppt denotes visible precipitation of the formulation)

| | | | Fresh | | 14 days | | 21 days | | 28 days | |
|---|---|---|---|---|---|---|---|---|---|---|
| | | | Z | | Z | | Z | | Z | |
| | | FFA | avg | | avg | | avg | | avg | |
| EMO/FFA | EMO % | oil % | (nm) | PDI | (nm) | PDI | (nm) | PDI | (nm) | PDI |
| Flaxseed/ | 75 | 25 | 155 ± 2 | 0.215 ± 0.024 | 154 ± 1 | 0.207 ± 0.013 | 157 ± 3 | 0.207 ± 0.019 | 149 ± 3 | 0.218 ± 0.028 |
| Fish | | | | | | | | | | |
| Flaxseed/ | 50 | 50 | 157 ± 2 | 0.214 ± 0.011 | 154 ± 1 | 0.195 ± 0.020 | Ppt | Ppt | Ppt | Ppt |
| Fish | | | | | | | | | | |
| Flaxseed/ | 25 | 75 | 162 ± 3 | 0.229 ± 0.013 | 165 ± 3 | 0.235 ± 0.024 | Ppt | Ppt | Ppt | Ppt |
| Fish | | | | | | | | | | |
| Flaxseed/ | 75 | 25 | 179 ± 2 | 0.227 ± 0.010 | 180 ± 3 | 0.227 ± 0.005 | 182 ± 1 | 0.217 ± 0.016 | 186 ± 3 | 0.222 ± 0.006 |
| Coconut | | | | | | | | | | |
| Flaxseed/ | 50 | 50 | 182 ± 1 | 0.232 ± 0.017 | 187 ± 6 | 0.232 ± 0.010 | 187 ± 4 | 0.231 ± 0.018 | 189 ± 3 | 0.227 ± 0.011 |
| Coconut | | | | | | | | | | |
| Flaxseed/ | 25 | 75 | 184 ± 2 | 0.242 ± 0.009 | Ppt | Ppt | Ppt | Ppt | Ppt | Ppt |
| Coconut | | | | | | | | | | |
| Flaxseed/ | 75 | 25 | 170 ± 4 | 0.179 ± 0.040 | 171 ± 7 | 0.155 ± 0.021 | 168 ± 2 | 0.175 ± 0.020 | 178 ± 5 | 0.173 ± 0.027 |
| rosehip | | | | | | | | | | |
| Flaxseed/ | 50 | 50 | 186 ± 6 | 0.187 ± 0.010 | 185 ± 4 | 0.173 ± 0.005 | 183 ± 3 | 0.172 ± 0.032 | 185 ± 7 | 0.221 ± 0.035 |
| rosehip | | | | | | | | | | |
| Flaxseed/ | 25 | 75 | 197 ± 2 | 0.208 ± 0.017 | 213 ± 1 | 0.197 ± 0.011 | 222 ± 3 | 0.240 ± 0.021 | 223 ± 4 | 0.274 ± 0.074 |
| rosehip | | | | | | | | | | |

TABLE 37
Stability of Nanoemulsions from Flaxseed EMO and FFAs of fish, coconut, and
rosehip stored at 4° C. (data represented as average ± SD from
at least three measurements; Ppt denotes visible precipitation of the formulation)

| | | | Fresh | | 14 days | | 21 days | | 28 days | |
|---|---|---|---|---|---|---|---|---|---|---|
| | | FFA | Z | | Z | | Z | | Z | |
| | | oil | avg | | avg | | avg | | avg | |
| EMO/FFA | EMO % | % | (nm) | PDI | (nm) | PDI | (nm) | PDI | (nm) | PDI |
| Flaxseed/ | 75 | 25 | 155 ± 2 | 0.215 ± 0.024 | 163 ± 7 | 0.292 ± 0.065 | Ppt | Ppt | Ppt | Ppt |
| Fish | | | | | | | | | | |
| Flaxseed/ | 50 | 50 | 157 ± 2 | 0.214 ± 0.011 | 176 ± 11 | 0.386 ± 0.030 | Ppt | Ppt | Ppt | Ppt |
| Fish | | | | | | | | | | |
| Flaxseed/ | 25 | 75 | 162 ± 3 | 0.229 ± 0.013 | 243 ± 22 | 0.493 ± 0.059 | Ppt | Ppt | Ppt | Ppt |
| Fish | | | | | | | | | | |
| Flaxseed/ | 75 | 25 | 179 ± 2 | 0.227 ± 0.010 | Ppt | Ppt | Ppt | Ppt | Ppt | Ppt |
| Coconut | | | | | | | | | | |
| Flaxseed/ | 50 | 50 | 182 ± 1 | 0.232 ± 0.017 | Ppt | Ppt | Ppt | Ppt | Ppt | Ppt |
| Coconut | | | | | | | | | | |
| Flaxseed/ | 25 | 75 | 184 ± 2 | 0.242 ± 0.009 | Ppt | Ppt | Ppt | Ppt | Ppt | Ppt |
| Coconut | | | | | | | | | | |
| Flaxseed/ | 75 | 25 | 170 ± 4 | 0.179 ± 0.040 | Ppt | Ppt | Ppt | Ppt | Ppt | Ppt |
| rosehip | | | | | | | | | | |
| Flaxseed/ | 50 | 50 | 186 ± 6 | 0.187 ± 0.010 | Ppt | Ppt | Ppt | Ppt | Ppt | Ppt |
| rosehip | | | | | | | | | | |
| Flaxseed/ | 25 | 75 | 197 ± 2 | 0.208 ± 0.017 | Ppt | Ppt | Ppt | Ppt | Ppt | Ppt |
| rosehip | | | | | | | | | | |

TABLE 38
Stability of Nanoemulsions from Fish EMO and FFAs of flaxseed and coconut stored at room temperature (data represented
as average ± SD from at least three measurements; Ppt denotes visible precipitation of the formulation)

| | | | Fresh | | 14 days | | 21 days | | 28 days | |
|---|---|---|---|---|---|---|---|---|---|---|
| | | | Z | | Z | | Z | | Z | |
| | | FFA | avg | | avg | | avg | | avg | |
| EMO/FFA | EMO % | oil % | (nm) | PDI | (nm) | PDI | (nm) | PDI | (nm) | PDI |
| Fish/ | 75 | 25 | 161 ± 4 | 0.233 ± 0.010 | 153 ± 4 | 0.238 ± 0.006 | Ppt | Ppt | Ppt | Ppt |
| Flaxseed | | | | | | | | | | |
| Fish/ | 50 | 50 | 164 ± 3 | 0.199 ± 0.013 | 160 ± 3 | 0.189 ± 0.014 | Ppt | Ppt | Ppt | Ppt |
| Flaxseed | | | | | | | | | | |
| Fish/ | 25 | 75 | 172 ± 3 | 0.197 ± 0.021 | 171 ± 1 | 0.210 ± 0.007 | 170 ± 2 | 0.209 ± 0.015 | 141 ± 1 | 0.206 ± 0.022 |
| Flaxseed | | | | | | | | | | |
| Fish/ | 75 | 25 | 177 ± 4 | 0.237 ± 0.017 | Ppt | Ppt | Ppt | Ppt | Ppt | Ppt |
| Coconut | | | | | | | | | | |
| Fish/ | 50 | 50 | 165 ± 4 | 0.230 ± 0.018 | 168 ± 3 | 0.228 ± 0.006 | 170 ± 6 | 0.209 ± 0.016 | 171 ± 2 | 0.210 ± 0.013 |
| Coconut | | | | | | | | | | |
| Fish/ | 25 | 75 | 164 ± 3 | 0.232 ± 0.013 | Ppt | Ppt | Ppt | Ppt | Ppt | Ppt |
| Coconut | | | | | | | | | | |

TABLE 39
Stability of Nanoemulsions from Fish EMO and FFAs of flaxseed and coconut
stored at 4° C. (data represented as average ± SD from at least
three measurements; Ppt denotes visible precipitation of the formulation)

| | | | Fresh | | 14 days | | 21 days | | 28 days | |
|---|---|---|---|---|---|---|---|---|---|---|
| | | | Z | | Z | | Z | | Z | |
| | | FFA | avg | | avg | | avg | | avg | |
| EMO/FFA | EMO % | oil % | (nm) | PDI | (nm) | PDI | (nm) | PDI | (nm) | PDI |
| Fish/ | 75 | 25 | 161 ± 4 | 0.233 ± 0.010 | 158 ± 1 | 0.263 ± 0.023 | Ppt | Ppt | Ppt | Ppt |
| Flaxseed | | | | | | | | | | |
| Fish/ | 50 | 50 | 164 ± 3 | 0.199 ± 0.013 | 165 ± 3 | 0.258 ± 0.046 | Ppt | Ppt | Ppt | Ppt |
| Flaxseed | | | | | | | | | | |
| Fish/ | 25 | 75 | 172 ± 3 | 0.197 ± 0.021 | 169 ± 3 | 0.202 ± 0.029 | Ppt | Ppt | Ppt | Ppt |
| Flaxseed | | | | | | | | | | |
| Fish/ | 75 | 25 | 177 ± 4 | 0.237 ± 0.017 | Ppt | Ppt | Ppt | Ppt | Ppt | Ppt |
| Coconut | | | | | | | | | | |
| Fish/ | 50 | 50 | 165 ± 4 | 0.230 ± 0.018 | Ppt | Ppt | Ppt | Ppt | Ppt | Ppt |
| Coconut | | | | | | | | | | |
| Fish/ | 25 | 75 | 164 ± 3 | 0.232 ± 0.013 | Ppt | Ppt | Ppt | Ppt | Ppt | Ppt |
| Coconut | | | | | | | | | | |

TABLE 40
Stability of Nanoemulsions from Coconut EMO and FFAs of flaxseed and rosehip stored at room temperature (data represented
as average ± SD from at least three measurements; Ppt denotes visible precipitation of the formulation)

| | | | Fresh | | 14 days | | 21 days | | 28 days | |
|---|---|---|---|---|---|---|---|---|---|---|
| | | | Z | | Z | | Z | | Z | |
| | | FFA | avg | | avg | | avg | | avg | |
| EMO/FFA | EMO % | oil % | (nm) | PDI | (nm) | PDI | (nm) | PDI | (nm) | PDI |
| Coconut/ | 75 | 25 | 145 ± 3 | 0.228 ± 0.022 | 158 ± 1 | 0.228 ± 0.017 | 158 ± 4 | 0.227 ± 0.012 | 165 ± 4 | 0.214 ± 0.012 |
| Flaxseed | | | | | | | | | | |
| Coconut/ | 50 | 50 | 165 ± 2 | 0.167 ± 0.039 | 188 ± 4 | 0.232 ± 0.016 | 194 ± 3 | 0.221 ± 0.009 | 197 ± 6 | 0.249 ± 0.005 |
| Flaxseed | | | | | | | | | | |
| Coconut/ | 25 | 75 | 188 ± 2 | 0.163 ± 0.006 | 203 ± 3 | 0.208 ± 0.023 | 212 ± 4 | 0.191 ± 0.035 | 229 ± 1 | 0.233 ± 0.018 |
| Flaxseed | | | | | | | | | | |
| Coconut/ | 75 | 25 | 153 ± 1 | 0.228 ± 0.024 | 165 ± 4 | 0.225 ± 0.017 | 164 ± 2 | 0.236 ± 0.015 | 171 ± 1 | 0.213 ± 0.012 |
| rosehip | | | | | | | | | | |
| Coconut/ | 50 | 50 | 165 ± 4 | 0.208 ± 0.031 | 181 ± 2 | 0.214 ± 0.004 | 186 ± 3 | 0.250 ± 0.014 | 195 ± 1 | 0.257 ± 0.008 |
| rosehip | | | | | | | | | | |
| Coconut/ | 25 | 75 | 190 ± 3 | 0.227 ± 0.024 | 210 ± 6 | 0.189 ± 0.039 | 211 ± 1 | 0.195 ± 0.018 | 218 ± 3 | 0.222 ± 0.019 |
| rosehip | | | | | | | | | | |

TABLE 41
Stability of Nanoemulsions from Coconut EMO and FFAs of flaxseed and rosehip
stored at 4° C. (data represented as average ± SD from at least
three measurements; Ppt denotes visible precipitation of the formulation)

| | | | Fresh | | 14 days | | 21 days | | 28 days | |
|---|---|---|---|---|---|---|---|---|---|---|
| | | | Z | | Z | | Z | | Z | |
| | | FFA | avg | | avg | | avg | | avg | |
| EMO/FFA | EMO % | oil % | (nm) | PDI | (nm) | PDI | (nm) | PDI | (nm) | PDI |
| Coconut/ | 75 | 25 | 145 ± 3 | 0.228 ± 0.022 | Ppt | Ppt | Ppt | Ppt | Ppt | Ppt |
| Flaxseed | | | | | | | | | | |
| Coconut/ | 50 | 50 | 165 ± 2 | 0.167 ± 0.039 | 184 ± 1 | 0.289 ± 0.027 | Ppt | Ppt | Ppt | Ppt |
| Flaxseed | | | | | | | | | | |
| Coconut/ | 25 | 75 | 188 ± 2 | 0.163 ± 0.006 | Ppt | Ppt | Ppt | Ppt | Ppt | Ppt |
| Flaxseed | | | | | | | | | | |
| Coconut/ | 75 | 25 | 153 ± 1 | 0.228 ± 0.024 | Ppt | Ppt | Ppt | Ppt | Ppt | Ppt |
| rosehip | | | | | | | | | | |
| Coconut/ | 50 | 50 | 165 ± 4 | 0.208 ± 0.031 | Ppt | Ppt | Ppt | Ppt | Ppt | Ppt |
| rosehip | | | | | | | | | | |
| Coconut/ | 25 | 75 | 190 ± 3 | 0.227 ± 0.024 | Ppt | Ppt | Ppt | Ppt | Ppt | Ppt |
| rosehip | | | | | | | | | | |

TABLE 42
Stability of Nanoemulsions from Rosehip EMO and coconut FFA stored at room temperature (data represented
as average ± SD from at least three measurements; Ppt denotes visible precipitation of the formulation)

| | | | Fresh | | 14 days | | 21 days | | 39 days | |
|---|---|---|---|---|---|---|---|---|---|---|
| | | | Z | | Z | | Z | | Z | |
| | | FFA | avg | | avg | | avg | | avg | |
| EMO/FFA | EMO % | oil % | (nm) | PDI | (nm) | PDI | (nm) | PDI | (nm) | PDI |
| Rosehip/ | 75 | 25 | 210 ± 3 | 0.260 ± 0.007 | 220 ± 5 | 0.248 ± 0.028 | 225 ± 3 | 0.257 ± 0.011 | 227 ± 3 | 0.260 ± 0.041 |
| coconut | | | | | | | | | | |
| Rosehip/ | 50 | 50 | 195 ± 2 | 0.244 ± 0.014 | 194 ± 9 | 0.288 ± 0.033 | 195 ± 2 | 0.254 ± 0.021 | 199 ± 3 | 0.250 ± 0.007 |
| coconut | | | | | | | | | | |
| Rosehip/ | 25 | 75 | 191 ± 2 | 0.224 ± 0.011 | Ppt | Ppt | Ppt | Ppt | Ppt | Ppt |
| coconut | | | | | | | | | | |

TABLE 43
Stability of Nanoemulsions from Rosehip EMO and flaxseed FFA stored at room temperature (data represented
as average ± SD from at least three measurements; Ppt denotes visible precipitation of the formulation)

| | | | Fresh | | 14 days | | 21 days | | 28 days | |
|---|---|---|---|---|---|---|---|---|---|---|
| | | | Z | | Z | | Z | | Z | |
| | | FFA | avg | | avg | | avg | | avg | |
| EMO/FFA | EMO % | oil % | (nm) | PDI | (nm) | PDI | (nm) | PDI | (nm) | PDI |
| Rosehip/ | 75 | 25 | 189 ± 3 | 0.184 ± 0.032 | 200 ± 3 | 0.205 ± 0.004 | 200 ± 4 | 0.221 ± 0.014 | 208 ± 3 | 0.253 ± 0.007 |
| flaxseed | | | | | | | | | | |
| Rosehip/ | 50 | 50 | 193 ± 3 | 0.177 ± 0.007 | 199 ± 5 | 0.205 ± 0.035 | 233 ± 2 | 0.260 ± 0.026 | 220 ± 7 | 0.203 ± 0.027 |
| flaxseed | | | | | | | | | | |
| Rosehip/ | 25 | 75 | 205 ± 2 | 0.203 ± 0.025 | 237 ± 3 | 0.186 ± 0.053 | 287 ± 3 | 0.311 ± 0.052 | 276 ± 10 | 0.229 ± 0.087 |
| flaxseed | | | | | | | | | | |

TABLE 44
Stability of Nanoemulsions from Algae EMO and FFAs of flaxseed and MCT stored at room temperature (data represented
as average ± SD from at least three measurements; Ppt denotes visible precipitation of the formulation)

| | | | Fresh | | 14 days | | 21 days | | 28 days | |
|---|---|---|---|---|---|---|---|---|---|---|
| | | | Z | | Z | | Z | | Z | |
| | | FFA | avg | | avg | | avg | | avg | |
| EMO/FFA | EMO % | oil % | (nm) | PDI | (nm) | PDI | (nm) | PDI | (nm) | PDI |
| Algae/ | 75 | 25 | 186 ± 4 | 0.197 ± 0.016 | Ppt | Ppt | Ppt | Ppt | Ppt | Ppt |
| flaxseed | | | | | | | | | | |
| Algae/ | 50 | 50 | 187 ± 4 | 0.180 ± 0.029 | Ppt | Ppt | Ppt | Ppt | Ppt | Ppt |
| flaxseed | | | | | | | | | | |
| Algae/ | 25 | 75 | 198 ± 1 | 0.187 ± 0.034 | 214 ± 2 | 0.199 ± 0.004 | 214 ± 1 | 0.195 ± 0.025 | 216 ± 3 | 0.163 ± 0.012 |
| flaxseed | | | | | | | | | | |
| Algae/ | 75 | 25 | 452 ± 49 | 0.504 ± 0.445 | Ppt | Ppt | Ppt | Ppt | Ppt | Ppt |
| MCT | | | | | | | | | | |
| Algae/ | 50 | 50 | 317 ± 17 | 0.077 ± 0.097 | 324 ± 7 | 0.135 ± 0.109 | 318 ± 13 | 0.316 ± 0.165 | Ppt | Ppt |
| MCT | | | | | | | | | | |
| Algae/ | 25 | 75 | 270 ± 5 | 0.244 ± 0.044 | 289 ± 4 | 0.282 ± 0.014 | 304 ± 9 | 0.160 ± 0.121 | 259 ± 6 | 0.213 ± 0.059 |
| MCT | | | | | | | | | | |

FIGS. 2A-2S show the Z average particle size for the nanoemulsions in Tables 27-44.

### Example 5: Preparation of Nanoemulsions from Unmodified Oils

In order to assess the effect of TAG content on making nanoemulsions of the present disclosure, olive oil (unmodified, TAG form) was mixed with olive FFA oil prepared as described above at different ratios, and the mixtures were prepared as nanoemulsions according to the procedure described above. Z average particle size and polydispersity index were measured for these samples and the results are provided below in Table 45 below.

TABLE 45
Particle size analysis of olive oil and olive FFA oil
nanoemulsions by dynamic light scattering (data represented
as average ± SD from at least three measurements)

| Oil % | FFA oil % | Z avg (nm) | PDI |
|---|---|---|---|
| 100 | 0 | 2 layers | |
| 75 | 25 | 399 ± 46 | 0.349 ± 0.388 |
| 70 | 30 | 349 ± 29 | 0.211 ± 0.203 |
| 60 | 40 | 343 ± 10 | 0.101 ± 0.088 |
| 50 | 50 | 248 ± 5 | 0.199 ± 0.021 |
| 25 | 75 | 219 ± 3 | 0.189 ± 0.030 |

As shown in Table 45, olive oil alone, which is essentially TAGs, was unable to form a nanoemulsion as it resulted in the formation of two separate layers. In addition, the size of the droplets decreased with increasing FFA content and decreasing TAG content. Specifically, up to 50% olive oil (essentially TAGs) still yielded nanoemulsions with a Z average size of less than 300 nm, while 60% olive oil yielded larger particles and the 100% TAG sample quickly separated into 2 layers.

Olive oil (unmodified, TAG form) was also blended with olive EMO, which includes MAGs, some DAGs and FFAs, prepared as described above at various ratios and nanoemulsions prepared therefrom as described above. Z average particle size and polydispersity index were measured for these samples and the results are provided in Table 46 below.

TABLE 46
Particle size analysis of olive oil and olive EMO
nanoemulsions by dynamic light scattering (data represented
as average ± SD from at least three measurements)

| Oil % | EMO % | FFA oil % | Z avg (nm) | PDI |
|---|---|---|---|---|
| 90 | 8 | 2 | 496 ± 47 | 0.520 ± 0.443 |
| 80 | 16 | 4 | 293 ± 13 | 0.130 ± 0.091 |
| 75 | 20 | 5 | 243 ± 5 | 0.228 ± 0.010 |
| 50 | 40 | 10 | 178 ± 3 | 0.215 ± 0.009 |
| 25 | 60 | 15 | 183 ± 4 | 0.201 ± 0.018 |

As shown in Table 46, up to 80% TAG was still able to yield Z average particle sizes below 300 nm.

The same procedure was performed using olive oil (unmodified, TAG form) blended with either oleic acid or glyceryl oleate at ratios of 75/25, 50/50 and 25/75. In no cases did the blend form a nanoemulsion, rather, 2 layers were obtained.

### Example 6: Preparation of Nanoemulsions Using Off-the-Shelf Ingredients

To assess nanoemulsions prepared from off-the-shelf ingredients, nanoemulsions were prepared from glyceryl oleate (MakingCosmetics, part #EMF-GLYOL-01) and/or oleic acid (Beantown Chemical, part #126125) at various ratios. Z average size and polydispersity index were measured as described above. The results are shown in Table 47 below.

TABLE 47
Particle size analysis of glyceryl oleate/oleic acid
nanoemulsions by dynamic light scattering (data represented
as average ± SD from at least three measurements)

| Glyceryl oleate % | Oleic acid % | Z avg (nm) | PDI |
|---|---|---|---|
| 100 | 0 | 629 ± 42 | 0.227 ± 0.047 |
| 75 | 25 | 627 ± 42 | 0.250 ± 0.021 |
| 50 | 50 | 330 ± 10* | 0.323 ± 0.030* |
| 25 | 75 | ** | ** |
| 0 | 100 | 572 ± 100 | 0.132 ± 0.119 |

*Analysis from liquid layer
** Forms a cream

These results can be compared to those in Table 5 for olive EMO nanoemulsions. In contrast, the glyceryl oleate/oleic acid nanoemulsions did not form nanoemulsions of less than 300 nm Z average droplet size. Both the 50/50 and 25/75 blends yielded creams and the other samples yielded large droplet sizes of about 600 nm or larger. Thus, as performed in this Example, off-the-shelf ingredients did not replicate the results with EMO.

### Example 7: Nanoemulsions from Multiple Oil Sources

Nanoemulsions were formed from EMOs and FFAs of rosehip, flaxseed and MCT oils as described in the previous examples. The size, PDI and zeta potential data are shown in Table 48 below.

TABLE 48
Nanoemulsions from EMOs and FFAs of rosehip, flaxseed, and
MCT oils (data represented as average ± SD from at
least three measurements); This is a representative blend
for saturated and unsaturated fats from three different sources

| | | FFA | Z | | Zeta |
|---|---|---|---|---|---|
| | EMO | oil | avg | | potential |
| Oils | % | % | (nm) | PDI | (mV) |
| Rosehip | 100 | 0 | 193 ± 2 | 0.228 ± 0.025 | −29.2 ± 0.4 |
| Flaxseed | | | | | |
| MCT | | | | | |
| Rosehip | 50 | 50 | 213 ± 5 | 0.288 ± 0.050 | −42.3 ± 0.4 |
| Flaxseed | | | | | |
| MCT | | | | | |

Nanoemulsions containing three different EMOs or three different EMOs plus their corresponding FFA at 50/50 ratios achieved particle sizes less than 300 nm and PDI less than 0.300.

### Example 8: Amount of Oil in Nanoemulsion

Nanoemulsions were prepared from rosehip EMO at various % oil out of the total weight of the nanoemulsion. Table 49 below provides the size and PDI data.

TABLE 49
Nanoemulsions from rosehip EMO at various % oil compositions (data
represented as average ± SD from at least three measurements)

| | EMO (% wt of the | | |
|---|---|---|---|
| EMO | total composition) | Z avg (nm) | PDI |
| Rosehip | 1 | 178 ± 4 | 0.215 ± 0.018 |
| Rosehip | 5 | 180 ± 3 | 0.180 ± 0.004 |
| Rosehip | 10 | 199 ± 1 | 0.144 ± 0.014 |
| Rosehip | 20 | 255 ± 3 | 0.239 ± 0.005 |
| Rosehip | 30 | 270 ± 5 | 0.277 ± 0.018 |
| Rosehip | 50 | Formed a cream | |
| Rosehip | 70 | Formed a cream | |

Rosehip EMO nanoemulsions could be formed when the percent weight of the oil was 1% to 30% of the total weight of the formulation. At 50% oil weight or greater, a cream was formed.

### Example 9: Production of a Nanoemulsion Containing CBD

Fatty acid oil was prepared by complete hydrolysis of the TAGs to FFA and glycerol and their subsequent separation. Almond fatty acid oil was prepared in a 20 L stirred, jacketed glass reactor. Citric acid buffer (100 mM, pH 5.8) was prepared by combining 133 grams of food grade citric acid with 75 g of food grade sodium hydroxide in 7,000 mL of distilled de-ionized water. Amano Lipase AY (5.8 g) was dissolved in the solution which was then heated to 33° C. and agitated at 300 rpm. After a brief (10 min.) vacuum degassing, 5,600 g of almond oil was added to the reactor. The reaction volume was overlaid with nitrogen and the reaction proceeded for 12 hours until all of the TAGs had been converted to fatty acids and glycerol.

Once the reaction was complete, the mixture was heated to 70° C. for 1 hour to inactivate the lipase. Agitation was stopped. After the two phases had separated, the upper phase (the fatty acid oil) was removed and stored at room temperature in opaque plastic bottles under nitrogen.

Components of the oil samples were separated using TLC plates (Analtech Uniplate Silica Gel GHL with inorganic binder, 20×20 cm, 250 μm). The solvent was hexane:diethyl ether:acetic acid (70:30:1) solution. Typical sample sizes were 0.2 μL. After the solvent front ran to near the top of the plate (˜1 cm), plates were removed from the TLC tank and the solvent evaporated in a fume hood. The components were visualized with iodine vapors (at room temperature) in a TLC tank and relative intensities estimated by colorimetric imaging (Amersham 600 Imager). After 15 minutes in the tank, plates were removed and photographed. The intensity of the spots diminished after 30-60 minutes.

EMO was produced from the fatty acid oil through the selective re-esterification of the FFA to glycerol to form MAG and (to a much lesser extent) DAG. Fatty acid oil as described above was combined with 10,260 g of food grade glycerol and heated to 30° C. with agitation (300 rpm). Vacuum was applied (˜720 mmHg) to degas the material and remove residual water. Amano Lipase G (5 g in 50 mL DI water) was then added to the reactor and the vacuum was reapplied (˜740 mmHg). The reaction was allowed to proceed for 72 hours with progress monitored by TLC.

Once the level of FFA was reduced to below 10%, the reaction was stopped by removing the vacuum, overlaying the reactor with nitrogen and heating the system to 70° C. for an hour to inactivate the enzyme.

Fatty acid concentrations were measured by titrimetry. Samples were dissolved in a solvent (95% ethanol/diethyl ether, 1/1, v/v) and titrated to neutrality (indicated with phenolphthalein) with 0.1M KOH in ethanol.

Table salt (sodium chloride) was then added to the reactor. Once the salt was suspended, the agitation was stopped. After the two phases had separated (3 hours), the upper phase (the acylglycerol oil) was removed. Antioxidant was added (tocopherol, 200 ppm) and the material was stored at room temperature in opaque plastic bottles under nitrogen.

MMN were prepared by combining the EMO and a cargo, CBD, and subsequent emulsification. CBD was chosen as it is essentially insoluble in water. The solubility of cannabinoids in water averages approximately 0.0015 mg/mL, which is too low for efficient delivery into the body, but high enough to cause rapid nanoemulsion destabilization through Ostwald ripening.

The cargo (2.5 g of 99.9% CBD isolate) was dissolved in 10 g of almond EMO (9.7% FFA) that had been pre-heated to 50° C. in a 50 mL conical tube. The mixture was warmed overnight to ensure complete dissolution.

A coarse emulsion was prepared by adding 8.8 mL of the warm (50° ° C.) EMO/CBD dropwise into 72 mL of warm (50° C.) deionized water in a 150 mL beaker. The mixture was constantly stirred while adding the oil using a high-sheer homogenizer for 5 minutes. The resulting product was a milky coarse emulsion.

The coarse emulsion was then transferred to QSonica Model Q700CA sonicator with a ½ inch probe. The beaker was cooled in an ice bath and processed for 24 cycles (5 minutes sonication/2 minutes cooling) and 90% power. This was sample 102919.1. The emulsion was then diluted 1/10 with water and subjected to 12 more cycles. This was sample 102919.1 3X.

The TAG/DAG/MAG/FFA ratio in the sample was estimated by TLC which yielded:

TABLE 50
Amounts of TAG/DAG/MAG/FFA in
CBD nanoemulsion preparation.

| | |
|---|---|
| <1% | TAG (below limits of detection) |
| 10% +/− 5% | DAG |
| 80% +/− 5% | MAG |
| 9.7% +/− 5% | FFA |

A Zetasizer Ultra (Malvern Panalytical) was used to measure the size and zeta potential of the nanoemulsions (102919.1 3X). Disposable sizing cells and disposable folded capillary cells were used for size and zeta potential measurements (in triplicate), respectively. As shown in FIG. 3, the average size of the particles was determined to be 163 nm, well below the target size of <300 nm.

The zeta potential of the MMN (102919.1 3X) was also determined as shown in FIG. 4. As shown, the zeta potential of the particles was measured to be −44.4+/−0.9 mV which is well below the target of at least −30 mV.

### Example 10: Preparation of a Nanoemulsion Containing CBD

In a separate experiment, 4.0 g of 99.9% CBD isolate were dissolved in 16 g of Almond EMO, prepared as described in Example 9, that had been pre-heated to 50° C. in a 50 mL conical tube. The mixture was warmed overnight at 50° C. to ensure complete dissolution.

A coarse emulsion was prepared by adding 10 mL of the warm (50° C.) EMO/CBD mixture dropwise into 90 mL of warm (50° C.) deionized water in a 150 mL beaker while the beaker was cooled in an ice bath and sonicated with a QSonica Model Q700CA sonicator with a ½ inch probe for 12 cycles (5 minutes sonication/2 minutes cooling) and 90% power. This produced a milky coarse emulsion.

The coarse emulsion was then subjected to 65 more sonication cycles (about 6 hours).

The resulting nanoemulsion (111819.1) was stable and found the have an average particle size of 169.2 nm, and a zeta potential of −42.6 mV. Size distribution measurements were performed in triplicate. The size distribution for the nanoemulsions are shown in FIG. 5.

### Example 11: Preparation of Nanoemulsions Using Canola Oil and Vitamin E

MMN will be prepared according to the procedure described above in Example 9 using canola oil as the starting oil instead of almond oil and Vitamin E as the cargo instead of CBD. MMN will also be prepared according to the procedure described above in Example 9 using almond oil with Vitamin E as the cargo. It is expected that similar results will be obtained.

### Example 12: Comparison of EMO Nanoemulsions, EMO Microemulsions, and TAG Microemulsions for Enhancing Drug Permeability Through Skin

The permeation profile of hydrocortisone across a synthetic, skin-like membrane (Strat-M®) when formulated different emulsion formats was assessed, including in sesame EMO nanoemulsions (nS50) and microemulsions (micro-S50) as well as sesame TAG nanoemulsions (nano-TAG).

Sesame EMO nanoemulsions were prepared from a combination of sesame EMO and FFA oil at different ratios. The content of the EMO oils is provided in Table 1 and the FFA oil was essentially 100% FFA. For nS25, sesame EMO (0.426 g) and sesame FFA oil (1.07 g) were weighed into a 50 mL metal beaker and heated to 60° C. Hydrocortisone (15 mg) (Acros Organics) was added to the oil mixture and gently mixed at 60° C. to form a fine dispersion. A coarse emulsion was prepared by adding 13.5 mL water (pre-heated to 60° C.) to the oil. The mixture was subjected to high-sheer homogenizer (Brinkmann Homogenizer Model PT 10/35) for 30 seconds, resulting in a milky coarse emulsion. The coarse emulsion was then transferred to QSonica Q700CA sonicator with ½ inch probe. The emulsion was processed for 2 cycles (5 minutes sonication/2 minutes cooling) at 90% power. The resulting nanoemulsion was analyzed by DLS (Z avg=222±7 nm; PDI=0.175 0.017).

nS50 was prepared using sesame EMO (0.852 g) and sesame FFA (0.648 g) and using the same procedure described above. The resulting nanoemulsion was analyzed by DLS (Z avg=185±5 nm; PDI=0.181±0.013).

nS75 was prepared using sesame EMO (1.278 g) and sesame FFA (0.222 g) and using the same procedure described above. The resulting nanoemulsion was analyzed by DLS (Z avg=179±1 nm; PDI=0.203±0.010).

Oat/flaxseed EMO nanoemulsions were prepared from a combination of oat EMO and flaxseed FFA oil at different ratios. The content of the EMO oils is provided in Table 1 and the FFA oil was essentially 100% FFA. For nOa/Fx25, oat EMO (0.375 g) and flaxseed FFA oil (1.125 g) were weighed into a 50 mL metal beaker and heated to 60° C. Hydrocortisone (15 mg) (Acros Organics) was added to the oil mixture and gently mixed at 60° C. to form a fine dispersion. A coarse emulsion was prepared by adding 13.5 mL water (pre-heated to 60° C.) to the oil. The mixture was subjected to high-sheer homogenizer for 30 seconds, resulting in a milky coarse emulsion. The coarse emulsion was then transferred to QSonica Q700CA sonicator with ½ inch probe. The emulsion was processed for 2 cycles (5 minutes sonication/2 minutes cooling) at 90% power. The resulting nanoemulsion was analyzed by DLS (Z avg=181±3 nm; PDI=0.157±0.013). nOa/Fx75 was prepared using oat EMO (1.125 g) and flaxseed FFA (0.375 g) and using the same procedure described above. The resulting nanoemulsion was analyzed by DLS (Z avg=160±3 nm; PDI=0.194±0.019). nOa/Fx50 was prepared using oat EMO (0.750 g) and flaxseed FFA (0.750 g) and using the same procedure described above. The resulting nanoemulsion was analyzed by DLS (Z avg=172±3 nm; PDI=0.165±0.041). nFx0 was prepared using flaxseed FFA (1.500 g) and using the same procedure described above. The resulting nanoemulsion was analyzed by DLS (Z avg=193±1 nm; PDI=0.147±0.032).

Sesame EMO microemulsions (micro-S50) were prepared from a combination of sesame EMO and FFA oil. Sesame EMO (0.852 g) and sesame FFA oil (0.648 g) were weighed into a 50 mL metal beaker and heated to 60° C. Hydrocortisone (15 mg) was added to the oil mixture and gently mixed at 60° C. to form a fine dispersion. A coarse emulsion was prepared by adding 13.5 mL water (pre-heated to 60° C.) to the oil. The mixture was subjected to high-sheer homogenizer for 30 seconds, resulting in a milky coarse emulsion.

TAG nanoemulsions (nano-TAG sesame) were prepared from a combination of unmodified sesame oil and span 80. Sesame oil (1.5 g) and span 80 (0.75 g) were weighed into a 50 mL metal beaker and heated to 60° C. Hydrocortisone (15 mg) was added to the oil mixture and gently mixed at 60° C. to form a fine dispersion. A coarse emulsion was prepared by adding 12.75 mL water (pre-heated to 60° C.) to the oil. The mixture was subjected to high-sheer homogenizer for 30 seconds, resulting in a milky coarse emulsion. The coarse emulsion was then transferred to QSonica Q700CA sonicator with ½ inch probe. The emulsion was processed for 5 minutes sonication at 90% power. The resulting nanoemulsion was analyzed by DLS (Z avg=262±1 nm; PDI=0.230±0.007).

The properties of the various formulations are provided in Table 51 below.

TABLE 51
Composition and size analysis of various drug-loaded EMO nanoemulsions. Size
and PDI analysis was performed using dynamic light scattering (data represented
as average ± SD from at least 3 measurements) for Examples 12-14.

| | | | | FFA | | | |
|---|---|---|---|---|---|---|---|
| | EMO | FFA | EMO | oil | | Size | |
| Formulation | (oil) | (oil) | (% wt) | (% wt) | Drug | (nm) | PDI |
| nS25 | Sesame | Sesame | 25 | 75 | HCT | 222 ± 7 | 0.175 ± 0.017 |
| nS50 | Sesame | Sesame | 50 | 50 | HCT | 185 ± 5 | 0.181 ± 0.013 |
| nS75 | Sesame | Sesame | 75 | 25 | HCT | 179 ± 1 | 0.203 ± 0.010 |
| nFx0 | None | Flaxseed | 0 | 100 | HCT | 193 ± 1 | 0.147 ± 0.032 |
| nOa/Fx25 | Oat | Flaxseed | 25 | 75 | HCT | 181 ± 3 | 0.157 ± 0.013 |
| nOa/Fx50 | Oat | Flaxseed | 50 | 50 | HCT | 172 ± 3 | 0.165 ± 0.041 |
| nOa/Fx75 | Oat | Flaxseed | 75 | 25 | HCT | 193 ± 1 | 0.147 ± 0.032 |
| nS50 | Sesame | Sesame | 50 | 50 | CBD | 186 ± 2 | 0.204 ± 0.007 |
| nAl25 | Almond | Almond | 25 | 75 | CLA | 258 ± 6 | 0.228 ± 0.033 |
| nAl50 | Almond | Almond | 50 | 50 | CLA | 196 ± 2 | 0.194 ± 0.018 |
| nAl75 | Almond | Almond | 75 | 25 | CLA | 183 ± 2 | 0.214 ± 0.021 |
| nCo50 | Coconut | Coconut | 50 | 50 | CLA | 138 ± 2 | 0.241 ± 0.009 |
| nMCT50 | MCT | MCT | 50 | 50 | CLA | 182 ± 3 | 0.265 ± 0.025 |

HCT = hydrocortisone
CBD = cannabadiol
CLA = Clarithromycin A

Free drug control was prepared by dissolving hydrocortisone in DMSO at 10 mg/mL and diluting 10-fold into water.

For non-loaded drug formulations, nS50 and micro-S50 were prepared as previously described but without the addition of HCT. A stock of HCT in DMSO at 10 mg/mL was prepared separately and then diluted 10-fold into the pre-formed nS50 or micro-S50 formulations and vortexed.

Strat-M® membrane was set in a Franz-type diffusion cell (1.77 cm² area). Receptor chamber was equipped with stir bar, filled with PBS (10 mL), and placed in 32° C. water bath. Hydrocortisone (HCT) formulations (1 mL) were added to the donor chamber. At each time point, 150 μL was removed from the receiver chamber and replaced with an equivalent volume of fresh PBS to maintain constant volume. The aliquot from each time point was analyzed by HPLC to determine amount of drug permeating through the membrane over time. The HPLC system consisted of an LC-20AD pump, SIL-20AC HT autosampler, CTO-20A column oven, and SPD-20A UV/vis detector (Shimadzu Scientific Instruments, Inc.). HPLC conditions were: 30 μL injection volume, isocratic elution of mobile phase consisting of 55% methanol and 45% water, flow rate of 1.000 mL/min, column temperature of 40° C., and detection at 244 nm. Column used was a SUPELCOSIL LC-18-T column (150 mm×4.6 mm; 3 μm particle size). The cumulative amount of drug (HCT) permeated per unit time for the drug-loaded formulations and free drug control are provided in FIG. 6.

The nS50 formulation provided the best permeation enhancement with cumulative drug amount of 11.8±0.6 μg/cm² at seven hours compared to micro-S50 (6.88 0.01 μg/cm²), nano-TAG sesame (1.80±0.38 μg/cm²), and free drug control (1.29±0.26 μg/cm²) (FIG. 6). These results suggest superior permeation enhancement properties of EMO oils compared to unmodified TAG oils when in emulsion form. Further, the EMO nanoemulsions allowed 1.7-fold more drug to permeate compared to the EMO microemulsions, indicating that despite having the same chemical composition, smaller particle sizes provide better permeation enhancement.

Non-loaded versions of EMO nano and microemulsions were also tested. For these formulations, hydrocortisone was spiked into the pre-formed nano and microemulsions. The results in FIG. 7 again show that EMO nanoemulsions provide better permeation enhancement compared to EMO microemulsions. However, it was surprisingly found that even when the active ingredient is added after formation of the emulsions, permeability was still improved with minimal differences seen between the loaded and non-loaded versions.

Blends of oat EMO and flaxseed FFA oil were also tested to support the use of other oils for drug permeation. FIG. 8 shows all formulations significantly enhanced hydrocortisone permeation across the Strat-M® membrane. Notably, the nFx0 (which is composed of flaxseed FFA) allowed the most drug permeation (30.6±1.9 μg/cm²) over 7 hours compared to the other formulations, possibly due to its higher FFA content. Indeed, formulations with higher FFA contents correlated with more drug permeation with a rank order of nFx0>nOa/Fx25>nOa/Fx50>nOa/Fx75. A similar trend was seen when comparing sesame EMO nanoemulsions at different ratios of EMO and FFA (FIG. 9). Again, formulations with higher FFA contents correlated with more drug permeation with a rank order of nS25>nS50>nS75.

Collectively, these data support the use of EMO emulsions for topical and transdermal delivery of drugs, and that oil type, FFA content, and particle size affect permeation enhancement.

### Example 13: CBD Formulations

Sesame EMO nanoemulsions were prepared from a combination of sesame EMO and FFA oil. Sesame EMO (0.852 g) and sesame FFA oil (0.648 g) were weighed into a 50 mL metal beaker and heated to 60° C. CBD (150 mg) was added to the oil mixture and gently mixed at 60° C. until CBD was completely dissolved. A coarse emulsion was prepared by adding 13.5 mL water (pre-heated to 60° C.) to the oil. The mixture was subjected to high-sheer homogenizer for 30 seconds, resulting in a milky coarse emulsion. The coarse emulsion was then transferred to QSonica Q700CA sonicator with ½ inch probe. The emulsion was processed for 2 cycles (5 minutes sonication/2 minutes cooling) at 90% power. The resulting nanoemulsion was analyzed by DLS (Z avg=186±2 nm; PDI=0.204 0.007).

### Example 14: Clarithromycin A Formulations

Almond EMO nanoemulsions (nA175, nA150, nA125) were prepared from a combination of almond EMO and FFA oil at different ratios. For nA175, almond EMO (1.667 g) and almond FFA (0.333 g) were weighed into a 50 mL metal beaker and heated to 60° C. Clarithromycin A (20 mg) was added to the oil mixture and gently mixed at 60° C. to form a fine dispersion. A coarse emulsion was prepared by adding 18.0 mL water (pre-heated to 60° C.) to the oil. The mixture was subjected to high-sheer homogenizer for 30 seconds, resulting in a milky coarse emulsion. The coarse emulsion was then transferred to QSonica Q700CA sonicator with ½ inch probe. The emulsion was processed for 2 cycles (5 minutes sonication/2 minutes cooling) at 90% power. The resulting nanoemulsion was analyzed by DLS (Z avg=183±2; PDI=0.214±0.021).

nA150 was prepared using almond EMO (1.111 g) and almond FFA (0.889 g) and using the same procedure described above. The resulting nanoemulsion was analyzed by DLS (Z avg=196±2; PDI=0.194±0.018).

nA125 was prepared using almond EMO (0.556 g) and almond FFA (1.444 g) and using the same procedure described above. The resulting nanoemulsion was analyzed by DLS (Z avg=258±6; PDI=0.228±0.033).

Coconut EMO nanoemulsion (nCo50) was prepared from a combination of coconut EMO and FFA oil. Coconut EMO (1.136 g) and coconut FFA (0.864 g) were weighed into a 50 mL metal beaker and heated to 60° C. Clarithromycin A (20 mg) was added to the oil mixture and gently mixed at 60° C. to form a fine dispersion. A coarse emulsion was prepared by adding 18.0 mL water (pre-heated to 60° C.) to the oil. The mixture was subjected to high-sheer homogenizer for 30 seconds, resulting in a milky coarse emulsion. The coarse emulsion was then transferred to QSonica Q700CA sonicator with ½ inch probe. The emulsion was processed for 2 cycles (5 minutes sonication/2 minutes cooling) at 90% power. The resulting nanoemulsion was analyzed by DLS (Z avg=138±2; PDI=0.241±0.009).

MCT EMO nanoemulsion (nMCT50) was prepared from a combination of MCT EMO and FFA oil. MCT EMO (1.266 g) and MCT FFA (0.734 g) were weighed into a 50 mL metal beaker and heated to 60° C. Clarithromycin A (20 mg) was added to the oil mixture and gently mixed at 60° C. to form a fine dispersion. A coarse emulsion was prepared by adding 18.0 mL water (pre-heated to 60° C.) to the oil. The mixture was subjected to high-sheer homogenizer for 30 seconds, resulting in a milky coarse emulsion. The coarse emulsion was then transferred to QSonica Q700CA sonicator with ½ inch probe. The emulsion was processed for 2 cycles (5 minutes sonication/2 minutes cooling) at 90% power. The resulting nanoemulsion was analyzed by DLS (Z avg=182±3; PDI=0.265±0.025).

### Example 15: In Vitro Skin Permeation Study of Hydrocortisone Using EMO Nanoemulsions

Strat-M® membrane (lot R1BB 11781) was set in a Franz-type diffusion cell (1.77 cm² area). Receptor chamber was equipped with stir bar, filled with PBS (10 mL), and placed in 32° C. water bath. Hydrocortisone (HCT) formulations (1 mL), described below, were added to the donor chamber. At each time point, 100 μL was removed from the receiver chamber and replaced with an equivalent volume of fresh PBS to maintain constant volume. The aliquot from each time point was analyzed by HPLC to determine amount of drug permeating through the membrane over time. The HPLC system consisted of an LC-20AD pump, SIL-20AC HT autosampler, CTO-20A column oven, and SPD-20A UV/vis detector (Shimadzu Scientific Instruments, Inc.). HPLC conditions were: 30 μL injection volume, isocratic elution of mobile phase consisting of 55% methanol and 45% water, flow rate of 1.000 mL/min, column temperature of 40° C., and detection at 244 nm. Column used was a SUPELCOSIL LC-18-T column (150 mm×4.6 mm; 3 μm particle size).

MCT EMO was derived from MCT oil (Viva Naturals) which is 55-65% C8, 35-45% C10, and 2% other. MCT EMO nanoemulsion was prepared from a combination of MCT EMO and FFA oil. MCT EMO (0.901 g) and MCT FFA (0.593 g) were weighed into a 50 mL metal beaker and heated to 60° C. A coarse emulsion was prepared by adding 13.5 mL water (pre-heated to 60° C.) to the oil. The mixture was subjected to high-sheer homogenizer for 30 seconds, resulting in a milky coarse emulsion.

Flaxseed EMO nanoemulsion was prepared from a combination of flaxseed EMO and FFA oil. Flaxseed EMO (0.914 g) and flaxseed FFA (0.585 g) were weighed into a 50 mL metal beaker and heated to 60° C. A coarse emulsion was prepared by adding 13.5 mL water (pre-heated to 60° C.) to the oil. The mixture was subjected to high-sheer homogenizer for 30 seconds, resulting in a milky coarse emulsion.

Oat EMO nanoemulsion was prepared from a combination of oat EMO and FFA oil. Oat EMO (0.862 g) and oat FFA (0.638 g) were weighed into a 50 mL metal beaker and heated to 60° C. A coarse emulsion was prepared by adding 13.5 mL water (pre-heated to 60° C.) to the oil. The mixture was subjected to high-sheer homogenizer for 30 seconds, resulting in a milky coarse emulsion.

Sesame EMO nanoemulsion was prepared from a combination of sesame EMO and FFA oil. Sesame EMO (0.852 g) and sesame FFA (0.648 g) were weighed into a 50 mL metal beaker and heated to 60° C. A coarse emulsion was prepared by adding 13.5 mL water (pre-heated to 60° C.) to the oil. The mixture was subjected to high-sheer homogenizer for 30 seconds, resulting in a milky coarse emulsion.

Rosehip EMO nanoemulsion was prepared from a combination of rosehip EMO and FFA oil. Rosehip EMO (0.893 g) and rosehip FFA (0.607 g) were weighed into a 50 mL metal beaker and heated to 60° C. A coarse emulsion was prepared by adding 13.5 mL water (pre-heated to 60° C.) to the oil. The mixture was subjected to high-sheer homogenizer for 30 seconds, resulting in a milky coarse emulsion.

Almond EMO nanoemulsion was prepared from a combination of almond EMO and FFA oil. Almond EMO (0.833 g) and rosehip FFA (0.667 g) were weighed into a 50 mL metal beaker and heated to 60° C. A coarse emulsion was prepared by adding 13.5 mL water (pre-heated to 60° C.) to the oil. The mixture was subjected to high-sheer homogenizer for 30 seconds, resulting in a milky coarse emulsion.

Hemp seed EMO nanoemulsion was prepared from a combination of hemp EMO and FFA oil. Hemp EMO (0.824 g) and MCT FFA (0.676 g) were weighed into a 50 mL metal beaker and heated to 60° C. A coarse emulsion was prepared by adding 13.5 mL water (pre-heated to 60° C.) to the oil. The mixture was subjected to high-sheer homogenizer for 30 seconds, resulting in a milky coarse emulsion.

A stock of HCT in DMSO at 10 mg/mL was prepared separately and then diluted 10-fold into the pre-formed nanoemulsion formulations and vortexed.

Free drug control was prepared by diluting the HCT DMSO stock 10-fold into water and vortexed.

All EMO nanoemulsions demonstrated significant permeation enhancement compared to the free drug control (HCT Control) as shown in FIG. 10. Notably, the MCT nanoemulsion provided the best permeation enhancement (166±13 ug/cm\hat{ }2). These data support the use of nanoemulsions of various EMO oil types for enhanced drug skin permeation.

### Example 16. In Vitro Skin Permeation Study of Cerave Hydrocortisone Cream with and without Oat EMO

Strat-M® membrane (lot R1BB 11781) was set in a Franz-type diffusion cell (1.77 cm² area). Receptor chamber was equipped with stir bar, filled with PBS (10 mL), and placed in 32° C. water bath. 100 mg of Cerave (1% HCT by weight) with and without the addition of 10% oat EMO were added to the donor chamber. At each time point, 100 μL was removed from the receiver chamber and replaced with an equivalent volume of fresh PBS to maintain constant volume. The aliquot from each time point was analyzed by HPLC to determine amount of drug permeating through the membrane over time. The HPLC system consisted of an LC-20AD pump, SIL-20AC HT autosampler, CTO-20A column oven, and SPD-20A UV/vis detector (Shimadzu Scientific Instruments, Inc.). HPLC conditions were: 30 μL injection volume, isocratic elution of mobile phase consisting of 55% methanol and 45% water, flow rate of 1.000 mL/min, column temperature of 40° C., and detection at 244 nm. Column used was a SUPELCOSIL LC-18-T column (150 mm×4.6 mm; 3 μm particle size).

As shown in FIG. 11, the addition of oat EMO (10% by weight in this particular example) to Cerave hydrocortisone cream improved the HCT permeation 18-fold at 7 hours compared to the Cerave cream without oat EMO. This supports the use of adding EMOs as individual ingredients in topical formulations to improve skin permeation of drugs.

### Example 17. In Vitro Skin Permeation Study of Salicylic Acid with and without Nanoemulsion of Sesame Oil EMO and FFA Oil (nS50)

Strat-M® membrane (lot R1DB96259) was set in a Franz-type diffusion cell (1.77 cm² area). Receptor chamber was equipped with stir bar, filled with PBS (10 mL), and placed in 32° C. water bath. Sesame EMO nanoemulsion having 50% FFA (labeled as nS50 although it was not 50% EMO) was prepared from a combination of sesame EMO and FFA oil to yield 35.5% MAG, 10.2% DAG, and 50% FFA from a 50/50 mix of Sesame EMO and Sesame FFA oil. Sesame EMO (0.852 g) and sesame FFA oil (0.648 g) were weighed into a 50 mL metal beaker and heated to 60° C. A coarse emulsion was prepared by adding 13.5 mL water (pre-heated to 60° C.) to the oil. The mixture was subjected to high-sheer homogenizer for 30 seconds, resulting in a milky coarse emulsion. Approximately 1 mL of salicylic acid solution (1 mg/mL in PBS, pH 5-6) or salicylic acid nanoemulsion (1 mg/mL in nS50, pH 5-6) were added to the donor chamber. At each time point, 80 μL was removed from the receiver chamber and replaced with an equivalent volume of fresh PBS to maintain constant volume. The aliquot from each time point was analyzed by HPLC to determine amount of drug permeating through the membrane over time. The HPLC system consisted of an LC-20AD pump, SIL-20AC HT autosampler, CTO-20A column oven, and SPD-20A UV/vis detector (Shimadzu Scientific Instruments, Inc.). HPLC conditions were: 30 μL injection volume, isocratic elution of mobile phase consisting of 50% methanol and 50% phosphoric acid (0.1%), flow rate of 1.000 mL/min, column temperature of 40° C., and detection at 275 nm. Column used was a SUPELCOSIL LC-18-T column (150 mm×4.6 mm; 3 μm particle size).

As shown in FIG. 12, no salicylic acid permeation was observed from the free drug control. However, the nanoemulsion formulation enabled up to 8 ug/cm\hat{ }2 permeation of salicylic acid over 7 hours. This is a remarkable finding since salicylic acid skin permeation is known to decrease when pH increases above pH 3. These data support the use of using EMO nanoemulsions for improving salicylic acid skin permeation at physiologically relevant pH (e.g., pH 5-6).

### Example 18. Permeability of Salicylic Acid in Acne Products

Commercial products containing salicylic acid were obtained and included: (1) CHARLOTTE'S WEB CBDMEDIC Acne Treatment Medicated Cream which contains 1% salicylic acid, (2) CERAVE Acne Control Gel which contains 2% salicylic acid; (3) CERAVE Body Wash for Rough & Bumpy Skin; (4) CLEARASIL Stubborn Acne+Marks 1 Minute Mask which contains 2% salicylic acid; (5) NEUTROGENA Rapid Clear Acne Eliminating Spot Gel which contains 2% salicylic acid; (6) CLEARASIL Rapid Rescue Spot Treatment Gel which contains 2% salicylic acid; and (7) CERAVE Psoraisis Moisturizing Cream containing 2% salicylic acid.

For the test articles, each product was used as is or supplemented with 10% EMO. For all products tested, oat EMO was used with the exception of sesame EMO used in the CHARLOTTE's WEB CBDMEDIC Acne Treatment Medicated Cream product. Test articles were prepared by adding EMO to cream product and manually mixing. For example, 100 mg EMO is added to 900 mg product and mixed to achieve a test article containing 10% EMO.

Strat-M® membrane (lot R1KB43611) was set in a Franz-type diffusion cell (1.77 cm² area). Receptor chamber was equipped with stir bar, filled with PBS (10 mL), and placed in 32° C. water bath. Approximately 100 mg of salicylic acid creams (Neutrogena Rapid Clear and Cerave Psoriasis) with and without 10% oat EMO were added to the donor chamber. At each time point, 80 μL was removed from the receiver chamber and replaced with an equivalent volume of fresh PBS to maintain constant volume. The aliquot from each time point was analyzed by HPLC to determine amount of drug permeating through the membrane over time. The HPLC system consisted of an LC-20AD pump, SIL-20AC HT autosampler, CTO-20A column oven, and SPD-20A UV/vis detector (Shimadzu Scientific Instruments, Inc.). HPLC conditions were: 30 μL injection volume, isocratic elution of mobile phase consisting of 50% methanol and 50% phosphoric acid (0.1%), flow rate of 1.000 mL/min, column temperature of 40° C., and detection at 275 nm. Column used was a SUPELCOSIL LC-18-T column (150 mm×4.6 mm; 3 μm particle size).

Cumulative salicylic acid permeation over time is depicted in FIGS. 12A-12G. As shown, the addition of EMO to these products yielded mixed results. For example, the addition of 10% sesame EMO showed no significant improvement in the CHARLOTTE'S WEB CBDMEDIC product (FIG. 13A) which may, without being bound to theory, be due to matrix effects. The addition of EMO to CERAVE Acne Control Gel, CERAVE Body Wash and CLEARASIL Stubborn Acne Control+Marks 1 Minute Mask was reduced compared to the unmodified products (FIGS. 13B-13D) which may, without being bound to theory, also be due to matrix and/or compatibility issues as thickening and clumping of these products was observed upon addition of EMO. It is expected, without being bound to theory, that using a different type of EMO or adding the EMO during the manufacturing process as opposed to mixing it after the commercial formulation is produced may improve the effects.

The addition of EMO to NEUTROGENA Rapid Rescue Spot Treatment Gel and CLEARASIL Rapid Rescue Spot Treatment Gel resulted in approximately a 2-fold improvement in salicylic acid permeation at the earliest time point (1 hour), but the permeation profiles mostly overlapped at 2 hours and beyond (FIGS. 13E-13F). The most substantial difference was observed with EMO added to the CERAVE Psoriasis Moisturizing Cream where a 4- to 5-fold improvement in salicylic acid permeation was observed over seven hours (4-fold at 2 hours and at 7 hours) (FIG. 13G).

### Example 19. Permeation Enhancement of EMO Versus EMO Nanoemulsions in a Cream Product

CERAVE Psoriasis Moisturizing Cream containing 2% salicylic acid was prepared with 50% water (control), 50% nOa/Fx-50 (5% total oil components, nanoemulsion, the nOA/Fx-50 was prepared as a 10% nanoemulsion in water and diluted to 5%), or 45% water and 5% Oa/Fx-50 (5% total EMO components, non-nanoemulsion). Oat EMO (Oa) and flaxseed FFA oil (Fx) were obtained as described in International Patent Application Nos. PCT/US2018/055583, filed Oct. 12, 2018, published as WO 2019/075307 and PCT/US2020/014182, filed Jan. 17, 2020, published as WO 2020/150661, and U.S. Patent Application Publication No. 2020/0146307 (U.S. Ser. No. 16/746,700), each of which is incorporated herein by reference in its entirety.

To prepare the nanoemulsion, 0.750 g of each oil (EMO and FFA oil) was added to a 50 mL metal beaker and heated to 60° C. A coarse emulsion was prepared by adding 13.5 mL of water (pre-heated to 60° C.) to the oil. The mixture was subjected to a high-shear homogenizer for 30 seconds, resulting in a milky coarse emulsion. The coarse emulsion was then transferred to a QSonica Q700CA sonicator with a ½ inch probe. The emulsion was processed for 2 cycles (5 minutes sonication/2 minutes cooling) at 90% power to obtain the nanoemulsion. The non-nanoemulsion was prepared by adding 4.5 mL of water to 5 g of product ream and homogenizing, followed by the addition of 2.5 g oat EMO and 0.25 g flaxseed FFA oil, followed by homogenizing.

Permeability was measured using the standard method already described. Strat-M® membrane (lot R1KB43611) was set in a Franz-type diffusion cell (1.77 cm² area). The receptor chamber was equipped with a stire bar and filled with PBS (10 ml), and placed in a 32° C. water bath.

As shown in FIG. 14, both the addition of the nanoemulsion and the EMO/FFA oil (non-nanoemulsion) significantly improved permeability of salicylic acid compared to the control (no EMO/FFA oil added) to a similar degree. It is expected, without being bound to theory, that this may be due to the fact that whether the EMO/FFA oil mixture is pre-homogenized before addition to the cream or after, the components of the EMO/FFA oil mixture are ultimately homogenized with the other lipid components of the cream in the discontinuous phase, resulting in a similar final product regardless of whether the EMO/FFA oil mixture was in nanoemulsion or non-nanoemulsion form before it was added.

### Example 20. Permeability of Bupivacaine HCl with and without Nanoemulsion of Sesame Oil EMO (nS100)

Strat-M® membrane (lot R1KB43611) was set in a Franz-type diffusion cell (1.77 cm² area). Receptor chamber was equipped with stir bar, filled with PBS (10 mL), and placed in 32° C. water bath. Sesame EMO nanoemulsion (nS100) was prepared from sesame oil EMO. Sesame EMO (1.50 g) was weighed into a 50 mL metal beaker and heated to 60° C. A coarse emulsion was prepared by adding 13.5 mL water (pre-heated to 60° C.) to the oil. The mixture was subjected to high-sheer homogenizer for 30 seconds, resulting in a milky coarse emulsion. The coarse emulsion was then transferred to QSonica Q700CA sonicator with 1%2 inch probe. The emulsion was processed for 2 cycles (5 minutes sonication/2 minutes cooling) at 90% power. Approximately 1 mL of bupivacaine solution (0.5 mg/mL in water) or bupivacaine nanoemulsion (0.5 mg/mL in nS100) were added to the donor chamber. At each time point, 70 μL was removed from the receiver chamber and replaced with an equivalent volume of fresh PBS to maintain constant volume. The aliquot from each time point was analyzed by HPLC to determine amount of drug permeating through the membrane over time. The HPLC system consisted of an LC-20AD pump, SIL-20AC HT autosampler, CTO-20A column oven, and SPD-20A UV/vis detector (Shimadzu Scientific Instruments, Inc.). HPLC conditions were: 30 μL injection volume, isocratic elution of mobile phase consisting of 75% methanol and 25% 10 mM sodium phosphate buffer (pH 7.8), flow rate of 1.000 mL/min, column temperature of 40° C., and detection at 263 nm. Column used was a SUPELCOSIL LC-18-T column (150 mm×4.6 mm; 3 μm particle size).

As shown in FIG. 15, the sesame EMO nanoemulsion significantly enhanced the skin permeation of bupivacaine compared to the free drug control. The nanoemulsion enabled faster permeation (0.45 ug/cm\hat{ }2 at 3 hours compared to undetectable permeation from the free drug control) and overall greater permeation (7-fold improvement at 7 hours). These data support the use of using EMO nanoemulsions for improving skin permeation of basic drug such as bupivacaine.

### Example 21. Permeability of Lidocaine

Strat-M® membrane (lot R1KB43611) was set in a Franz-type diffusion cell (1.77 cm² area). Receptor chamber was equipped with stir bar, filled with PBS (10 mL), and placed in 32° C. water bath. Approximately 100 mg of ASPERCREME with and without the addition of 5% sesame EMO, 10% sesame EMO, or 10% oat EMO were added to the donor chamber. At each time point, 70 μL was removed from the receiver chamber and replaced with an equivalent volume of fresh PBS to maintain constant volume. The aliquot from each time point was analyzed by HPLC to determine amount of drug permeating through the membrane over time. The HPLC system consisted of an LC-20AD pump, SIL-20AC HT autosampler, CTO-20A column oven, and SPD-20A UV/vis detector (Shimadzu Scientific Instruments, Inc.). HPLC conditions were: 30 μL injection volume, isocratic elution of mobile phase consisting of 70% methanol and 30% 10 mM sodium phosphate buffer (pH 7.8), flow rate of 1.000 mL/min, column temperature of 40° C., and detection at 263 nm. Column used was a SUPELCOSIL LC-18-T column (150 mm×4.6 mm; 3 μm particle size).

As shown in FIG. 16A, the addition of 10% oat EMO or 10% sesame EMO resulted in significantly faster permeation (36 ug/cm\hat{ }2 at 1 hr) compared to ASPERCREME (7.8 ug/cm\hat{ }2 at 1 hr). There was no difference in permeation enhancement between 10% oat EMO and 10% sesame EMO. Interestingly, the addition of 5% sesame EMO produces equivalent overall permeation compared to 10% sesame EMO, which was 3-fold better than the ASPERCREME alone. Samples were also prepared with 2% or 1% sesame EMO and subjected to the same experimental procedure, the results of which are shown in FIG. 16B, which shows that 5% and 10% sesame EMO enhanced permeability significantly while 1% and 2% sesame EMO did not enhance permeability to the same degree.

Lidocaine carbomer formulation was prepared by dissolving lidocaine free base in ethanol and cetyl alcohol and adding to a 0.4% carbomer 934p solution, followed by dilution in water and vortexing. The formulation contained ethanol (15%), carbomer 934p (0.4%), cetyl alcohol (1%), lidocaine (3.5%; equivalent to 4% lidocaine HCl), and water.

Lidocaine carbomer formulation with sesame EMO was prepared by dissolving lidocaine in ethanol, cetyl alcohol, and sesame EMO and adding to a 0.4% carbomer 934p solution, followed by dilution in water and vortexing. The formulation contained ethanol (15%), carbomer 934p (0.4%), cetyl alcohol (1%), lidocaine (0.875%, 1.75%, or 3.5%; equivalent to 1, 2, and 4% lidocaine HCl), sesame EMO (10%), and water.

Strat-M® membrane (lot R1KB43611) was set in a Franz-type diffusion cell (1.77 cm² area). Receptor chamber was equipped with stir bar, filled with PBS (10 mL), and placed in 32° C. water bath. Approximately 100 mg of formulations were added to the donor chamber. At each time point, 70 μL was removed from the receiver chamber and replaced with an equivalent volume of fresh PBS to maintain constant volume. The aliquot from each time point was analyzed by HPLC to determine amount of drug permeating through the membrane over time. The HPLC system consisted of an LC-20AD pump, SIL-20AC HT autosampler, CTO-20A column oven, and SPD-20A UV/vis detector (Shimadzu Scientific Instruments, Inc.). HPLC conditions were: 30 μL injection volume, isocratic elution of mobile phase consisting of 70% methanol and 30% 10 mM sodium phosphate buffer (pH 7.8), flow rate of 1.000 mL/min, column temperature of 40° C., and detection at 263 nm. Column used was a SUPELCOSIL LC-18-T column (150 mm×4.6 mm; 3 μm particle size).

As shown in FIG. 16C, the generic in-house carbomer formulation achieved a 2-fold improvement in lidocaine permeation compared to ASPERCREME, despite both having the same concentration of lidocaine. Remarkably, the addition of 10% sesame EMO to the carbomer formulation significantly enhanced lidocaine permeation, with a 3.8-fold improvement over the carbomer formulation and a 8-fold improvement over ASPERCREME.

To determine the effects of drug concentration on permeation performance, formulas containing 1% and 2% lidocaine were made, and their permeation was compared to the 4% lidocaine formula and ASPERCREME (FIG. 16D). There was observed a positive correlation between drug concentration and overall drug permeation. Overall, the generic carbomer lidocaine formulations containing 10% sesame EMO and 50-75% drug reduction were equivalent to ASPERCREME. At the early time points (up to 2 hours), the 2% lidocaine formulation matches ASPERCREME. At the later time points (2-5 hours), the 1% lidocaine formulation matches ASPERCREME. Therefore, the addition of EMO can be used reduce drug dose, and presumably toxicities, while maintaining drug exposure at therapeutic concentrations.

These data support the use of adding EMOs as individual ingredients in topical formulations to improve skin permeation of basic drugs such as lidocaine.

### Example 22. In Vitro Skin Permeation Study of EMLA (Prilocaine and Lidocaine) Cream with and without Sesame EMO

Strat-M® membrane (lot R1KB43611) was set in a Franz-type diffusion cell (1.77 cm² area). Receptor chamber was equipped with stir bar, filled with PBS (10 mL), and placed in 32° C. water bath. Approximately 100 mg of EMLA with and without the addition of 0.5, 1, 2.5, 5, and 10% sesame EMO were added to the donor chamber. At each time point, 70 μL was removed from the receiver chamber and replaced with an equivalent volume of fresh PBS to maintain constant volume. The aliquot from each time point was analyzed by HPLC to determine amount of drug permeating through the membrane over time. The HPLC system consisted of an LC-20AD pump, SIL-20AC HT autosampler, CTO-20A column oven, and SPD-20A UV/vis detector (Shimadzu Scientific Instruments, Inc.). HPLC conditions were: 30 μL injection volume, isocratic elution of mobile phase consisting of 70% methanol and 30% 10 mM sodium phosphate buffer (pH 7.8), flow rate of 1.000 mL/min, column temperature of 40° C., and detection at 263 nm. Column used was a SUPELCOSIL LC-18-T column (150 mm×4.6 mm; 3 μm particle size).

As shown in FIGS. 17A-17B, the addition of sesame EMO to EMLA cream improved permeation of both lidocaine and prilocaine in all cases. With 0.5% EMO addition, permeation of prilocaine and lidocaine were increased more than 3-fold compared to EMLA cream without EMO. Addition of >0.5% sesame EMO resulted in greater than 4-fold increases in prilocaine and lidocaine. These data support the use of adding EMOs as individual ingredients in topical formulations to improve skin permeation of drugs such as lidocaine and prilocaine.

### Example 23. In Vitro Permeation Study of Voltaren Diclofenac Cream with and without Sesame EMO

Strat-M® membrane (lot R1KB43611) was set in a Franz-type diffusion cell (1.77 cm² area). Receptor chamber was equipped with stir bar, filled with PBS (10 mL), and placed in 32° C. water bath. 100 mg of Voltaren with and without the addition of 10% sesame EMO were added to the donor chamber. At each time point, 70 μL was removed from the receiver chamber and replaced with an equivalent volume of fresh PBS to maintain constant volume. The aliquot from each time point was analyzed by HPLC to determine amount of drug permeating through the membrane over time. The HPLC system consisted of an LC-20AD pump, SIL-20AC HT autosampler, CTO-20A column oven, and SPD-20A UV/vis detector (Shimadzu Scientific Instruments, Inc.). HPLC conditions were: 30 μL injection volume, isocratic elution of mobile phase consisting of 70% methanol and 30% phosphoric acid (0.1%), flow rate of 1.000 mL/min, column temperature of 40° C., and detection at 280 nm. Column used was a SUPELCOSIL LC-18-T column (150 mm×4.6 mm; 3 μm particle size).

As shown in FIG. 18, the addition of sesame EMO (10% by weight in this particular example) to VOLTAREN diclofenac cream improved the diclofenac permeation 1.5-fold at 5 hours compared to the Voltaren cream without sesame EMO. The addition of EMO also accelerated permeation, as evidenced by the 6-fold improvement in permeation at the earliest time point (0.5 h). These data support the use of adding EMOs as individual ingredients in topical formulations to improve skin permeation of drugs such as VOLTAREN.

### Example 24. In Vitro Skin Permeation Study of Cannabidiol (CBD) Cream with and without EMO

Strat-M® membrane (lot R1KB43611) was set in a Franz-type diffusion cell (1.77 cm² area). Receptor chamber was equipped with stir bar, filled with 0.5% brij 98 solution (10 mL), and placed in 32° C. water bath. 100 mg of GARDEN OF LIFE CBD Intensive Recovery cream with and without the addition of 10% sesame EMO, 10% coconut EMO, or 10% sunflower EMO were added to the donor chamber. At each time point, 70 μL was removed from the receiver chamber and replaced with an equivalent volume of fresh 0.5% brij 98 solution to maintain constant volume. The aliquot from each time point was analyzed by HPLC to determine amount of drug permeating through the membrane over time. The HPLC system consisted of an LC-20AD pump, SIL-20AC HT autosampler, CTO-20A column oven, and SPD-20A UV/vis detector (Shimadzu Scientific Instruments, Inc.). HPLC conditions were: 30 μL injection volume, isocratic elution of mobile phase consisting of 75% methanol and 25% water, flow rate of 1.000 mL/min, column temperature of 40° C., and detection at 275 nm. Column used was a SUPELCOSIL LC-18-T column (150 mm×4.6 mm; 3 μm particle size).

As shown in FIG. 19, the CBD cream showed no permeation of CBD over 5 hours. However, the addition of 10% coconut or sunflower EMOs achieved 1.4 ug/cm\hat{ }2 CBD permeation, and the addition of 10% sesame EMO achieved 2.0 ug/cm\hat{ }2 CBD permeation over 5 hours. These data support the use of adding EMOs as individual ingredients in topical formulations to improve skin permeation of hydrophobic drugs such as CBD.

### Example 25. In Vitro Skin Permeation Study of Resveratrol Cream with and without Sesame EMO

Strat-M® membrane (lot R1KB43611) was set in a Franz-type diffusion cell (1.77 cm² area). Receptor chamber was equipped with stir bar, filled with 0.5% brij 98 solution (10 mL), and placed in 32° C. water bath. Approximately 100 mg of SkinCeuticals Resveratrol BE cream with and without the addition of 10% sesame EMO were added to the donor chamber. At each time point, 70 μL was removed from the receiver chamber and replaced with an equivalent volume of fresh 0.5% brij 98 solution to maintain constant volume. The aliquot from each time point was analyzed by HPLC to determine amount of drug permeating through the membrane over time. The HPLC system consisted of an LC-20AD pump, SIL-20AC HT autosampler, CTO-20A column oven, and SPD-20A UV/vis detector (Shimadzu Scientific Instruments, Inc.). HPLC conditions were: 30 μL injection volume, isocratic elution of mobile phase consisting of 45% methanol and 55% phosphoric acid (0.1%), flow rate of 1.000 mL/min, column temperature of 40° C., and detection at 303 nm. Column used was a SUPELCOSIL LC-18-T column (150 mm×4.6 mm; 3 μm particle size).

As shown in FIG. 20, the addition of 10% sesame EMO achieved 4-fold and 5-fold increases in resveratrol permeation at 8 hours and 24 hours, respectively, compared to the cream alone. These data support the use of adding EMOs as individual ingredients in topical formulations to improve skin permeation of hydrophobic drugs such as resveratrol.

### Example 26. In Vitro Skin Permeation Study of Retinyl Palmitate Creams with and without EMO

Strat-M® membrane (lot R1KB43611) was set in a Franz-type diffusion cell (1.77 cm² area). Receptor chamber was equipped with stir bar, filled with 0.5% brij 98 solution (10 mL), and placed in 32° C. water bath. Approximately 100 mg of creams (Revitalift anti-wrinkle eye cream, Revitalift anti-wrinkle moisturizer, Revitalift Cicacream) with and without the addition of 10% sesame EMO were added to the donor chamber. At each time point, 70 μL was removed from the receiver chamber and replaced with an equivalent volume of fresh 0.5% brij 98 solution to maintain constant volume. The aliquot from each time point was analyzed by HPLC to determine amount of drug permeating through the membrane over time. The HPLC system consisted of an LC-20AD pump, SIL-20AC HT autosampler, CTO-20A column oven, and SPD-20A UV/vis detector (Shimadzu Scientific Instruments, Inc.). HPLC conditions were: 30 μL injection volume, isocratic elution of mobile phase consisting of 98% methanol and 2% water, flow rate of 1.000 mL/min, column temperature of 40° C., and detection at 325 nm. Column used was a Ascentis® Express C8 column (150 mm×4.6 mm; 2.7 μm particle size).

As shown in FIGS. 21A-21C, no permeation of retinyl palmitate was observed from any of the three products tested. However, the addition of 10% sesame EMO enabled permeation of the super hydrophobic retinyl palmitate in all three products and achieved as high as nearly 9 ug/cm\hat{ }2 after 24 hours. These data support the use of adding EMOs as individual ingredients in topical formulations to improve skin permeation of super hydrophobic drugs such as retinyl palmitate.

### Example 27. In Vitro Skin Permeation Study of Retinol Creams with and without EMO

Strat-M® membrane (lot R1KB43611) was set in a Franz-type diffusion cell (1.77 cm² area). Receptor chamber was equipped with stir bar, filled with 0.5% brij 98 solution (10 mL), and placed in 32° C. water bath. Approximately 100 mg of creams (Neutrogena Ageless Intensives Anti-wrinkle and Neutrogena Healthy Skin Anti-wrinkle Night) with and without the addition of 10% sesame EMO were added to the donor chamber. At each time point, 70 μL was removed from the receiver chamber and replaced with an equivalent volume of fresh 0.5% brij 98 solution to maintain constant volume. The aliquot from each time point was analyzed by HPLC to determine amount of drug permeating through the membrane over time. The HPLC system consisted of an LC-20AD pump, SIL-20AC HT autosampler, CTO-20A column oven, and SPD-20A UV/vis detector (Shimadzu Scientific Instruments, Inc.). HPLC conditions were: 30 μL injection volume, isocratic elution of mobile phase consisting of 90% methanol and 10% water, flow rate of 1.000 mL/min, column temperature of 40° C., and detection at 325 nm. Column used was a SUPELCOSIL LC-18-T column (150 mm×4.6 mm; 3 μm particle size).

As shown in FIGS. 22A-22B, the addition of sesame EMO improved the retinol permeation 8-fold and 2-fold at 6 hours and 24 hours, respectively, compared to the Ageless Intensive product alone. For the Healthy Skin product, the addition of EMO improved retinol permeation 9-fold at 7 hours and 24 hours. This supports the use of adding EMOs as individual ingredients in topical formulations to improve skin permeation of hydrophobic vitamins such as retinol.

### Example 28. In Vitro Skin Permeation Study of Trifluoroacetyl Tripeptide-2 with and without Nanoemulsion of Sesame Oil EMO and FFA Oil

Strat-M® membrane (lot R1KB43611) was set in a Franz-type diffusion cell (1.77 cm² area). Receptor chamber was equipped with stir bar, filled with PBS (10 mL), and placed in 32° C. water bath. Sesame EMO nanoemulsion (nS50) was prepared from a combination of sesame EMO and FFA oil. Sesame EMO (0.852 g) and sesame FFA (0.648 g) were weighed into a 50 mL metal beaker and heated to 60° C. A coarse emulsion was prepared by adding 13.5 mL water (pre-heated to 60° C.) to the oil. The mixture was subjected to high-sheer homogenizer for 30 seconds, resulting in a milky coarse emulsion. The coarse emulsion was then transferred to QSonica Q700CA sonicator with ½ inch probe. The emulsion was processed for 2 cycles (5 minutes sonication/2 minutes cooling) at 90% power. Approximately 1 mL of trifluoroacetyl tripeptide-2 (Cayman Chemical Company) solution (10 mg/mL in 10% DMSO in water) or trifluoroacetyl tripeptide-2 nanoemulsion (10 mg/mL in 10% DMSO in nS50) were added to the donor chamber. At each time point, 70 μL was removed from the receiver chamber and replaced with an equivalent volume of fresh PBS to maintain constant volume. The aliquot from each time point was analyzed by HPLC to determine amount of drug permeating through the membrane over time. The HPLC system consisted of an LC-20AD pump, SIL-20AC HT autosampler, CTO-20A column oven, and SPD-20A UV/vis detector (Shimadzu Scientific Instruments, Inc.). HPLC conditions were: 30 μL injection volume, isocratic elution of mobile phase consisting of 55% methanol and 45% phosphoric acid (0.1%), flow rate of 1.000 mL/min, column temperature of 40° C., and detection at 210 nm. Column used was a SUPELCOSIL LC-18-T column (150 mm×4.6 mm; 3 μm particle size).

As shown in FIG. 23, no peptide permeation was observed from the free peptide control at 6 hours and only 1.1 ug/cm\hat{ }2 after 24 hours. However, the nanoemulsion formulation significantly accelerated permeation as evidenced by detectable peptide permeation at the earliest time point (1 hour). The nanoemulsion formulation also significantly enhanced overall skin permeation, achieving a remarkable 38-fold improvement in peptide permeation over 24 hours. These data support the use of using EMO nanoemulsions for improving peptide skin permeation.

### Example 29. In Vitro Skin Permeation Study of Hexapeptide-11 with and without Nanoemulsion of Sesame Oil EMO and FFA Oil (nS50)

Strat-M® membrane (lot R1KB43611) was set in a Franz-type diffusion cell (1.77 cm² area). Receptor chamber was equipped with stir bar, filled with PBS (10 mL), and placed in 32° C. water bath. Sesame EMO nanoemulsion (nS50) was prepared from a combination of sesame EMO and FFA oil. Sesame EMO (0.852 g) and sesame FFA (0.648 g) were weighed into a 50 mL metal beaker and heated to 60° C. A coarse emulsion was prepared by adding 13.5 mL water (pre-heated to 60° C.) to the oil. The mixture was subjected to high-sheer homogenizer for 30 seconds, resulting in a milky coarse emulsion. The coarse emulsion was then transferred to QSonica Q700CA sonicator with ½ inch probe. The emulsion was processed for 2 cycles (5 minutes sonication/2 minutes cooling) at 90% power. Approximately 1 mL of hexapeptide-11 (Active Peptide) solution (10 mg/mL in 10% DMSO in water) or hexapeptide-11 nanoemulsion (10 mg/mL in 10% DMSO in nS50) were added to the donor chamber. At each time point, 70 μL was removed from the receiver chamber and replaced with an equivalent volume of fresh PBS to maintain constant volume. The aliquot from each time point was analyzed by HPLC to determine amount of drug permeating through the membrane over time. The HPLC system consisted of an LC-20AD pump, SIL-20AC HT autosampler, CTO-20A column oven, and SPD-20A UV/vis detector (Shimadzu Scientific Instruments, Inc.). HPLC conditions were: 30 μL injection volume, isocratic elution of mobile phase consisting of 60% methanol and 40% phosphoric acid (0.1%), flow rate of 1.000 mL/min, column temperature of 40° C., and detection at 210 nm. Column used was a SUPELCOSIL LC-18-T column (150 mm×4.6 mm; 3 μm particle size).

As shown in FIG. 24, there was no peptide permeation observed from the free peptide control over 24 hours. However, the nanoemulsion formulation significantly enhanced overall skin permeation, achieving a remarkable 111 ug/cm\hat{ }2 over 24 hours. These data support the use of using EMO nanoemulsions for improving peptide skin permeation.

### Example 30. In Vitro Skin Permeation Study of 5-Fluorouracil (5-FU) Cream with and without Sesame EMO

Strat-M® membrane (lot R1KB43611) was set in a Franz-type diffusion cell (1.77 cm² area). Receptor chamber was equipped with stir bar, filled with PBS (10 mL), and placed in 32° C. water bath. Approximately 100 mg of prescription 5-FU cream (5% 5-FU; Mayne Pharma) with and without the addition of 10% sesame EMO were added to the donor chamber. At each time point, 70 μL was removed from the receiver chamber and replaced with an equivalent volume of fresh PBS solution to maintain constant volume. The aliquot from each time point was analyzed by HPLC to determine amount of drug permeating through the membrane over time. The HPLC system consisted of an LC-20AD pump, SIL-20AC HT autosampler, CTO-20A column oven, and SPD-20A UV/vis detector (Shimadzu Scientific Instruments, Inc.). HPLC conditions were: 30 μL injection volume, isocratic elution of mobile phase consisting of 4% methanol and 96% phosphoric acid (0.1%), flow rate of 1.000 mL/min, column temperature of 40° C., and detection at 265 nm. Column used was a SUPELCOSIL LC-18-T column (150 mm×4.6 mm; 3 μm particle size).

As shown in FIG. 25, the addition of 10% sesame EMO achieved 13-fold and 8-fold increases in 5-FU permeation at 3 hours and 24 hours, respectively, compared to the cream alone. These data support the use of adding EMOs as individual ingredients in topical formulations to improve skin permeation of chemotherapeutic drugs such as 5-FU.

### Example 31. Analysis of Non-oil Ingredients in Enzyme-modified Oils

Samples of canola oil or canola EMO were derivatized with 3-picolylamide for LC/MS/MS analysis. The analysis was performed with in positive ionization mode for total fatty acid and lipodomic and negative ionization for lipodomics. Thermo Scientific Freestyle & LipidSearch was used for analysis and the Orbitrap fusion method included data dependent acquisition MS (total fatty acid) & MS. As shown in FIG. 26, non-oil ingredients were generally preserved or enhanced. Coenzyme was also found to be enhanced.

### Example 32. In Vitro Skin Permeation Study of ASPERCREME (Lidocaine) with EMO, EMO FFA Oil, FFA Oil, or TAG Oil

Strat-M® membrane (lot R1KB43611) was set in a Franz-type diffusion cell (1.77 cm² area). Receptor chamber was equipped with stir bar, filled with PBS (10 mL), and placed in 32° C. water bath. Approximately 100 mg of Aspercreme with and without the following:
10% sesame EMO
10% of a mixture of 50% sesame EMO sesame FFA oil (50% total FFA) (S50)
10% sesame FFA oil
10% sesame oil (triglyceride; TAG)
   were added to the donor chamber. At each time point, 70 μL was removed from the receiver chamber and replaced with an equivalent volume of fresh PBS to maintain constant volume. The aliquot from each time point was analyzed by HPLC to determine amount of drug permeating through the membrane over time. The HPLC system consisted of an LC-20AD pump, SIL-20AC HT autosampler, CTO-20A column oven, and SPD-20A UV/vis detector (Shimadzu Scientific Instruments, Inc.). HPLC conditions were: 30 μL injection volume, isocratic elution of mobile phase consisting of 70% methanol and 30% 10 mM sodium phosphate buffer (pH 7.8), flow rate of 1.000 mL/min, column temperature of 40° C., and detection at 263 nm. Column used was a SUPELCOSIL LC-18-T column (150 mm×4.6 mm; 3 μm particle size).

FIG. 27 shows cumulative permeated lidocaine over 5 hours.

Only the addition of sesame EMO significantly enhanced the permeation of lidocaine compared to the Aspercreme product. The addition of sesame TAG oil had virtually no effect on lidocaine permeation. Surprisingly, the addition of a mixture of sesame EMO/FFA or sesame FFA significantly decreased the permeation of lidocaine.

### Example 33. In Vitro Skin Permeation Study of CERAVE Hydrocortisone Cream with EMO, EMO FFA Oil, FFA Oil, or TAG Oil

Strat-M® membrane (lot R1KB43611) was set in a Franz-type diffusion cell (1.77 cm² area). Receptor chamber was equipped with stir bar, filled with PBS (10 mL), and placed in 32° C. water bath. Approximately 100 mg of Cerave hydrocortisone cream with and without the following:
10% sesame EMO
10% of a mixture of 50% sesame EMO and sesame FFA oil (50% total FFA) (S50)
10% sesame FFA oil
10% sesame oil (triglyceride; TAG)
   were added to the donor chamber. At each time point, 70 μL was removed from the receiver chamber and replaced with an equivalent volume of fresh PBS to maintain constant volume. The aliquot from each time point was analyzed by HPLC to determine amount of drug permeating through the membrane over time. The HPLC system consisted of an LC-20AD pump, SIL-20AC HT autosampler, CTO-20A column oven, and SPD-20A UV/vis detector (Shimadzu Scientific Instruments, Inc.). HPLC conditions were: 30 μL injection volume, isocratic elution of mobile phase consisting of 55% methanol and 45% water, flow rate of 1.000 mL/min, column temperature of 40° C., and detection at 244 nm. Column used was a SUPELCOSIL LC-18-T column (150 mm×4.6 mm; 3 μm particle size).

FIG. 28 shows cumulative permeated hydrocortisone over 5 hours.

The addition of either sesame EMO or a mixture of sesame EMO and sesame FFA resulted in the greatest improvement in hydrocortisone permeation. Addition of FFA only also resulted in significant permeation enhancement. Addition of sesame TAG oil resulted in only minor improvements compared to the cream product.

The foregoing description of specific embodiments of the present disclosure has been presented for purpose of illustration and description. The exemplary embodiments were chosen and described in order to best explain the principles of the disclosure and its practical application, to thereby enable others skilled in the art to best utilize the subject matter and various embodiments with various modifications are suited to the particular use contemplated. Different features and disclosures of the various embodiments within the present disclosure may be combined within the scope of the present disclosure.

What is claimed is:

1. A composition comprising an oil dispersed in a liquid, wherein the oil is in the form of droplets or particles, wherein the oil comprises free fatty acids (FFA) in an amount of at least 5% by weight out of the total weight of the oil, wherein the free fatty acids comprise two or more different fatty acids, wherein the oil is substantially free of triacylglycerols (TAGs), and wherein the droplets or particles have a Z average size of about 300 nm or less as measured by dynamic light scattering (DLS).

2. The composition of claim 1, wherein the composition does not include any exogenous additive with surfactant and/or emulsifier properties that is not naturally present in the oil in an amount sufficient to enhance droplet stability.

3. The composition of claim 1, wherein the composition does not include any exogenous additive with thickening or crystallization inhibiting properties that is not naturally present in the oil in an amount sufficient to thicken the composition or inhibit crystallization formation, respectively.

4. The composition of claim 1, wherein the droplets or particles have an average zeta potential of less than −30 mV.

5. The composition of claim 1, wherein the droplets or particles have a polydispersity index (PDI) of less than 0.3.

6. The composition of claim 1, wherein the Z average size of the droplets or particles does not change by more than 15% over 28 days when stored at room temperature.

7. The composition of claim 1, further comprising an active ingredient.

8. An oil composition, comprising:
monoacylglycerols (MAGs) in an amount from about 10% to about 80% by weight of the total weight of the oil;
free fatty acids (FFAs) in an amount from about 20% to about 90% by weight of the total weight of the oil;
wherein the FFAs comprise two or more different fatty acids and wherein the oil is substantially free of triacylglycerols (TAGs).

9. The composition of claim 8, wherein the oil is derived from MCT oil, and wherein the two or more different fatty acids comprise C8:0 in an amount from about 50% to about 65% out of the total fatty acid content of the oil and C10 in an amount from about 35% to about 50% out of the total fatty acid content of the oil.

10. The composition of claim 8, wherein the oil is derived from canola oil, and wherein the two or more different fatty acid comprise C18:1 in an amount from about 55% to about 70% out of the total fatty acid content and C18:2 in an amount from about 15% to about 30% out of the total fatty acid content.

11. The composition of claim 8, further comprising an active ingredient.

12. The composition of claim 11, wherein the active ingredient is selected from the group consisting of a steroid drug, a nonsteroidal drug, an analgesic drug, a plant extract, an antioxidant, and a vitamin or nutritional supplement.

13. The composition of claim 11, wherein the active ingredient is selected from the group consisting of bupivacaine, lidocaine, prilocaine, and combinations thereof.

14. A composition comprising a first component, and a second component, wherein the first component is selected from the group consisting of a cream, a lotion, a body wash, or a lubricant; and wherein the second component is an additive comprising the composition of claim 9.

15. The composition of claim 14 further comprising a third component; wherein the third component is an active ingredient selected from the group consisting of a steroid drug, a nonsteroidal drug, an analgesic drug, a plant extract, an antioxidant, and a vitamin or nutritional supplement.

16. The composition of claim 15, wherein the first component is a cream and the third component is the steroid, wherein the steroid is hydrocortisone, and wherein the hydrocortisone has a skin permeability of at least 6 μg/cm² per 7 hour period.

17. The composition of claim 15, wherein the first component is a lubricant and the third components is the analgesic drug, and the analgesic drug is selected from the group consisting of bupivacaine, lidocaine, prilocaine, and combinations thereof.

18. The composition of claim 15, wherein the analgesic drug has a skin permeability of at least 6 μg/cm² per 7 hour period.

19. The composition of claim 14, wherein the first component is the body wash.
